(19) United States
(12) Patent Application Publication
(10) Pub. No.: US 2020/0294642 A1
(43) Pub. Date: Sep. 17, 2020

(54) METHODS AND SYSTEMS FOR A PHARMACOLOGICAL TRACKING AND REPORTING PLATFORM

(71) Applicant: hc1.com Inc., Indianapolis, IN (US)

(72) Inventors: Bradley A. Bostic, Indianapolis, IN (US); Laura S. Breedlove, Indianapolis, IN (US); Charles J. Clarke, Indianapolis, IN (US); Jason P. Wolfgang, Indianapolis, IN (US); Mark L. Preston, Indianapolis, IN (US); Richard J. Oder, Indianapolis, IN (US); William E. Robinson, Speedway, IN (US); Ryan C. Kennedy, Carmel, IN (US); Ioannis Pandelidis, Indianapolis, IN (US)

(73) Assignee: hc1.com Inc., Indianapolis, IN (US)

(21) Appl. No.: 16/778,377

(22) Filed: Jan. 31, 2020

Related U.S. Application Data
(63) Continuation-in-part of application No. 16/535,863, filed on Aug. 8, 2019.
(60) Provisional application No. 62/716,090, filed on Aug. 8, 2018.
(60) Provisional application No. 62/800,086, filed on Feb. 1, 2019.

(51) Int. Cl. G16H 20/10; G16H 50/70; G16H 50/20; G16H 10/40; G16H 10/60; G06Q 40/08; G06K 9/62; G06N 20/00

(52) U.S. Cl. CPC G16H 20/10; G16H 50/70; G16H 50/20; G16H 10/40; G16H 10/60; G06N 20/00; G06K 9/6223; G06K 9/623; G06K 9/6268; G06K 9/6289; G06Q 40/08

(57) ABSTRACT

The present disclosure generally includes methods and systems for recommending a lab test for a patient is disclosed. The method includes obtaining a proposed prescription for the patient from an external data source. The method further includes obtaining patient attributes for the patient, including a diagnosis of the patient. The method also includes determining whether to recommend one or more different lab tests for the patient prior to the patient beginning the proposed prescription based on the proposed prescription and the patient attributes, and providing a testing recommendation to a customer relationship management system, wherein the testing recommendation indicates the one or more different tests that are recommended for the patient in response to determining to recommend one or more different lab tests for the patient, wherein the customer relationship management system transmits the testing recommendation to a healthcare system of a clinic of the patient.

### CROSS-REFERENCE TO RELATED APPLICATIONS

This application is a continuation-in-part of U.S. application Ser. No. 16/535,863 filed Aug. 8, 2019, entitled Methods and Systems for a Pharmacological Tracking and Reporting Platform, which claims priority to U.S. provisional application No. 62/716,090, filed on Aug. 8, 2018. This application claims priority to U.S. provisional application No. 62/800,086, filed on Feb. 1, 2019. Each of the above applications is hereby incorporated by reference as if fully set forth herein in its entirety.

### FIELD

The present disclosure relates to a pharmacological tracking platform.

### BACKGROUND

It is estimated that approximately 80% of Americans are prescribed at least one pharmaceutical drug. Many people who are prescribed pharmaceutical drugs, however, may be prescribed the wrong drug, which can lead to adverse reactions, ineffective treatment, or even death. In some scenarios, a patient may be taking two medications that are not compatible with one another. In other scenarios, the patient may be physiologically unable to metabolize or otherwise process one of the active ingredients in the medication. These conditions may be averted if the patient is prescribed appropriate tests prior to being prescribed a treatment.

Moreover, many patients that are prescribed medications are misusing their drugs. In some scenarios, patients may be abusing the medication they are prescribed (e.g., opiates, amphetamines, and/or benzodiazepines). In other scenarios, patients may be using the drug with an incompatible over the counter medication or may be using the medication improperly (e.g., taking the medication too infrequently or without following the instructions). In other cases, prescribed medications may be diverted for use by individual's other than the one for whom the medication is prescribed, such as for sale on the black market or for unprescribed use by friends or family members. In any of these scenarios, a patient's health may be adversely affected and/or the costs of treating the patient may increase due to the improper use of the medication.

A need exists for improved methods and systems for detecting and addressing situations involving improper prescription of medication, improper utilization of prescribed medications, and diversion of prescribed medications to unprescribed uses.

### SUMMARY

Improved methods, systems, components, processes, modules, and other elements (collectively referred to alternatively herein as the “pharmacological tracking platform,” or simply as the “platform”) for detecting and addressing situations involving improper prescription of medication, improper utilization of prescribed medications, and diversion of prescribed medications to unprescribed uses.

According to some embodiments of the present disclosure, a computer implemented method for detecting misuse of a controlled medication of a patient is disclosed. The computer implemented method includes obtaining, by a processing system, lab test results of the patient from a lab testing system. The computer implemented method also includes obtaining, by the processing system, patient attributes of the patient from one or more patient data sources. The computer implemented method further includes generating, by the processing system, a usage profile corresponding to the patient based on the lab test results of the patient and the patient attributes. The computer implemented method also includes determining, by the processing system, whether the usage profile is indicative of potential misuse of the controlled medication based on one or more features of the usage profile. Furthermore, in response to determining potential misuse of the controlled medication, the computer implemented method includes transmitting a notification that indicates the potential misuse by the patient.

In some embodiments, the potential misuse of the controlled medication is overuse of the controlled medication.

In some embodiments, the potential misuse of the controlled medication is underuse of the controlled medication.

In some embodiments, generating the usage profile includes combining multiple test results of the patient to obtain a history of lab results of the patient.

In some embodiments, the patient attributes include two or more of: an age of the patient, a weight of the patient, a body type of the patient, and an activity level of a patient. In some of these embodiments, the patient attributes are obtained from an electronic medical record database of a healthcare system associated with a clinic of the patient. In some embodiments, the patient attributes are obtained from an insurer database of an insurance system associated with an insurance provider of the patient.

In some embodiments, determining whether the usage profile is indicative of potential misuse includes: identifying a set of features based on the usage profile; inputting the set of features into a machine learned classification model that is trained to classify instances of potential misuse of the classified medication; obtaining a classification from the machine learned classification model and a confidence score indicating a degree of confidence in the classification determined by the machine learned classification model; and determining whether the usage profile is indicative of the potential misuse based on the classification and the confidence score.

In some embodiments, determining whether the usage profile is indicative of potential misuse includes: identifying a set of features based on the usage profile; clustering the usage profile with a plurality of other usage profiles using a clustering algorithm, each other usage profile respectively corresponding to a respective previous patient that was prescribed the controlled medication and deemed either to be indicative of potential misuse of the controlled medication or proper use of the controlled medication; determining a cluster of the usage profile of the patient to which the usage profile was clustered, wherein the cluster includes a subset of the plurality of other usage profiles; and determining whether the usage profile is indicative of potential misuse of the controlled medication based on the other usage profiles in the subset of the plurality of other usage profiles.

In some embodiments, determining whether the usage profile is indicative of potential misuse includes: identifying a set of features based on the usage profile; and applying a set of rules to the features to determine whether the usage profile is indicative of potential misuse.

In some embodiments, the lab test results include results from a urine analysis test. In some embodiments, the lab test results include results from a blood test. In some embodiments, the lab test results include results from a buccal swab.

According to some embodiments of the present disclosure, a computer implemented method for recommending a lab test for a patient is disclosed. The computer implemented method includes obtaining, by a processing system, a proposed prescription for the patient from an external data source, the proposed prescription indicating a medication. The computer implemented method further includes obtaining, by the processing system, patient attributes for the patient, including a diagnosis of the patient. The computer implemented method also includes determining, by the processing system, whether to recommend one or more different lab tests for the patient prior to the patient beginning the proposed prescription based on the proposed prescription and the patient attributes. The computer implemented method also includes providing, by the processing system, a testing recommendation to a customer relationship management system, wherein the testing recommendation indicates the one or more different tests that are recommended for the patient in response to determining to recommend one or more different lab tests for the patient, wherein the customer relationship management system transmits the testing recommendation to a healthcare system of a clinic of the patient.

In some embodiments, determining whether to recommend the one or more different lab tests includes: identifying a set of features based on the proposed prescription and the patient attributes; inputting the set of features into one or more machine learned models that are respectively trained to determine whether to recommend a respective lab test; obtaining one or more respective recommendations from the one or more respective machine learned models based on the set of features, wherein each respective recommendation indicates whether the respective lab test should be performed for the patient given the patient attributes and has a confidence score that indicates a degree of confidence in the recommendation; and for each recommendation, determining whether to recommend the respective lab test indicated therein based on the confidence score of the recommendation. In some of these embodiments, each of the one or more machine learned models corresponds to the medication. In some embodiments, each of the one or more machine learned models is trained on a plurality of training data samples that respectively correspond to a plurality of previous patients that were prescribed the medication, wherein each training data sample includes respective patient attributes of the respective previous patient and an outcome related to the medication for the previous patient. In some of these embodiments, each training data sample further includes one or more lab test results of the respective previous patient.

In some embodiments, determining whether to recommend the one or more different lab tests includes: identifying a set of features based on the proposed prescription and the patient attributes; inputting the set of features into a machine learned model that is trained to determine whether to recommend one or more of a plurality of different lab tests given a set of patient attributes; obtaining a recommendation and a confidence score corresponding to the recommendation from the machine learned model based on the set of features, wherein the recommendation indicates any of the plurality of different lab tests that should be performed for the patient given the patient attributes, and the confidence score indicates a degree of confidence in the recommendation; and determining whether to accept the recommendation based on the confidence score of the recommendation. In some of these embodiments, the machine learned model corresponds to the medication. In some embodiments, the machine learned model is trained on a plurality of training data samples that respectively correspond to a plurality of previous patients that were prescribed the medication, wherein each training data sample includes respective patient attributes of the respective previous patient and an outcome related to the medication for the previous patient. In some embodiments, each training data sample further includes one or more lab test results of the respective previous patient.

In some embodiments, determining whether to recommend the one or more different lab tests includes: identifying a set of features based on the usage profile, and applying a set of rules to the features to determine whether to recommend the one or more different lab tests based on the set of features and one or more conditions defined in the set of rules.

In some embodiments, the different lab tests include at least one of a genetic test, a blood test, and a urine analysis test.

In embodiments, the various systems and methods of the present disclosure include a medical records system configured for analyzing workflow for clinical professionals. The system can include a healthcare database configured to store a plurality of medical records. The medical records include: demographic records including one or more of height, weight, sex, gender, ethnicity, and age of a plurality of patients; diagnosis records including medical diagnoses of the patients; prescription records including drugs previously prescribed to the patient; and testing records including tests ordered for the patients, tests performed on the patients, and results of tests performed on the patients, the testing history including names and codes used to identify the tests by health care centers and labs ordering and/or administering the tests. The system can include a machine learning device in communication with the healthcare database and configured to receive the demographic records, the diagnosis records, the prescription records, and the testing records from the healthcare database. The machine learning device is configured to train an artificial intelligence based on the demographic records, the diagnosis records, the prescription records, and the testing records. The artificial intelligence is trained to identify inconsistencies in the names and/or codes used by the health care centers and labs and normalize the names and codes used to identify the tests by health care centers such that similar tests are identified by consistent names and/or codes within the healthcare database. The artificial intelligence is trained to analyze the tests being ordered by individual health care centers of the health care centers to determine redundancies in testing by the individual health care centers. The artificial intelligence is also trained to analyze the demographic records, diagnosis records, prescription records, and testing records to identify inconsistencies in diagnosing, drug prescribing, and test ordering practices of the health care facilities, identify potential improvements to the diagnosing, drug prescribing, and test ordering practices of the health care facilities, and identify medically unnecessary and/or redundant diagnosing, drug prescribing, and test ordering practices of the health care facilities.

In embodiments, the various systems and methods of the present disclosure include a medical records system configured for integrating traditional medical records with lifestyle, wellness, and physiological monitoring information and disseminating the same. The system includes a healthcare database configured to store a plurality of medical records. The medical records include demographic records including one or more of height, weight, sex, gender, ethnicity, and age of a plurality of patients; diagnosis records including medical diagnoses of the patients; prescription records including drugs previously prescribed to the patient; testing records including tests ordered for the patients, tests performed on the patients, and results of tests performed on the patients, the testing history including names and codes used to identify the tests by health care centers and labs ordering and/or administering the tests. The healthcare database is further configured to store lifestyle and wellness records of the patients, the lifestyle and wellness information including information related to one or more of diet, smoking, drinking, and exercise habits. The healthcare database is configured to transmit the medical records and the lifestyle and wellness records to health care facilities and to third parties.

In embodiments, the various systems and methods of the present disclosure include a medical records system configured for identifying abuse and/or misuse practices of a medical patient and a healthcare database in communication with a state-based prescription drug monitoring program database and configured to store a plurality of medical records. The medical records include demographic records including one or more of height, weight, sex, gender, ethnicity, and age of the patient; diagnosis records including medical diagnoses of the patient; and prescription records including drugs previously prescribed to the patient, drugs being taken by the patient as reported by the patient, and drug prescription records of the patient received from the prescription drug monitoring program database. The medical records system is configured to identify abuse and/or misuse of prescription drugs by the patient based on the drugs previously prescribed, the drugs being taken, and the drug prescription records received from the prescription drug monitoring program database.

According to various implementations of the present disclosure, a computer implemented method for prescription drug management program reporting is disclosed. The method can include receiving, at a computing device having one or more processors, laboratory test results from a laboratory. The laboratory test results can correspond to a patient and be indicative of a toxicology screen of the patient. The method can also include retrieving, by the computing device and from a prescription drug management program data source, controlled substance prescription data for the patient. For example only, the controlled substance prescription data can include prescriptions of controlled substances issued to the patient for a relevant time period. The method can further include analyzing, by the computing device, the controlled substance prescription data and the laboratory test results to determine a daily morphine milligram equivalent of the patient for a given time period, an overdose risk score, and a drug consistency assessment. The daily morphine milligram equivalent of the patient for the given time period can correspond to a cumulative intake of opioid class drugs by the patient on a daily basis for the given time period. The overdose risk score can be indicative of a likelihood of an unintentional overdose by the patient. The drug consistency assessment can be representative of a match between the controlled substance prescription data and the laboratory test results for the patient. An enhanced toxicology report can be generated, by the computing device, and can correspond to the patient based on the determined daily morphine milligram equivalent of the patient for the given time period, the overdose risk score, and the drug consistency assessment. The enhanced toxicology report can be output, by the computing device, to a requestor computing device.

In some aspects, the method can additionally or alternatively include obtaining, by the computing device, patient attributes of the patient from one or more patient data sources. The patient attributes can correspond to one or more of an age of the patient, a weight of the patient, a body type of the patient, an activity level of the patient, and a diagnosis of the patient. The enhanced toxicology report can be further based on the patient attributes.

In some aspects, the enhanced toxicology report corresponding to the patient can include a historical trend of the determined daily morphine milligram equivalent for the patient. The historical trend can be presented in a graphical format or other formats.

Additionally or alternatively, the enhanced toxicology report can include one or more drug consistency scores based on the drug consistency assessment. Each particular drug consistency score can be indicative of a match between a particular drug identified in either or both of the controlled substance prescription data and the laboratory test results for the patient. Each particular drug consistency score can indicate: (i) a prescribed and detected condition in which the particular drug is identified in both of the controlled substance prescription data and the laboratory test results for the patient; (ii) a detected but not prescribed condition in which the particular drug is identified in the laboratory test results for the patient but not the controlled substance prescription data; and/or (iii) an inconsistent condition in which (a) the particular drug is a drug metabolite of a parent drug and is identified in the laboratory test results for the patient and the controlled substance prescription data indicates a prescription for the parent drug, or (b) the particular drug is identified in the controlled substance prescription data and the laboratory test results for the patient indicate that the particular drug is not present at a prescribed amount in the patient.

In certain aspects, the enhanced toxicology report corresponding to the patient can include a graphical element indicative of prescriptions of controlled substances issued to the patient for the relevant time period. The graphical element can include a list of prescribers that issued the prescriptions. Further, the graphical element can illustrate an overlap of prescriptions of controlled substances issued to the patient for the relevant time period from multiple prescribers.

Additionally or alternatively, in certain implementations the enhanced toxicology report corresponding to the patient can include a historical trend of the overdose risk score for the patient.

According to various implementations of the present disclosure, a computing system for prescription drug management program reporting is disclosed. The computing system can include one or more processors and a non-transitory computer-readable storage medium having a plurality of instructions stored thereon, which, when executed by the one or more processors, cause the one or more processors to perform operations. The operations can include receiving laboratory test results from a laboratory. The laboratory test results can correspond to a patient and be indicative of a toxicology screen of the patient. The operations can also include retrieving, from a prescription drug management program data source, controlled substance prescription data for the patient. For example only, the controlled substance prescription data can include prescriptions of controlled substances issued to the patient for a relevant time period. The operations can further include analyzing the controlled substance prescription data and the laboratory test results to determine a daily morphine milligram equivalent of the patient for a given time period, an overdose risk score, and a drug consistency assessment. The daily morphine milligram equivalent of the patient for the given time period can correspond to a cumulative intake of opioid class drugs by the patient on a daily basis for the given time period. The overdose risk score can be indicative of a likelihood of an unintentional overdose by the patient. The drug consistency assessment can be representative of a match between the controlled substance prescription data and the laboratory test results for the patient. An enhanced toxicology report can be generated, by the computing system, and can correspond to the patient based on the determined daily morphine milligram equivalent of the patient for the given time period, the overdose risk score, and the drug consistency assessment. The enhanced toxicology report can be output, by the computing system, to a requestor computing device.

In some aspects, the operations can additionally or alternatively include obtaining patient attributes of the patient from one or more patient data sources. The patient attributes can correspond to one or more of an age of the patient, a weight of the patient, a body type of the patient, an activity level of the patient, and a diagnosis of the patient. The enhanced toxicology report can be further based on the patient attributes.

In some aspects, the enhanced toxicology report corresponding to the patient can include a historical trend of the determined daily morphine milligram equivalent for the patient. The historical trend can be presented in a graphical format or other formats.

Additionally or alternatively, the enhanced toxicology report can include one or more drug consistency scores based on the drug consistency assessment. Each particular drug consistency score can be indicative of a match between a particular drug identified in either or both of the controlled substance prescription data and the laboratory test results for the patient. Each particular drug consistency score can indicate: (i) a prescribed and detected condition in which the particular drug is identified in both of the controlled substance prescription data and the laboratory test results for the patient; (ii) a detected but not prescribed condition in which the particular drug is identified in the laboratory test results for the patient but not the controlled substance prescription data; and/or (iii) an inconsistent condition in which (a) the particular drug is a drug metabolite of a parent drug and is identified in the laboratory test results for the patient and the controlled substance prescription data indicates a prescription for the parent drug, or (b) the particular drug is identified in the controlled substance prescription data and the laboratory test results for the patient indicate that the particular drug is not present at a prescribed amount in the patient.

In certain aspects, the enhanced toxicology report corresponding to the patient can include a graphical element indicative of prescriptions of controlled substances issued to the patient for the relevant time period. The graphical element can include a list of prescribers that issued the prescriptions. Further, the graphical element can illustrate an overlap of prescriptions of controlled substances issued to the patient for the relevant time period from multiple prescribers. Additionally or alternatively, in certain implementations the enhanced toxicology report corresponding to the patient can include a historical trend of the overdose risk score for the patient.

A more complete understanding of the disclosure will be appreciated from the description and accompanying drawings and the claims, which follow.

### BRIEF DESCRIPTION OF THE DRAWINGS

The accompanying drawings, which are included to provide a better understanding of the disclosure, illustrate embodiments of the disclosure and, together with the description, serve to explain the principle of the disclosure. In the drawings:

FIG. 1 is a schematic illustrating an example environment of a pharmacological tracking platform according to some embodiments of the present disclosure;

FIG. 2 is a schematic illustrating an example set of components of a customer relationship management (CRM) system of a pharmacological tracking platform according to some embodiments of the present disclosure;

FIG. 3 is a schematic illustrating an example set of components of a test management system of a pharmacological tracking platform according to some embodiments of the present disclosure;

FIG. 4 is a schematic illustrating an example set of components of a prescription monitoring system of a pharmacological tracking platform according to some embodiments of the present disclosure;

FIG. 5 is a schematic illustrating an example reporting system environment of a pharmacological tracking platform according to some embodiments of the present disclosure;

FIG. 6 is an illustration of an example of an enhanced toxicology report according to some embodiments of the present disclosure;

FIG. 7 is an illustration of another example of an enhanced toxicology report according to some embodiments of the present disclosure;

FIG. 8 is an illustration of yet another example of an enhanced toxicology report according to some embodiments of the present disclosure;

FIG. 9 is an illustration of a further example of an enhanced toxicology report according to some embodiments of the present disclosure;

FIG. 10 is an illustration of an additional example of an enhanced toxicology report according to some embodiments of the present disclosure;

FIG. 11 is a diagram of an example computing system including an example computing device and an example server computing device according to some implementations of the present disclosure; and

FIG. 12 is a functional block diagram of the example computing device of FIG. 11.

### DETAILED DESCRIPTION

As mentioned above, there is a need for improved methods and systems for detecting and addressing situations involving improper prescription of medication, improper utilization of prescribed medications, and diversion of prescribed medications to unprescribed uses. As mentioned above, there is a need for improved methods and systems for detecting and addressing situations involving improper prescription of medication, improper utilization of prescribed medications, and diversion of prescribed medications to unprescribed uses. In the United States, for example, prescription drug monitoring programs (PDMPs) are utilized to track prescriptions of controlled drugs.

In order to address the above noted need for improved methods and systems for detecting and addressing situations involving improper prescription of medication, improper utilization of prescribed medications, and diversion of prescribed medications to unprescribed uses, the present disclosure is directed to an improved pharmacological tracking platform. The pharmacological tracking platform can be utilized to perform various functions, including but not limited to a report generation and outputting function, a misuse of a controlled medication function, and a laboratory test recommendation function. Each of these functions can be performed separately or in various combinations to address the above noted and other needs associated with the goal of preventing adverse drug-related events.

With respect to the report generation and outputting function, the present disclosure provides for generating an enhanced toxicology report corresponding to a patient. The enhanced toxicology report is designed as a simple and easy to understand summary of the use and potential misuse of controlled substances for a patient. As more fully described herein, the enhanced toxicology report can include various graphical elements to present information related to the use and potential misuse of controlled substances for a patient. These graphical elements can include, but are not limited to, graphs of historical trends or changes over time, a graph of prescriptions issued to the patient by prescriber by time periods, numerical scores, and color indicators. The enhanced toxicology report can be requested by prescribers, pharmacists, and other healthcare professionals to assist in treating a patient, as more fully described below.

In order to generate the enhanced toxicology report, laboratory test results from a laboratory and controlled substance prescription data for the patient are analyzed. The laboratory test results are indicative of a toxicology screen of the patient and the controlled substance prescription data includes prescriptions of controlled substances issued to the patient for a relevant time period. From this information, a daily morphine milligram equivalent of the patient for a given time period, an overdose risk score, and a drug consistency assessment are determined. The daily morphine milligram equivalent of the patient for the given time period corresponds to a cumulative intake of opioid class drugs by the patient on a daily basis for the given time period. The overdose risk score is indicative of a likelihood of an unintentional overdose by the patient, and the drug consistency assessment is representative of a match between the controlled substance prescription data and the laboratory test results for the patient. It should be appreciated that other scores, assessments, measurements, calculations, etc. can be determined.

With respect to the misuse of controlled medication function, the present disclosure provides for techniques for determining whether a usage profile of a patient is indicative of potential misuse of one or more controlled medications. A machine learning model or other forms of artificial intelligence are utilized to generate a potential misuse score or similar measurement of the likelihood that a patient is or has the potential for misusing a controlled substance. In some aspects, laboratory test results from a laboratory that are indicative of a toxicology screen of the patient are utilized, in conjunction with patient attributes of the patient, to generate the usage profile of the patient. Various features of the usage profile can be utilized with the artificial intelligence system to determine the likelihood that the patient is or has the potential for misusing a controlled substance. In response to determining that the patient is or has the potential for misusing a controlled substance, or when a healthcare professional is otherwise treating the patient, a notification or report of the patient's potential for misusing a controlled substance can be provided in order to assist with the treatment of the patient.

With respect to the laboratory test recommendation function, the present disclosure provides for techniques for determining whether to recommend one or more laboratory tests for a patient, e.g., at a time of prescribing a controlled substance. A machine learning model or other forms of artificial intelligence are utilized to generate a laboratory test recommendation or similar measurement of the likelihood that the patient would benefit from one or more specific laboratory tests before the patient is given a proposed prescription. In some aspects, a proposed prescription for the patient is utilized, in conjunction with patient attributes of the patient (e.g., a diagnosis), to determine whether to recommend one or more specific laboratory tests. Various features of the proposed prescription and patient attributes can be utilized with the artificial intelligence system to determine the likelihood that the patient would benefit from a specific laboratory test before beginning the prescription of the controlled substance. In response to determining that the patient would benefit from one or more specific laboratory tests, or when a healthcare professional is otherwise treating the patient, a notification, report, or laboratory test recommendation for the patient can be provided in order to assist with the treatment of the patient.

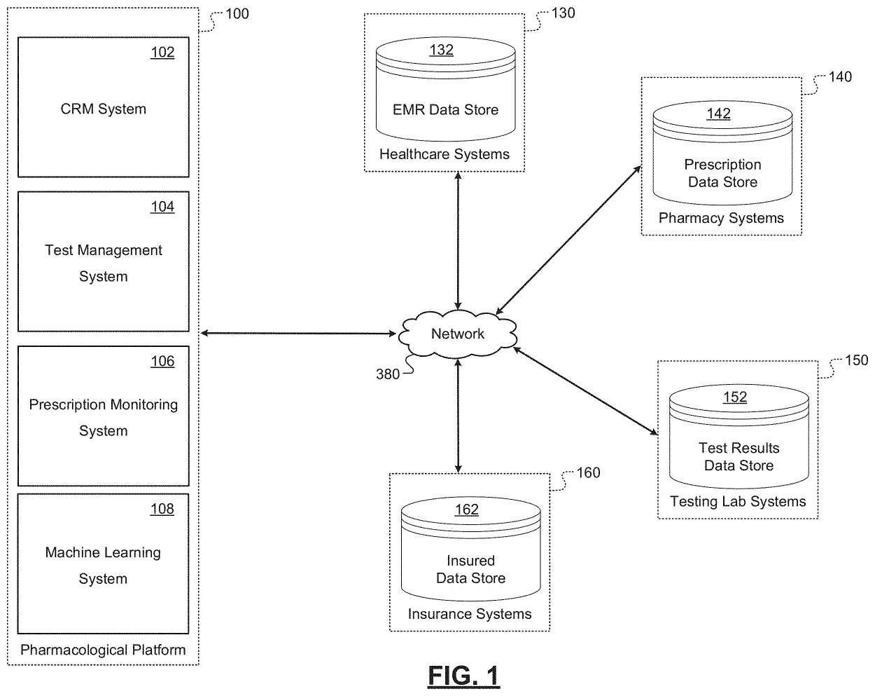

FIG. 1 illustrates an example pharmacological tracking platform 100 according to some embodiments of the present disclosure. In embodiments, the pharmacological tracking platform 100 is configured to collect and monitor data relating to laboratory tests (“lab tests” or “tests”) collected in connection with a treatment of a patient and/or data relating to prescription medications that are prescribed to patients. The pharmacological tracking platform 100 may obtain data from multiple external sources, including electronic medical records (EMRs), insurer databases, pharmacy databases, testing lab databases, prescription drug monitoring programs, and/or other suitable data sources. The pharmacological tracking platform 100 may use the obtained data to: make recommendations relating to the types of lab tests patients should undertake before beginning a potential prescription; determine whether a patient may be misusing a controlled medication (e.g., an opiate, a benzodiazepine, or amphetamine); determine whether a physician is overprescribing a controlled medication; determine whether a physician or clinic is over-ordering or under-ordering lab tests for their patients; and/or assess the quality of a testing lab. The pharmacological tracking platform 100 may perform additional or alternative tasks without departing from the scope of the disclosure.

As shown in FIG. 1, the pharmacological tracking platform 100 may communicate with external sources such as electronic medical records (EMRs), insurer databases, pharmacy databases, testing lab databases, and/or prescription drug monitoring programs, as well as other computing device(s), systems, data sources, applications, and platforms, via a network 380. It should be appreciated that the network 380 can take the form of any communication network suitable for communicatively linking computing devices and/or components thereof, including, without limitation, a virtual private network, the Internet, a Local Area Network, a Wide Area Network, a cellular network, and an intranet or other private networks.

In embodiments, the pharmacological tracking platform 100 may use the collected data to determine whether a patient should have one or more lab tests ordered prior to beginning a proposed prescription. In some of these embodiments, the platform 100 may obtain data relating to the patient, including the proposed prescription, as well as outcome data from previous patients that have taken the prescription in the past that includes lab tests associated with those patients, attributes of those patients (e.g., age, sex, weight, body type), and the result of the treatment (e.g., was the prescription effective). In this way, the patient may be prescribed a medication that is more likely to be effective prior to beginning the treatment. Furthermore, in embodiments, the pharmacological tracking platform 100 may recommend one or more different tests for the patient during or after the treatment, to ensure that the patient is receiving effective treatment.

In embodiments, the pharmacological tracking platform 100 may monitor test results of respective patients to determine whether the respective patients are misusing a controlled medication (e.g., an opiate, a benzodiazepine, or an amphetamine). Misusing a controlled medication may include overusing/abusing the medication, underusing the medication (which may be indicative of someone illegally distributing the medication), using the medication with other controlled medications, or improperly using the medication (e.g., not taking the medication at the correct times). In some of these embodiments, the pharmacological tracking platform 100 may analyze a patient's lab test results (e.g., toxicology screens such as blood tests or urine analysis tests) to determine if the patient is potentially misusing a controlled medication. In embodiments, the pharmacological tracking platform 100 may consider a patient's lab tests in their totality (e.g., over the period when the patient is prescribed the medication) and/or in view of lab tests of other patients (e.g., lab tests of patients that properly use the medication and lab tests of patients that misuse the medication) and attributes of those patients.

In embodiments, the pharmacological tracking platform 100 may provide notifications and/or recommendations to appropriate third parties, such as healthcare organizations (e.g., hospitals and/or clinics), physicians, pharmacies, insurers, and the like. In some of these embodiments, the platform 100 may provide customer relationship management capabilities, whereby the platform 100 may leverage these capabilities to provide the notifications and/or recommendations.

In embodiments, the pharmacological tracking platform 100 may include a customer relationship management (CRM) system 102, a test management system 104, a prescription monitoring system 106, and/or a machine learning system 108. The pharmacological tracking platform 100 may include additional or alternative systems without departing from the scope of the disclosure.

In embodiments, the test management system 104 may determine whether to recommend lab testing for a patient given a proposed treatment of the patient. In response to the test management system 104 determining to recommend lab testing for a patient, the CRM system 102 may provide a mechanism (e.g., a GUI) by which a user (e.g., a representative of a lab testing organization) may provide the notification recommending lab testing to a healthcare provider (e.g., the treating physician or the office thereof), a pharmacy, and/or an insurance provider. In embodiments, the test management system 104 may also perform various analytics on captured data to determine when physicians are overusing or underusing lab tests in their respective practices. In these embodiments, the test management system 104 may monitor the ordering of tests from a group of physicians to determine instances where a physician's ordering of tests is anomalous. The test management system 104 may provide other features as well, such as quality assessment relating to testing labs.

In embodiments, the prescription monitoring system 106 monitors test results of patients prescribed certain prescription medications to determine whether the respective patients are misusing the prescription medication (e.g., overusing/abusing, or underusing the prescription medication). In response to the prescription monitoring system 106 determining a likely case of misuse, the CRM system 102 may provide a mechanism by which a user (e.g., a representative of a lab testing organization) may provide the notification of potential misuse to a healthcare provider (e.g., the treating physician or the office thereof), a pharmacy, and/or an insurance provider.

In embodiments, the CRM system 102 may be accessed by users associated with a testing lab system 150. In embodiments, the CRM system 102 may allow these users to manage relationships and communications with healthcare providers associated with healthcare systems 130, pharmacy employees associated with pharmacy systems 140, and/or insurance providers associated with insurance systems 160. In embodiments, the CRM system 102 may receive recommendations and/or notifications from the test management system 104 and/or the prescription monitoring system 106. The CRM system 102 may perform additional or alternative tasks, such as obtaining data from external data sources (e.g., healthcare systems 130, pharmacy systems 140, testing lab systems 150, and/or insurance system 160) and may structure the obtained data into different types of records according to respective schemas.

In embodiments, the pharmacological tracking platform 100 may include a machine learning system 108 that is configured to train machine learned models that are leveraged by the test management system 104 and/or the prescription monitoring system 106. The machine learning system 108 may train any suitable type of model, including neural networks, deep neural networks, recurrent neural networks, Hidden Markov Models, Bayesian models, regression models, and the like. The machine learning system 108 may train the models in a supervised, unsupervised, or semi-supervised manner. In embodiments, the machine learning system 108 may collect training data from one or more data sources. Depending on the purpose of the model, the data types included in the training data will vary. For example, models used to recommend testing for a patient prior to the patient undergoing a particular treatment (e.g., prescription) may be trained on training data that includes prescription data of respective patients, outcome data relating to the respective patients' treatments, and lab test results of the respective patients that correspond to the outcome data. Models used to classify a patient's misuse of a medication may be trained on training data that includes lab test results of patients that were deemed to be misusing a particular medication and patient information relating to those patients, and lab test results of patients that were deemed to be using the medication properly and patient information relating to those patients.

A healthcare system 130 may refer to a collection of one or more computing devices, including client user devices and/or server devices that are used in connection with a healthcare organization (e.g., one or more hospitals, doctor offices, etc.). In embodiments, a healthcare system 130 may include an EMR data store 132. An EMR data store 132 may include one or more databases that store and/or index electronic medical records. A respective electronic medical record may store or reference patient data of a respective patient of the healthcare organization. An electronic medical record may include a patient identifier, one or more physician identifiers that indicate respective physicians of a patient, physician notes relating to the patient, prescription data indicating treatments that were prescribed to a patient, test results of the patient, and the like. The EMR data store 132 may store additional or alternative data without departing from the scope of the disclosure.

A pharmacy system 140 may refer to a collection of one or more computing devices, including client user devices and/or server devices that are used in connection with a pharmacy organization (e.g., a pharmacy or chain of pharmacies). In embodiments, the pharmacy system 140 may include a prescription data store 142. The prescription data store 142 may include one or more databases that store and/or index prescription records. A respective prescription record may store a prescription ID that uniquely identifies a prescription of a patient, a patient ID identifying the patient to whom the prescription corresponds, a physician ID that identifies the physician that wrote the prescription, a medication ID that identifies the medication that was prescribed, a quantity of the medication that is prescribed, a dosage of the medication that is prescribed, a date on which the medication was prescribed, a date on which the prescription expires, and the like. The prescription data store 142 may store additional or alternative data without departing from the scope of the disclosure.

An insurance system 160 may refer to a collection of one or more computing devices, including client user devices and/or server devices that are used in connection with an insurance organization (e.g., a health insurance provider). The insurance system 160 may be configured to process claims, payout medical bills, collect medical data relating to insured patients, and the like. In embodiments, an insurance system 160 may include an insured data store 1622. The insured data store 1622 may include one or more databases that store and/or index insured records. A respective insured record corresponds to a respective insured person and may store an insured ID that uniquely identifies the person being insured, a policy ID that identifies the policy of the insured, one or more healthcare system IDs that identify one or more respective healthcare systems that the insured visits or has visited, one or more physician IDs that respectively identify a physician of the insured, one or more prescription IDs that respectively identify one or more respective prescriptions of the insured, billing information of the patient, a medical history of the patient, and the like. The insured data store 1622 may store additional or alternative data without departing from the scope of the disclosure.

A testing lab system 150 may refer to a collection of one or more computing devices, including client user devices and/or server devices that are used in connection with an organization that performs medical testing (e.g., a blood testing, a urine analysis, genetic testing, and the like). In embodiments, the testing lab system 150 may include a test results data store 152. The test results data store 152 may include one or more databases that store and/or index test result records that respectively correspond to testing administered by the organization. In embodiments, a test results record may include a test ID that identifies the test that was performed, a test type ID that identifies the type of test performed, a subject ID that indicates a subject of the test (e.g., a patient), a requestor ID that indicates an organization that ordered the test (e.g., a healthcare organization or physician), results data (e.g., the results of the test), and a date (e.g., a date on which the test was administered). The test results data store 152 may store additional or alternative data without departing from the scope of the disclosure.

In embodiments, the CRM system 102 is configured to provide a framework for users associated with a testing lab organization to manage relationships with healthcare organizations, insurance organizations, and/or pharmacy organizations. In embodiments, the CRM system 102 may allow a user to send communications (e.g., emails, text messages, directed messages on social media platforms, and the like) to a healthcare provider, pharmacy, and/or insurance provider. In embodiments, the CRM system 102 may notify a user of the CRM system 102 when a healthcare provider is prescribing a treatment that may benefit from a test (e.g., a genetic test, a blood test, a urine test, etc.). For example, the test management system 104 (discussed below) may recommend the patient undergoing a genetic test before beginning a specific medication to see if the specific medication is likely to be effective in treating the patient suffering from a specific ailment. In these scenarios, the user may opt to send the recommendation to a healthcare provider (e.g., treating physician), the pharmacy, and/or the insurance provider of the patient.

In embodiments, the CRM system 102 may allow a user to send communications (e.g., emails, text messages, directed messages, and the like) to a third party when a patient is suspected of misusing a prescription medication (e.g., overusing/abusing or underusing the prescription medication). In these embodiments, the CRM system 102 may receive a notification of a detected misuse. In response, the CRM system 102 may identify a healthcare provider of the patient misusing a medication and may transmit a notification to the healthcare provider indicating the detected misuse. In some embodiments, the notification to the healthcare provider is sent automatically upon a detected misuse. In some embodiments, a user associated with the testing lab may be provided the option of sending the notification to the healthcare provider.

In embodiments, the test management system 104 is configured to assist users to identify patients that should undergo tests in connection with a prescribed treatment. For example, the test management system 104 may recommend a patient undergo genetic testing in response to a patient being prescribed a particular medication given the particular medication, one or more attributes of the patient, and the ailment of the patient. In this way, the patient may be prescribed the correct medication, rather than have a period where the patient is prescribed a treatment that is unlikely to be effective.

In embodiments, the test management system 104 leverages one or more machine learned models to determine whether to recommend testing for a patient that has been prescribed a specific treatment. In some of these embodiments, the test management system 104 may receive a prescribed treatment (e.g., a medication identifier) for the patient and one or more attributes of the patient (e.g., a patient's age, a patient's sex, a patient's body type, a patient's other medications). The test management system 104 may input these features into a machine learned model that determines whether a particular test (e.g., a genetic test, a blood test, etc.) should be performed. In these embodiments, the test management system 104 may leverage multiple models, such that each respective model may correspond to a different type of test. In some embodiments, the test management system 104 may receive a prescribed treatment (e.g., a medication identifier) for the patient and one or more attributes of the patient (e.g., a patient's age, a patient's sex, a patient's body type, a patient's other medications) and may input these features into a machine learned model that determines any tests that should be performed.

In embodiments, the test management system 104 may be configured to employ a rules-based approach to determine whether any tests should be performed on a patient given a prescribed medication. In these embodiments, the test management system 104 may assess the patient with a set of rules that correspond to a medication that is prescribed to the patient. The conditions which trigger certain rules may be learned using analytics that are derived from outcomes relating to the medication. For example, a certain medication may be ineffective for people over the age of sixty having a certain genetic characteristic, but effective for all other segments of the population. In this example, if the patient is over the age of sixty, the test management system 104 may determine that the patient should undergo genetic testing to determine whether the patient exhibits the certain genetic characteristic.

Upon determining that one or more tests should be performed for a patient, the test management system 104 may output the recommended tests to the CRM system 102. As discussed, in embodiments, a user associated with the lab testing organization may elect to provide the recommendation to an appropriate recipient. In other embodiments, the CRM system 102 may provide the recommendation to the appropriate recipient automatically.

In embodiments, the prescription monitoring system 106 receives lab test results for patients (e.g., blood tests, urine analysis tests) and determines whether it is likely that the respective patients are misusing a prescription medication. In some embodiments, the prescription monitoring system 106 may obtain lab test results of a patient, medication identifiers of any prescription medications that the patient is prescribed or currently using, and relevant patient data (e.g., an ailment of the patient, the age of the patient, weight of the patient, height of the patient, body fat percentage of the patient, and the like). The prescription monitoring system 106 may then determine whether a patient is misusing a prescription medication based on the lab test results, the medication identifiers, and the relevant patient data.

In embodiments, the prescription monitoring system 106 may utilize machine learning and AI techniques to determine if a patient is misusing a prescription medication. In some of these embodiments, the prescription monitoring system 106 may leverage one or more machine learned models to determine if a patient is misusing a prescription medication. For example, in embodiments, a machine learned model may be trained to identify when a patient is likely abusing a particular prescription medication (e.g., an opiate, a benzodiazepine, or amphetamine). These models may be trained on training data samples relating to patients that were determined to be abusing the medication and patients that were determined to be using the prescription medication properly. In these embodiments, the prescription monitoring system 106 may obtain lab test results of the patient (e.g., a blood test and/or a urine analysis test), a prescription medication that the patient is being prescribed, and relevant patient information (e.g., an ailment of the patient, other prescriptions of the patient, an age of the patient, a gender of the patient, a weight of the patient, a body fat percentage of the patient, and the like). In embodiments, the machine learned model may output a classification relating to the patient that indicates whether the patient is likely abusing the medication or using the medication properly. In embodiments, the classification may include a confidence score, whereby a higher confidence score indicates a higher degree of confidence in the classification. In some of these embodiments, the machine learned model(s) may be trained to identify the type of abuse of a medication (e.g., overuse/addiction, use with other controlled substances, and the like). In embodiments, the prescription monitoring system 106 may leverage other machine learned models. For instance, the prescription monitoring system 106 may leverage a machine learned model that is trained to identify when a patient is underusing a prescription medication, which may be indicative of a patient illegally distributing the prescription medication to other people.

In some embodiments, the prescription monitoring system 106 may be configured to apply a rules-based approach for detecting misuse. In these embodiments, the prescription monitoring system 106 may obtain lab test results of the patient (e.g., a blood test and/or a urine analysis test), a prescription medication that the patient is being prescribed, and relevant patient data (e.g., an ailment of the patient, other prescriptions of the patient, an age of the patient, a gender of the patient, a weight of the patient, a body fat percentage of the patient, and the like), which may be analyzed in view of a set of one or more conditions. In these embodiments, the one or more conditions may be indicative of one or more types of abuse. For example, conditions may define maximum allowances of detected opiates, benzodiazepines, and/or amphetamines in a patient's blood or urine. These allowances may vary depending on the patient's prescription and metabolic factors (e.g., ailments, age, body fat, weight, etc.). For example, conditions for a patient with a relatively higher prescribed dosage and/or a relatively higher body fat percentage may define relatively higher allowances. Similarly, conditions may define minimum levels of detected opiates, benzodiazepines, and/or amphetamines for patients, which may vary based on one or more factors (e.g., prescribed amounts and/or metabolic factors). In these examples, the conditions may be tailored to identify underuse of the prescription medication. Upon receiving one or more test results and/or a notification of a prescription relating to a patient, the prescription monitoring system 106 may apply the one or more rules to determine if the patient is misusing the prescription medication.

Upon determining that a patient is likely misusing a prescription medication, the prescription monitoring system 106 may output a notification of the detected misuse to the CRM system 102. As discussed, in embodiments, a user associated with the lab testing organization may elect to provide the notification to an appropriate recipient(s) (e.g., the treating physician) or the CRM system 102 may provide the notification to the appropriate recipient(s) automatically.

The prescription monitoring system 106 may monitor other entities as well. For example, in embodiments, the prescription monitoring system 106 may monitor a physician's prescription history to determine if the physician if overprescribing certain medications.

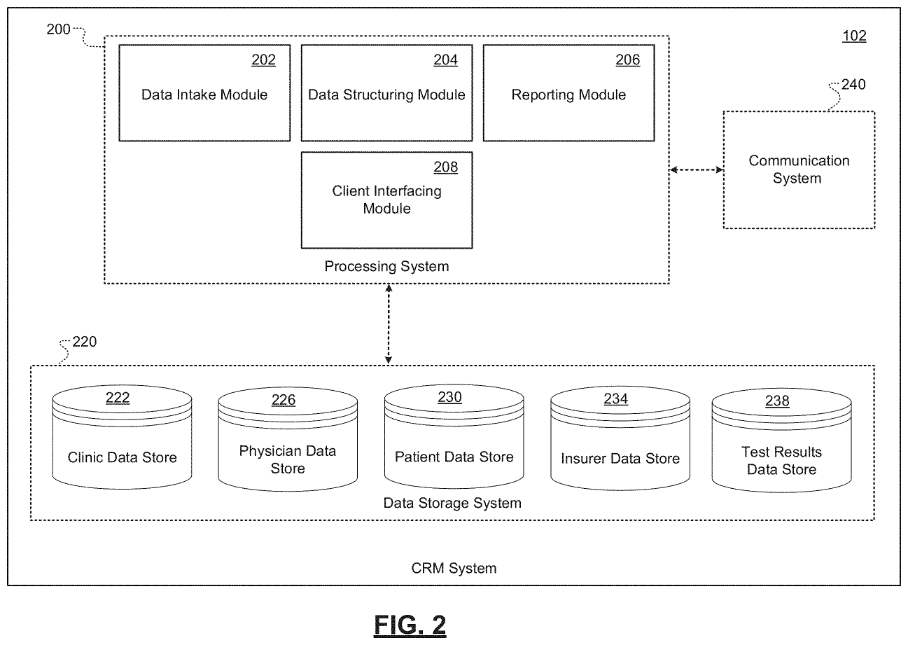

FIG. 2 illustrates a set of example components of a CRM system 102 according to some embodiments of the present disclosure. In embodiments, the CRM system 102 may include a processing system 200, a data storage system 220, and a communication system 240.

The processing system 200 may include memory (e.g., RAM and/or ROM) that stores computer-executable instructions. In embodiments, the processing system 200 executes a data intake module 202, a data structuring module 204, a reporting module 206, and/or a client interfacing module 208, each of which is discussed in further detail below. The processing system 200 may execute additional or alternative modules without departing from the scope of the disclosure.

The data storage system 220 may include one or more storage devices (e.g., hard disk drive, flash drive, etc.) that store data. In embodiments, the data storage system 220 may store a clinic data store 222, a physician data store 226, a patient data store 230, an insurer data store 234, and a test results data store 238, all of which are discussed in further detail below.

The communication system 240 may include one or more communication devices that interface with a communication network (e.g., the internet). The one or more communication devices may effectuate wired or wireless communication.

In embodiments, the clinic data store 222 stores clinic related data. A clinic may refer to any sized healthcare organization (e.g., a hospital system, a multi-physician office, a single physician office) and/or units within an organization (e.g., a cardio-unit of a hospital, an oncology unit of a hospital, a surgery unit of a hospital, an intensive care unit of a hospital, etc.). In embodiments, the clinic data store 222 stores a clinic database that stores and/or indexes clinic records. Each clinic record may correspond to a respective clinic. A clinic record may include and/or may be related to a clinic ID that identifies the clinic, one or more physician IDs that identify physicians associated with the clinic, one or more administrator IDs that identify administrators associated with the clinic (e.g., contacts/decision makers at the clinic), ordering data that indicates orders made by the clinic (e.g., lab tests ordered by the clinic, the lab testing organization that performed each lab test, and dates of each order), and/or suitable metadata. It is noted that the list of items included in a clinic record is provided for example only and may include additional or alternative types of data.

In embodiments, the physician data store 226 stores physician-related data. In embodiments, the physician data store 226 stores a physician database that stores and/or indexes physician records. Each physician record may correspond to a respective physician or other healthcare providers. A physician record may include and/or may be related to a physician ID that identifies the physician, a set of patient IDs that identify a set of patients that see the physician, a set of clinic IDs that indicate any clinics that the physician is associated with, ordering data that indicates orders made by the physician (e.g., lab tests ordered by the physician, the lab testing organization that performed each lab test, and dates of each order), prescription data indicating prescriptions written by the physician (including, dosages, amounts, and dates of each prescription), and/or suitable metadata. It is noted that the list of items included in a physician record is provided for example and may include additional or alternative types of data.

In embodiments, the patient data store 230 stores patient-related data. In embodiments, the patient data store 230 stores a patient database that stores and/or indexes patient records. Each patient record may correspond to a respective patient. A patient record may include and/or may be related to a patient ID that identifies the patient, a set of physician IDs that identify a set of physicians that treat or have treated the patient, a set of clinic IDs that indicate any clinics that the patient has visited, ordered test data that indicates any tests ordered for the patient (e.g., lab tests ordered for the patient, the results of the tests, the lab testing organization that performed each lab test, and the date of each test), prescription data indicating prescriptions written for the patient (including, dosages, amounts, and dates of each prescription, the prescribing physician, etc.), diagnosis data indicating respective diagnosis of the patient (e.g., for which ailments they are being treated), and/or suitable metadata (e.g., age of the patient, height of the patient, weight of the patient, sex of the patient, body fat percentage of the patient, and the like). It is noted that the list of items included in a patient record is provided for example only and may include additional or alternative types of data.

In embodiments, the insurer data store 234 may store insurer-related data. In embodiments, the insurer data store 234 stores an insurer database that stores and/or indexes insurer records. In embodiments, each insurer record may correspond to a respective insurance organization or other healthcare payor. An insurer record may include and/or may be related to an insurer ID that identifies the insurer, a set of clinic IDs that indicate healthcare organizations that the insurer is associated with, a set of patient IDs indicating patients that are customers of the insurer, and the like. It is noted that the list of items included in an insurer record are provided for example only and may include additional or alternative types of data.

In embodiments, the insurer data store 234 may include a claim database that stores and/or indexes claim records. Each claim record may correspond to an insurance-related event. An insurance related event may be any billable or non-billable occurrence that an insurance organization handles on behalf of an insured patient. In embodiments, a claim record may include and/or may be related to a claim ID that identifies the insurance-related event, a patient ID of the patient involved in the event, a clinic ID of the clinic that the patient visited, a physician ID of the provider who treated the patient, diagnosis data that indicates the provider's medical diagnosis, prescription data that indicates any prescriptions that were prescribed in relation to the event, including dosages, amounts, etc., lab test data that indicates any tests that were ordered in relation to the event including the results thereof, and associated metadata (e.g., date of the event on which the insurance-related event occurred). It is noted that the list of items included in a claim record is provided for example only and may include additional or alternative types of data.

In embodiments, the test results data store 238 may store test result-related data. In embodiments, the test results data store 238 stores a test results database that stores and/or indexes test result records. Each test result record may correspond to a respective lab test (e.g., genetic test, blood test, urine test, etc.). A test result record may include and/or be related to a test ID that identifies the test, a lab ID that identifies the testing lab that performed the test, a physician ID that identifies a physician that ordered the test, a test type that indicates the type of test, test result data that indicates the results of the test, an insurer ID that indicates an insurance organization of the patient that underwent the lab test, and any suitable metadata (e.g., a date of the test, an employee that processed the test, the machine that performed the test, etc.). It is noted that the list of items included in a test result record is provided for example only and may include additional or alternative types of data.

In embodiments, the data intake module 202 obtains data from one or more data sources (e.g., healthcare systems 130, pharmacy systems 140, testing lab systems 150 and/or insurance systems 160). The data intake module 202 may implement one or more public or private APIs (e.g., SOAP, RESTful APIs, and the like). The APIs may be passive (i.e., the data sources push data to the data intake module 202) and/or active (i.e., the data intake module 202 pulls data from the data sources).

In embodiments, the data intake module 202 includes an interception module that obtains prescription-related information relating to a clinic or physician. In some embodiments, the interception module may retrieve prescription-related data relating to prescriptions written by a physician or from a clinic. In some of these embodiments, the interception module may obtain prescription-related data from a prescription drug monitoring program (PDMP), whereby the interception module may obtain information relating to prescriptions of specific classes of medications written by respective physicians. In embodiments, the interception module may output any obtained prescription-related information to the data structuring module 204, including any metadata surrounding the prescription (e.g., the physician that wrote the prescription, the dosage amount, the clinic of the physician, the patient, the date of the prescription, the pharmacy at which the prescription is filled, etc.).

In embodiments, the data intake module 202 includes an interaction monitoring module that monitors communications between testing labs and customers (e.g., healthcare organizations, clinics, and/or physicians). In embodiments, the interaction monitoring module may determine each time a sales or service representative of a lab testing organization interacts with a customer (e.g., healthcare organizations, clinics, and/or physicians). In embodiments, the interaction monitoring module may also determine each time a customer orders a test from a lab. In these embodiments, the interaction monitoring module may output determined instances of interactions and/or orders to the data structuring module 204, including any metadata surrounding the interaction and/or order (e.g., the sales representative, the physician or representative of client that made an order, the date of the order, a patient corresponding to the order, etc.).

In embodiments, the data intake module 202 includes an insurer interface module. In embodiments, the insurer interface module interfaces with insurance systems 160. The insurer interface module may be configured to retrieve insurer-related information from an insurance system 160. The insurer-related information may include events related to a healthcare organization, clinic, and a physician. In some embodiments, the insurer interface module may retrieve physician-related information from the insured data store 1622. For example, the insurer interface module may request all claims and/or prescriptions that a physician, or a clinic thereof, billed to insurance company.

In embodiments, the data intake module 202 includes an EMR interface module that interfaces with healthcare systems 130. In embodiments, the EMR interface module may obtain physician-related data and/or patient-related data from the EMR data stores 132 of a healthcare system. For example, the EMR interface module may obtain prescriptions written by physicians, prescriptions written for patients, tests ordered by physicians and/or for patients, test results of patients, diagnoses of patients, patient metadata (e.g., age, sex, weight, body fat percentage), and/or any other suitable data. In embodiments, Fast Healthcare Interoperability Resources can be deployed to exchange resources and other data formats and elements through one or more application programming interfaces or suitable interfaces for exchanging electronic health records and/or electronic medical records. In embodiments, Fast Healthcare Interoperability Resources can be deployed or other suitable standard promulgated by the Health Level Seven International (HL7) health-care standards organization. In embodiments, Fast Healthcare Interoperability Resources can be deployed to build on or incorporate previous data format standards from HL7 and also deploy HTTP-based RESTful protocol, HTML and Cascading Style Sheets for user interface integration. In examples, discrete data elements can be accessed as services such that basic elements of healthcare like patients, admissions, diagnostic reports and medications can each be retrieved and manipulated via their own resource URLs or other suitable network connections. By way of these examples, the various protocols can deploy JSON, XML RDF and others for data representation. It will be appreciated in light of the disclosure that Fast Healthcare Interoperability Resources and other exchange resources can facilitate interoperation between legacy health care systems. This type of data exchange can also facilitate easier deployment of health care information to health care providers and individuals on various connected devices.

The data intake module 202 may obtain additional or alternative data relating to pharmacies, physicians, clinics, insurers, patients, lab testing facilities, and the like from any suitable data sources. Furthermore, it is noted that the data intake module 202, and the pharmacological tracking platform 100 as a whole, may be configured to conform to regulations concerning patient data privacy and personally identifiable information (PII). For example, the data intake module 202 may be configured to be HIPAA (Health Insurance Portability and Accountability Act) compliant.

In embodiments, the data structuring module 204 structures data collected by the data intake module 202. In embodiments, the data structuring module 204 structures the data into data records, such as clinic records, physician records, patient records, insurer records, and/or any other suitable types of records. The data structuring module 204 may create new records when necessary (e.g., when a new entity is detected), and can update preexisting records when a record corresponding to the collected data already exists (e.g., a previously detected entity). For example, if a physician that does not have a physician record corresponding thereto writes a new prescription, the data structuring module 204 may generate a new physician record based on the collected data. Likewise, when the physician writes a subsequent prescription, the data structuring module 204 may update the physician record of the physician based on the new prescription.

In embodiments, data structuring module 204 may generate physician profiles (e.g., physician records) based on the collected data. In some of these embodiments, the data structuring module 204 may use data obtained by the data intake module 202 to generate the physician profiles. In embodiments, a physician's name, clinic that he or she operates out of, and other metadata may be obtained from a healthcare system 130 or insurance systems 160 associated with the physician. In embodiments, the data structuring module 204 may include prescription-related data obtained by the interception module to track a physician's history of writing prescriptions for controlled medications (e.g., opiates, benzodiazepines, amphetamines) in the physician profile. In embodiments, the data structuring module 204 may include ordering histories of the physician in the physician profile based on data obtained by the interaction monitoring module, the insurer interface module, and/or the EMR module. The data structuring module 204 may include other suitable data in the physician profile, including discipline events that indicate instances where the physician was previously disciplined. In embodiments, the data structuring module 204 may be configured to correlate the obtained data to the proper physician. For example, the data structuring module 204 may analyze the obtained data to identify various instances of data that match the physician's profile in order to properly attribute newly obtained data to the physician's profile.

In embodiments, the data structuring module 204 may generate patient profiles (e.g., patient records) based on the collected data. In some of these embodiments, the data structuring module 204 may use data obtained by the data intake module 202 to generate the patient profiles. In embodiments, a patient's name, clinics that the patient has been treated at, physicians that have seen the patient, and other metadata regarding the patient (e.g., age, sex, weight, height, body fat percentage, etc.) may be obtained from healthcare systems 130 or insurance systems 160 associated with the patient. In embodiments, the data structuring module 204 may include prescription-related data obtained by the interception module to track the prescriptions of controlled medications (e.g., opiates, benzodiazepines, amphetamines) written for the patient in the patient profile. This information may include the medication that was prescribed, the physician who wrote each prescription, when the prescription was written, the pharmacy that filled the prescription, and other relevant prescription-related data items In embodiments, the data structuring module 204 may include an ordering history of the patient in the patient profile based on data obtained by the interaction monitoring module, the insurer interface module, and/or the EMR module. The patient profile may indicate which tests were ordered, the physician that ordered the tests, when the tests were ordered, and the results of the tests. In embodiments, the data structuring module 204 may be configured to correlate the obtained data to the proper patient. For example, the data structuring module 204 may analyze the obtained data to identify various instances of data that match the patient's profile in order to properly attribute newly obtained data to the patient's profile.

In embodiments, the reporting module 206 is configured to report notifications and/or recommendations to third parties. Third parties may include healthcare organizations (e.g., hospitals, clinics), pharmacies, testing labs, and/or insurance organizations. Examples of notifications may include a notification that a patient is likely misusing a prescription medication, a notification that a patient has had superfluous tests performed on them, a notification that a physician is prescribing too many controlled medications, a notification that a physician is over ordering lab tests, and the like. Examples of recommendations may include recommendations to order tests for a patient prior to or after a prescribed treatment. In some embodiments, the reporting module 206 reports notifications on behalf of a user (e.g., a sales or service representative of a testing lab). In these embodiments, the reporting module 206 may present the recommendation or notification to a user, whereby the user may select to send the notification or recommendation. In embodiments, the notification module 206 reports notifications and/or recommendations to third parties automatically. In these embodiments, a third party (e.g., healthcare organization or insurer) may request such information from the pharmacological tracking platform 100 and/or a user may elect to have notifications and/or recommendations sent automatically.

In embodiments, the client interfacing module 208 provides an interface for users to contact, message, and communicate with third parties (e.g., customers and potential customers). In some embodiments, the client interfacing module 208 provides a mechanism by which a healthcare supplier (e.g., a lab test facility) may conduct business with its customers and/or potential customers. The client interfacing module 208 may provide a graphical user interface that allows a user to draft messages, set workflows, and share information with third parties.

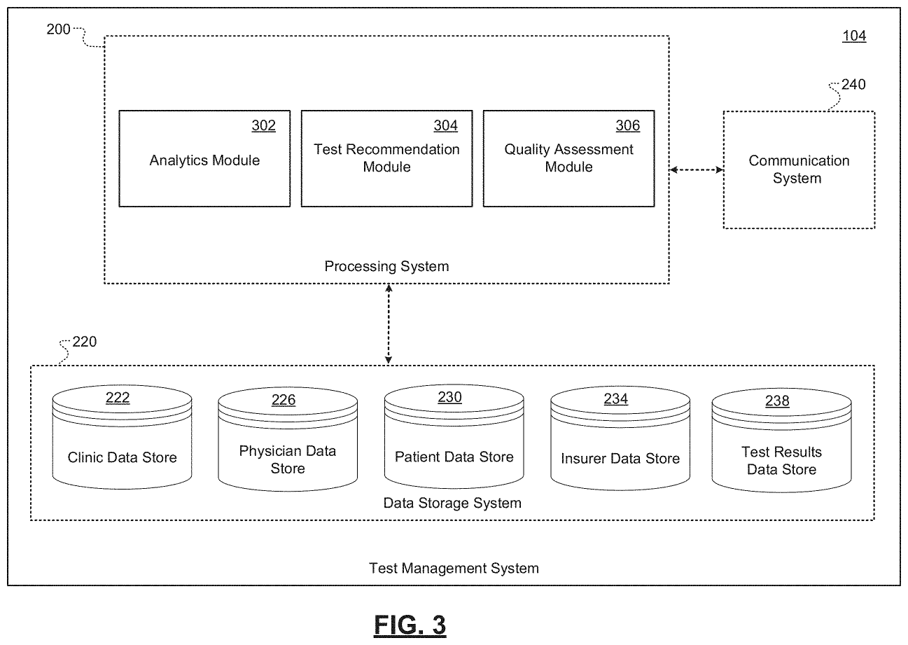

FIG. 3 illustrates an example of a test management system 104 according to some embodiments of the present disclosure. In embodiments, the test management system 104 may include a processing system 200, a data storage system 220, and a communication system 240. In some embodiments, a test management system 104 shares these same hardware resources (e.g., executed by the same set of server devices) as the CRM system 102. In some embodiments, a test management system 104 does not share the hardware resources with the CRM system 102 and may be hosted on an independent computing environment that communicate over a public network via one or more APIs.

In embodiments, the processing system 200 may execute an analytics module 302, a testing recommendation module 304, and/or a quality assessment module 306. The processing system 200 may execute additional or alternative modules without departing from the scope of the disclosure. In embodiments, the data storage system 220 may store a clinic data store 222, a physician data store 226, a patient data store 230, an insurer data store 234, and a test results data store 238, as was described with respect to the CRM system 102.

In embodiments, the analytics module 302 performs various analytics tasks relating to ordered lab tests. For example, the analytics module 302 may perform predictive and/or descriptive data analytics on a physician's or clinic's ordering history of lab tests. Such analytics may uncover unnecessary ordering of tests or even fraudulent ordering patterns. In another example, these types of data analytics may identify physicians or clinics that are potentially underutilizing lab tests in their practices.

In embodiments, the analytics module 302 analyzes physician profiles to determine if a physician is over-ordering or under-ordering lab tests. In some of these embodiments, the analytics module 302 performs statistical analytics techniques on a set of physician profiles to determine whether a physician is over-ordering or under-ordering lab tests. In some of these embodiments, the physician records may be clustered using K-nearest neighbors or K-means clustering to identify physician records that appear in the same cluster(s) as physician records that have been deemed to be anomalous (i.e., under-ordering or over-ordering). In these embodiments, the analytics module 302 may cluster the records based on a set of features, which may include the amount of patients seen, the amount of tests ordered during a time period, the types of tests ordered, the amount charged to insurance companies and/or the patient to run the tests, the diagnosis of the patients, and/or other suitable features. As most physicians having the same or similar specialties, they will have consistent ordering histories, physicians deviating from the normal patterns may be identified based on the clusters to which they belong. The analytics module 302 may perform other types of statistical analysis on physician records, such as deep learning and statistical regressions, amongst others.

In embodiments, the analytics module 302 may analyze the ordering practices of clinics as well. In these embodiments, the analytics module 302 (or another suitable module of the platform 100) may group physician records together based on which clinic they operate from (or primary clinic they operate from) to obtain a clinic profile. In embodiments, the analytics module 302 may then perform statistical analysis on the clinic profiles to identify clinics that are collectively over-ordering or under-ordering lab tests (e.g., as described with respect to physician profiles). Furthermore, if a clinic is found to have a statistical anomaly, the physician records corresponding to the individual physicians of the clinic may be analyzed individually to ensure that the anomaly is not attributable to a single or small set of physicians.

Upon determining that a physician and/or a clinic exhibits an anomalous ordering history, the analytics module 302 may output a notification indicating the anomaly. In some embodiments, the analytics module 302 may output the notification to the reporting module 206 of the CRM platform 102. In other embodiments, the analytics module 302 may output notifications directly to third parties, such as insurance companies or regulatory agencies.

In embodiments, the testing recommendation module 304 recommends lab tests for patients. In some embodiments, the testing recommendation module 304 recommends one or more tests for patients given a prescribed treatment, so as to improve the likelihood that the treatment is effective to the patient. In these embodiments, the testing recommendation module 304 may receive a proposed prescription for a patient from an external system (e.g., a healthcare system or healthcare organization of a patient being prescribed or an insurance system of an insurer of the patient) or from the CRM system 102. In embodiments, the proposed prescription may indicate the patient being prescribed and/or may include information relating to the patient. In the former scenario, the testing recommendation module 304 may retrieve the relevant information relating to the patient from the patient datastore 230 based on an identifier of the patient.

In response to the proposed prescription and the patient information, the testing recommendation module 304 may determine whether to recommend preliminary lab tests before the patient undergoes the prescribed treatment based on the proposed prescription and information relating to the patient. In embodiments, a recommendation may indicate one or more tests that are recommended for the patient given the proposed prescription.

In embodiments, the testing recommendation module 304 may employ a machine learning approach to determine whether to recommend one or more preliminary lab tests. In some of these embodiments, the testing recommendation module 304 may generate a set of features (e.g., a feature vector) based on the proposed prescription (e.g., a medication identifier, a dosage amount, a number of pills, etc.) for the patient and the patient information (e.g., a patient's age, a patient's sex, a patient's weight, a patient's body type, a patient's medication history). In these embodiments, the testing recommendation module 304 may input these features into one or more machine learned models that determine whether a particular test (e.g., a genetic test, a blood test, etc.) or set of tests should be performed. The machine learned model may be any suitable type of machine learned model, such as a neural network, a deep neural network, a recurrent neural network, a Hidden Markov Model, a regression based model, a decision tree (e.g., a classification tree), and the like. In some embodiments, different machine learned models may be trained for different types of medication or classes of medications. In this way, the testing recommendation module 304 may select one or more models to leverage based on the type of medication in the potential prescription.

In some embodiments, a machine learned model may correspond to a specific type of test (e.g., a genetic test, a blood test, or a urine analysis test) and may output a recommendation as to whether the specific type of test should be performed. In these embodiments, the test management system 104 may leverage multiple models, such that each respective model may correspond to a different type of test and may output a recommendation corresponding to the respective type of test. In embodiments, the recommendation may include a confidence score that indicates a degree of confidence in the recommendation. The testing recommendation module 304 may determine whether to select a recommendation made by a model based on the confidence score corresponding to the recommendation (e.g., when the confidence score exceeds a threshold).

In some embodiments, a machine learned model may be trained to recommend multiple types of tests. In these embodiments, the testing recommendation module 304 may input the set of features to the model, which outputs a confidence score for each type of potential test given the features (e.g., the features extracted from the proposed prescription and the patient information). In response, the testing recommendation module 304 may select one or more tests based on the respective confidence scores thereof (e.g., any test having a confidence score greater than a threshold).

In embodiments, the test management system 104 may be configured to employ a rules-based approach to determine whether any tests should be performed on a patient given a prescribed medication. In these embodiments, test management system 104 may assess the patient with a set of rules that are determined for a respective medication. The conditions which trigger certain rules may be learned using analytics that are derived from outcomes pertaining to the medication. For example, a certain medication may be ineffective for people over the age of sixty having a certain genetic characteristic, but effective for all other segments of the population. In this example, if the patient is over the age of sixty, the test management system 104 may determine that the patient should undergo genetic testing to determine whether the patient exhibits the certain genetic characteristic.

Upon determining one or more recommended tests for the patient given the proposed prescription, the testing recommendation module 304 may output a recommendation that includes a respective test type indicator for each respective test that is being recommended. In some embodiments, the testing recommendation module 304 may output the recommendation to the reporting module 206 of the CRM system 102. In other embodiments, the testing recommendation module 304 may output recommendations directly to third parties, such as healthcare providers (e.g., physicians, nurses, etc.). In embodiments, a computing device associated with one or more third parties may provide outcome data, indicating whether the test was performed, the prescription that was ultimately prescribed to the patient, and whether the prescription was effective in treating the patient. For example, this information may be obtained from the healthcare system 130 and/or the insurance system 160. In embodiments, this outcome data may be used to reinforce the models that are used to make the recommendation.

In some embodiments, the testing recommendation module 304 recommends one or more tests for patients undergoing or having undergone a treatment, so as to improve the post-treatment monitoring of the patient.

In embodiments, the quality assessment module 306 measures the quality and/or effectiveness of respective lab testing organizations. The quality assessment module 306 may utilize machine learning, statistical analysis, and/or rules-based analysis to assess the quality of a lab testing organization.

In embodiments, the quality assessment module 306 may generate a lab quality profile of a testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then automatically generate plain-language textual summaries (e.g., using natural language generation) corresponding to the testing lab, where the summaries are grouped by aggregated laboratory issues.

In embodiments, the quality assessment module 306 is configured to generate a lab quality profile of a testing lab that includes a textual summary of the testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then automatically generate plain-language textual summaries (e.g., using natural language generation) corresponding to the testing lab, where the summaries are grouped by aggregated laboratory issues.

In embodiments, the quality assessment module 306 is configured to generate an improvement plan for a testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then automatically generate a plain-language textual improvement plan (e.g., using natural language generation) corresponding to the testing lab based on the identified laboratory issues.

In embodiments, the quality assessment module 306 is configured to identify a primary cause of an identified lab issue. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues. The quality assessment module 306 may receive and parse human-input information relating to each laboratory issue. In some embodiments, the quality assessment module 306 may combine differently worded descriptions that have similar meanings or the same meaning. The quality assessment module 306 may then map the identified issues to an ontology that includes different types of entities that relate to the platform 100 (e.g., testing labs, physicians, clinics, etc.). The quality assessment module 306 may then automatically generate an indication of the most likely entity that was the cause of each respective issue.

In embodiments, the quality assessment module 306 is configured to generate statistics relating to the employees of the testing lab. In some of these embodiments, the quality assessment module 306 may aggregate transaction histories of a collection of testing lab organizations (e.g., order histories of respective testing labs) over a period of time. The quality assessment module 306 may analyze the volumes and types of the tests indicated in each respective transaction history, and may compile data sets relating to pre-analytical, analytical, and/or post-analytical laboratory issues, test speeds, and/or performance metrics. The quality assessment module 306 may then compile time utilization and/or workload statistics for each testing lab and/or the lab workers employed therein. In some embodiments, the quality assessment module 306 may activate a workflow that identifies a source of a respective test that proceeded a drop in productivity and activates a quality review of the source. Additionally or alternatively, the quality assessment module 306 may activate a workflow that identifies one or more pieces of equipment used during a test that resulted in an issue, and activates a quality review of the one or more pieces of equipment.

The test management system 104 may include additional or alternative components that perform various tasks related to the oversight and/or management of lab tests.

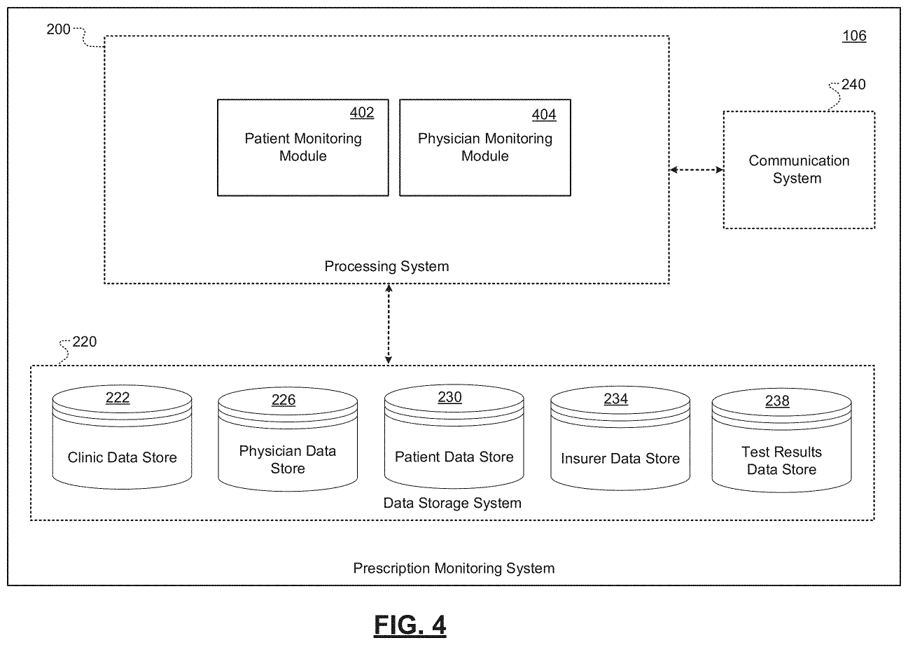

FIG. 4 illustrates an example of a prescription monitoring system 106 according to some embodiments of the present disclosure. In embodiments, the prescription monitoring system 106 may include processing system 200, a data storage system 220, and a communication system 240. In some embodiments, a test management system 104 shares these same hardware resources (e.g., executed by the same set of server devices) as a CRM system 102 and/or a test management system 104. In some embodiments, a prescription monitoring system 106 does not share the hardware resources with a CRM system 102 and/or a test management system 104, and may be hosted on an independent computing environment.

In embodiments, the processing system 200 may execute a patient monitoring module 402 and/or a physician monitoring module 404. The processing system 200 may execute additional or alternative modules without departing from the scope of the disclosure. In embodiments, the data storage system 220 may store a clinic data store 222, a physician data store 226, a patient data store 230, an insurer data store 234, and a test results data store 238, as was described with respect to the CRM system 102.

In embodiments, the patient monitoring module 402 monitors test results associated with patients that are prescribed controlled medications, such as opiates, amphetamines, and/or benzodiazepines to determine whether a patient is misusing the prescribed medication. In these embodiments, the patient monitoring module 402 may begin monitoring a patient's use of a controlled medication when the patient is initially prescribed the controlled medication. The patient monitoring module 402 may determine a patient is initially prescribed a controlled medication from, for example, a PDMP, a healthcare system 130, and/or an insurance system 160. In certain scenarios, the treating physician may require the patient to undergo testing (e.g., urine analysis or blood testing) while prescribed the medication. In embodiments, the results of the test corresponding to a patient may be stored in the test results data store 238, and may be associated with the patient record of the patient. Each time new test results are received for a patient, the patient monitoring module 402 may analyze the test results to determine if the patient is likely misusing the medication. As previously discussed, misusing a medication may refer to overusing/abusing the medication and/or underusing the medication.

In some embodiments, the patient monitoring module 402 may generate a usage profile of the patient based on the collection of the prescriptions of the controlled medication that have been written for the patient, a collection of test results corresponding to the patient during the time period where the patient was taking the medication, and patient information of the patient (e.g., patient's age, weight, body type, sex, activity rate). In these embodiments, the patient monitoring module 402 may determine if a patient is misusing the controlled medication based on the usage history of the patient. The patient monitoring module 402 may identify misuse in any suitable manner.

In embodiments, the patient monitoring module 402 may employ machine learned classification models that are trained to classify misuse of controlled medications. In some of these embodiments, the machine learning system 108 may train the machine learned classifications on training data sets that include usage profiles of patients that were deemed to be using a controlled medication properly and patients that were deemed to be misusing the controlled medication (e.g., usage profiles of patients) that were deemed to be underusing the medication (which may suggest selling of medication or no need for the medication) and/or usage profiles of patients that were deemed to be overusing/abusing the medication. In embodiments, the patient monitoring module 402 may generate a set of features (e.g., a feature vector) from the usage profile of the patient being monitored and may input the set of features into the classification model. The classification model may output a classification corresponding to the patient that indicates whether there is potential misuse detected, and in some of these embodiments, a type of misuse (e.g., overuse/abuse or underuse). In embodiments, the classification may include a confidence score in the classification, whereby the patient monitoring module 402 may select a classification based on the confidence score thereof (e.g., when the confidence score exceeds a threshold). In the event the patient monitoring module 402 determines that the classification indicates misuse, the patient monitoring module 402 may output a notification indicating the potential misuse (which may also include the type of misuse).

In embodiments, the patient monitoring module 402 may perform analytics to identify potential misuse. In some of these embodiments, the patient monitoring module 402 may perform a statistical analysis on the usage profile to determine trends in the patient's dosage amounts and test results that would indicate overuse or underuse. In some embodiments, the patient monitoring module 402 may analyze the usage profile of the patient in relation to the usage profiles of other patients, including patients deemed to be misusing the medication that the patient is prescribed and patients deemed to be using the medication properly. In some of these embodiments, the patient monitoring module 402 may implement a clustering technique (e.g., K-nearest neighbors or K-means clustering) to determine whether the patient belongs to a cluster where the usage profiles indicated misuse or to a cluster where the usage profiles indicated proper use. In the event the usage profile of the patient is clustered to a cluster where the usage profiles indicated misuse, the patient monitoring module 402 may determine that the patient is likely misusing the medication and may output a notification indicating the potential misuse (which may also include the type of misuse).

The patient monitoring module 402 may monitor patients for potential misuse of a medication in other suitable manners. For example, the patient monitoring module 402 may implement a rules-based approach to identify potential misuse.

Upon determining that a patient is likely misusing a controlled medication, the patient monitoring module 402 may output a notification indicating that the patient is likely misusing the controlled medication (which may also indicate the type of misuse). In some embodiments, the patient monitoring module 402 may output the notification to the reporting module 206 of the CRM system 102. In other embodiments, the patient monitoring module 402 may output notifications directly to third parties, such as the treating physician of the patient.

In embodiments, the physician monitoring module 404 monitors a physician's prescribing history and/or the lab tests of the physician's patients to determine if a physician is likely overprescribing controlled medications. In some of these embodiments, the physician monitoring module 404 may generate a prescribing profile for any physician that prescribes controlled medications. In embodiments, the prescribing profile may indicate each instance that the physician prescribed a controlled medication and, in some of these embodiments, the diagnosis leading to the prescription. In some of these embodiments, the prescribing profile may also indicate test results of patients of the physician that were prescribed the controlled medication and/or the determinations made by the patient monitoring module 402 as to whether the patient was misusing the medication or properly using the medication. In these embodiments, a physician who has higher ratios of patients who are likely misusing the medication versus patients that are likely using the medication properly may be more likely to be flagged as overprescribing the medication.

In embodiments, the physician monitoring module 404 may determine whether a physician is likely overprescribing a controlled medication based on the prescribing profile of the physician. The physician monitoring module 404 may implement any suitable technique to determine whether the physician is likely overprescribing a controlled medication.

In embodiments, the physician monitoring module 404 may implement machine learning techniques to determine whether the physician is likely overprescribing a controlled medication. For example, the physician monitoring module 404 may generate a set of features based on the physician's prescribing profile and may input the set of features into a machine learned classification model that is trained to identify instances where a physician is likely overprescribing a controlled medication. In some of these embodiments, the machine learned classification model may be trained on training data sets that include prescribing profiles where the respective physician was deemed to be overprescribing controlled medications and prescribing profiles where the respective physician was deemed not to be overprescribing (e.g., the physician's practices were deemed normal or less than normal).

In embodiments, the physician monitoring module 404 may implement statistical analysis to determine whether the physician is likely overprescribing a controlled medication. In some of these embodiments, the physician monitoring module 404 may cluster (e.g., K-nearest neighbors or K-means clustering) prescribing profiles of physicians to determine whether the physician being monitored is likely overprescribing the medication. The physician monitoring module 404 may use additional or alternative statistical analysis techniques to determine whether the physician is likely overprescribing a controlled medication. In some embodiments, the physician monitoring module 404 may implement a rules-based approach to determine whether the physician is likely overprescribing a controlled medication.

Upon determining that a physician is likely overprescribing a controlled medication, the physician monitoring module 404 may output a notification indicating that the physician is likely overprescribing a controlled medication. In some embodiments, the physician monitoring module 404 may output the notification to the reporting module 206 of the CRM system 102. In other embodiments, the physician monitoring module 404 may output notifications directly to third parties, such as insurance companies or regulatory agencies.

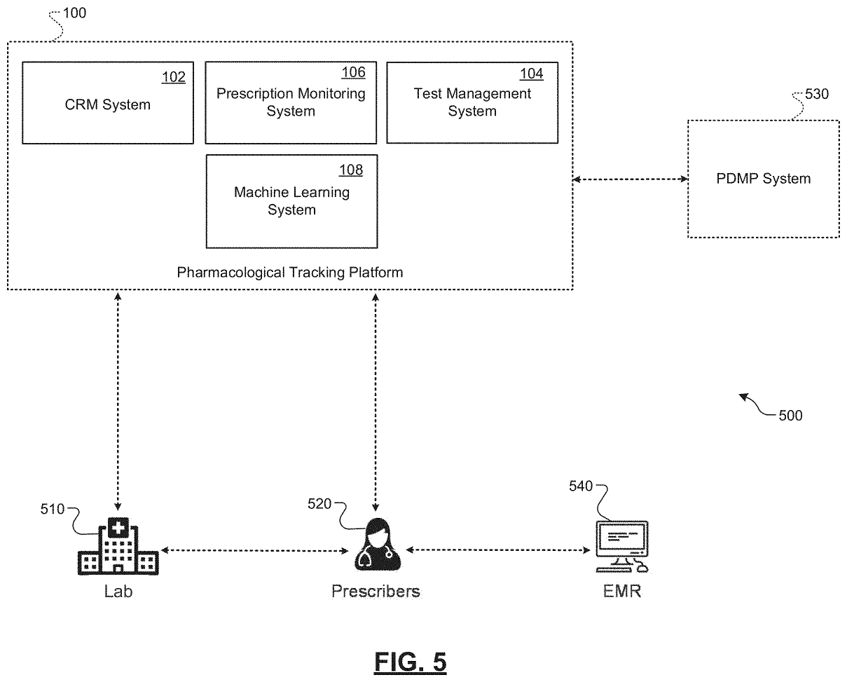

Various example implementations of the report generation and outputting function of the pharmacological tracking platform 100 will be described in reference to FIG. 5-FIG. 10. As mentioned above, the present disclosure provides for generating an enhanced toxicology report corresponding to a patient that is a simple and easy to understand summary of the use and potential misuse of controlled substances for a patient. An example reporting system environment 500 can include the pharmacological tracking platform 100, a laboratory or laboratory system 510 (referred to herein as “laboratory 510”), a user or user system 520 (referred to herein as “user 520”), a PDMP or PDMP system 530 (referred to herein as “PDMP 530”), and an EMR or EMR system or database 540 (referred to herein as “EMR 540”). Each of the pharmacological tracking platform 100, the laboratory 510, the user 520, the PDMP 530, the EMR 540 can comprise one or computing devices operating to perform the techniques described herein. Further, the pharmacological tracking platform 100, the laboratory 510, the user 520, the PDMP 530, the EMR 540 can be in communication with any of the other components of the environment 500, for example, via a network (such as network 380). In some aspects, the pharmacological tracking platform 100, the laboratory 510, the user 520, the PDMP 530, and the EMR 540 can share various information, assessments, calculations, records, determinations, etc. (as described below) to assist in the creation, storage, and maintenance of an enhanced toxicology report 600, which is described more fully below.

The pharmacological tracking platform 100 can receive laboratory test results from the laboratory 510, e.g., based on an order from the user 520 for a toxicology screen of a patient. The laboratory test results received by the pharmacological tracking platform 100 can correspond to the patient and be indicative of the toxicology screen of the patient. The toxicology screen can be one or more of the various known drug tests that determine the type and approximate amount of certain drugs and medications that the patient has taken. In certain circumstances, the user 520 will be a healthcare care professional (a doctor, a nurse, a dentist, a physician's assistant, or the like) that has ordered the toxicology screen to assist in the treatment of the patient, although other possibilities are within the scope of the present disclosure. The laboratory 510 can proactively provide the laboratory test results to the pharmacological tracking platform 100. Alternatively, the pharmacological tracking platform 100 can request the laboratory test results from the laboratory 510, e.g., upon being notified by the user 520 of the toxicology screen.

The pharmacological tracking platform 100 can further retrieve controlled substance prescription data for the patient from the PDMP 530. The controlled substance prescription data can include prescriptions of controlled substances issued to the patient for a relevant time period (e.g., the previous two or more years). In some aspects, the pharmacological tracking platform 100 can retrieve the controlled substance prescription data upon being notified by the user 520 of the toxicology screen and/or upon receiving the laboratory test results for the patient from the laboratory 510.

The pharmacological tracking platform 100 can analyze the controlled substance prescription data and the laboratory test results to determine various factors, measurements, calculations, etc. relating to the use and potential misuse of controlled substances for the patient. For example only, the pharmacological tracking platform 100 can determine a daily morphine milligram equivalent of the patient for a given time period, an overdose risk score, and a drug consistency assessment. The daily morphine milligram equivalent of the patient for the given time period can correspond to a cumulative intake of opioid class drugs by the patient on a daily basis for the given time period. The overdose risk score can be a number, grade, or other scoring indexes that are indicative of a likelihood of an unintentional overdose by the patient, as further described below. The drug consistency assessment is representative of a match between the controlled substance prescription data and the laboratory test results for the patient.

In certain aspects, the pharmacological tracking platform 100 can also or alternatively obtain patient attributes of the patient from one or more patient data sources (e.g., such as a data storage system 220). Examples of such patient attributes can correspond to an age of the patient, a weight of the patient, a body type of the patient, an activity level of the patient, and a diagnosis of the patient, although this is not an exhaustive list of patient attributes. In such implementations, the enhanced toxicology report can be further based on any or any combination of these patient attributes, which may assist the pharmacological tracking platform 100 in more accurately determining the various indications relating to the use and potential misuse of controlled substances for the patient.

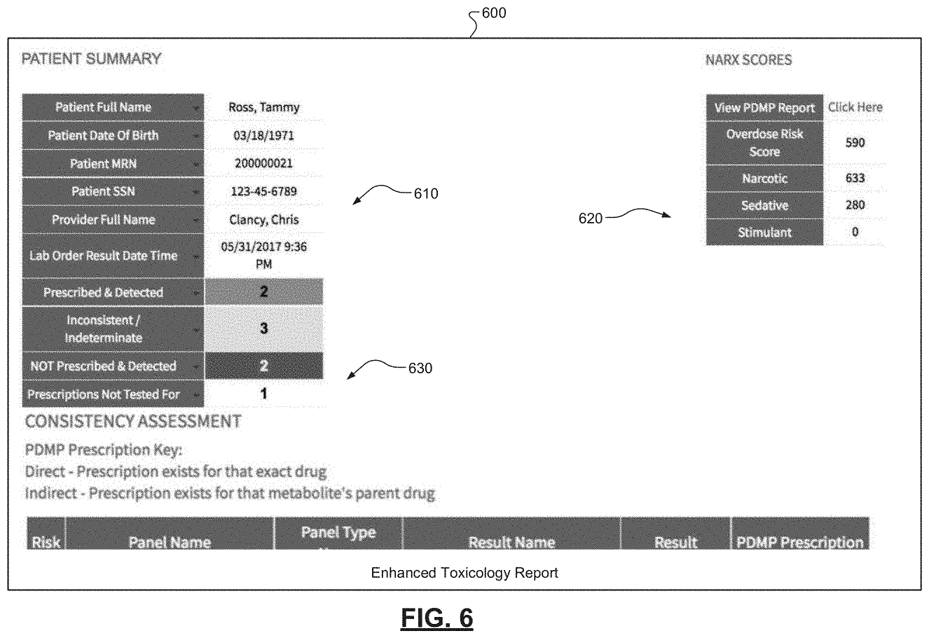

Based on the determined factors relating to the use and potential misuse of controlled substances for the patient (e.g., the daily morphine milligram equivalent of the patient for a given time period, the overdose risk score, and the drug consistency assessment), the pharmacological tracking platform 100 can generate an enhanced toxicology report corresponding to the patient. An example of such an enhanced toxicology report 600 is illustrated in FIG. 6-FIG. 10. While each of FIG. 6-FIG. 10 shows a specific view or presentation of the information in the enhanced toxicology report 600, it should be appreciated that the enhanced toxicology report 600 can include more or less information depending on the specific implementation of the pharmacological tracking platform 100.

Referring now to FIG. 6, a first view of the example enhanced toxicology report 600 is shown. The example enhanced toxicology report 600 can include patient identification information 610, such as the patient name, date of birth, medical record number, and/or social security number. Further, the example enhanced toxicology report 600 can include one or more of the determined overdose risk scores 620. As shown in FIG. 6, the illustrated overdose risk scores 620 include individual overdose risk scores for various drug types (e.g., narcotic, sedative, stimulant), as well as an overall overdose risk score that is independent of drug type. As more fully described below, the enhanced toxicology report 600 shown in FIG. 6 can also include a drug consistency assessment 630 that is representative of a match between the controlled substance prescription data and the laboratory test results for the patient.

The drug consistency assessment 630 shown in FIG. 6 includes multiple drug consistency scores based on the drug consistency assessment, wherein each particular drug consistency score is indicative of a match between a particular drug identified in either or both of the controlled substance prescription data and the laboratory test results for the patient. For example only, a particular drug consistency score can indicate one of the following circumstances: (i) a prescribed and detected condition in which the particular drug is identified in both of the controlled substance prescription data and the laboratory test results for the patient; (ii) a detected but not prescribed condition in which the particular drug is identified in the laboratory test results for the patient but not the controlled substance prescription data; (iii) an inconsistent condition in which (a) the particular drug is a drug metabolite of a parent drug and is identified in the laboratory test results for the patient and the controlled substance prescription data indicates a prescription for the parent drug, or (b) the particular drug is identified in the controlled substance prescription data and the laboratory test results for the patient indicate that the particular drug is not present at a prescribed amount in the patient; and (iv) a particular drug is identified in the controlled substance prescription data but no corresponding laboratory test was ordered to detect the particular drug. A recommendation to the user could be made to order a lab test to check for the presence of the particular drug. As shown in FIG. 6, each of these conditions can be separately indicated, e.g., by a number and/or by a color or other indication. For example only, in some implementations, the drug consistency assessment 630 can also include a confidence score indicative of a confidence level in the determined assessment of drug consistency.

In the various examples herein, PDMPs can be state-run programs which collect and/or distribute data about the prescription and dispensation of federally controlled substances. In some implementations, PDMPs are electronic databases that allow healthcare providers to see patients' prescription histories, thereby allowing doctors and other drug prescribers to check whether a patient has been prescribed and dispensed controlled drugs, such as opioids, before prescribing others to the patient. Some PDMPs also track non-fatal and fatal opioid overdoses, identify risk factors for fatal overdoses in patients, and track toxicology testing. The US federal government provides funding to the states so that each state can fund its own PDMP program.

The goal of PDMPs is to help to prevent adverse drug-related events through opioid overdoses, drug diversion, and/or substance abuse by decreasing the amount and/or frequency of opioid prescribing. Such PDMPs may be accessed and utilized by physicians, physician assistants, nurse practitioners, dentists, and/or other prescribers, pharmacists, and/or pharmacy support staff, as well as law-enforcement agencies and research agencies. These parties may act individually or collaborate together to support the legitimate medical use of controlled substances, while limiting their abuse and/or diversion, as further described herein.

Pharmacies and dispensing prescribers of controlled substances may be required to register with their respective state PDMPs and/or to report the dispensation of such prescriptions to an associated electronic online database. For example only, when a pharmacist dispenses drugs to a patient or is about to dispense drugs to a patient, the pharmacy logs the dispensation with the PDMP. In some states, pharmacies are required to log drug dispensation with the PDMP in real-time or substantially real-time. In other states, pharmacies log drug dispensation daily, weekly, monthly, or at some other interval. Once dispensation of a drug has been logged, a record of the dispensation is accessible by one or more of doctors, other healthcare providers, state insurance programs, healthcare licensure boards, state health departments, and first responders and other law enforcement personnel. In some cases, PDMP information is shared between states, and/or is used by the federal government, such as to improve statistical gathering and legislation to combat opioid abuse.

As briefly mentioned above, PDMP information can be used by doctors and other providers of prescriptions to help prevent patients from seeing different doctors to receive redundant drug prescriptions from each of the doctors, which is sometimes referred to as “doctor shopping.” If a doctor views PDMP prescription logs for a patient before prescribing an opioid to the patient, the doctor may see that the patient has already been prescribed one or more opioids recently by other doctors and may take the appropriate action, e.g., refusing to provide one or more additional opioid prescriptions to the patient. By reducing such “doctor shopping,” PDMPs can assist in curbing opioid addiction.

PDMP information can also be used by lawmakers and administrative agencies to assist in drafting legislation to curb opioid addiction. The lawmakers and administrative agencies can use PDMP log information to inform themselves about general opioid prescribing practices in states, regions, or other geographical areas. The lawmakers and administrative agencies can then pass legislation and regulations using real data about prescribing practices to accurately target trends and issues with the current healthcare system in the geographical area(s) under consideration. For example only, state lawmakers and administrative agencies can access PDMP logs to acquire data regarding opioid prescribing practices within their state, and federal lawmakers and administrative agencies can access PDMP logs of multiple states to acquire data regarding opioid prescribing practices and trends between states and in the whole United States.

In some aspects, PDMP information can also be used by law enforcement agencies and first responders to assist in handling cases of opioid addiction, overdose, and withdrawal. For example only, the law enforcement agencies and first responders can check PDMP logs for an individual who is addicted to opioids or is experiencing opioid overdose or withdrawal to accurately ascertain an extent of the individual's opioid use and thereby provide proper assistance, such as by providing drugs that block the effects of opioids, e.g., Naloxone.

In yet another use case, PDMP information can be used by healthcare personnel (such as anesthesiologists and nurses) to assist in medical procedures that do not generally involve opioids. For example, prior to a surgical procedure, a doctor, nurse, or anesthesiologist can check PDMP logs to determine whether a patient is currently taking opioids. The doctor, nurse, or anesthesiologist can then more accurately prepare the patient for surgery, such as by raising or lowering levels of anesthetic used during the surgery to account for interactions between opioids and anesthesia. In some cases, patients may be reluctant to disclose opioid use or addiction to healthcare personnel due to stigma, embarrassment, personal issues, or other reasons. In such cases, it may be important for the healthcare personnel to determine an extent of opioid use by the patient in order to foresee complications regarding the interactions between opioids and anesthesia or other drugs used during care.

While the above discussion of PDMPs has been limited to PDMPs as implemented in the United States, it should be appreciated that similar programs exist in many other countries and regions, some of which are described below. For example only, several European countries have implemented national drug prescription tracking and information sharing. In France, the National Agency for the Safety of Medicines and Health Products (ANSM) develops several activities both in France and on behalf of the European Union to track prescribing practices and help develop strategies for curbing opioid abuse, such as regulation of prescription and dispensing conditions and reductions in prescription periods. The ANSM has an online reporting tool for use by healthcare professionals, pharmacists, and patients to report use and overuse of opioids, methods of use of opioids, prescribing practices of opioids, and compliance or noncompliance with the laws and regulations regarding opioids. Spain and Germany have similar national systems for providing information, conducting research, and receiving incident reports regarding opioid drugs and abuse thereof.

Internationally, the European Union (EU) drug agency in Lisbon (European Monitoring Centre for Drugs and Drug Addiction) has established the EU4MD database to track prescription drug importation and exportation to and from countries neighboring the EU, known as “neighborhood countries.” The neighborhood countries include Belarus, Ukraine, Moldova, Georgia, Armenia, Azerbaijan, Lebanon, Israel, Palestine, Jordan, Egypt, Libya, Tunisia, Algeria, and Morocco. The EU4MD seeks to establish a better understanding of drug markets, capacity for development for forensic analysis, assessment of the environmental impact of drug production, identification of drug problem “hot spots,” mapping of production and trafficking dynamics, technological innovations, threat assessment, and responses to emerging issues to support the EU and neighboring countries.

In Denmark, the Register of Medicinal Product Statistics includes data on all drugs sold in primary care or purchased for use in Danish hospitals. Aggregate data on gross sales of drugs are freely available, and individual-level data on prescriptions filled by Danish residents at community pharmacies are available as an independent sub-registry known as the National Prescription Registry. However, the National Prescription Registry does not provide information regarding drugs used during hospital admissions, drugs used by certain institutionalized individuals, such as individuals institutionalized with psychiatric illness, and drugs supplied directly by hospitals or treatment centers.

In Finland, a service called Kanta allows healthcare personnel and pharmacies to record information regarding drug prescribing. The records are available to patients and can be made publicly available with patient consent. Prescriptions issued by healthcare professionals in Finland are accessible in Kanta and are recorded for at least twenty years. Information regarding deceased individuals is available for up to twelve years after the patient's death. Information stored by Kanta is available to pharmacies and healthcare providers in Finland, and in some cases is also accessible in other European countries.

The Norwegian Prescription Database (NorPD) monitors drugs dispensed by prescription in Norway. NorPD collects and processes data on drug consumption in Norway to map usage trends and monitor trends over time, and can be used as a resource for research, as well as to give health authorities a statistical management tool for quality control of drug use and to give prescribers a basis for internal control and quality improvement of their prescribing practices. NorPD data sorted by demographic, such as sex, age, or region, is publicly available, but information about a patient's name, address, or national identification is not stored.

In Sweden, the Swedish Prescribed Drug Register (SPDR) contains information about age, sex, and unique identifier of each patient to whom a drug has been prescribed. The SPDR also includes information regarding drug names, costs, the professional and training of the prescriber, the prescribed amount of drug, the date of prescription, the date of collection, and other similar information. The information stored by the SPDR is available to researchers, journalists, city council investigators and authorities, and pharmaceutical industry representatives. Personal information such as patient name and identifier are private.

In China, the China Food and Drug Administration has implemented the Chinese Electronic Drug Monitoring Network (CEDMN) to track prescription drug products. Information is tracked and exchanged in the CEDMN via XML data. CEDMN information tracks prescription drugs from manufacturers, to warehouses, and to pharmacies. Pharmacists or other drug dispensers and patients can check the CEDMN database to trace drugs to their sources. Drugs are also tracked and logged in the CEDMN using barcode scanning, RFID identifiers, and Electronic Data Interchange. In Japan, the National Database of Health Insurance Claims and Specific Health Checkups of Japan (NDB) provides information regarding prescription drugs and health insurance claims to the public. The Japanese Ministry of Health, Labour, and Welfare also disseminates information and statistics regarding prescription drugs.

For ease of description, the terms “prescription drug management program/programs” and “PDMP/PDMPs” as used herein will refer to any and all of the above programs and any other similar programs that exist now or in the future directed to the monitoring, managing, etc. of prescription drugs in any region, nation, or other jurisdiction.

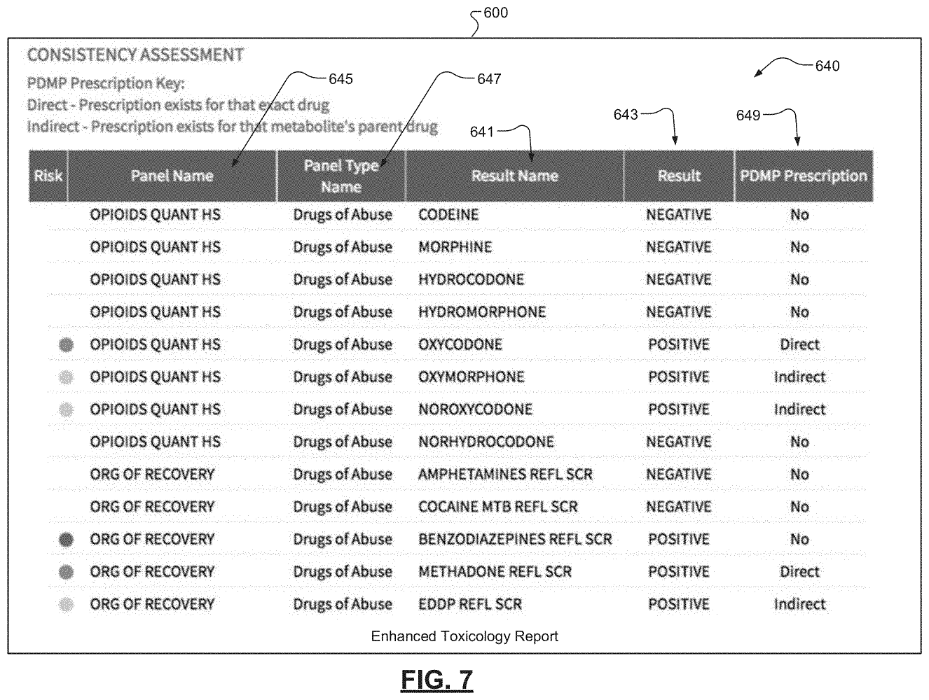

With further reference to FIG. 7, which illustrates a second view of the example enhanced toxicology report 600, in certain aspects the enhanced toxicology report 600 can include a toxicology screen report breakdown 640. The toxicology screen report breakdown 640 can include a detailed list 641 of the various drugs/controlled substances that were tested by the laboratory 510, as well as the results 643 of those tests. The toxicology screen report breakdown 640 can further include a panel name of the test 645, a type of the panel tested 647, and a PDMP prescription section 649. The PDMP prescription section 649 can be indicative of the correlation between the tested drugs/controlled substances and the controlled substance prescription data from the PDMP 530.

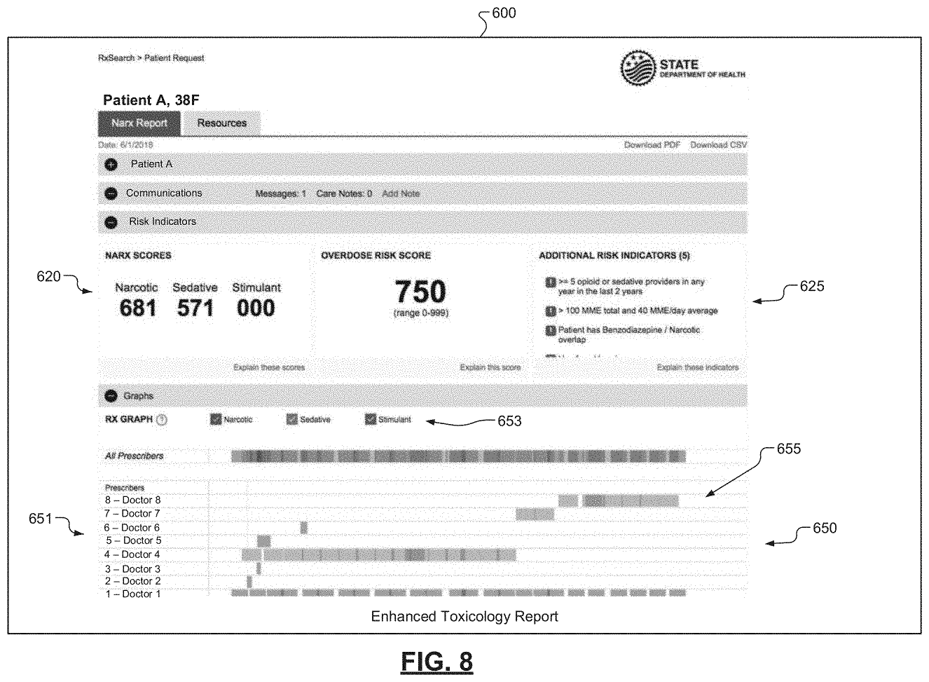

In yet another view (illustrated in FIG. 8), the enhanced toxicology report 600 can include a graphical element 650 that is indicative of prescriptions of controlled substances issued to the patient for a relevant time period. The graphical element 650 as shown includes a list 651 of prescribers that issued the prescriptions, a legend 653 for identifying the drug identity for each prescription, as well as a graph 655 that illustrates the overlap of prescriptions of controlled substances issued to the patient for the relevant time period from multiple prescribers. The enhanced toxicology report 600 shown in FIG. 8 also includes a different representation of the overdose risk score 620 in which one or more additional risk indicators 625 are included. These additional risk indicators 625 are risk categories to which the patient may match and can include, for example only, drug inconsistency, doctor shopping, and/or dangerous drug combinations.

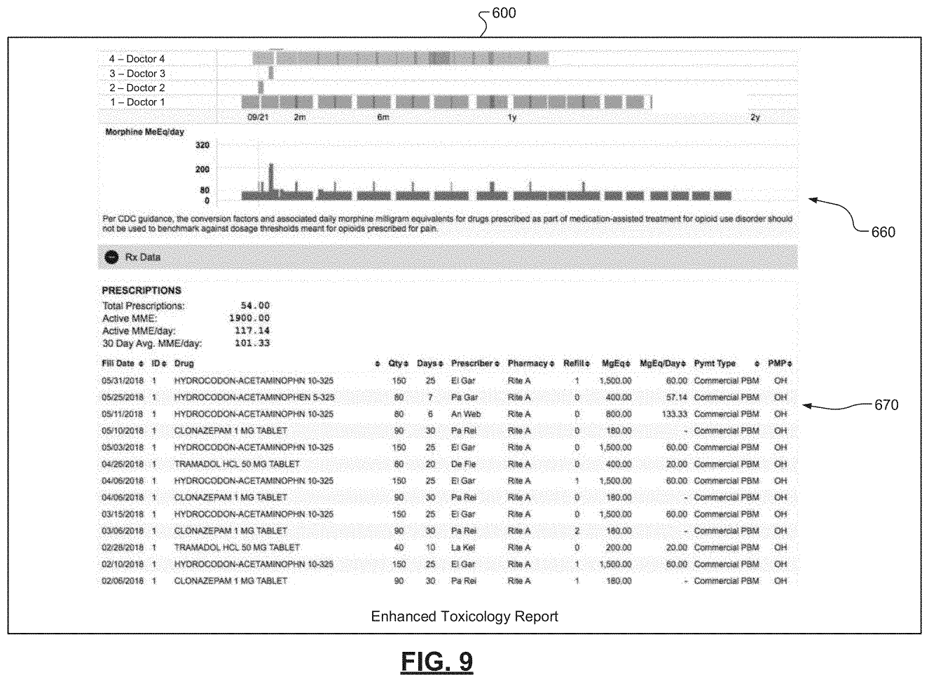

With further reference to FIG. 9, the enhanced toxicology report 600 can include an indication of the determined daily morphine milligram equivalent for the patient for a given time period. For example only, the enhanced toxicology report 600 can include a historical trend 660 of the determined daily morphine milligram equivalent for the patient for a given time period (such as over the previous two years). In some aspects, this historical trend 660 of the determined daily morphine milligram equivalent for the patient can be presented in a graphical format as shown in FIG. 9, in which the historical trend 660 is shown in a bar graph. Other formats for indicating the determined daily morphine milligram equivalent for the patient for a given time period are contemplated by the present disclosure. In certain implementations, the enhanced toxicology report 600 can also include a detailed prescription history 670 of the patient. The detailed prescription history 670 can include various details of the prescriptions issued to the patient, including but not limited to the date of prescription, the drug type/name, the quantity, the number of days of prescription provided, the prescriber, the filling pharmacy, an indication of the number of refills, and the strength.

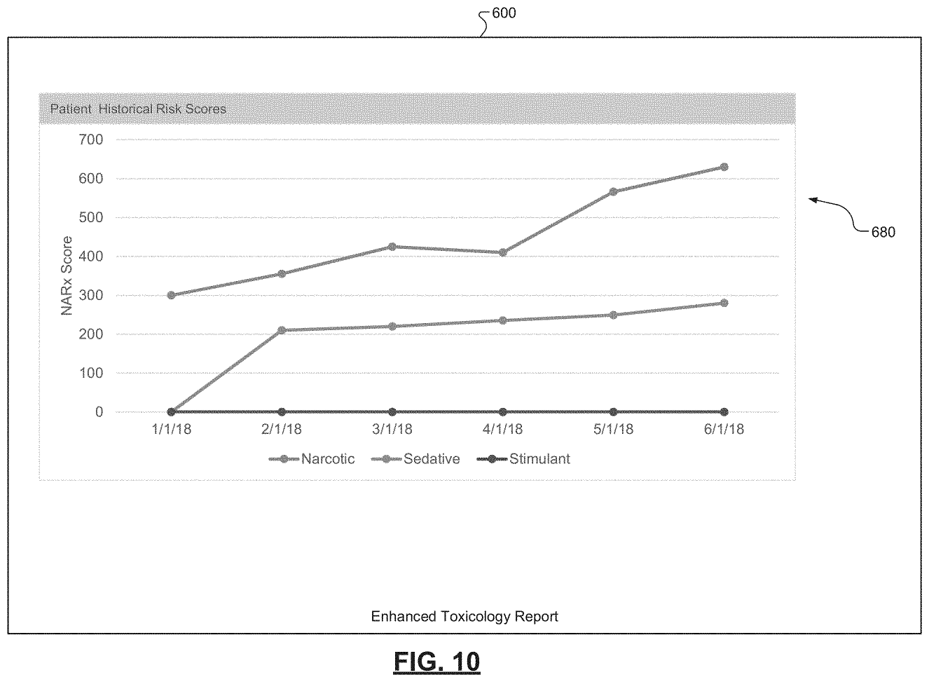

In certain implementations, the enhanced toxicology report 600 can also or alternatively include a historical trend 680 of the determined overdose risk scores 620 of the patient as shown in FIG. 10. The historical trend 680 of the overdose risk score 620 can be a line graph (as shown in FIG. 10) or any other suitable format that provides a simple, visual indication of the changes in the determined overdose risk scores 620 of the patient. It should be appreciated that the enhanced toxicology report 600 can include any one or any combination of the features illustrated in FIG. 6-FIG. 10. Further, in some aspects a recipient of the enhanced toxicology report 600 can originally be presented with a brief summary of the information contained within the enhanced toxicology report 600. Through interaction with links, tabs, or other user interface elements similar to a webpage, the recipient may switch between various views and information in the enhanced toxicology report 600. The ability to switch between various views and presentations of the information can be beneficial to the various recipients of the enhanced toxicology report 600, e.g., in order to quickly and simply find the information most relevant to a particular recipient.

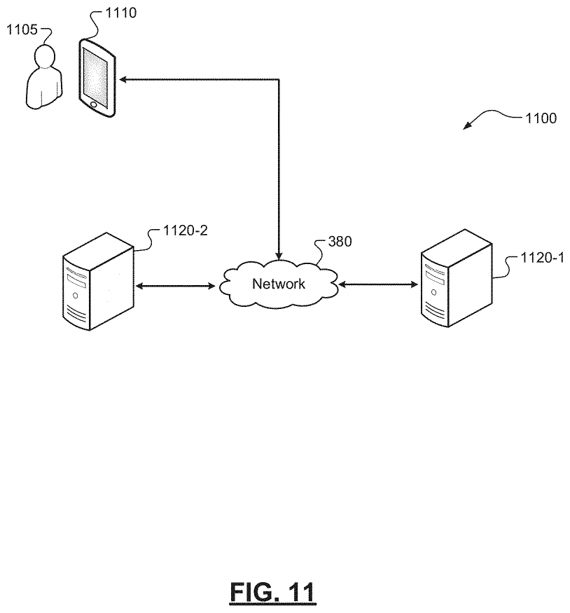

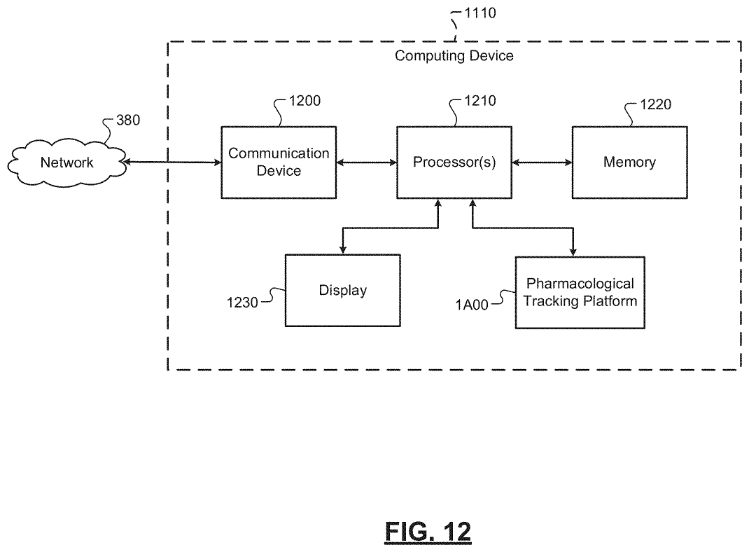

Referring now to FIG. 11, a diagram of an example computing system 1100 is illustrated. The computing system 1100 can be configured to implement the pharmacological tracking platform 100 described herein, e.g., amongst a plurality of users 1105 via their computing devices. The computing system 1100 can include one or more example computing devices 1110 and one or more example servers 1120 that communicate via a network 380 (as described above) according to some implementations of the present disclosure. For ease of description, in this application and as shown in FIG. 11, one example computing device 1110 and two example server computing devices 1120 (server computing devices 1120-1 and 1120-2) are illustrated and described. It should be appreciated, however, that there can be more computing devices 1110 and more or less server computing devices 1120 than is illustrated. While illustrated as a mobile phone (a “smart” phone), each computing device 1110 can be any type of suitable computing device, such as a desktop computer, a tablet computer, a laptop computer, a wearable computing device such as eyewear, a watch or other piece of jewelry, or clothing that incorporates a computing device. A functional block diagram of an example computing device 1110 is illustrated in FIG. 12.

The computing device 1110 is shown as including a communication device 1200, one more processors 1210, a memory 1220, a display device 1230, and the pharmacological tracking platform 100. The processor(s) 1210 can control the operation of the computing device 1110, including implementing at least a portion of the techniques of the present disclosure. The term “processor” as used herein is intended to refer to both a single processor and multiple processors operating together, e.g., in a parallel or distributed architecture.

The communication device 1200 can be configured for communication with other devices (e.g., the server computing devices 1120 or other computing devices 1110) via the network 380. One non-limiting example of the communication device 1200 is a transceiver, although other forms of hardware are within the scope of the present disclosure. The memory 1220 can be any suitable storage medium (flash, hard disk, etc.) configured to store information. For example, the memory 1220 may store a set of instructions that are executable by the processor 1210, which cause the computing device 1110 to perform operations (e.g., such as the operations of the present disclosure). The display device 1130 can display information to the user 1105. In some implementations, the display device 1230 can comprise a touch-sensitive display device (such as a capacitive touchscreen and the like), although non-touch display devices are within the scope of the present disclosure.

It should be appreciated that the example server computing devices 1120 can include the same or similar components as the computing device 1110, and thus can be configured to perform some or all of the techniques of the present disclosure. Further, while the techniques of the present disclosure are described herein in the context of the pharmacological tracking platform 100, which is illustrated as being a component of the computing device 1110, it is specifically contemplated that each feature of the techniques may be performed by a single computing device 1110 alone, a plurality of computing devices 1110 operating together, a server computing device 1120 alone, a plurality of server computing devices 1120 operating together, and a combination of one or more computing devices 110 and one or more server computing devices 1120 operating together.

In embodiments, the platform 100 may be configured to predict diabetes. The platform 100 may also be configured to predict prediabetes. By way of these examples, the platform may predict in a patient prior to diagnosis of diabetes or prediabetes by a medical professional. In these embodiments, the platform may predict diabetes or prediabetes based holistic medical data, based on data from human interaction techniques, and one or more combinations thereof. The holistic medical data and the human interaction techniques may include one or more of customer data matching, panel matching, incentivization or activities, and overlays of health care data. In some embodiments, the determination of diabetes or prediabetes can be based on the matching and similarities found in the holistic medical data and data from human interaction techniques that may be derived with and implemented by using deep learning techniques.

In embodiments, the platform 100 may be configured to calculate a clinical diabetes risk score. The platform 100 may also be configured to calculate a clinical prediabetes risk score. The platform may be configured to identify characteristics indicative of prediabetes or diabetes such as by using one or more of clinical variables, biological variables, and polymorphisms. By way of these examples, the platform can be configured to provide a clinical diabetes risk score based on the identification of characteristics that predict later diabetes using variables available in the clinical setting as well as biological variables and polymorphisms. In embodiments, the platform has been shown to facilitate conclusions that can be shown to support that one very effective clinical predictor of diabetes is adiposity and baseline glucose can be a very effective biological predictor. In many examples, observations of adiposity and baseline glucose may be shown to outweigh conclusions based on clinical and biological predictors subject to gender differences or affected by genetic polymorphisms.

In many instances, there are many examples of detailing the risk for diabetes or prediabetes such as those based on the anthropometric variables associated with diabetic levels of fasting glucose and found that BMI, waist circumference, and waist-to-hip ratio, and other suitable holistic medical data and data from various human interaction techniques.

In embodiments, the platform 100 may be configured to calculate patient risk regarding a plurality of medical conditions, such as diabetes, heart disease, cancer, and osteoporosis. The platform may be configured to calculate the patient risk via one or more of panel data matching, multistage analytics, semantic model building, axiomatic model building, and data analysis, monitoring, and filtering, such as over a period of time. In some embodiments, the platform may use data derived from a health risk assessment (HRA) to calculate the patient risk. The HRA may include one or more of a questionnaire, a risk score, and a report including feedback, the feedback including potential areas of improvement. The questionnaire may include one or more questions directed to the patient regarding nutrition, fitness, biometrics such as blood pressure and/or cholesterol, stress, sleep, and mental health. The report including feedback may include feedback related risk of chronic conditions such as heart disease, diabetes, cancer, and obesity.

In embodiments, the platform may be configured to perform multi-stage analysis to handle and parse patient data. The multi-stage analysis may include analysis using an empirical model such as a machine learning model, a deep learning model, or a combination thereof. The multi-stage analysis may include analysis using a semantic model, such as a model that allows for deep semantic information to be constructed relating to data. The multi-stage analysis may include rules relating to application of the semantic model. The multi-stage analysis may include rules implemented by one or more medical technologists. In some embodiments, the semantic model may be or include one or more abstracted semantics-based descriptions of data, the capturing and/or representing a meaning of data and/or how data is shared and/or propagated through the platform.

In embodiments, the various systems and methods of the present disclosure include a medical records system configured for analyzing workflow for clinical professionals. The system can include a healthcare database configured to store a plurality of medical records. The medical records include: demographic records including one or more of height, weight, sex, gender, ethnicity, and age of a plurality of patients; diagnosis records including medical diagnoses of the patients; prescription records including drugs previously prescribed to the patient; and testing records including tests ordered for the patients, tests performed on the patients, and results of tests performed on the patients, the testing history including names and codes used to identify the tests by health care centers and labs ordering and/or administering the tests. The system can include a machine learning device in communication with the healthcare database and configured to receive the demographic records, the diagnosis records, the prescription records, and the testing records from the healthcare database. The machine learning device is configured to train an artificial intelligence based on the demographic records, the diagnosis records, the prescription records, and the testing records. The artificial intelligence is trained to identify inconsistencies in the names and/or codes used by the health care centers and labs and normalize the names and codes used to identify the tests by health care centers such that similar tests are identified by consistent names and/or codes within the healthcare database. The artificial intelligence is trained to analyze the tests being ordered by individual health care centers of the health care centers to determine redundancies in testing by the individual health care centers. The artificial intelligence is also trained to analyze the demographic records, diagnosis records, prescription records, and testing records to identify inconsistencies in diagnosing, drug prescribing, and test ordering practices of the health care facilities, identify potential improvements to the diagnosing, drug prescribing, and test ordering practices of the health care facilities, and identify medically unnecessary and/or redundant diagnosing, drug prescribing, and test ordering practices of the health care facilities.

In embodiments, the platform 100 may store data such as patient data and/or clinical data in one or more arrays and process data in a flexible manner such that data is readily accessible to end users for querying. The platform may be configured such that an end user may query the data using one or more taxonomy-based query tools, such as SQL or SparQL, thereby facilitating data query by graphic user interfaces and/or natural language-based query interfaces.

In embodiments, the platform 100 may include a convolutional neural network configured to identify patterns in data and use the patterns to make determinations, such as determinations related to a patient, a healthcare provider, and/or a treatment plan or regimen. The determinations may relate to evaluation of efficacy of a healthcare provider and/or a treatment plan or regimen, and/or to prediction or and/or recommendation of a healthcare provider and/or treatment plan or regimen. The convolutional neural network may combine patient data such as genetics and prescription data to identify patterns. In some embodiments, the convolutional neural network may also use data related to patient behavior to identify patterns. In some embodiments, the convolutional neural network may use one or more underlying factors such as economic wealth, education and/or general health and lifestyle of a patient to recognize patterns and/or make determinations. In some embodiments, the platform may be configured to use the convolutional neural network and patterns derived therefrom to determine optimal outcomes for a patient and/or determine paths to the determined optimal outcome, such as patient recovery, disease remission, and/or elimination or substantial decrease of patient risk factors.

In embodiments, the platform includes and/or implements a feedback loop to analyze and/or organize health care and patient data from one or more healthcare providers and patients. The feedback loop may intake data related to treatment acceptance, social media response, one or more responses and/or reactions to a medication and/or a treatment program, or any other suitable type of information.

In embodiments, the various systems and methods of the present disclosure include a medical records system configured for identifying abuse and/or misuse practices of a medical patient and a healthcare database in communication with a state-based prescription drug monitoring program database and configured to store a plurality of medical records. The medical records include demographic records including one or more of height, weight, sex, gender, ethnicity, and age of the patient; diagnosis records including medical diagnoses of the patient; and prescription records including drugs previously prescribed to the patient, drugs being taken by the patient as reported by the patient, and drug prescription records of the patient received from the prescription drug monitoring program database. The medical records system is configured to identify abuse and/or misuse of prescription drugs by the patient based on the drugs previously prescribed, the drugs being taken, and the drug prescription records received from the prescription drug monitoring program database.

In embodiments, the platform may include a data exchange revenue management module configured to provide optionality to a patient regarding treatment options versus price.

In embodiments, the platform is configured to store a demographic dataset that facilitates matching of clinical trials to suitable patients therefor based on demographics of the patients. The demographics may be related to patients within regional boundaries and/or may include one or more of optimization factors, economic wealth, population demographics, and outcome prediction.

In embodiments, the platform may include a referral engine configured to facilitate, track, and/or analyze referrals of patients to and from health care providers. The referral engine may implement one or more balancing factors to account for economic considerations related to patient referrals.

In embodiments, the platform may include a master record configured to serve as a reliable reference related to one or more of patients, health care providers, treatments plans, and any other suitable data. The master record may include handled data such as analyzed, filtered, and/or deduplicated data. In some embodiments, analyzing, filtering, and/or deduplicating via the platform for the master record may include use of machine learning, fuzzy logic, neural networks, or any other suitable process for data handling. The platform may aggregate and analyze, filter, and/or deduplicate data from a plurality of sources, such as health care databases, electronic medical records, state prescription drug monitoring programs (PDMPs), or any other suitable source of data. For example, the platform may be configured to process patient data related to a particular patient name, the patient name being related to one or more other categories of patient information, such as physiological data, treatment history, and prescription history, and the patient name and patient information being aggregated from different and inconsistent sources. The platform may analyze the patient name and the related patient information via a neural network and make a determination to consolidate, deduplicate, correct, and/or normalize the patient name and patient information such that the patient name and the patient information are reliably correlated with one another and stored in the master record. In some embodiments, patient information stored in the master record and handled data related thereto may include genetic data. The platform may be configured to use genetic data to predict and/or determine family and/or lineage of one or patients and may use determined family and/or lineage information for further predictions and analyses. In some embodiments, the platform may be configured to determine one or more ethnic origins of a patient name or collection of related patient names and use determined ethnic origins to identify and correct for misspellings and/or other inconsistencies between possibly related patient data.

In some embodiments, the platform may include a panel matching engine configured to identify, account for, and/or correct inconsistencies in treatment and/or test panel records. Healthcare providers may use inconsistent names and/or identifiers for one or more treatment panels and/or test panels that are substantially equivalent. The panel matching engine is configured to analyze treatment and/or test panel data to consolidate, deduplicate, and/or normalize test panels. In some embodiments, the panel matching engine may implement one or more of machine learning, fuzzy logic, and/or neural networks to handle treatment and/or test panel data. For example, where a particular healthcare system uses inconsistent identifiers for a standard test panel, or where two different healthcare systems use inconsistent identifiers for the standard test panel, the panel matching engine may intake the inconsistent identifiers, determine that each of the inconsistent identifiers is used to identify the standard test panel, and correlate each of the inconsistent identifiers to the standard test panel within databases of the platform such that the platform and modules and engines thereof may consistently process and analyze the test panel and information related thereto.

In embodiments, the platform may be configured to integrate patient data related to a lifestyle of a patient with other patient data. The platform may predict lifestyle data and integrated predicted lifestyle data with other patient data. Other patient data integrated with lifestyle data may include demographic data, prescription history, treatment history, diagnosis history, genetic information, name, age, social security number, and/or any other suitable patient data.

In embodiments, the platform may be configured to recommend test panels for one or more patients. Recommendations of test panels may be derived from patient data, previous panel results, costs of test panels to patients and/or health care providers, or any other suitable information. In some embodiments, the platform may implement machine learning, fuzzy logic, and/or a neural network in test pane recommendation.

In embodiments, the various systems and methods of the present disclosure include a medical records system configured for integrating traditional medical records with lifestyle, wellness, and physiological monitoring information and disseminating the same. The system includes a healthcare database configured to store a plurality of medical records. The medical records include demographic records including one or more of height, weight, sex, gender, ethnicity, and age of a plurality of patients; diagnosis records including medical diagnoses of the patients; prescription records including drugs previously prescribed to the patient; testing records including tests ordered for the patients, tests performed on the patients, and results of tests performed on the patients, the testing history including names and codes used to identify the tests by health care centers and labs ordering and/or administering the tests. The healthcare database is further configured to store lifestyle and wellness records of the patients, the lifestyle and wellness information including information related to one or more of diet, smoking, drinking, and exercise habits. The healthcare database is configured to transmit the medical records and the lifestyle and wellness records to health care facilities and to third parties.

In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises an insurance module that collects physician information from insurance records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises a hospital module that collects physician information from hospital records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises an insurance module that collects physician information from insurance records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises a hospital module that collects physician information from hospital records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises an incident tracking module that collects physician information from disciplinary records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises a waste module that determines if the patient has taken unnecessary or redundant tests. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises a prediction module that analyzes tests the patient has taken, their results, and comparisons with other aggregate information, and recommends additional tests the user could take in order to detect additional conditions. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system activates a workflow to identify the source of the tests that preceded the drop in productivity and automatically activates a quality review of the source. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system activates a workflow to identify the equipment used and automatically orders a quality review of specific testing equipment. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further suggests panels related to the existing tests done. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further suggests additional diagnostics relevant to a patient based on 3rd party integrated information aggregated about the patient in question. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further presents pop-down menus containing auto-completion fields derived from the annotations. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system presents auto-suggest candidates unique to the laboratory, the doctor, or the type of test. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the method further comprises activating a workflow to follow up with hospitals or physicians based on affect analysis. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system applies rules to recommend a review of test equipment function. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system applies rules to recommend a review of personnel efficiency. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system applies rules to recommend a review of the ordering patterns of a physician, an ER, a location, a patient, a hospital, or a lab. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the rules consist of a sequence of testing that should be followed. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further analyzes the cost impact of the rule violation. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the rules relate to efficient blood utilization. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further analyzes relatively better treatment outcomes for the costs of delivering services comparing utilization of one or more tests relative to under-utilization or omission of the one or more tests. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where additional information related to the nature of the event is acquired from a news source. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where a physician or other human agent is prompted to indicate if a large emergency event has occurred and to characterize the type of event. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the event is selected from a list comprising fire, earthquake, mass shooting, explosion, automobile accident, building collapse, hurricane. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the workflow notifies a physician that a match has been done and provides a countdown for how soon the blood must be utilized. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where a workflow collects further information to analyze the likelihood that a blood transfusion was correctly ordered. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the workflow notifies a physician how much time is left to return a blood bag to a container. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the workflow requests additional information from a physician as to why a transfusion was ordered. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises an education module that informs the prescribing physician which metabolites can be expected based on the patients known prescriptions. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the system further comprises a recommendation module that provides the physician with an indicated likelihood that the patient is abusing and a risk report for the physician to work with the patient. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the workflow provides intervention or drug protocol instructions to a physician to apply to a patient. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the data analyzed is X, Y and the pathology is diabetes. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for normalizing data in a medical database comprising, using machine learning to characterize a set of normalized data, applying an ML classification to a new set of unlabeled data, generating metadata and adding labelling metadata to the uncharacterized data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the metadata is a type of physician and the data used to identify the physician is their billing pattern. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the metadata is the classification of a test code in a hierarchy. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the metadata includes a specific set of fast Healthcare interoperability data formats and elements, other HL7 data format and elements, or the like. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for generating inferences comprising a parser, a customer identification module, a health inference engine, and a genetic conflict inference engine. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and where the data sets are selected from the group comprising health care clinical, claims, genomic, immunochemistry, transactional, ordering data, demographic data, Lifestyle and social health data, patient relationships, and patient behaviors. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a memory; and a processor, the processor configured at least to: identify a requested laboratory report; associate the laboratory report with a PDMP; identify one or more laboratory result data; trigger a Drug Consistency Awareness (DCA) service corresponding to the PDMP; send the one or more laboratory result data to a destination corresponding to the laboratory report; and send one or more parameters associated with the DCA to the destination corresponding to the laboratory report. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and providing patient matching comprising collecting a set of patient demographic information from a. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and raising an exception when an unmapped value comes to the PDMP provider. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and mapping test names to PDMP values on the gateway data provider. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and mapping test names to PDMP values. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and dynamically adjusting CRM Layout, Dashboards, or Reports in response to customer activations. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and technique for detecting doc shopping. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, and a correlation module that ensures that the data is associated with the correct physician record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises an insurance module that collects physician information from insurance records. In embodiments, provided herein is a system where the system further comprises an insurance module that collects physician information from insurance records and where the system further comprises a hospital module that collects physician information from hospital records. In embodiments, provided herein is a system where the system further comprises an insurance module that collects physician information from insurance records and for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous. In embodiments, provided herein is a system where the system further comprises an insurance module that collects physician information from insurance records and where the system further comprises an insurance module that collects physician information from insurance records. In embodiments, provided herein is a system where the system further comprises an insurance module that collects physician information from insurance records and where the system further comprises a hospital module that collects physician information from hospital records. In embodiments, provided herein is a system where the system further comprises an insurance module that collects physician information from insurance records and where the system further comprises an incident tracking module that collects physician information from disciplinary records. In embodiments, provided herein is a system where the system further comprises an insurance module that collects physician information from insurance records and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system where the system further comprises an insurance module that collects physician information from insurance records and where the system further comprises a waste module that determines if the patient has taken unnecessary or redundant tests. In embodiments, provided herein is a system where the system further comprises an insurance module that collects physician information from insurance records and where the system further comprises a prediction module that analyzes tests the patient has taken, their results, and comparisons with other aggregate information, and recommends additional tests the user could take in order to detect additional conditions. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises an insurance module that collects physician information from insurance records and where the system activates a workflow to identify the source of the tests that preceded the drop in productivity and automatically activates a quality review of the source. In embodiments, provided herein is a system where the system further comprises an insurance module that collects physician information from insurance records and where the system activates a workflow to identify the equipment used and automatically orders a quality review of specific testing equipment. In embodiments, provided herein is a system where the system further comprises an insurance module that collects physician information from insurance records and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises an insurance module that collects physician information from insurance records and where the system further suggests panels related to the existing tests done. In embodiments, provided herein is a system where the system further comprises an insurance module that collects physician information from insurance records and where the system further suggests additional diagnostics relevant to a patient based on 3rd party integrated information aggregated about the patient in question. In embodiments, provided herein is a system where the system further comprises an insurance module that collects physician information from insurance records and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises an insurance module that collects physician information from insurance records and where the system further presents pop-down menus containing auto-completion fields derived from the annotations. In embodiments, provided herein is a system where the system further comprises an insurance module that collects physician information from insurance records and where the system presents auto-suggest candidates unique to the laboratory, the doctor, or the type of test. In embodiments, provided herein is a system where the system further comprises an insurance module that collects physician information from insurance records and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises an insurance module that collects physician information from insurance records and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises an insurance module that collects physician information from insurance records and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises an insurance module that collects physician information from insurance records and where the method further comprises activating a workflow to follow up with hospitals or physicians based on affect analysis. In embodiments, provided herein is a system where the system further comprises an insurance module that collects physician information from insurance records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises an insurance module that collects physician information from insurance records and where the system applies rules to recommend a review of test equipment function. In embodiments, provided herein is a system where the system further comprises an insurance module that collects physician information from insurance records and where the system applies rules to recommend a review of personnel efficiency. In embodiments, provided herein is a system where the system further comprises an insurance module that collects physician information from insurance records and where the system applies rules to recommend a review of the ordering patterns of a physician, an ER, a location, a patient, a hospital, or a lab. In embodiments, provided herein is a system where the system further comprises an insurance module that collects physician information from insurance records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises an insurance module that collects physician information from insurance records and where the rules consist of a sequence of testing that should be followed. In embodiments, provided herein is a system where the system further comprises an insurance module that collects physician information from insurance records and where the system further analyzes the cost impact of the rule violation. In embodiments, provided herein is a system where the system further comprises an insurance module that collects physician information from insurance records and where the rules relate to efficient blood utilization. In embodiments, provided herein is a system where the system further comprises an insurance module that collects physician information from insurance records and where the system further analyzes relatively better treatment outcomes for the costs of delivering services comparing utilization of one or more tests relative to under-utilization or omission of the one or more tests. In embodiments, provided herein is a system where the system further comprises an insurance module that collects physician information from insurance records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises an insurance module that collects physician information from insurance records and where additional information related to the nature of the event is acquired from a news source. In embodiments, provided herein is a system where the system further comprises an insurance module that collects physician information from insurance records and where a physician or other human agent is prompted to indicate if a large emergency event has occurred and to characterize the type of event. In embodiments, provided herein is a system where the system further comprises an insurance module that collects physician information from insurance records and where the event is selected from a list comprising fire, earthquake, mass shooting, explosion, automobile accident, building collapse, hurricane. In embodiments, provided herein is a system where the system further comprises an insurance module that collects physician information from insurance records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises an insurance module that collects physician information from insurance records and where the workflow notifies a physician that a match has been done and provides a countdown for how soon the blood must be utilized. In embodiments, provided herein is a system where the system further comprises an insurance module that collects physician information from insurance records and where a workflow collects further information to analyze the likelihood that a blood transfusion was correctly ordered. In embodiments, provided herein is a system where the system further comprises an insurance module that collects physician information from insurance records and where the workflow notifies a physician how much time is left to return a blood bag to a container. In embodiments, provided herein is a system where the system further comprises an insurance module that collects physician information from insurance records and where the workflow requests additional information from a physician as to why a transfusion was ordered. In embodiments, provided herein is a system where the system further comprises an insurance module that collects physician information from insurance records and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises an insurance module that collects physician information from insurance records and where the system further comprises an education module that informs the prescribing physician which metabolites can be expected based on the patients known prescriptions. In embodiments, provided herein is a system where the system further comprises an insurance module that collects physician information from insurance records and where the system further comprises a recommendation module that provides the physician with an indicated likelihood that the patient is abusing and a risk report for the physician to work with the patient. In embodiments, provided herein is a system where the system further comprises an insurance module that collects physician information from insurance records and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises an insurance module that collects physician information from insurance records and where the workflow provides intervention or drug protocol instructions to a physician to apply to a patient. In embodiments, provided herein is a system where the system further comprises an insurance module that collects physician information from insurance records and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises an insurance module that collects physician information from insurance records and where the data analyzed is X, Y and the pathology is diabetes. In embodiments, provided herein is a system where the system further comprises an insurance module that collects physician information from insurance records and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises an insurance module that collects physician information from insurance records and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises an insurance module that collects physician information from insurance records and where the metadata is a type of physician and the data used to identify the physician is their billing pattern. In embodiments, provided herein is a system where the system further comprises an insurance module that collects physician information from insurance records and where the metadata is the classification of a test code in a hierarchy. In embodiments, provided herein is a system where the system further comprises an insurance module that collects physician information from insurance records and where the metadata includes a specific set of fast Healthcare interoperability data formats and elements, other HL7 data format and elements, or the like. In embodiments, provided herein is a system where the system further comprises an insurance module that collects physician information from insurance records and for generating inferences comprising a parser, a customer identification module, a health inference engine, and a genetic conflict inference engine. In embodiments, provided herein is a system where the system further comprises an insurance module that collects physician information from insurance records and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises an insurance module that collects physician information from insurance records and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises an insurance module that collects physician information from insurance records and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises an insurance module that collects physician information from insurance records and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises an insurance module that collects physician information from insurance records and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises an insurance module that collects physician information from insurance records and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises an insurance module that collects physician information from insurance records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises an insurance module that collects physician information from insurance records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises an insurance module that collects physician information from insurance records and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises an insurance module that collects physician information from insurance records and providing patient matching comprising collecting a set of patient demographic information from a. In embodiments, provided herein is a system where the system further comprises an insurance module that collects physician information from insurance records and raising an exception when an unmapped value comes to the PDMP provider. In embodiments, provided herein is a system where the system further comprises an insurance module that collects physician information from insurance records and mapping test names to PDMP values on the gateway data provider. In embodiments, provided herein is a system where the system further comprises an insurance module that collects physician information from insurance records and mapping test names to PDMP values. In embodiments, provided herein is a system where the system further comprises an insurance module that collects physician information from insurance records and dynamically adjusting CRM Layout, Dashboards, or Reports in response to customer activations. In embodiments, provided herein is a system where the system further comprises an insurance module that collects physician information from insurance records and technique for detecting doc shopping. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises an insurance module that collects physician information from insurance records and ingesting, by a healthcare insight computing device, patient data of a patient received from one of a plurality of patient data providers; determining, by to a raw data cluster, wherein ingesting the patient data received from the patient data provider comprises staging the patient data in a data staging area. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises an insurance module that collects physician information from insurance records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises an insurance module that collects physician information from insurance records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises an insurance module that collects physician information from insurance records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises a hospital module that collects physician information from hospital records. In embodiments, provided herein is a system where the system further comprises a hospital module that collects physician information from hospital records and for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous. In embodiments, provided herein is a system where the system further comprises a hospital module that collects physician information from hospital records and where the system further comprises an insurance module that collects physician information from insurance records. In embodiments, provided herein is a system where the system further comprises a hospital module that collects physician information from hospital records and where the system further comprises a hospital module that collects physician information from hospital records. In embodiments, provided herein is a system where the system further comprises a hospital module that collects physician information from hospital records and where the system further comprises an incident tracking module that collects physician information from disciplinary records. In embodiments, provided herein is a system where the system further comprises a hospital module that collects physician information from hospital records and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system where the system further comprises a hospital module that collects physician information from hospital records and where the system further comprises a waste module that determines if the patient has taken unnecessary or redundant tests. In embodiments, provided herein is a system where the system further comprises a hospital module that collects physician information from hospital records and where the system further comprises a prediction module that analyzes tests the patient has taken, their results, and comparisons with other aggregate information, and recommends additional tests the user could take in order to detect additional conditions. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises a hospital module that collects physician information from hospital records and where the system activates a workflow to identify the source of the tests that preceded the drop in productivity and automatically activates a quality review of the source. In embodiments, provided herein is a system where the system further comprises a hospital module that collects physician information from hospital records and where the system activates a workflow to identify the equipment used and automatically orders a quality review of specific testing equipment. In embodiments, provided herein is a system where the system further comprises a hospital module that collects physician information from hospital records and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises a hospital module that collects physician information from hospital records and where the system further suggests panels related to the existing tests done. In embodiments, provided herein is a system where the system further comprises a hospital module that collects physician information from hospital records and where the system further suggests additional diagnostics relevant to a patient based on 3rd party integrated information aggregated about the patient in question. In embodiments, provided herein is a system where the system further comprises a hospital module that collects physician information from hospital records and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises a hospital module that collects physician information from hospital records and where the system further presents pop-down menus containing auto-completion fields derived from the annotations. In embodiments, provided herein is a system where the system further comprises a hospital module that collects physician information from hospital records and where the system presents auto-suggest candidates unique to the laboratory, the doctor, or the type of test. In embodiments, provided herein is a system where the system further comprises a hospital module that collects physician information from hospital records and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises a hospital module that collects physician information from hospital records and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises a hospital module that collects physician information from hospital records and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises a hospital module that collects physician information from hospital records and where the method further comprises activating a workflow to follow up with hospitals or physicians based on affect analysis. In embodiments, provided herein is a system where the system further comprises a hospital module that collects physician information from hospital records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises a hospital module that collects physician information from hospital records and where the system applies rules to recommend a review of test equipment function. In embodiments, provided herein is a system where the system further comprises a hospital module that collects physician information from hospital records and where the system applies rules to recommend a review of personnel efficiency. In embodiments, provided herein is a system where the system further comprises a hospital module that collects physician information from hospital records and where the system applies rules to recommend a review of the ordering patterns of a physician, an ER, a location, a patient, a hospital, or a lab. In embodiments, provided herein is a system where the system further comprises a hospital module that collects physician information from hospital records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises a hospital module that collects physician information from hospital records and where the rules consist of a sequence of testing that should be followed. In embodiments, provided herein is a system where the system further comprises a hospital module that collects physician information from hospital records and where the system further analyzes the cost impact of the rule violation. In embodiments, provided herein is a system where the system further comprises a hospital module that collects physician information from hospital records and where the rules relate to efficient blood utilization. In embodiments, provided herein is a system where the system further comprises a hospital module that collects physician information from hospital records and where the system further analyzes relatively better treatment outcomes for the costs of delivering services comparing utilization of one or more tests relative to under-utilization or omission of the one or more tests. In embodiments, provided herein is a system where the system further comprises a hospital module that collects physician information from hospital records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises a hospital module that collects physician information from hospital records and where additional information related to the nature of the event is acquired from a news source. In embodiments, provided herein is a system where the system further comprises a hospital module that collects physician information from hospital records and where a physician or other human agent is prompted to indicate if a large emergency event has occurred and to characterize the type of event. In embodiments, provided herein is a system where the system further comprises a hospital module that collects physician information from hospital records and where the event is selected from a list comprising fire, earthquake, mass shooting, explosion, automobile accident, building collapse, hurricane. In embodiments, provided herein is a system where the system further comprises a hospital module that collects physician information from hospital records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises a hospital module that collects physician information from hospital records and where the workflow notifies a physician that a match has been done and provides a countdown for how soon the blood must be utilized. In embodiments, provided herein is a system where the system further comprises a hospital module that collects physician information from hospital records and where a workflow collects further information to analyze the likelihood that a blood transfusion was correctly ordered. In embodiments, provided herein is a system where the system further comprises a hospital module that collects physician information from hospital records and where the workflow notifies a physician how much time is left to return a blood bag to a container. In embodiments, provided herein is a system where the system further comprises a hospital module that collects physician information from hospital records and where the workflow requests additional information from a physician as to why a transfusion was ordered. In embodiments, provided herein is a system where the system further comprises a hospital module that collects physician information from hospital records and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises a hospital module that collects physician information from hospital records and where the system further comprises an education module that informs the prescribing physician which metabolites can be expected based on the patients known prescriptions. In embodiments, provided herein is a system where the system further comprises a hospital module that collects physician information from hospital records and where the system further comprises a recommendation module that provides the physician with an indicated likelihood that the patient is abusing and a risk report for the physician to work with the patient. In embodiments, provided herein is a system where the system further comprises a hospital module that collects physician information from hospital records and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises a hospital module that collects physician information from hospital records and where the workflow provides intervention or drug protocol instructions to a physician to apply to a patient. In embodiments, provided herein is a system where the system further comprises a hospital module that collects physician information from hospital records and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises a hospital module that collects physician information from hospital records and where the data analyzed is X, Y and the pathology is diabetes. In embodiments, provided herein is a system where the system further comprises a hospital module that collects physician information from hospital records and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises a hospital module that collects physician information from hospital records and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises a hospital module that collects physician information from hospital records and where the metadata is a type of physician and the data used to identify the physician is their billing pattern. In embodiments, provided herein is a system where the system further comprises a hospital module that collects physician information from hospital records and where the metadata is the classification of a test code in a hierarchy. In embodiments, provided herein is a system where the system further comprises a hospital module that collects physician information from hospital records and where the metadata includes a specific set of fast Healthcare interoperability data formats and elements, other HL7 data format and elements, or the like. In embodiments, provided herein is a system where the system further comprises a hospital module that collects physician information from hospital records and for generating inferences comprising a parser, a customer identification module, a health inference engine, and a genetic conflict inference engine. In embodiments, provided herein is a system where the system further comprises a hospital module that collects physician information from hospital records and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises a hospital module that collects physician information from hospital records and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises a hospital module that collects physician information from hospital records and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises a hospital module that collects physician information from hospital records and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises a hospital module that collects physician information from hospital records and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises a hospital module that collects physician information from hospital records and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises a hospital module that collects physician information from hospital records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises a hospital module that collects physician information from hospital records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises a hospital module that collects physician information from hospital records and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises a hospital module that collects physician information from hospital records and providing patient matching comprising collecting a set of patient demographic information from a. In embodiments, provided herein is a system where the system further comprises a hospital module that collects physician information from hospital records and raising an exception when an unmapped value comes to the PDMP provider. In embodiments, provided herein is a system where the system further comprises a hospital module that collects physician information from hospital records and mapping test names to PDMP values on the gateway data provider. In embodiments, provided herein is a system where the system further comprises a hospital module that collects physician information from hospital records and mapping test names to PDMP values. In embodiments, provided herein is a system where the system further comprises a hospital module that collects physician information from hospital records and dynamically adjusting CRM Layout, Dashboards, or Reports in response to customer activations. In embodiments, provided herein is a system where the system further comprises a hospital module that collects physician information from hospital records and technique for detecting doc shopping. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises a hospital module that collects physician information from hospital records and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises a hospital module that collects physician information from hospital records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises a hospital module that collects physician information from hospital records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises a hospital module that collects physician information from hospital records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises an insurance module that collects physician information from insurance records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises a hospital module that collects physician information from hospital records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises an incident tracking module that collects physician information from disciplinary records. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises a waste module that determines if the patient has taken unnecessary or redundant tests. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises a prediction module that analyzes tests the patient has taken, their results, and comparisons with other aggregate information, and recommends additional tests the user could take in order to detect additional conditions. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system activates a workflow to identify the source of the tests that preceded the drop in productivity and automatically activates a quality review of the source. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system activates a workflow to identify the equipment used and automatically orders a quality review of specific testing equipment. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further suggests panels related to the existing tests done. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further suggests additional diagnostics relevant to a patient based on 3rd party integrated information aggregated about the patient in question. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further presents pop-down menus containing auto-completion fields derived from the annotations. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system presents auto-suggest candidates unique to the laboratory, the doctor, or the type of test. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the method further comprises activating a workflow to follow up with hospitals or physicians based on affect analysis. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system applies rules to recommend a review of test equipment function. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system applies rules to recommend a review of personnel efficiency. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system applies rules to recommend a review of the ordering patterns of a physician, an ER, a location, a patient, a hospital, or a lab. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the rules consist of a sequence of testing that should be followed. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further analyzes the cost impact of the rule violation. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the rules relate to efficient blood utilization. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further analyzes relatively better treatment outcomes for the costs of delivering services comparing utilization of one or more tests relative to under-utilization or omission of the one or more tests. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where additional information related to the nature of the event is acquired from a news source. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where a physician or other human agent is prompted to indicate if a large emergency event has occurred and to characterize the type of event. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the event is selected from a list comprising fire, earthquake, mass shooting, explosion, automobile accident, building collapse, hurricane. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the workflow notifies a physician that a match has been done and provides a countdown for how soon the blood must be utilized. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where a workflow collects further information to analyze the likelihood that a blood transfusion was correctly ordered. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the workflow notifies a physician how much time is left to return a blood bag to a container. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the workflow requests additional information from a physician as to why a transfusion was ordered. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises an education module that informs the prescribing physician which metabolites can be expected based on the patients known prescriptions. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the system further comprises a recommendation module that provides the physician with an indicated likelihood that the patient is abusing and a risk report for the physician to work with the patient. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the workflow provides intervention or drug protocol instructions to a physician to apply to a patient. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the data analyzed is X, Y and the pathology is diabetes. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for normalizing data in a medical database comprising, using machine learning to characterize a set of normalized data, applying an ML classification to a new set of unlabeled data, generating metadata and adding labelling metadata to the uncharacterized data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the metadata is a type of physician and the data used to identify the physician is their billing pattern. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the metadata is the classification of a test code in a hierarchy. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the metadata includes a specific set of fast Healthcare interoperability data formats and elements, other HL7 data format and elements, or the like. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for generating inferences comprising a parser, a customer identification module, a health inference engine, and a genetic conflict inference engine. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and where the data sets are selected from the group comprising health care clinical, claims, genomic, immunochemistry, transactional, ordering data, demographic data, Lifestyle and social health data, patient relationships, and patient behaviors. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and providing patient matching comprising collecting a set of patient demographic information from a. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and raising an exception when an unmapped value comes to the PDMP provider. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and mapping test names to PDMP values on the gateway data provider. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and mapping test names to PDMP values. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and dynamically adjusting CRM Layout, Dashboards, or Reports in response to customer activations. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and technique for detecting doc shopping. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for characterizing the activities of an individual physician in a health care drug prescription system, comprising: an interception module for retrieving information from a PDMP relating to the physician, an interaction module identifying each sales and service representatives that the physician has interacted with, an ordering module identifying each of the organizations the physician orders from, a correlation module that ensures that the data is associated with the correct physician record, and an analytics module that analyzes the lab ordering pattern of the physician in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises an insurance module that collects physician information from insurance records. In embodiments, provided herein is a system where the system further comprises an insurance module that collects physician information from insurance records and where the system further comprises a hospital module that collects physician information from hospital records. In embodiments, provided herein is a system where the system further comprises an insurance module that collects physician information from insurance records and where the system further comprises an incident tracking module that collects physician information from disciplinary records. In embodiments, provided herein is a system where the system further comprises an insurance module that collects physician information from insurance records and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system where the system further comprises an insurance module that collects physician information from insurance records and where the system further comprises a waste module that determines if the patient has taken unnecessary or redundant tests. In embodiments, provided herein is a system where the system further comprises an insurance module that collects physician information from insurance records and where the system further comprises a prediction module that analyzes tests the patient has taken, their results, and comparisons with other aggregate information, and recommends additional tests the user could take in order to detect additional conditions. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises an insurance module that collects physician information from insurance records and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises an insurance module that collects physician information from insurance records and where the system activates a workflow to identify the source of the tests that preceded the drop in productivity and automatically activates a quality review of the source. In embodiments, provided herein is a system where the system further comprises an insurance module that collects physician information from insurance records and where the system activates a workflow to identify the equipment used and automatically orders a quality review of specific testing equipment. In embodiments, provided herein is a system where the system further comprises an insurance module that collects physician information from insurance records and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises an insurance module that collects physician information from insurance records and where the system further suggests panels related to the existing tests done. In embodiments, provided herein is a system where the system further comprises an insurance module that collects physician information from insurance records and where the system further suggests additional diagnostics relevant to a patient based on 3rd party integrated information aggregated about the patient in question. In embodiments, provided herein is a system where the system further comprises an insurance module that collects physician information from insurance records and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises an insurance module that collects physician information from insurance records and where the system further presents pop-down menus containing auto-completion fields derived from the annotations. In embodiments, provided herein is a system where the system further comprises an insurance module that collects physician information from insurance records and where the system presents auto-suggest candidates unique to the laboratory, the doctor, or the type of test. In embodiments, provided herein is a system where the system further comprises an insurance module that collects physician information from insurance records and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises an insurance module that collects physician information from insurance records and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises an insurance module that collects physician information from insurance records and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises an insurance module that collects physician information from insurance records and where the method further comprises activating a workflow to follow up with hospitals or physicians based on affect analysis. In embodiments, provided herein is a system where the system further comprises an insurance module that collects physician information from insurance records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises an insurance module that collects physician information from insurance records and where the system applies rules to recommend a review of test equipment function. In embodiments, provided herein is a system where the system further comprises an insurance module that collects physician information from insurance records and where the system applies rules to recommend a review of personnel efficiency. In embodiments, provided herein is a system where the system further comprises an insurance module that collects physician information from insurance records and where the system applies rules to recommend a review of the ordering patterns of a physician, an ER, a location, a patient, a hospital, or a lab. In embodiments, provided herein is a system where the system further comprises an insurance module that collects physician information from insurance records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises an insurance module that collects physician information from insurance records and where the rules consist of a sequence of testing that should be followed. In embodiments, provided herein is a system where the system further comprises an insurance module that collects physician information from insurance records and where the system further analyzes the cost impact of the rule violation. In embodiments, provided herein is a system where the system further comprises an insurance module that collects physician information from insurance records and where the rules relate to efficient blood utilization. In embodiments, provided herein is a system where the system further comprises an insurance module that collects physician information from insurance records and where the system further analyzes relatively better treatment outcomes for the costs of delivering services comparing utilization of one or more tests relative to under-utilization or omission of the one or more tests. In embodiments, provided herein is a system where the system further comprises an insurance module that collects physician information from insurance records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises an insurance module that collects physician information from insurance records and where additional information related to the nature of the event is acquired from a news source. In embodiments, provided herein is a system where the system further comprises an insurance module that collects physician information from insurance records and where a physician or other human agent is prompted to indicate if a large emergency event has occurred and to characterize the type of event. In embodiments, provided herein is a system where the system further comprises an insurance module that collects physician information from insurance records and where the event is selected from a list comprising fire, earthquake, mass shooting, explosion, automobile accident, building collapse, hurricane. In embodiments, provided herein is a system where the system further comprises an insurance module that collects physician information from insurance records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises an insurance module that collects physician information from insurance records and where the workflow notifies a physician that a match has been done and provides a countdown for how soon the blood must be utilized. In embodiments, provided herein is a system where the system further comprises an insurance module that collects physician information from insurance records and where a workflow collects further information to analyze the likelihood that a blood transfusion was correctly ordered. In embodiments, provided herein is a system where the system further comprises an insurance module that collects physician information from insurance records and where the workflow notifies a physician how much time is left to return a blood bag to a container. In embodiments, provided herein is a system where the system further comprises an insurance module that collects physician information from insurance records and where the workflow requests additional information from a physician as to why a transfusion was ordered. In embodiments, provided herein is a system where the system further comprises an insurance module that collects physician information from insurance records and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises an insurance module that collects physician information from insurance records and where the system further comprises an education module that informs the prescribing physician which metabolites can be expected based on the patients known prescriptions. In embodiments, provided herein is a system where the system further comprises an insurance module that collects physician information from insurance records and where the system further comprises a recommendation module that provides the physician with an indicated likelihood that the patient is abusing and a risk report for the physician to work with the patient. In embodiments, provided herein is a system where the system further comprises an insurance module that collects physician information from insurance records and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises an insurance module that collects physician information from insurance records and where the workflow provides intervention or drug protocol instructions to a physician to apply to a patient. In embodiments, provided herein is a system where the system further comprises an insurance module that collects physician information from insurance records and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises an insurance module that collects physician information from insurance records and where the data analyzed is X, Y and the pathology is diabetes. In embodiments, provided herein is a system where the system further comprises an insurance module that collects physician information from insurance records and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises an insurance module that collects physician information from insurance records and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises an insurance module that collects physician information from insurance records and where the metadata is a type of physician and the data used to identify the physician is their billing pattern. In embodiments, provided herein is a system where the system further comprises an insurance module that collects physician information from insurance records and where the metadata is the classification of a test code in a hierarchy. In embodiments, provided herein is a system where the system further comprises an insurance module that collects physician information from insurance records and where the metadata includes a specific set of fast Healthcare interoperability data formats and elements, other HL7 data format and elements, or the like. In embodiments, provided herein is a system where the system further comprises an insurance module that collects physician information from insurance records and for generating inferences comprising a parser, a customer identification module, a health inference engine, and a genetic conflict inference engine. In embodiments, provided herein is a system where the system further comprises an insurance module that collects physician information from insurance records and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises an insurance module that collects physician information from insurance records and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises an insurance module that collects physician information from insurance records and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises an insurance module that collects physician information from insurance records and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises an insurance module that collects physician information from insurance records and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises an insurance module that collects physician information from insurance records and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises an insurance module that collects physician information from insurance records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises an insurance module that collects physician information from insurance records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises an insurance module that collects physician information from insurance records and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises an insurance module that collects physician information from insurance records and providing patient matching comprising collecting a set of patient demographic information from a. In embodiments, provided herein is a system where the system further comprises an insurance module that collects physician information from insurance records and raising an exception when an unmapped value comes to the PDMP provider. In embodiments, provided herein is a system where the system further comprises an insurance module that collects physician information from insurance records and mapping test names to PDMP values on the gateway data provider. In embodiments, provided herein is a system where the system further comprises an insurance module that collects physician information from insurance records and mapping test names to PDMP values. In embodiments, provided herein is a system where the system further comprises an insurance module that collects physician information from insurance records and dynamically adjusting CRM Layout, Dashboards, or Reports in response to customer activations. In embodiments, provided herein is a system where the system further comprises an insurance module that collects physician information from insurance records and technique for detecting doc shopping. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises an insurance module that collects physician information from insurance records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises an insurance module that collects physician information from insurance records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises an insurance module that collects physician information from insurance records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises an insurance module that collects physician information from insurance records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises an insurance module that collects physician information from insurance records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises an insurance module that collects physician information from insurance records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises a hospital module that collects physician information from hospital records. In embodiments, provided herein is a system where the system further comprises a hospital module that collects physician information from hospital records and where the system further comprises an incident tracking module that collects physician information from disciplinary records. In embodiments, provided herein is a system where the system further comprises a hospital module that collects physician information from hospital records and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system where the system further comprises a hospital module that collects physician information from hospital records and where the system further comprises a waste module that determines if the patient has taken unnecessary or redundant tests. In embodiments, provided herein is a system where the system further comprises a hospital module that collects physician information from hospital records and where the system further comprises a prediction module that analyzes tests the patient has taken, their results, and comparisons with other aggregate information, and recommends additional tests the user could take in order to detect additional conditions. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises a hospital module that collects physician information from hospital records and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises a hospital module that collects physician information from hospital records and where the system activates a workflow to identify the source of the tests that preceded the drop in productivity and automatically activates a quality review of the source. In embodiments, provided herein is a system where the system further comprises a hospital module that collects physician information from hospital records and where the system activates a workflow to identify the equipment used and automatically orders a quality review of specific testing equipment. In embodiments, provided herein is a system where the system further comprises a hospital module that collects physician information from hospital records and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises a hospital module that collects physician information from hospital records and where the system further suggests panels related to the existing tests done. In embodiments, provided herein is a system where the system further comprises a hospital module that collects physician information from hospital records and where the system further suggests additional diagnostics relevant to a patient based on 3rd party integrated information aggregated about the patient in question. In embodiments, provided herein is a system where the system further comprises a hospital module that collects physician information from hospital records and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises a hospital module that collects physician information from hospital records and where the system further presents pop-down menus containing auto-completion fields derived from the annotations. In embodiments, provided herein is a system where the system further comprises a hospital module that collects physician information from hospital records and where the system presents auto-suggest candidates unique to the laboratory, the doctor, or the type of test. In embodiments, provided herein is a system where the system further comprises a hospital module that collects physician information from hospital records and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises a hospital module that collects physician information from hospital records and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises a hospital module that collects physician information from hospital records and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises a hospital module that collects physician information from hospital records and where the method further comprises activating a workflow to follow up with hospitals or physicians based on affect analysis. In embodiments, provided herein is a system where the system further comprises a hospital module that collects physician information from hospital records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises a hospital module that collects physician information from hospital records and where the system applies rules to recommend a review of test equipment function. In embodiments, provided herein is a system where the system further comprises a hospital module that collects physician information from hospital records and where the system applies rules to recommend a review of personnel efficiency. In embodiments, provided herein is a system where the system further comprises a hospital module that collects physician information from hospital records and where the system applies rules to recommend a review of the ordering patterns of a physician, an ER, a location, a patient, a hospital, or a lab. In embodiments, provided herein is a system where the system further comprises a hospital module that collects physician information from hospital records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises a hospital module that collects physician information from hospital records and where the rules consist of a sequence of testing that should be followed. In embodiments, provided herein is a system where the system further comprises a hospital module that collects physician information from hospital records and where the system further analyzes the cost impact of the rule violation. In embodiments, provided herein is a system where the system further comprises a hospital module that collects physician information from hospital records and where the rules relate to efficient blood utilization. In embodiments, provided herein is a system where the system further comprises a hospital module that collects physician information from hospital records and where the system further analyzes relatively better treatment outcomes for the costs of delivering services comparing utilization of one or more tests relative to under-utilization or omission of the one or more tests. In embodiments, provided herein is a system where the system further comprises a hospital module that collects physician information from hospital records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises a hospital module that collects physician information from hospital records and where additional information related to the nature of the event is acquired from a news source. In embodiments, provided herein is a system where the system further comprises a hospital module that collects physician information from hospital records and where a physician or other human agent is prompted to indicate if a large emergency event has occurred and to characterize the type of event. In embodiments, provided herein is a system where the system further comprises a hospital module that collects physician information from hospital records and where the event is selected from a list comprising fire, earthquake, mass shooting, explosion, automobile accident, building collapse, hurricane. In embodiments, provided herein is a system where the system further comprises a hospital module that collects physician information from hospital records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises a hospital module that collects physician information from hospital records and where the workflow notifies a physician that a match has been done and provides a countdown for how soon the blood must be utilized. In embodiments, provided herein is a system where the system further comprises a hospital module that collects physician information from hospital records and where a workflow collects further information to analyze the likelihood that a blood transfusion was correctly ordered. In embodiments, provided herein is a system where the system further comprises a hospital module that collects physician information from hospital records and where the workflow notifies a physician how much time is left to return a blood bag to a container. In embodiments, provided herein is a system where the system further comprises a hospital module that collects physician information from hospital records and where the workflow requests additional information from a physician as to why a transfusion was ordered. In embodiments, provided herein is a system where the system further comprises a hospital module that collects physician information from hospital records and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises a hospital module that collects physician information from hospital records and where the system further comprises an education module that informs the prescribing physician which metabolites can be expected based on the patients known prescriptions. In embodiments, provided herein is a system where the system further comprises a hospital module that collects physician information from hospital records and where the system further comprises a recommendation module that provides the physician with an indicated likelihood that the patient is abusing and a risk report for the physician to work with the patient. In embodiments, provided herein is a system where the system further comprises a hospital module that collects physician information from hospital records and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises a hospital module that collects physician information from hospital records and where the workflow provides intervention or drug protocol instructions to a physician to apply to a patient. In embodiments, provided herein is a system where the system further comprises a hospital module that collects physician information from hospital records and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises a hospital module that collects physician information from hospital records and where the data analyzed is X, Y and the pathology is diabetes. In embodiments, provided herein is a system where the system further comprises a hospital module that collects physician information from hospital records and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises a hospital module that collects physician information from hospital records and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises a hospital module that collects physician information from hospital records and where the metadata is a type of physician and the data used to identify the physician is their billing pattern. In embodiments, provided herein is a system where the system further comprises a hospital module that collects physician information from hospital records and where the metadata is the classification of a test code in a hierarchy. In embodiments, provided herein is a system where the system further comprises a hospital module that collects physician information from hospital records and where the metadata includes a specific set of fast Healthcare interoperability data formats and elements, other HL7 data format and elements, or the like. In embodiments, provided herein is a system where the system further comprises a hospital module that collects physician information from hospital records and for generating inferences comprising a parser, a customer identification module, a health inference engine, and a genetic conflict inference engine. In embodiments, provided herein is a system where the system further comprises a hospital module that collects physician information from hospital records and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises a hospital module that collects physician information from hospital records and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises a hospital module that collects physician information from hospital records and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises a hospital module that collects physician information from hospital records and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises a hospital module that collects physician information from hospital records and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises a hospital module that collects physician information from hospital records and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises a hospital module that collects physician information from hospital records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises a hospital module that collects physician information from hospital records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises a hospital module that collects physician information from hospital records and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises a hospital module that collects physician information from hospital records and providing patient matching comprising collecting a set of patient demographic information from a. In embodiments, provided herein is a system where the system further comprises a hospital module that collects physician information from hospital records and raising an exception when an unmapped value comes to the PDMP provider. In embodiments, provided herein is a system where the system further comprises a hospital module that collects physician information from hospital records and mapping test names to PDMP values on the gateway data provider. In embodiments, provided herein is a system where the system further comprises a hospital module that collects physician information from hospital records and mapping test names to PDMP values. In embodiments, provided herein is a system where the system further comprises a hospital module that collects physician information from hospital records and dynamically adjusting CRM Layout, Dashboards, or Reports in response to customer activations. In embodiments, provided herein is a system where the system further comprises a hospital module that collects physician information from hospital records and technique for detecting doc shopping. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises a hospital module that collects physician information from hospital records and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises a hospital module that collects physician information from hospital records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises a hospital module that collects physician information from hospital records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises a hospital module that collects physician information from hospital records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises a hospital module that collects physician information from hospital records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises a hospital module that collects physician information from hospital records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises a hospital module that collects physician information from hospital records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises an incident tracking module that collects physician information from disciplinary records. In embodiments, provided herein is a system where the system further comprises an incident tracking module that collects physician information from disciplinary records and for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system where the system further comprises an incident tracking module that collects physician information from disciplinary records and where the system further comprises a waste module that determines if the patient has taken unnecessary or redundant tests. In embodiments, provided herein is a system where the system further comprises an incident tracking module that collects physician information from disciplinary records and where the system further comprises a prediction module that analyzes tests the patient has taken, their results, and comparisons with other aggregate information, and recommends additional tests the user could take in order to detect additional conditions. In embodiments, provided herein is a system where the system further comprises an incident tracking module that collects physician information from disciplinary records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises an incident tracking module that collects physician information from disciplinary records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises an incident tracking module that collects physician information from disciplinary records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises an incident tracking module that collects physician information from disciplinary records and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises an incident tracking module that collects physician information from disciplinary records and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises an incident tracking module that collects physician information from disciplinary records and where the system activates a workflow to identify the source of the tests that preceded the drop in productivity and automatically activates a quality review of the source. In embodiments, provided herein is a system where the system further comprises an incident tracking module that collects physician information from disciplinary records and where the system activates a workflow to identify the equipment used and automatically orders a quality review of specific testing equipment. In embodiments, provided herein is a system where the system further comprises an incident tracking module that collects physician information from disciplinary records and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises an incident tracking module that collects physician information from disciplinary records and where the system further suggests panels related to the existing tests done. In embodiments, provided herein is a system where the system further comprises an incident tracking module that collects physician information from disciplinary records and where the system further suggests additional diagnostics relevant to a patient based on 3rd party integrated information aggregated about the patient in question. In embodiments, provided herein is a system where the system further comprises an incident tracking module that collects physician information from disciplinary records and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises an incident tracking module that collects physician information from disciplinary records and where the system further presents pop-down menus containing auto-completion fields derived from the annotations. In embodiments, provided herein is a system where the system further comprises an incident tracking module that collects physician information from disciplinary records and where the system presents auto-suggest candidates unique to the laboratory, the doctor, or the type of test. In embodiments, provided herein is a system where the system further comprises an incident tracking module that collects physician information from disciplinary records and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises an incident tracking module that collects physician information from disciplinary records and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises an incident tracking module that collects physician information from disciplinary records and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises an incident tracking module that collects physician information from disciplinary records and where the method further comprises activating a workflow to follow up with hospitals or physicians based on affect analysis. In embodiments, provided herein is a system where the system further comprises an incident tracking module that collects physician information from disciplinary records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises an incident tracking module that collects physician information from disciplinary records and where the system applies rules to recommend a review of test equipment function. In embodiments, provided herein is a system where the system further comprises an incident tracking module that collects physician information from disciplinary records and where the system applies rules to recommend a review of personnel efficiency. In embodiments, provided herein is a system where the system further comprises an incident tracking module that collects physician information from disciplinary records and where the system applies rules to recommend a review of the ordering patterns of a physician, an ER, a location, a patient, a hospital, or a lab. In embodiments, provided herein is a system where the system further comprises an incident tracking module that collects physician information from disciplinary records and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises an incident tracking module that collects physician information from disciplinary records and where the rules consist of a sequence of testing that should be followed. In embodiments, provided herein is a system where the system further comprises an incident tracking module that collects physician information from disciplinary records and where the system further analyzes the cost impact of the rule violation. In embodiments, provided herein is a system where the system further comprises an incident tracking module that collects physician information from disciplinary records and where the rules relate to efficient blood utilization. In embodiments, provided herein is a system where the system further comprises an incident tracking module that collects physician information from disciplinary records and where the system further analyzes relatively better treatment outcomes for the costs of delivering services comparing utilization of one or more tests relative to under-utilization or omission of the one or more tests. In embodiments, provided herein is a system where the system further comprises an incident tracking module that collects physician information from disciplinary records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises an incident tracking module that collects physician information from disciplinary records and where additional information related to the nature of the event is acquired from a news source. In embodiments, provided herein is a system where the system further comprises an incident tracking module that collects physician information from disciplinary records and where a physician or other human agent is prompted to indicate if a large emergency event has occurred and to characterize the type of event. In embodiments, provided herein is a system where the system further comprises an incident tracking module that collects physician information from disciplinary records and where the event is selected from a list comprising fire, earthquake, mass shooting, explosion, automobile accident, building collapse, hurricane. In embodiments, provided herein is a system where the system further comprises an incident tracking module that collects physician information from disciplinary records and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises an incident tracking module that collects physician information from disciplinary records and where the workflow notifies a physician that a match has been done and provides a countdown for how soon the blood must be utilized. In embodiments, provided herein is a system where the system further comprises an incident tracking module that collects physician information from disciplinary records and where a workflow collects further information to analyze the likelihood that a blood transfusion was correctly ordered. In embodiments, provided herein is a system where the system further comprises an incident tracking module that collects physician information from disciplinary records and where the workflow notifies a physician how much time is left to return a blood bag to a container. In embodiments, provided herein is a system where the system further comprises an incident tracking module that collects physician information from disciplinary records and where the workflow requests additional information from a physician as to why a transfusion was ordered. In embodiments, provided herein is a system where the system further comprises an incident tracking module that collects physician information from disciplinary records and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises an incident tracking module that collects physician information from disciplinary records and where the system further comprises an education module that informs the prescribing physician which metabolites can be expected based on the patients known prescriptions. In embodiments, provided herein is a system where the system further comprises an incident tracking module that collects physician information from disciplinary records and where the system further comprises a recommendation module that provides the physician with an indicated likelihood that the patient is abusing and a risk report for the physician to work with the patient. In embodiments, provided herein is a system where the system further comprises an incident tracking module that collects physician information from disciplinary records and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises an incident tracking module that collects physician information from disciplinary records and where the workflow provides intervention or drug protocol instructions to a physician to apply to a patient. In embodiments, provided herein is a system where the system further comprises an incident tracking module that collects physician information from disciplinary records and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises an incident tracking module that collects physician information from disciplinary records and where the data analyzed is X, Y and the pathology is diabetes. In embodiments, provided herein is a system where the system further comprises an incident tracking module that collects physician information from disciplinary records and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises an incident tracking module that collects physician information from disciplinary records and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises an incident tracking module that collects physician information from disciplinary records and where the metadata is a type of physician and the data used to identify the physician is their billing pattern. In embodiments, provided herein is a system where the system further comprises an incident tracking module that collects physician information from disciplinary records and where the metadata is the classification of a test code in a hierarchy. In embodiments, provided herein is a system where the system further comprises an incident tracking module that collects physician information from disciplinary records and where the metadata includes a specific set of fast Healthcare interoperability data formats and elements, other HL7 data format and elements, or the like. In embodiments, provided herein is a system where the system further comprises an incident tracking module that collects physician information from disciplinary records and for generating inferences comprising a parser, a customer identification module, a health inference engine, and a genetic conflict inference engine. In embodiments, provided herein is a system where the system further comprises an incident tracking module that collects physician information from disciplinary records and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises an incident tracking module that collects physician information from disciplinary records and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises an incident tracking module that collects physician information from disciplinary records and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises an incident tracking module that collects physician information from disciplinary records and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises an incident tracking module that collects physician information from disciplinary records and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises an incident tracking module that collects physician information from disciplinary records and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises an incident tracking module that collects physician information from disciplinary records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises an incident tracking module that collects physician information from disciplinary records and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises an incident tracking module that collects physician information from disciplinary records and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises an incident tracking module that collects physician information from disciplinary records and providing patient matching comprising collecting a set of patient demographic information from a. In embodiments, provided herein is a system where the system further comprises an incident tracking module that collects physician information from disciplinary records and raising an exception when an unmapped value comes to the PDMP provider. In embodiments, provided herein is a system where the system further comprises an incident tracking module that collects physician information from disciplinary records and mapping test names to PDMP values on the gateway data provider. In embodiments, provided herein is a system where the system further comprises an incident tracking module that collects physician information from disciplinary records and mapping test names to PDMP values. In embodiments, provided herein is a system where the system further comprises an incident tracking module that collects physician information from disciplinary records and dynamically adjusting CRM Layout, Dashboards, or Reports in response to customer activations. In embodiments, provided herein is a system where the system further comprises an incident tracking module that collects physician information from disciplinary records and technique for detecting doc shopping. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises an incident tracking module that collects physician information from disciplinary records and ingesting, by a healthcare insight computing device, patient data of a patient received from one of a plurality of patient data providers; determining, by data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises an incident tracking module that collects physician information from disciplinary records and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises an incident tracking module that collects physician information from disciplinary records and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises an incident tracking module that collects physician information from disciplinary records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises an incident tracking module that collects physician information from disciplinary records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises an incident tracking module that collects physician information from disciplinary records and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises an incident tracking module that collects physician information from disciplinary records and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further comprises a waste module that determines if the patient has taken unnecessary or redundant tests. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further comprises a prediction module that analyzes tests the patient has taken, their results, and comparisons with other aggregate information, and recommends additional tests the user could take in order to detect additional conditions. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system activates a workflow to identify the source of the tests that preceded the drop in productivity and automatically activates a quality review of the source. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system activates a workflow to identify the equipment used and automatically orders a quality review of specific testing equipment. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further suggests panels related to the existing tests done. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further suggests additional diagnostics relevant to a patient based on 3rd party integrated information aggregated about the patient in question. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further presents pop-down menus containing auto-completion fields derived from the annotations. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system presents auto-suggest candidates unique to the laboratory, the doctor, or the type of test. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the method further comprises activating a workflow to follow up with hospitals or physicians based on affect analysis. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system applies rules to recommend a review of test equipment function. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system applies rules to recommend a review of personnel efficiency. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system applies rules to recommend a review of the ordering patterns of a physician, an ER, a location, a patient, a hospital, or a lab. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the rules consist of a sequence of testing that should be followed. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further analyzes the cost impact of the rule violation. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the rules relate to efficient blood utilization. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further analyzes relatively better treatment outcomes for the costs of delivering services comparing utilization of one or more tests relative to under-utilization or omission of the one or more tests. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where additional information related to the nature of the event is acquired from a news source. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where a physician or other human agent is prompted to indicate if a large emergency event has occurred and to characterize the type of event. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the event is selected from a list comprising fire, earthquake, mass shooting, explosion, automobile accident, building collapse, hurricane. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the workflow notifies a physician that a match has been done and provides a countdown for how soon the blood must be utilized. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where a workflow collects further information to analyze the likelihood that a blood transfusion was correctly ordered. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the workflow notifies a physician how much time is left to return a blood bag to a container. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the workflow requests additional information from a physician as to why a transfusion was ordered. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further comprises an education module that informs the prescribing physician which metabolites can be expected based on the patients known prescriptions. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the system further comprises a recommendation module that provides the physician with an indicated likelihood that the patient is abusing and a risk report for the physician to work with the patient. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the workflow provides intervention or drug protocol instructions to a physician to apply to a patient. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the data analyzed is X, Y and the pathology is diabetes. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for normalizing data in a medical database comprising, using machine learning to characterize a set of normalized data, applying an ML classification to a new set of unlabeled data, generating metadata and adding labelling metadata to the uncharacterized data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the metadata is a type of physician and the data used to identify the physician is their billing pattern. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the metadata is the classification of a test code in a hierarchy. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the metadata includes a specific set of fast Healthcare interoperability data formats and elements, other HL7 data format and elements, or the like. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for generating inferences comprising a parser, a customer identification module, a health inference engine, and a genetic conflict inference engine. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and where the data sets are selected from the group comprising health care clinical, claims, genomic, immunochemistry, transactional, ordering data, demographic data, Lifestyle and social health data, patient relationships, and patient behaviors. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and providing patient matching comprising collecting a set of patient demographic information from a. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and raising an exception when an unmapped value comes to the PDMP provider. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and mapping test names to PDMP values on the gateway data provider. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and mapping test names to PDMP values. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and dynamically adjusting CRM Layout, Dashboards, or Reports in response to customer activations. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and technique for detecting doc shopping. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for characterizing the activities of an individual patients in a health care system, comprising: an interception module for retrieving information from a PDMP relating to the patient, a correlation module that ensures that the data is associated with the correct patient record, and an analytics module that analyzes the lab ordering pattern for the patient in comparison and indicates if a subset of the ordering pattern is anomalous and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises a waste module that determines if the patient has taken unnecessary or redundant tests. In embodiments, provided herein is a system where the system further comprises a waste module that determines if the patient has taken unnecessary or redundant tests and where the system further comprises a prediction module that analyzes tests the patient has taken, their results, and comparisons with other aggregate information, and recommends additional tests the user could take in order to detect additional conditions. In embodiments, provided herein is a system where the system further comprises a waste module that determines if the patient has taken unnecessary or redundant tests and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises a waste module that determines if the patient has taken unnecessary or redundant tests and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises a waste module that determines if the patient has taken unnecessary or redundant tests and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises a waste module that determines if the patient has taken unnecessary or redundant tests and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises a waste module that determines if the patient has taken unnecessary or redundant tests and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises a waste module that determines if the patient has taken unnecessary or redundant tests and where the system activates a workflow to identify the source of the tests that preceded the drop in productivity and automatically activates a quality review of the source. In embodiments, provided herein is a system where the system further comprises a waste module that determines if the patient has taken unnecessary or redundant tests and where the system activates a workflow to identify the equipment used and automatically orders a quality review of specific testing equipment. In embodiments, provided herein is a system where the system further comprises a waste module that determines if the patient has taken unnecessary or redundant tests and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises a waste module that determines if the patient has taken unnecessary or redundant tests and where the system further suggests panels related to the existing tests done. In embodiments, provided herein is a system where the system further comprises a waste module that determines if the patient has taken unnecessary or redundant tests and where the system further suggests additional diagnostics relevant to a patient based on 3rd party integrated information aggregated about the patient in question. In embodiments, provided herein is a system where the system further comprises a waste module that determines if the patient has taken unnecessary or redundant tests and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises a waste module that determines if the patient has taken unnecessary or redundant tests and where the system further presents pop-down menus containing auto-completion fields derived from the annotations. In embodiments, provided herein is a system where the system further comprises a waste module that determines if the patient has taken unnecessary or redundant tests and where the system presents auto-suggest candidates unique to the laboratory, the doctor, or the type of test. In embodiments, provided herein is a system where the system further comprises a waste module that determines if the patient has taken unnecessary or redundant tests and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises a waste module that determines if the patient has taken unnecessary or redundant tests and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises a waste module that determines if the patient has taken unnecessary or redundant tests and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises a waste module that determines if the patient has taken unnecessary or redundant tests and where the method further comprises activating a workflow to follow up with hospitals or physicians based on affect analysis. In embodiments, provided herein is a system where the system further comprises a waste module that determines if the patient has taken unnecessary or redundant tests and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises a waste module that determines if the patient has taken unnecessary or redundant tests and where the system applies rules to recommend a review of test equipment function. In embodiments, provided herein is a system where the system further comprises a waste module that determines if the patient has taken unnecessary or redundant tests and where the system applies rules to recommend a review of personnel efficiency. In embodiments, provided herein is a system where the system further comprises a waste module that determines if the patient has taken unnecessary or redundant tests and where the system applies rules to recommend a review of the ordering patterns of a physician, an ER, a location, a patient, a hospital, or a lab. In embodiments, provided herein is a system where the system further comprises a waste module that determines if the patient has taken unnecessary or redundant tests and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises a waste module that determines if the patient has taken unnecessary or redundant tests and where the rules consist of a sequence of testing that should be followed. In embodiments, provided herein is a system where the system further comprises a waste module that determines if the patient has taken unnecessary or redundant tests and where the system further analyzes the cost impact of the rule violation. In embodiments, provided herein is a system where the system further comprises a waste module that determines if the patient has taken unnecessary or redundant tests and where the rules relate to efficient blood utilization. In embodiments, provided herein is a system where the system further comprises a waste module that determines if the patient has taken unnecessary or redundant tests and where the system further analyzes relatively better treatment outcomes for the costs of delivering services comparing utilization of one or more tests relative to under-utilization or omission of the one or more tests. In embodiments, provided herein is a system where the system further comprises a waste module that determines if the patient has taken unnecessary or redundant tests and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises a waste module that determines if the patient has taken unnecessary or redundant tests and where additional information related to the nature of the event is acquired from a news source. In embodiments, provided herein is a system where the system further comprises a waste module that determines if the patient has taken unnecessary or redundant tests and where a physician or other human agent is prompted to indicate if a large emergency event has occurred and to characterize the type of event. In embodiments, provided herein is a system where the system further comprises a waste module that determines if the patient has taken unnecessary or redundant tests and where the event is selected from a list comprising fire, earthquake, mass shooting, explosion, automobile accident, building collapse, hurricane. In embodiments, provided herein is a system where the system further comprises a waste module that determines if the patient has taken unnecessary or redundant tests and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises a waste module that determines if the patient has taken unnecessary or redundant tests and where the workflow notifies a physician that a match has been done and provides a countdown for how soon the blood must be utilized. In embodiments, provided herein is a system where the system further comprises a waste module that determines if the patient has taken unnecessary or redundant tests and where a workflow collects further information to analyze the likelihood that a blood transfusion was correctly ordered. In embodiments, provided herein is a system where the system further comprises a waste module that determines if the patient has taken unnecessary or redundant tests and where the workflow notifies a physician how much time is left to return a blood bag to a container. In embodiments, provided herein is a system where the system further comprises a waste module that determines if the patient has taken unnecessary or redundant tests and where the workflow requests additional information from a physician as to why a transfusion was ordered. In embodiments, provided herein is a system where the system further comprises a waste module that determines if the patient has taken unnecessary or redundant tests and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises a waste module that determines if the patient has taken unnecessary or redundant tests and where the system further comprises an education module that informs the prescribing physician which metabolites can be expected based on the patients known prescriptions. In embodiments, provided herein is a system where the system further comprises a waste module that determines if the patient has taken unnecessary or redundant tests and where the system further comprises a recommendation module that provides the physician with an indicated likelihood that the patient is abusing and a risk report for the physician to work with the patient. In embodiments, provided herein is a system where the system further comprises a waste module that determines if the patient has taken unnecessary or redundant tests and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises a waste module that determines if the patient has taken unnecessary or redundant tests and where the workflow provides intervention or drug protocol instructions to a physician to apply to a patient. In embodiments, provided herein is a system where the system further comprises a waste module that determines if the patient has taken unnecessary or redundant tests and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises a waste module that determines if the patient has taken unnecessary or redundant tests and where the data analyzed is X, Y and the pathology is diabetes. In embodiments, provided herein is a system where the system further comprises a waste module that determines if the patient has taken unnecessary or redundant tests and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises a waste module that determines if the patient has taken unnecessary or redundant tests and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises a waste module that determines if the patient has taken unnecessary or redundant tests and where the metadata is a type of physician and the data used to identify the physician is their billing pattern. In embodiments, provided herein is a system where the system further comprises a waste module that determines if the patient has taken unnecessary or redundant tests and where the metadata is the classification of a test code in a hierarchy. In embodiments, provided herein is a system where the system further comprises a waste module that determines if the patient has taken unnecessary or redundant tests and where the metadata includes a specific set of fast Healthcare interoperability data formats and elements, other HL7 data format and elements, or the like. In embodiments, provided herein is a system where the system further comprises a waste module that determines if the patient has taken unnecessary or redundant tests and for generating inferences comprising a parser, a customer identification module, a health inference engine, and a genetic conflict inference engine. In embodiments, provided herein is a system where the system further comprises a waste module that determines if the patient has taken unnecessary or redundant tests and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises a waste module that determines if the patient has taken unnecessary or redundant tests and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises a waste module that determines if the patient has taken unnecessary or redundant tests and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises a waste module that determines if the patient has taken unnecessary or redundant tests and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises a waste module that determines if the patient has taken unnecessary or redundant tests and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises a waste module that determines if the patient has taken unnecessary or redundant tests and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises a waste module that determines if the patient has taken unnecessary or redundant tests and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises a waste module that determines if the patient has taken unnecessary or redundant tests and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises a waste module that determines if the patient has taken unnecessary or redundant tests and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises a waste module that determines if the patient has taken unnecessary or redundant tests and providing patient matching comprising collecting a set of patient demographic information from a. In embodiments, provided herein is a system where the system further comprises a waste module that determines if the patient has taken unnecessary or redundant tests and raising an exception when an unmapped value comes to the PDMP provider. In embodiments, provided herein is a system where the system further comprises a waste module that determines if the patient has taken unnecessary or redundant tests and mapping test names to PDMP values on the gateway data provider. In embodiments, provided herein is a system where the system further comprises a waste module that determines if the patient has taken unnecessary or redundant tests and mapping test names to PDMP values. In embodiments, provided herein is a system where the system further comprises a waste module that determines if the patient has taken unnecessary or redundant tests and dynamically adjusting CRM Layout, Dashboards, or Reports in response to customer activations. In embodiments, provided herein is a system where the system further comprises a waste module that determines if the patient has taken unnecessary or redundant tests and technique for detecting doc shopping. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises a waste module that determines if the patient has taken unnecessary or redundant tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises a waste module that determines if the patient has taken unnecessary or redundant tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises a waste module that determines if the patient has taken unnecessary or redundant tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises a waste module that determines if the patient has taken unnecessary or redundant tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises a waste module that determines if the patient has taken unnecessary or redundant tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises a waste module that determines if the patient has taken unnecessary or redundant tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises a prediction module that analyzes tests the patient has taken, their results, and comparisons with other aggregate information, and recommends additional tests the user could take in order to detect additional conditions. In embodiments, provided herein is a system where the system further comprises a prediction module that analyzes tests the patient has taken, their results, and comparisons with other aggregate information, and recommends additional tests the user could take in order to detect additional conditions and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a system where the system further comprises a prediction module that analyzes tests the patient has taken, their results, and comparisons with other aggregate information, and recommends additional tests the user could take in order to detect additional conditions and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a system where the system further comprises a prediction module that analyzes tests the patient has taken, their results, and comparisons with other aggregate information, and recommends additional tests the user could take in order to detect additional conditions and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a system where the system further comprises a prediction module that analyzes tests the patient has taken, their results, and comparisons with other aggregate information, and recommends additional tests the user could take in order to detect additional conditions and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a system where the system further comprises a prediction module that analyzes tests the patient has taken, their results, and comparisons with other aggregate information, and recommends additional tests the user could take in order to detect additional conditions and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a system where the system further comprises a prediction module that analyzes tests the patient has taken, their results, and comparisons with other aggregate information, and recommends additional tests the user could take in order to detect additional conditions and where the system activates a workflow to identify the source of the tests that preceded the drop in productivity and automatically activates a quality review of the source. In embodiments, provided herein is a system where the system further comprises a prediction module that analyzes tests the patient has taken, their results, and comparisons with other aggregate information, and recommends additional tests the user could take in order to detect additional conditions and where the system activates a workflow to identify the equipment used and automatically orders a quality review of specific testing equipment. In embodiments, provided herein is a system where the system further comprises a prediction module that analyzes tests the patient has taken, their results, and comparisons with other aggregate information, and recommends additional tests the user could take in order to detect additional conditions and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a system where the system further comprises a prediction module that analyzes tests the patient has taken, their results, and comparisons with other aggregate information, and recommends additional tests the user could take in order to detect additional conditions and where the system further suggests panels related to the existing tests done. In embodiments, provided herein is a system where the system further comprises a prediction module that analyzes tests the patient has taken, their results, and comparisons with other aggregate information, and recommends additional tests the user could take in order to detect additional conditions and where the system further suggests additional diagnostics relevant to a patient based on 3rd party integrated information aggregated about the patient in question. In embodiments, provided herein is a system where the system further comprises a prediction module that analyzes tests the patient has taken, their results, and comparisons with other aggregate information, and recommends additional tests the user could take in order to detect additional conditions and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a system where the system further comprises a prediction module that analyzes tests the patient has taken, their results, and comparisons with other aggregate information, and recommends additional tests the user could take in order to detect additional conditions and where the system further presents pop-down menus containing auto-completion fields derived from the annotations. In embodiments, provided herein is a system where the system further comprises a prediction module that analyzes tests the patient has taken, their results, and comparisons with other aggregate information, and recommends additional tests the user could take in order to detect additional conditions and where the system presents auto-suggest candidates unique to the laboratory, the doctor, or the type of test. In embodiments, provided herein is a system where the system further comprises a prediction module that analyzes tests the patient has taken, their results, and comparisons with other aggregate information, and recommends additional tests the user could take in order to detect additional conditions and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system where the system further comprises a prediction module that analyzes tests the patient has taken, their results, and comparisons with other aggregate information, and recommends additional tests the user could take in order to detect additional conditions and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system where the system further comprises a prediction module that analyzes tests the patient has taken, their results, and comparisons with other aggregate information, and recommends additional tests the user could take in order to detect additional conditions and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system where the system further comprises a prediction module that analyzes tests the patient has taken, their results, and comparisons with other aggregate information, and recommends additional tests the user could take in order to detect additional conditions and where the method further comprises activating a workflow to follow up with hospitals or physicians based on affect analysis. In embodiments, provided herein is a system where the system further comprises a prediction module that analyzes tests the patient has taken, their results, and comparisons with other aggregate information, and recommends additional tests the user could take in order to detect additional conditions and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system where the system further comprises a prediction module that analyzes tests the patient has taken, their results, and comparisons with other aggregate information, and recommends additional tests the user could take in order to detect additional conditions and where the system applies rules to recommend a review of test equipment function. In embodiments, provided herein is a system where the system further comprises a prediction module that analyzes tests the patient has taken, their results, and comparisons with other aggregate information, and recommends additional tests the user could take in order to detect additional conditions and where the system applies rules to recommend a review of personnel efficiency. In embodiments, provided herein is a system where the system further comprises a prediction module that analyzes tests the patient has taken, their results, and comparisons with other aggregate information, and recommends additional tests the user could take in order to detect additional conditions and where the system applies rules to recommend a review of the ordering patterns of a physician, an ER, a location, a patient, a hospital, or a lab. In embodiments, provided herein is a system where the system further comprises a prediction module that analyzes tests the patient has taken, their results, and comparisons with other aggregate information, and recommends additional tests the user could take in order to detect additional conditions and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system where the system further comprises a prediction module that analyzes tests the patient has taken, their results, and comparisons with other aggregate information, and recommends additional tests the user could take in order to detect additional conditions and where the rules consist of a sequence of testing that should be followed. In embodiments, provided herein is a system where the system further comprises a prediction module that analyzes tests the patient has taken, their results, and comparisons with other aggregate information, and recommends additional tests the user could take in order to detect additional conditions and where the system further analyzes the cost impact of the rule violation. In embodiments, provided herein is a system where the system further comprises a prediction module that analyzes tests the patient has taken, their results, and comparisons with other aggregate information, and recommends additional tests the user could take in order to detect additional conditions and where the rules relate to efficient blood utilization. In embodiments, provided herein is a system where the system further comprises a prediction module that analyzes tests the patient has taken, their results, and comparisons with other aggregate information, and recommends additional tests the user could take in order to detect additional conditions and where the system further analyzes relatively better treatment outcomes for the costs of delivering services comparing utilization of one or more tests relative to under-utilization or omission of the one or more tests. In embodiments, provided herein is a system where the system further comprises a prediction module that analyzes tests the patient has taken, their results, and comparisons with other aggregate information, and recommends additional tests the user could take in order to detect additional conditions and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system where the system further comprises a prediction module that analyzes tests the patient has taken, their results, and comparisons with other aggregate information, and recommends additional tests the user could take in order to detect additional conditions and where additional information related to the nature of the event is acquired from a news source. In embodiments, provided herein is a system where the system further comprises a prediction module that analyzes tests the patient has taken, their results, and comparisons with other aggregate information, and recommends additional tests the user could take in order to detect additional conditions and where a physician or other human agent is prompted to indicate if a large emergency event has occurred and to characterize the type of event. In embodiments, provided herein is a system where the system further comprises a prediction module that analyzes tests the patient has taken, their results, and comparisons with other aggregate information, and recommends additional tests the user could take in order to detect additional conditions and where the event is selected from a list comprising fire, earthquake, mass shooting, explosion, automobile accident, building collapse, hurricane. In embodiments, provided herein is a system where the system further comprises a prediction module that analyzes tests the patient has taken, their results, and comparisons with other aggregate information, and recommends additional tests the user could take in order to detect additional conditions and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system where the system further comprises a prediction module that analyzes tests the patient has taken, their results, and comparisons with other aggregate information, and recommends additional tests the user could take in order to detect additional conditions and where the workflow notifies a physician that a match has been done and provides a countdown for how soon the blood must be utilized. In embodiments, provided herein is a system where the system further comprises a prediction module that analyzes tests the patient has taken, their results, and comparisons with other aggregate information, and recommends additional tests the user could take in order to detect additional conditions and where a workflow collects further information to analyze the likelihood that a blood transfusion was correctly ordered. In embodiments, provided herein is a system where the system further comprises a prediction module that analyzes tests the patient has taken, their results, and comparisons with other aggregate information, and recommends additional tests the user could take in order to detect additional conditions and where the workflow notifies a physician how much time is left to return a blood bag to a container. In embodiments, provided herein is a system where the system further comprises a prediction module that analyzes tests the patient has taken, their results, and comparisons with other aggregate information, and recommends additional tests the user could take in order to detect additional conditions and where the workflow requests additional information from a physician as to why a transfusion was ordered. In embodiments, provided herein is a system where the system further comprises a prediction module that analyzes tests the patient has taken, their results, and comparisons with other aggregate information, and recommends additional tests the user could take in order to detect additional conditions and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system where the system further comprises a prediction module that analyzes tests the patient has taken, their results, and comparisons with other aggregate information, and recommends additional tests the user could take in order to detect additional conditions and where the system further comprises an education module that informs the prescribing physician which metabolites can be expected based on the patients known prescriptions. In embodiments, provided herein is a system where the system further comprises a prediction module that analyzes tests the patient has taken, their results, and comparisons with other aggregate information, and recommends additional tests the user could take in order to detect additional conditions and where the system further comprises a recommendation module that provides the physician with an indicated likelihood that the patient is abusing and a risk report for the physician to work with the patient. In embodiments, provided herein is a system where the system further comprises a prediction module that analyzes tests the patient has taken, their results, and comparisons with other aggregate information, and recommends additional tests the user could take in order to detect additional conditions and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises a prediction module that analyzes tests the patient has taken, their results, and comparisons with other aggregate information, and recommends additional tests the user could take in order to detect additional conditions and where the workflow provides intervention or drug protocol instructions to a physician to apply to a patient. In embodiments, provided herein is a system where the system further comprises a prediction module that analyzes tests the patient has taken, their results, and comparisons with other aggregate information, and recommends additional tests the user could take in order to detect additional conditions and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises a prediction module that analyzes tests the patient has taken, their results, and comparisons with other aggregate information, and recommends additional tests the user could take in order to detect additional conditions and where the data analyzed is X, Y and the pathology is diabetes. In embodiments, provided herein is a system where the system further comprises a prediction module that analyzes tests the patient has taken, their results, and comparisons with other aggregate information, and recommends additional tests the user could take in order to detect additional conditions and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises a prediction module that analyzes tests the patient has taken, their results, and comparisons with other aggregate information, and recommends additional tests the user could take in order to detect additional conditions and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises a prediction module that analyzes tests the patient has taken, their results, and comparisons with other aggregate information, and recommends additional tests the user could take in order to detect additional conditions and where the metadata is a type of physician and the data used to identify the physician is their billing pattern. In embodiments, provided herein is a system where the system further comprises a prediction module that analyzes tests the patient has taken, their results, and comparisons with other aggregate information, and recommends additional tests the user could take in order to detect additional conditions and where the metadata is the classification of a test code in a hierarchy. In embodiments, provided herein is a system where the system further comprises a prediction module that analyzes tests the patient has taken, their results, and comparisons with other aggregate information, and recommends additional tests the user could take in order to detect additional conditions and where the metadata includes a specific set of fast Healthcare interoperability data formats and elements, other HL7 data format and elements, or the like. In embodiments, provided herein is a system where the system further comprises a prediction module that analyzes tests the patient has taken, their results, and comparisons with other aggregate information, and recommends additional tests the user could take in order to detect additional conditions and for generating inferences comprising a parser, a customer identification module, a health inference engine, and a genetic conflict inference engine. In embodiments, provided herein is a system where the system further comprises a prediction module that analyzes tests the patient has taken, their results, and comparisons with other aggregate information, and recommends additional tests the user could take in order to detect additional conditions and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises a prediction module that analyzes tests the patient has taken, their results, and comparisons with other aggregate information, and recommends additional tests the user could take in order to detect additional conditions and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises a prediction module that analyzes tests the patient has taken, their results, and comparisons with other aggregate information, and recommends additional tests the user could take in order to detect additional conditions and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises a prediction module that analyzes tests the patient has taken, their results, and comparisons with other aggregate information, and recommends additional tests the user could take in order to detect additional conditions and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises a prediction module that analyzes tests the patient has taken, their results, and comparisons with other aggregate information, and recommends additional tests the user could take in order to detect additional conditions and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises a prediction module that analyzes tests the patient has taken, their results, and comparisons with other aggregate information, and recommends additional tests the user could take in order to detect additional conditions and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises a prediction module that analyzes tests the patient has taken, their results, and comparisons with other aggregate information, and recommends additional tests the user could take in order to detect additional conditions and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises a prediction module that analyzes tests the patient has taken, their results, and comparisons with other aggregate information, and recommends additional tests the user could take in order to detect additional conditions and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises a prediction module that analyzes tests the patient has taken, their results, and comparisons with other aggregate information, and recommends additional tests the user could take in order to detect additional conditions and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises a prediction module that analyzes tests the patient has taken, their results, and comparisons with other aggregate information, and recommends additional tests the user could take in order to detect additional conditions and providing patient matching comprising collecting a set of patient demographic information from a. In embodiments, provided herein is a system where the system further comprises a prediction module that analyzes tests the patient has taken, their results, and comparisons with other aggregate information, and recommends additional tests the user could take in order to detect additional conditions and raising an exception when an unmapped value comes to the PDMP provider. In embodiments, provided herein is a system where the system further comprises a prediction module that analyzes tests the patient has taken, their results, and comparisons with other aggregate information, and recommends additional tests the user could take in order to detect additional conditions and mapping test names to PDMP values on the gateway data provider. In embodiments, provided herein is a system where the system further comprises a prediction module that analyzes tests the patient has taken, their results, and comparisons with other aggregate information, and recommends additional tests the user could take in order to detect additional conditions and mapping test names to PDMP values. In embodiments, provided herein is a system where the system further comprises a prediction module that analyzes tests the patient has taken, their results, and comparisons with other aggregate information, and recommends additional tests the user could take in order to detect additional conditions and dynamically adjusting CRM Layout, Dashboards, or Reports in response to customer activations. In embodiments, provided herein is a system where the system further comprises a prediction module that analyzes tests the patient has taken, their results, and comparisons with other aggregate information, and recommends additional tests the user could take in order to detect additional conditions and technique for detecting doc shopping. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises a prediction module that analyzes tests the patient has taken, their results, and comparisons with other aggregate information, and recommends additional tests the user could take in order to detect additional conditions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises a prediction module that analyzes tests the patient has taken, their results, and comparisons with other aggregate information, and recommends additional tests the user could take in order to detect additional conditions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises a prediction module that analyzes tests the patient has taken, their results, and comparisons with other aggregate information, and recommends additional tests the user could take in order to detect additional conditions and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises a prediction module that analyzes tests the patient has taken, their results, and comparisons with other aggregate information, and recommends additional tests the user could take in order to detect additional conditions and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises a prediction module that analyzes tests the patient has taken, their results, and comparisons with other aggregate information, and recommends additional tests the user could take in order to detect additional conditions and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises a prediction module that analyzes tests the patient has taken, their results, and comparisons with other aggregate information, and recommends additional tests the user could take in order to detect additional conditions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system activates a workflow to identify the source of the tests that preceded the drop in productivity and automatically activates a quality review of the source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system activates a workflow to identify the equipment used and automatically orders a quality review of specific testing equipment. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further suggests panels related to the existing tests done. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further suggests additional diagnostics relevant to a patient based on 3rd party integrated information aggregated about the patient in question. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further presents pop-down menus containing auto-completion fields derived from the annotations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system presents auto-suggest candidates unique to the laboratory, the doctor, or the type of test. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the method further comprises activating a workflow to follow up with hospitals or physicians based on affect analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system applies rules to recommend a review of test equipment function. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system applies rules to recommend a review of personnel efficiency. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system applies rules to recommend a review of the ordering patterns of a physician, an ER, a location, a patient, a hospital, or a lab. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the rules consist of a sequence of testing that should be followed. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further analyzes the cost impact of the rule violation. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the rules relate to efficient blood utilization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further analyzes relatively better treatment outcomes for the costs of delivering services comparing utilization of one or more tests relative to under-utilization or omission of the one or more tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where additional information related to the nature of the event is acquired from a news source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where a physician or other human agent is prompted to indicate if a large emergency event has occurred and to characterize the type of event. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the event is selected from a list comprising fire, earthquake, mass shooting, explosion, automobile accident, building collapse, hurricane. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the workflow notifies a physician that a match has been done and provides a countdown for how soon the blood must be utilized. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where a workflow collects further information to analyze the likelihood that a blood transfusion was correctly ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the workflow notifies a physician how much time is left to return a blood bag to a container. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the workflow requests additional information from a physician as to why a transfusion was ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further comprises an education module that informs the prescribing physician which metabolites can be expected based on the patients known prescriptions. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the system further comprises a recommendation module that provides the physician with an indicated likelihood that the patient is abusing and a risk report for the physician to work with the patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the workflow provides intervention or drug protocol instructions to a physician to apply to a patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the data analyzed is X, Y and the pathology is diabetes. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the metadata is a type of physician and the data used to identify the physician is their billing pattern. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the metadata is the classification of a test code in a hierarchy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the metadata includes a specific set of fast Healthcare interoperability data formats and elements, other HL7 data format and elements, or the like. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for generating inferences comprising a parser, a customer identification module, a health inference engine, and a genetic conflict inference engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and providing patient matching comprising collecting a set of patient demographic information from a. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and raising an exception when an unmapped value comes to the PDMP provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and mapping test names to PDMP values on the gateway data provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and mapping test names to PDMP values. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and dynamically adjusting CRM Layout, Dashboards, or Reports in response to customer activations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and technique for detecting doc shopping. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summaries grouped with aggregated issues and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system activates a workflow to identify the source of the tests that preceded the drop in productivity and automatically activates a quality review of the source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system activates a workflow to identify the equipment used and automatically orders a quality review of specific testing equipment. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further suggests panels related to the existing tests done. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further suggests additional diagnostics relevant to a patient based on 3rd party integrated information aggregated about the patient in question. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further presents pop-down menus containing auto-completion fields derived from the annotations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system presents auto-suggest candidates unique to the laboratory, the doctor, or the type of test. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the method further comprises activating a workflow to follow up with hospitals or physicians based on affect analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system applies rules to recommend a review of test equipment function. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system applies rules to recommend a review of personnel efficiency. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system applies rules to recommend a review of the ordering patterns of a physician, an ER, a location, a patient, a hospital, or a lab. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the rules consist of a sequence of testing that should be followed. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further analyzes the cost impact of the rule violation. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the rules relate to efficient blood utilization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further analyzes relatively better treatment outcomes for the costs of delivering services comparing utilization of one or more tests relative to under-utilization or omission of the one or more tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where additional information related to the nature of the event is acquired from a news source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where a physician or other human agent is prompted to indicate if a large emergency event has occurred and to characterize the type of event. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the event is selected from a list comprising fire, earthquake, mass shooting, explosion, automobile accident, building collapse, hurricane. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the workflow notifies a physician that a match has been done and provides a countdown for how soon the blood must be utilized. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where a workflow collects further information to analyze the likelihood that a blood transfusion was correctly ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the workflow notifies a physician how much time is left to return a blood bag to a container. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the workflow requests additional information from a physician as to why a transfusion was ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further comprises an education module that informs the prescribing physician which metabolites can be expected based on the patients known prescriptions. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the system further comprises a recommendation module that provides the physician with an indicated likelihood that the patient is abusing and a risk report for the physician to work with the patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the workflow provides intervention or drug protocol instructions to a physician to apply to a patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the data analyzed is X, Y and the pathology is diabetes. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the metadata is a type of physician and the data used to identify the physician is their billing pattern. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the metadata is the classification of a test code in a hierarchy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the metadata includes a specific set of fast Healthcare interoperability data formats and elements, other HL7 data format and elements, or the like. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for generating inferences comprising a parser, a customer identification module, a health inference engine, and a genetic conflict inference engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and providing patient matching comprising collecting a set of patient demographic information from a. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and raising an exception when an unmapped value comes to the PDMP provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and mapping test names to PDMP values on the gateway data provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and mapping test names to PDMP values. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and dynamically adjusting CRM Layout, Dashboards, or Reports in response to customer activations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and technique for detecting doc shopping. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual summary of the issues with a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system activates a workflow to identify the source of the tests that preceded the drop in productivity and automatically activates a quality review of the source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system activates a workflow to identify the equipment used and automatically orders a quality review of specific testing equipment. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further suggests panels related to the existing tests done. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further suggests additional diagnostics relevant to a patient based on 3rd party integrated information aggregated about the patient in question. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further presents pop-down menus containing auto-completion fields derived from the annotations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system presents auto-suggest candidates unique to the laboratory, the doctor, or the type of test. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the method further comprises activating a workflow to follow up with hospitals or physicians based on affect analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system applies rules to recommend a review of test equipment function. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system applies rules to recommend a review of personnel efficiency. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system applies rules to recommend a review of the ordering patterns of a physician, an ER, a location, a patient, a hospital, or a lab. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the rules consist of a sequence of testing that should be followed. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further analyzes the cost impact of the rule violation. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the rules relate to efficient blood utilization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further analyzes relatively better treatment outcomes for the costs of delivering services comparing utilization of one or more tests relative to under-utilization or omission of the one or more tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where additional information related to the nature of the event is acquired from a news source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where a physician or other human agent is prompted to indicate if a large emergency event has occurred and to characterize the type of event. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the event is selected from a list comprising fire, earthquake, mass shooting, explosion, automobile accident, building collapse, hurricane. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the workflow notifies a physician that a match has been done and provides a countdown for how soon the blood must be utilized. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where a workflow collects further information to analyze the likelihood that a blood transfusion was correctly ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the workflow notifies a physician how much time is left to return a blood bag to a container. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the workflow requests additional information from a physician as to why a transfusion was ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further comprises an education module that informs the prescribing physician which metabolites can be expected based on the patients known prescriptions. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the system further comprises a recommendation module that provides the physician with an indicated likelihood that the patient is abusing and a risk report for the physician to work with the patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the workflow provides intervention or drug protocol instructions to a physician to apply to a patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the data analyzed is X, Y and the pathology is diabetes. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the metadata is a type of physician and the data used to identify the physician is their billing pattern. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the metadata is the classification of a test code in a hierarchy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the metadata includes a specific set of fast Healthcare interoperability data formats and elements, other HL7 data format and elements, or the like. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for generating inferences comprising a parser, a customer identification module, a health inference engine, and a genetic conflict inference engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and providing patient matching comprising collecting a set of patient demographic information from a. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and raising an exception when an unmapped value comes to the PDMP provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and mapping test names to PDMP values on the gateway data provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and mapping test names to PDMP values. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and dynamically adjusting CRM Layout, Dashboards, or Reports in response to customer activations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and technique for detecting doc shopping. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, and automatically generating a plain-language textual improvement plan report for a particular laboratory and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system activates a workflow to identify the source of the tests that preceded the drop in productivity and automatically activates a quality review of the source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system activates a workflow to identify the equipment used and automatically orders a quality review of specific testing equipment. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further suggests panels related to the existing tests done. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further suggests additional diagnostics relevant to a patient based on 3rd party integrated information aggregated about the patient in question. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further presents pop-down menus containing auto-completion fields derived from the annotations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system presents auto-suggest candidates unique to the laboratory, the doctor, or the type of test. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the method further comprises activating a workflow to follow up with hospitals or physicians based on affect analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system applies rules to recommend a review of test equipment function. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system applies rules to recommend a review of personnel efficiency. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system applies rules to recommend a review of the ordering patterns of a physician, an ER, a location, a patient, a hospital, or a lab. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the rules consist of a sequence of testing that should be followed. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further analyzes the cost impact of the rule violation. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the rules relate to efficient blood utilization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further analyzes relatively better treatment outcomes for the costs of delivering services comparing utilization of one or more tests relative to under-utilization or omission of the one or more tests. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where additional information related to the nature of the event is acquired from a news source. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where a physician or other human agent is prompted to indicate if a large emergency event has occurred and to characterize the type of event. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the event is selected from a list comprising fire, earthquake, mass shooting, explosion, automobile accident, building collapse, hurricane. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the workflow notifies a physician that a match has been done and provides a countdown for how soon the blood must be utilized. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where a workflow collects further information to analyze the likelihood that a blood transfusion was correctly ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the workflow notifies a physician how much time is left to return a blood bag to a container. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the workflow requests additional information from a physician as to why a transfusion was ordered. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further comprises an education module that informs the prescribing physician which metabolites can be expected based on the patients known prescriptions. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the system further comprises a recommendation module that provides the physician with an indicated likelihood that the patient is abusing and a risk report for the physician to work with the patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the workflow provides intervention or drug protocol instructions to a physician to apply to a patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the data analyzed is X, Y and the pathology is diabetes. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the metadata is a type of physician and the data used to identify the physician is their billing pattern. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the metadata is the classification of a test code in a hierarchy. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the metadata includes a specific set of fast Healthcare interoperability data formats and elements, other HL7 data format and elements, or the like. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for generating inferences comprising a parser, a customer identification module, a health inference engine, and a genetic conflict inference engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and providing patient matching comprising collecting a set of patient demographic information from a. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and raising an exception when an unmapped value comes to the PDMP provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and mapping test names to PDMP values on the gateway data provider. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and mapping test names to PDMP values. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and dynamically adjusting CRM Layout, Dashboards, or Reports in response to customer activations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and technique for detecting doc shopping. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to pre-analytical, analytical, and post-analytical laboratory issues, parsing human-input information relating each of the laboratory issues, combining differently worded descriptions that have the same meaning, mapping the issues to an ontology/entity module containing descriptions of the actors (labs, doctors, ERs, hospitals) in the system, and automatically generating an indication of the most likely entity whose actions was the primary cause of the issue and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system activates a workflow to identify the source of the tests that preceded the drop in productivity and automatically activates a quality review of the source. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system activates a workflow to identify the equipment used and automatically orders a quality review of specific testing equipment. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further suggests panels related to the existing tests done. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further suggests additional diagnostics relevant to a patient based on 3rd party integrated information aggregated about the patient in question. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further presents pop-down menus containing auto-completion fields derived from the annotations. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system presents auto-suggest candidates unique to the laboratory, the doctor, or the type of test. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the method further comprises activating a workflow to follow up with hospitals or physicians based on affect analysis. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system applies rules to recommend a review of test equipment function. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system applies rules to recommend a review of personnel efficiency. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system applies rules to recommend a review of the ordering patterns of a physician, an ER, a location, a patient, a hospital, or a lab. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the rules consist of a sequence of testing that should be followed. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further analyzes the cost impact of the rule violation. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the rules relate to efficient blood utilization. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further analyzes relatively better treatment outcomes for the costs of delivering services comparing utilization of one or more tests relative to under-utilization or omission of the one or more tests. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where additional information related to the nature of the event is acquired from a news source. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where a physician or other human agent is prompted to indicate if a large emergency event has occurred and to characterize the type of event. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the event is selected from a list comprising fire, earthquake, mass shooting, explosion, automobile accident, building collapse, hurricane. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the workflow notifies a physician that a match has been done and provides a countdown for how soon the blood must be utilized. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where a workflow collects further information to analyze the likelihood that a blood transfusion was correctly ordered. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the workflow notifies a physician how much time is left to return a blood bag to a container. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the workflow requests additional information from a physician as to why a transfusion was ordered. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further comprises an education module that informs the prescribing physician which metabolites can be expected based on the patients known prescriptions. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the system further comprises a recommendation module that provides the physician with an indicated likelihood that the patient is abusing and a risk report for the physician to work with the patient. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the workflow provides intervention or drug protocol instructions to a physician to apply to a patient. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the data analyzed is X, Y and the pathology is diabetes. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the metadata is a type of physician and the data used to identify the physician is their billing pattern. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the metadata is the classification of a test code in a hierarchy. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the metadata includes a specific set of fast Healthcare interoperability data formats and elements, other HL7 data format and elements, or the like. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for generating inferences comprising a parser, a customer identification module, a health inference engine, and a genetic conflict inference engine. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and providing patient matching comprising collecting a set of patient demographic information from a. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and raising an exception when an unmapped value comes to the PDMP provider. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and mapping test names to PDMP values on the gateway data provider. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and mapping test names to PDMP values. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and dynamically adjusting CRM Layout, Dashboards, or Reports in response to customer activations. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and technique for detecting doc shopping. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and ingesting, by a healthcare insight computing device, patient data of a patient received from one of a plurality of patient data providers; determining, by the to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the quality or effectiveness of a laboratory comprising aggregating transaction information about a plurality of laboratories over time, analyzing volume and type of test, compiling a set of signals relating to test issues, speed, turnaround time, performance, or personnel, and compiling time utilization and workload statistics for each laboratory and each lab worker and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system activates a workflow to identify the source of the tests that preceded the drop in productivity and automatically activates a quality review of the source. In embodiments, provided herein is a method where the system activates a workflow to identify the source of the tests that preceded the drop in productivity and automatically activates a quality review of the source and where the system activates a workflow to identify the equipment used and automatically orders a quality review of specific testing equipment. In embodiments, provided herein is a method where the system activates a workflow to identify the source of the tests that preceded the drop in productivity and automatically activates a quality review of the source and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method where the system activates a workflow to identify the source of the tests that preceded the drop in productivity and automatically activates a quality review of the source and where the system further suggests panels related to the existing tests done. In embodiments, provided herein is a method where the system activates a workflow to identify the source of the tests that preceded the drop in productivity and automatically activates a quality review of the source and where the system further suggests additional diagnostics relevant to a patient based on 3rd party integrated information aggregated about the patient in question. In embodiments, provided herein is a method where the system activates a workflow to identify the source of the tests that preceded the drop in productivity and automatically activates a quality review of the source and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method where the system activates a workflow to identify the source of the tests that preceded the drop in productivity and automatically activates a quality review of the source and where the system further presents pop-down menus containing auto-completion fields derived from the annotations. In embodiments, provided herein is a method where the system activates a workflow to identify the source of the tests that preceded the drop in productivity and automatically activates a quality review of the source and where the system presents auto-suggest candidates unique to the laboratory, the doctor, or the type of test. In embodiments, provided herein is a method where the system activates a workflow to identify the source of the tests that preceded the drop in productivity and automatically activates a quality review of the source and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method where the system activates a workflow to identify the source of the tests that preceded the drop in productivity and automatically activates a quality review of the source and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method where the system activates a workflow to identify the source of the tests that preceded the drop in productivity and automatically activates a quality review of the source and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the system activates a workflow to identify the source of the tests that preceded the drop in productivity and automatically activates a quality review of the source and where the method further comprises activating a workflow to follow up with hospitals or physicians based on affect analysis. In embodiments, provided herein is a method where the system activates a workflow to identify the source of the tests that preceded the drop in productivity and automatically activates a quality review of the source and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the system activates a workflow to identify the source of the tests that preceded the drop in productivity and automatically activates a quality review of the source and where the system applies rules to recommend a review of test equipment function. In embodiments, provided herein is a method where the system activates a workflow to identify the source of the tests that preceded the drop in productivity and automatically activates a quality review of the source and where the system applies rules to recommend a review of personnel efficiency. In embodiments, provided herein is a method where the system activates a workflow to identify the source of the tests that preceded the drop in productivity and automatically activates a quality review of the source and where the system applies rules to recommend a review of the ordering patterns of a physician, an ER, a location, a patient, a hospital, or a lab. In embodiments, provided herein is a method where the system activates a workflow to identify the source of the tests that preceded the drop in productivity and automatically activates a quality review of the source and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system activates a workflow to identify the source of the tests that preceded the drop in productivity and automatically activates a quality review of the source and where the rules consist of a sequence of testing that should be followed. In embodiments, provided herein is a method where the system activates a workflow to identify the source of the tests that preceded the drop in productivity and automatically activates a quality review of the source and where the system further analyzes the cost impact of the rule violation. In embodiments, provided herein is a method where the system activates a workflow to identify the source of the tests that preceded the drop in productivity and automatically activates a quality review of the source and where the rules relate to efficient blood utilization. In embodiments, provided herein is a method where the system activates a workflow to identify the source of the tests that preceded the drop in productivity and automatically activates a quality review of the source and where the system further analyzes relatively better treatment outcomes for the costs of delivering services comparing utilization of one or more tests relative to under-utilization or omission of the one or more tests. In embodiments, provided herein is a method where the system activates a workflow to identify the source of the tests that preceded the drop in productivity and automatically activates a quality review of the source and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system activates a workflow to identify the source of the tests that preceded the drop in productivity and automatically activates a quality review of the source and where additional information related to the nature of the event is acquired from a news source. In embodiments, provided herein is a method where the system activates a workflow to identify the source of the tests that preceded the drop in productivity and automatically activates a quality review of the source and where a physician or other human agent is prompted to indicate if a large emergency event has occurred and to characterize the type of event. In embodiments, provided herein is a method where the system activates a workflow to identify the source of the tests that preceded the drop in productivity and automatically activates a quality review of the source and where the event is selected from a list comprising fire, earthquake, mass shooting, explosion, automobile accident, building collapse, hurricane. In embodiments, provided herein is a method where the system activates a workflow to identify the source of the tests that preceded the drop in productivity and automatically activates a quality review of the source and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system activates a workflow to identify the source of the tests that preceded the drop in productivity and automatically activates a quality review of the source and where the workflow notifies a physician that a match has been done and provides a countdown for how soon the blood must be utilized. In embodiments, provided herein is a method where the system activates a workflow to identify the source of the tests that preceded the drop in productivity and automatically activates a quality review of the source and where a workflow collects further information to analyze the likelihood that a blood transfusion was correctly ordered. In embodiments, provided herein is a method where the system activates a workflow to identify the source of the tests that preceded the drop in productivity and automatically activates a quality review of the source and where the workflow notifies a physician how much time is left to return a blood bag to a container. In embodiments, provided herein is a method where the system activates a workflow to identify the source of the tests that preceded the drop in productivity and automatically activates a quality review of the source and where the workflow requests additional information from a physician as to why a transfusion was ordered. In embodiments, provided herein is a method where the system activates a workflow to identify the source of the tests that preceded the drop in productivity and automatically activates a quality review of the source and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system activates a workflow to identify the source of the tests that preceded the drop in productivity and automatically activates a quality review of the source and where the system further comprises an education module that informs the prescribing physician which metabolites can be expected based on the patients known prescriptions. In embodiments, provided herein is a method where the system activates a workflow to identify the source of the tests that preceded the drop in productivity and automatically activates a quality review of the source and where the system further comprises a recommendation module that provides the physician with an indicated likelihood that the patient is abusing and a risk report for the physician to work with the patient. In embodiments, provided herein is a method where the system activates a workflow to identify the source of the tests that preceded the drop in productivity and automatically activates a quality review of the source and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system activates a workflow to identify the source of the tests that preceded the drop in productivity and automatically activates a quality review of the source and where the workflow provides intervention or drug protocol instructions to a physician to apply to a patient. In embodiments, provided herein is a method where the system activates a workflow to identify the source of the tests that preceded the drop in productivity and automatically activates a quality review of the source and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system activates a workflow to identify the source of the tests that preceded the drop in productivity and automatically activates a quality review of the source and where the data analyzed is X, Y and the pathology is diabetes. In embodiments, provided herein is a method where the system activates a workflow to identify the source of the tests that preceded the drop in productivity and automatically activates a quality review of the source and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system activates a workflow to identify the source of the tests that preceded the drop in productivity and automatically activates a quality review of the source and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system activates a workflow to identify the source of the tests that preceded the drop in productivity and automatically activates a quality review of the source and where the metadata is a type of physician and the data used to identify the physician is their billing pattern. In embodiments, provided herein is a method where the system activates a workflow to identify the source of the tests that preceded the drop in productivity and automatically activates a quality review of the source and where the metadata is the classification of a test code in a hierarchy. In embodiments, provided herein is a method where the system activates a workflow to identify the source of the tests that preceded the drop in productivity and automatically activates a quality review of the source and where the metadata includes a specific set of fast Healthcare interoperability data formats and elements, other HL7 data format and elements, or the like. In embodiments, provided herein is a method where the system activates a workflow to identify the source of the tests that preceded the drop in productivity and automatically activates a quality review of the source and for generating inferences comprising a parser, a customer identification module, a health inference engine, and a genetic conflict inference engine. In embodiments, provided herein is a method where the system activates a workflow to identify the source of the tests that preceded the drop in productivity and automatically activates a quality review of the source and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system activates a workflow to identify the source of the tests that preceded the drop in productivity and automatically activates a quality review of the source and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system activates a workflow to identify the source of the tests that preceded the drop in productivity and automatically activates a quality review of the source and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system activates a workflow to identify the source of the tests that preceded the drop in productivity and automatically activates a quality review of the source and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system activates a workflow to identify the source of the tests that preceded the drop in productivity and automatically activates a quality review of the source and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system activates a workflow to identify the source of the tests that preceded the drop in productivity and automatically activates a quality review of the source and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system activates a workflow to identify the source of the tests that preceded the drop in productivity and automatically activates a quality review of the source and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system activates a workflow to identify the source of the tests that preceded the drop in productivity and automatically activates a quality review of the source and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system activates a workflow to identify the source of the tests that preceded the drop in productivity and automatically activates a quality review of the source and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system activates a workflow to identify the source of the tests that preceded the drop in productivity and automatically activates a quality review of the source and providing patient matching comprising collecting a set of patient demographic information from a. In embodiments, provided herein is a method where the system activates a workflow to identify the source of the tests that preceded the drop in productivity and automatically activates a quality review of the source and raising an exception when an unmapped value comes to the PDMP provider. In embodiments, provided herein is a method where the system activates a workflow to identify the source of the tests that preceded the drop in productivity and automatically activates a quality review of the source and mapping test names to PDMP values on the gateway data provider. In embodiments, provided herein is a method where the system activates a workflow to identify the source of the tests that preceded the drop in productivity and automatically activates a quality review of the source and mapping test names to PDMP values. In embodiments, provided herein is a method where the system activates a workflow to identify the source of the tests that preceded the drop in productivity and automatically activates a quality review of the source and dynamically adjusting CRM Layout, Dashboards, or Reports in response to customer activations. In embodiments, provided herein is a method where the system activates a workflow to identify the source of the tests that preceded the drop in productivity and automatically activates a quality review of the source and technique for detecting doc shopping. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system activates a workflow to identify the source of the tests that preceded the drop in productivity and automatically activates a quality review of the source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system activates a workflow to identify the source of the tests that preceded the drop in productivity and automatically activates a quality review of the source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system activates a workflow to identify the source of the tests that preceded the drop in productivity and automatically activates a quality review of the source and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system activates a workflow to identify the source of the tests that preceded the drop in productivity and automatically activates a quality review of the source and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system activates a workflow to identify the source of the tests that preceded the drop in productivity and automatically activates a quality review of the source and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system activates a workflow to identify the source of the tests that preceded the drop in productivity and automatically activates a quality review of the source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system activates a workflow to identify the equipment used and automatically orders a quality review of specific testing equipment. In embodiments, provided herein is a method where the system activates a workflow to identify the equipment used and automatically orders a quality review of specific testing equipment and for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method where the system activates a workflow to identify the equipment used and automatically orders a quality review of specific testing equipment and where the system further suggests panels related to the existing tests done. In embodiments, provided herein is a method where the system activates a workflow to identify the equipment used and automatically orders a quality review of specific testing equipment and where the system further suggests additional diagnostics relevant to a patient based on 3rd party integrated information aggregated about the patient in question. In embodiments, provided herein is a method where the system activates a workflow to identify the equipment used and automatically orders a quality review of specific testing equipment and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method where the system activates a workflow to identify the equipment used and automatically orders a quality review of specific testing equipment and where the system further presents pop-down menus containing auto-completion fields derived from the annotations. In embodiments, provided herein is a method where the system activates a workflow to identify the equipment used and automatically orders a quality review of specific testing equipment and where the system presents auto-suggest candidates unique to the laboratory, the doctor, or the type of test. In embodiments, provided herein is a method where the system activates a workflow to identify the equipment used and automatically orders a quality review of specific testing equipment and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method where the system activates a workflow to identify the equipment used and automatically orders a quality review of specific testing equipment and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method where the system activates a workflow to identify the equipment used and automatically orders a quality review of specific testing equipment and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the system activates a workflow to identify the equipment used and automatically orders a quality review of specific testing equipment and where the method further comprises activating a workflow to follow up with hospitals or physicians based on affect analysis. In embodiments, provided herein is a method where the system activates a workflow to identify the equipment used and automatically orders a quality review of specific testing equipment and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the system activates a workflow to identify the equipment used and automatically orders a quality review of specific testing equipment and where the system applies rules to recommend a review of test equipment function. In embodiments, provided herein is a method where the system activates a workflow to identify the equipment used and automatically orders a quality review of specific testing equipment and where the system applies rules to recommend a review of personnel efficiency. In embodiments, provided herein is a method where the system activates a workflow to identify the equipment used and automatically orders a quality review of specific testing equipment and where the system applies rules to recommend a review of the ordering patterns of a physician, an ER, a location, a patient, a hospital, or a lab. In embodiments, provided herein is a method where the system activates a workflow to identify the equipment used and automatically orders a quality review of specific testing equipment and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system activates a workflow to identify the equipment used and automatically orders a quality review of specific testing equipment and where the rules consist of a sequence of testing that should be followed. In embodiments, provided herein is a method where the system activates a workflow to identify the equipment used and automatically orders a quality review of specific testing equipment and where the system further analyzes the cost impact of the rule violation. In embodiments, provided herein is a method where the system activates a workflow to identify the equipment used and automatically orders a quality review of specific testing equipment and where the rules relate to efficient blood utilization. In embodiments, provided herein is a method where the system activates a workflow to identify the equipment used and automatically orders a quality review of specific testing equipment and where the system further analyzes relatively better treatment outcomes for the costs of delivering services comparing utilization of one or more tests relative to under-utilization or omission of the one or more tests. In embodiments, provided herein is a method where the system activates a workflow to identify the equipment used and automatically orders a quality review of specific testing equipment and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system activates a workflow to identify the equipment used and automatically orders a quality review of specific testing equipment and where additional information related to the nature of the event is acquired from a news source. In embodiments, provided herein is a method where the system activates a workflow to identify the equipment used and automatically orders a quality review of specific testing equipment and where a physician or other human agent is prompted to indicate if a large emergency event has occurred and to characterize the type of event. In embodiments, provided herein is a method where the system activates a workflow to identify the equipment used and automatically orders a quality review of specific testing equipment and where the event is selected from a list comprising fire, earthquake, mass shooting, explosion, automobile accident, building collapse, hurricane. In embodiments, provided herein is a method where the system activates a workflow to identify the equipment used and automatically orders a quality review of specific testing equipment and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system activates a workflow to identify the equipment used and automatically orders a quality review of specific testing equipment and where the workflow notifies a physician that a match has been done and provides a countdown for how soon the blood must be utilized. In embodiments, provided herein is a method where the system activates a workflow to identify the equipment used and automatically orders a quality review of specific testing equipment and where a workflow collects further information to analyze the likelihood that a blood transfusion was correctly ordered. In embodiments, provided herein is a method where the system activates a workflow to identify the equipment used and automatically orders a quality review of specific testing equipment and where the workflow notifies a physician how much time is left to return a blood bag to a container. In embodiments, provided herein is a method where the system activates a workflow to identify the equipment used and automatically orders a quality review of specific testing equipment and where the workflow requests additional information from a physician as to why a transfusion was ordered. In embodiments, provided herein is a method where the system activates a workflow to identify the equipment used and automatically orders a quality review of specific testing equipment and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system activates a workflow to identify the equipment used and automatically orders a quality review of specific testing equipment and where the system further comprises an education module that informs the prescribing physician which metabolites can be expected based on the patients known prescriptions. In embodiments, provided herein is a method where the system activates a workflow to identify the equipment used and automatically orders a quality review of specific testing equipment and where the system further comprises a recommendation module that provides the physician with an indicated likelihood that the patient is abusing and a risk report for the physician to work with the patient. In embodiments, provided herein is a method where the system activates a workflow to identify the equipment used and automatically orders a quality review of specific testing equipment and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system activates a workflow to identify the equipment used and automatically orders a quality review of specific testing equipment and where the workflow provides intervention or drug protocol instructions to a physician to apply to a patient. In embodiments, provided herein is a method where the system activates a workflow to identify the equipment used and automatically orders a quality review of specific testing equipment and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system activates a workflow to identify the equipment used and automatically orders a quality review of specific testing equipment and where the data analyzed is X, Y and the pathology is diabetes. In embodiments, provided herein is a method where the system activates a workflow to identify the equipment used and automatically orders a quality review of specific testing equipment and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system activates a workflow to identify the equipment used and automatically orders a quality review of specific testing equipment and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system activates a workflow to identify the equipment used and automatically orders a quality review of specific testing equipment and where the metadata is a type of physician and the data used to identify the physician is their billing pattern. In embodiments, provided herein is a method where the system activates a workflow to identify the equipment used and automatically orders a quality review of specific testing equipment and where the metadata is the classification of a test code in a hierarchy. In embodiments, provided herein is a method where the system activates a workflow to identify the equipment used and automatically orders a quality review of specific testing equipment and where the metadata includes a specific set of fast Healthcare interoperability data formats and elements, other HL7 data format and elements, or the like. In embodiments, provided herein is a method where the system activates a workflow to identify the equipment used and automatically orders a quality review of specific testing equipment and for generating inferences comprising a parser, a customer identification module, a health inference engine, and a genetic conflict inference engine. In embodiments, provided herein is a method where the system activates a workflow to identify the equipment used and automatically orders a quality review of specific testing equipment and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system activates a workflow to identify the equipment used and automatically orders a quality review of specific testing equipment and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system activates a workflow to identify the equipment used and automatically orders a quality review of specific testing equipment and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system activates a workflow to identify the equipment used and automatically orders a quality review of specific testing equipment and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system activates a workflow to identify the equipment used and automatically orders a quality review of specific testing equipment and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system activates a workflow to identify the equipment used and automatically orders a quality review of specific testing equipment and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system activates a workflow to identify the equipment used and automatically orders a quality review of specific testing equipment and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system activates a workflow to identify the equipment used and automatically orders a quality review of specific testing equipment and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system activates a workflow to identify the equipment used and automatically orders a quality review of specific testing equipment and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system activates a workflow to identify the equipment used and automatically orders a quality review of specific testing equipment and providing patient matching comprising collecting a set of patient demographic information from a. In embodiments, provided herein is a method where the system activates a workflow to identify the equipment used and automatically orders a quality review of specific testing equipment and raising an exception when an unmapped value comes to the PDMP provider. In embodiments, provided herein is a method where the system activates a workflow to identify the equipment used and automatically orders a quality review of specific testing equipment and mapping test names to PDMP values on the gateway data provider. In embodiments, provided herein is a method where the system activates a workflow to identify the equipment used and automatically orders a quality review of specific testing equipment and mapping test names to PDMP values. In embodiments, provided herein is a method where the system activates a workflow to identify the equipment used and automatically orders a quality review of specific testing equipment and dynamically adjusting CRM Layout, Dashboards, or Reports in response to customer activations. In embodiments, provided herein is a method where the system activates a workflow to identify the equipment used and automatically orders a quality review of specific testing equipment and technique for detecting doc shopping. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system activates a workflow to identify the equipment used and automatically orders a quality review of specific testing equipment and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system activates a workflow to identify the equipment used and automatically orders a quality review of specific testing equipment and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system activates a workflow to identify the equipment used and automatically orders a quality review of specific testing equipment and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system activates a workflow to identify the equipment used and automatically orders a quality review of specific testing equipment and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system activates a workflow to identify the equipment used and automatically orders a quality review of specific testing equipment and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system activates a workflow to identify the equipment used and automatically orders a quality review of specific testing equipment and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further suggests panels related to the existing tests done. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further suggests additional diagnostics relevant to a patient based on 3rd party integrated information aggregated about the patient in question. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further presents pop-down menus containing auto-completion fields derived from the annotations. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system presents auto-suggest candidates unique to the laboratory, the doctor, or the type of test. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the method further comprises activating a workflow to follow up with hospitals or physicians based on affect analysis. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system applies rules to recommend a review of test equipment function. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system applies rules to recommend a review of personnel efficiency. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system applies rules to recommend a review of the ordering patterns of a physician, an ER, a location, a patient, a hospital, or a lab. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the rules consist of a sequence of testing that should be followed. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further analyzes the cost impact of the rule violation. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the rules relate to efficient blood utilization. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further analyzes relatively better treatment outcomes for the costs of delivering services comparing utilization of one or more tests relative to under-utilization or omission of the one or more tests. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where additional information related to the nature of the event is acquired from a news source. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where a physician or other human agent is prompted to indicate if a large emergency event has occurred and to characterize the type of event. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the event is selected from a list comprising fire, earthquake, mass shooting, explosion, automobile accident, building collapse, hurricane. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the workflow notifies a physician that a match has been done and provides a countdown for how soon the blood must be utilized. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where a workflow collects further information to analyze the likelihood that a blood transfusion was correctly ordered. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the workflow notifies a physician how much time is left to return a blood bag to a container. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the workflow requests additional information from a physician as to why a transfusion was ordered. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further comprises an education module that informs the prescribing physician which metabolites can be expected based on the patients known prescriptions. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the system further comprises a recommendation module that provides the physician with an indicated likelihood that the patient is abusing and a risk report for the physician to work with the patient. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the workflow provides intervention or drug protocol instructions to a physician to apply to a patient. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the data analyzed is X, Y and the pathology is diabetes. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the metadata is a type of physician and the data used to identify the physician is their billing pattern. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the metadata is the classification of a test code in a hierarchy. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the metadata includes a specific set of fast Healthcare interoperability data formats and elements, other HL7 data format and elements, or the like. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for generating inferences comprising a parser, a customer identification module, a health inference engine, and a genetic conflict inference engine. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and where the data sets are selected from the group comprising health care clinical, claims, genomic, immunochemistry, transactional, ordering data, demographic data, Lifestyle and social health data, patient relationships, and patient behaviors. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and providing patient matching comprising collecting a set of patient demographic information from a. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and raising an exception when an unmapped value comes to the PDMP provider. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and mapping test names to PDMP values on the gateway data provider. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and mapping test names to PDMP values. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and dynamically adjusting CRM Layout, Dashboards, or Reports in response to customer activations. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and technique for detecting doc shopping. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for increasing the efficiency of a doctor ordering lab services with a user interface comprising presenting a summary of lab services already ordered, determining related supplies and presenting them in an interface for adding to an order, analyzing existing lab orders to suggest additional tests that could be performed based on samples already taken, suggesting new tests that could be performed that have relevance to the panels being ordered, and providing real-time test results and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system further suggests panels related to the existing tests done. In embodiments, provided herein is a method where the system further suggests panels related to the existing tests done and where the system further suggests additional diagnostics relevant to a patient based on 3rd party integrated information aggregated about the patient in question. In embodiments, provided herein is a method where the system further suggests panels related to the existing tests done and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method where the system further suggests panels related to the existing tests done and where the system further presents pop-down menus containing auto-completion fields derived from the annotations. In embodiments, provided herein is a method where the system further suggests panels related to the existing tests done and where the system presents auto-suggest candidates unique to the laboratory, the doctor, or the type of test. In embodiments, provided herein is a method where the system further suggests panels related to the existing tests done and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method where the system further suggests panels related to the existing tests done and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method where the system further suggests panels related to the existing tests done and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the system further suggests panels related to the existing tests done and where the method further comprises activating a workflow to follow up with hospitals or physicians based on affect analysis. In embodiments, provided herein is a method where the system further suggests panels related to the existing tests done and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the system further suggests panels related to the existing tests done and where the system applies rules to recommend a review of test equipment function. In embodiments, provided herein is a method where the system further suggests panels related to the existing tests done and where the system applies rules to recommend a review of personnel efficiency. In embodiments, provided herein is a method where the system further suggests panels related to the existing tests done and where the system applies rules to recommend a review of the ordering patterns of a physician, an ER, a location, a patient, a hospital, or a lab. In embodiments, provided herein is a method where the system further suggests panels related to the existing tests done and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system further suggests panels related to the existing tests done and where the rules consist of a sequence of testing that should be followed. In embodiments, provided herein is a method where the system further suggests panels related to the existing tests done and where the system further analyzes the cost impact of the rule violation. In embodiments, provided herein is a method where the system further suggests panels related to the existing tests done and where the rules relate to efficient blood utilization. In embodiments, provided herein is a method where the system further suggests panels related to the existing tests done and where the system further analyzes relatively better treatment outcomes for the costs of delivering services comparing utilization of one or more tests relative to under-utilization or omission of the one or more tests. In embodiments, provided herein is a method where the system further suggests panels related to the existing tests done and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system further suggests panels related to the existing tests done and where additional information related to the nature of the event is acquired from a news source. In embodiments, provided herein is a method where the system further suggests panels related to the existing tests done and where a physician or other human agent is prompted to indicate if a large emergency event has occurred and to characterize the type of event. In embodiments, provided herein is a method where the system further suggests panels related to the existing tests done and where the event is selected from a list comprising fire, earthquake, mass shooting, explosion, automobile accident, building collapse, hurricane. In embodiments, provided herein is a method where the system further suggests panels related to the existing tests done and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system further suggests panels related to the existing tests done and where the workflow notifies a physician that a match has been done and provides a countdown for how soon the blood must be utilized. In embodiments, provided herein is a method where the system further suggests panels related to the existing tests done and where a workflow collects further information to analyze the likelihood that a blood transfusion was correctly ordered. In embodiments, provided herein is a method where the system further suggests panels related to the existing tests done and where the workflow notifies a physician how much time is left to return a blood bag to a container. In embodiments, provided herein is a method where the system further suggests panels related to the existing tests done and where the workflow requests additional information from a physician as to why a transfusion was ordered. In embodiments, provided herein is a method where the system further suggests panels related to the existing tests done and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system further suggests panels related to the existing tests done and where the system further comprises an education module that informs the prescribing physician which metabolites can be expected based on the patients known prescriptions. In embodiments, provided herein is a method where the system further suggests panels related to the existing tests done and where the system further comprises a recommendation module that provides the physician with an indicated likelihood that the patient is abusing and a risk report for the physician to work with the patient. In embodiments, provided herein is a method where the system further suggests panels related to the existing tests done and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system further suggests panels related to the existing tests done and where the workflow provides intervention or drug protocol instructions to a physician to apply to a patient. In embodiments, provided herein is a method where the system further suggests panels related to the existing tests done and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system further suggests panels related to the existing tests done and where the data analyzed is X, Y and the pathology is diabetes. In embodiments, provided herein is a method where the system further suggests panels related to the existing tests done and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system further suggests panels related to the existing tests done and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system further suggests panels related to the existing tests done and where the metadata is a type of physician and the data used to identify the physician is their billing pattern. In embodiments, provided herein is a method where the system further suggests panels related to the existing tests done and where the metadata is the classification of a test code in a hierarchy. In embodiments, provided herein is a method where the system further suggests panels related to the existing tests done and where the metadata includes a specific set of fast Healthcare interoperability data formats and elements, other HL7 data format and elements, or the like. In embodiments, provided herein is a method where the system further suggests panels related to the existing tests done and for generating inferences comprising a parser, a customer identification module, a health inference engine, and a genetic conflict inference engine. In embodiments, provided herein is a method where the system further suggests panels related to the existing tests done and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system further suggests panels related to the existing tests done and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system further suggests panels related to the existing tests done and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system further suggests panels related to the existing tests done and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system further suggests panels related to the existing tests done and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system further suggests panels related to the existing tests done and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system further suggests panels related to the existing tests done and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system further suggests panels related to the existing tests done and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system further suggests panels related to the existing tests done and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system further suggests panels related to the existing tests done and providing patient matching comprising collecting a set of patient demographic information from a. In embodiments, provided herein is a method where the system further suggests panels related to the existing tests done and raising an exception when an unmapped value comes to the PDMP provider. In embodiments, provided herein is a method where the system further suggests panels related to the existing tests done and mapping test names to PDMP values on the gateway data provider. In embodiments, provided herein is a method where the system further suggests panels related to the existing tests done and mapping test names to PDMP values. In embodiments, provided herein is a method where the system further suggests panels related to the existing tests done and dynamically adjusting CRM Layout, Dashboards, or Reports in response to customer activations. In embodiments, provided herein is a method where the system further suggests panels related to the existing tests done and technique for detecting doc shopping. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system further suggests panels related to the existing tests done and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system further suggests panels related to the existing tests done and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system further suggests panels related to the existing tests done and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system further suggests panels related to the existing tests done and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system further suggests panels related to the existing tests done and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system further suggests panels related to the existing tests done and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system further suggests additional diagnostics relevant to a patient based on 3rd party integrated information aggregated about the patient in question. In embodiments, provided herein is a method where the system further suggests additional diagnostics relevant to a patient based on 3rd party integrated information aggregated about the patient in question and for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method where the system further suggests additional diagnostics relevant to a patient based on 3rd party integrated information aggregated about the patient in question and where the system further presents pop-down menus containing auto-completion fields derived from the annotations. In embodiments, provided herein is a method where the system further suggests additional diagnostics relevant to a patient based on 3rd party integrated information aggregated about the patient in question and where the system presents auto-suggest candidates unique to the laboratory, the doctor, or the type of test. In embodiments, provided herein is a method where the system further suggests additional diagnostics relevant to a patient based on 3rd party integrated information aggregated about the patient in question and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method where the system further suggests additional diagnostics relevant to a patient based on 3rd party integrated information aggregated about the patient in question and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method where the system further suggests additional diagnostics relevant to a patient based on 3rd party integrated information aggregated about the patient in question and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the system further suggests additional diagnostics relevant to a patient based on 3rd party integrated information aggregated about the patient in question and where the method further comprises activating a workflow to follow up with hospitals or physicians based on affect analysis. In embodiments, provided herein is a method where the system further suggests additional diagnostics relevant to a patient based on 3rd party integrated information aggregated about the patient in question and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the system further suggests additional diagnostics relevant to a patient based on 3rd party integrated information aggregated about the patient in question and where the system applies rules to recommend a review of test equipment function. In embodiments, provided herein is a method where the system further suggests additional diagnostics relevant to a patient based on 3rd party integrated information aggregated about the patient in question and where the system applies rules to recommend a review of personnel efficiency. In embodiments, provided herein is a method where the system further suggests additional diagnostics relevant to a patient based on 3rd party integrated information aggregated about the patient in question and where the system applies rules to recommend a review of the ordering patterns of a physician, an ER, a location, a patient, a hospital, or a lab. In embodiments, provided herein is a method where the system further suggests additional diagnostics relevant to a patient based on 3rd party integrated information aggregated about the patient in question and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system further suggests additional diagnostics relevant to a patient based on 3rd party integrated information aggregated about the patient in question and where the rules consist of a sequence of testing that should be followed. In embodiments, provided herein is a method where the system further suggests additional diagnostics relevant to a patient based on 3rd party integrated information aggregated about the patient in question and where the system further analyzes the cost impact of the rule violation. In embodiments, provided herein is a method where the system further suggests additional diagnostics relevant to a patient based on 3rd party integrated information aggregated about the patient in question and where the rules relate to efficient blood utilization. In embodiments, provided herein is a method where the system further suggests additional diagnostics relevant to a patient based on 3rd party integrated information aggregated about the patient in question and where the system further analyzes relatively better treatment outcomes for the costs of delivering services comparing utilization of one or more tests relative to under-utilization or omission of the one or more tests. In embodiments, provided herein is a method where the system further suggests additional diagnostics relevant to a patient based on 3rd party integrated information aggregated about the patient in question and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system further suggests additional diagnostics relevant to a patient based on 3rd party integrated information aggregated about the patient in question and where additional information related to the nature of the event is acquired from a news source. In embodiments, provided herein is a method where the system further suggests additional diagnostics relevant to a patient based on 3rd party integrated information aggregated about the patient in question and where a physician or other human agent is prompted to indicate if a large emergency event has occurred and to characterize the type of event. In embodiments, provided herein is a method where the system further suggests additional diagnostics relevant to a patient based on 3rd party integrated information aggregated about the patient in question and where the event is selected from a list comprising fire, earthquake, mass shooting, explosion, automobile accident, building collapse, hurricane. In embodiments, provided herein is a method where the system further suggests additional diagnostics relevant to a patient based on 3rd party integrated information aggregated about the patient in question and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system further suggests additional diagnostics relevant to a patient based on 3rd party integrated information aggregated about the patient in question and where the workflow notifies a physician that a match has been done and provides a countdown for how soon the blood must be utilized. In embodiments, provided herein is a method where the system further suggests additional diagnostics relevant to a patient based on 3rd party integrated information aggregated about the patient in question and where a workflow collects further information to analyze the likelihood that a blood transfusion was correctly ordered. In embodiments, provided herein is a method where the system further suggests additional diagnostics relevant to a patient based on 3rd party integrated information aggregated about the patient in question and where the workflow notifies a physician how much time is left to return a blood bag to a container. In embodiments, provided herein is a method where the system further suggests additional diagnostics relevant to a patient based on 3rd party integrated information aggregated about the patient in question and where the workflow requests additional information from a physician as to why a transfusion was ordered. In embodiments, provided herein is a method where the system further suggests additional diagnostics relevant to a patient based on 3rd party integrated information aggregated about the patient in question and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system further suggests additional diagnostics relevant to a patient based on 3rd party integrated information aggregated about the patient in question and where the system further comprises an education module that informs the prescribing physician which metabolites can be expected based on the patients known prescriptions. In embodiments, provided herein is a method where the system further suggests additional diagnostics relevant to a patient based on 3rd party integrated information aggregated about the patient in question and where the system further comprises a recommendation module that provides the physician with an indicated likelihood that the patient is abusing and a risk report for the physician to work with the patient. In embodiments, provided herein is a method where the system further suggests additional diagnostics relevant to a patient based on 3rd party integrated information aggregated about the patient in question and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system further suggests additional diagnostics relevant to a patient based on 3rd party integrated information aggregated about the patient in question and where the workflow provides intervention or drug protocol instructions to a physician to apply to a patient. In embodiments, provided herein is a method where the system further suggests additional diagnostics relevant to a patient based on 3rd party integrated information aggregated about the patient in question and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system further suggests additional diagnostics relevant to a patient based on 3rd party integrated information aggregated about the patient in question and where the data analyzed is X, Y and the pathology is diabetes. In embodiments, provided herein is a method where the system further suggests additional diagnostics relevant to a patient based on 3rd party integrated information aggregated about the patient in question and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system further suggests additional diagnostics relevant to a patient based on 3rd party integrated information aggregated about the patient in question and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system further suggests additional diagnostics relevant to a patient based on 3rd party integrated information aggregated about the patient in question and where the metadata is a type of physician and the data used to identify the physician is their billing pattern. In embodiments, provided herein is a method where the system further suggests additional diagnostics relevant to a patient based on 3rd party integrated information aggregated about the patient in question and where the metadata is the classification of a test code in a hierarchy. In embodiments, provided herein is a method where the system further suggests additional diagnostics relevant to a patient based on 3rd party integrated information aggregated about the patient in question and where the metadata includes a specific set of fast Healthcare interoperability data formats and elements, other HL7 data format and elements, or the like. In embodiments, provided herein is a method where the system further suggests additional diagnostics relevant to a patient based on 3rd party integrated information aggregated about the patient in question and for generating inferences comprising a parser, a customer identification module, a health inference engine, and a genetic conflict inference engine. In embodiments, provided herein is a method where the system further suggests additional diagnostics relevant to a patient based on 3rd party integrated information aggregated about the patient in question and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system further suggests additional diagnostics relevant to a patient based on 3rd party integrated information aggregated about the patient in question and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system further suggests additional diagnostics relevant to a patient based on 3rd party integrated information aggregated about the patient in question and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system further suggests additional diagnostics relevant to a patient based on 3rd party integrated information aggregated about the patient in question and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system further suggests additional diagnostics relevant to a patient based on 3rd party integrated information aggregated about the patient in question and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system further suggests additional diagnostics relevant to a patient based on 3rd party integrated information aggregated about the patient in question and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system further suggests additional diagnostics relevant to a patient based on 3rd party integrated information aggregated about the patient in question and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system further suggests additional diagnostics relevant to a patient based on 3rd party integrated information aggregated about the patient in question and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system further suggests additional diagnostics relevant to a patient based on 3rd party integrated information aggregated about the patient in question and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system further suggests additional diagnostics relevant to a patient based on 3rd party integrated information aggregated about the patient in question and providing patient matching comprising collecting a set of patient demographic information from a. In embodiments, provided herein is a method where the system further suggests additional diagnostics relevant to a patient based on 3rd party integrated information aggregated about the patient in question and raising an exception when an unmapped value comes to the PDMP provider. In embodiments, provided herein is a method where the system further suggests additional diagnostics relevant to a patient based on 3rd party integrated information aggregated about the patient in question and mapping test names to PDMP values on the gateway data provider. In embodiments, provided herein is a method where the system further suggests additional diagnostics relevant to a patient based on 3rd party integrated information aggregated about the patient in question and mapping test names to PDMP values. In embodiments, provided herein is a method where the system further suggests additional diagnostics relevant to a patient based on 3rd party integrated information aggregated about the patient in question and dynamically adjusting CRM Layout, Dashboards, or Reports in response to customer activations. In embodiments, provided herein is a method where the system further suggests additional diagnostics relevant to a patient based on 3rd party integrated information aggregated about the patient in question and technique for detecting doc shopping. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system further suggests additional diagnostics relevant to a patient based on 3rd party integrated information aggregated about the patient in question and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system further suggests additional diagnostics relevant to a patient based on 3rd party integrated information aggregated about the patient in question and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system further suggests additional diagnostics relevant to a patient based on 3rd party integrated information aggregated about the patient in question and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system further suggests additional diagnostics relevant to a patient based on 3rd party integrated information aggregated about the patient in question and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system further suggests additional diagnostics relevant to a patient based on 3rd party integrated information aggregated about the patient in question and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system further suggests additional diagnostics relevant to a patient based on 3rd party integrated information aggregated about the patient in question and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system further presents pop-down menus containing auto-completion fields derived from the annotations. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system presents auto-suggest candidates unique to the laboratory, the doctor, or the type of test. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the method further comprises activating a workflow to follow up with hospitals or physicians based on affect analysis. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system applies rules to recommend a review of test equipment function. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system applies rules to recommend a review of personnel efficiency. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system applies rules to recommend a review of the ordering patterns of a physician, an ER, a location, a patient, a hospital, or a lab. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the rules consist of a sequence of testing that should be followed. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system further analyzes the cost impact of the rule violation. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the rules relate to efficient blood utilization. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system further analyzes relatively better treatment outcomes for the costs of delivering services comparing utilization of one or more tests relative to under-utilization or omission of the one or more tests. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where additional information related to the nature of the event is acquired from a news source. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where a physician or other human agent is prompted to indicate if a large emergency event has occurred and to characterize the type of event. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the event is selected from a list comprising fire, earthquake, mass shooting, explosion, automobile accident, building collapse, hurricane. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the workflow notifies a physician that a match has been done and provides a countdown for how soon the blood must be utilized. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where a workflow collects further information to analyze the likelihood that a blood transfusion was correctly ordered. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the workflow notifies a physician how much time is left to return a blood bag to a container. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the workflow requests additional information from a physician as to why a transfusion was ordered. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system further comprises an education module that informs the prescribing physician which metabolites can be expected based on the patients known prescriptions. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the system further comprises a recommendation module that provides the physician with an indicated likelihood that the patient is abusing and a risk report for the physician to work with the patient. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the workflow provides intervention or drug protocol instructions to a physician to apply to a patient. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the data analyzed is X, Y and the pathology is diabetes. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the metadata is a type of physician and the data used to identify the physician is their billing pattern. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the metadata is the classification of a test code in a hierarchy. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the metadata includes a specific set of fast Healthcare interoperability data formats and elements, other HL7 data format and elements, or the like. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for generating inferences comprising a parser, a customer identification module, a health inference engine, and a genetic conflict inference engine. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and where the data sets are selected from the group comprising health care clinical, claims, genomic, immunochemistry, transactional, ordering data, demographic data, Lifestyle and social health data, patient relationships, and patient behaviors. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and providing patient matching comprising collecting a set of patient demographic information from a. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and raising an exception when an unmapped value comes to the PDMP provider. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and mapping test names to PDMP values on the gateway data provider. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and mapping test names to PDMP values. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and dynamically adjusting CRM Layout, Dashboards, or Reports in response to customer activations. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and technique for detecting doc shopping. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for increasing the efficiency of a laboratory data entry system comprising a configurable workflow that receives free-form text annotations about a plurality of orders, parses the text, creates an index of statistically improbable n-grams (words and phrases) from the plurality of free-form text annotations, and auto-suggests phrases to a user of the system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system further presents pop-down menus containing auto-completion fields derived from the annotations. In embodiments, provided herein is a method where the system further presents pop-down menus containing auto-completion fields derived from the annotations and where the system presents auto-suggest candidates unique to the laboratory, the doctor, or the type of test. In embodiments, provided herein is a method where the system further presents pop-down menus containing auto-completion fields derived from the annotations and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method where the system further presents pop-down menus containing auto-completion fields derived from the annotations and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method where the system further presents pop-down menus containing auto-completion fields derived from the annotations and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the system further presents pop-down menus containing auto-completion fields derived from the annotations and where the method further comprises activating a workflow to follow up with hospitals or physicians based on affect analysis. In embodiments, provided herein is a method where the system further presents pop-down menus containing auto-completion fields derived from the annotations and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the system further presents pop-down menus containing auto-completion fields derived from the annotations and where the system applies rules to recommend a review of test equipment function. In embodiments, provided herein is a method where the system further presents pop-down menus containing auto-completion fields derived from the annotations and where the system applies rules to recommend a review of personnel efficiency. In embodiments, provided herein is a method where the system further presents pop-down menus containing auto-completion fields derived from the annotations and where the system applies rules to recommend a review of the ordering patterns of a physician, an ER, a location, a patient, a hospital, or a lab. In embodiments, provided herein is a method where the system further presents pop-down menus containing auto-completion fields derived from the annotations and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system further presents pop-down menus containing auto-completion fields derived from the annotations and where the rules consist of a sequence of testing that should be followed. In embodiments, provided herein is a method where the system further presents pop-down menus containing auto-completion fields derived from the annotations and where the system further analyzes the cost impact of the rule violation. In embodiments, provided herein is a method where the system further presents pop-down menus containing auto-completion fields derived from the annotations and where the rules relate to efficient blood utilization. In embodiments, provided herein is a method where the system further presents pop-down menus containing auto-completion fields derived from the annotations and where the system further analyzes relatively better treatment outcomes for the costs of delivering services comparing utilization of one or more tests relative to under-utilization or omission of the one or more tests. In embodiments, provided herein is a method where the system further presents pop-down menus containing auto-completion fields derived from the annotations and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system further presents pop-down menus containing auto-completion fields derived from the annotations and where additional information related to the nature of the event is acquired from a news source. In embodiments, provided herein is a method where the system further presents pop-down menus containing auto-completion fields derived from the annotations and where a physician or other human agent is prompted to indicate if a large emergency event has occurred and to characterize the type of event. In embodiments, provided herein is a method where the system further presents pop-down menus containing auto-completion fields derived from the annotations and where the event is selected from a list comprising fire, earthquake, mass shooting, explosion, automobile accident, building collapse, hurricane. In embodiments, provided herein is a method where the system further presents pop-down menus containing auto-completion fields derived from the annotations and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system further presents pop-down menus containing auto-completion fields derived from the annotations and where the workflow notifies a physician that a match has been done and provides a countdown for how soon the blood must be utilized. In embodiments, provided herein is a method where the system further presents pop-down menus containing auto-completion fields derived from the annotations and where a workflow collects further information to analyze the likelihood that a blood transfusion was correctly ordered. In embodiments, provided herein is a method where the system further presents pop-down menus containing auto-completion fields derived from the annotations and where the workflow notifies a physician how much time is left to return a blood bag to a container. In embodiments, provided herein is a method where the system further presents pop-down menus containing auto-completion fields derived from the annotations and where the workflow requests additional information from a physician as to why a transfusion was ordered. In embodiments, provided herein is a method where the system further presents pop-down menus containing auto-completion fields derived from the annotations and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system further presents pop-down menus containing auto-completion fields derived from the annotations and where the system further comprises an education module that informs the prescribing physician which metabolites can be expected based on the patients known prescriptions. In embodiments, provided herein is a method where the system further presents pop-down menus containing auto-completion fields derived from the annotations and where the system further comprises a recommendation module that provides the physician with an indicated likelihood that the patient is abusing and a risk report for the physician to work with the patient. In embodiments, provided herein is a method where the system further presents pop-down menus containing auto-completion fields derived from the annotations and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system further presents pop-down menus containing auto-completion fields derived from the annotations and where the workflow provides intervention or drug protocol instructions to a physician to apply to a patient. In embodiments, provided herein is a method where the system further presents pop-down menus containing auto-completion fields derived from the annotations and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system further presents pop-down menus containing auto-completion fields derived from the annotations and where the data analyzed is X, Y and the pathology is diabetes. In embodiments, provided herein is a method where the system further presents pop-down menus containing auto-completion fields derived from the annotations and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system further presents pop-down menus containing auto-completion fields derived from the annotations and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system further presents pop-down menus containing auto-completion fields derived from the annotations and where the metadata is a type of physician and the data used to identify the physician is their billing pattern. In embodiments, provided herein is a method where the system further presents pop-down menus containing auto-completion fields derived from the annotations and where the metadata is the classification of a test code in a hierarchy. In embodiments, provided herein is a method where the system further presents pop-down menus containing auto-completion fields derived from the annotations and where the metadata includes a specific set of fast Healthcare interoperability data formats and elements, other HL7 data format and elements, or the like. In embodiments, provided herein is a method where the system further presents pop-down menus containing auto-completion fields derived from the annotations and for generating inferences comprising a parser, a customer identification module, a health inference engine, and a genetic conflict inference engine. In embodiments, provided herein is a method where the system further presents pop-down menus containing auto-completion fields derived from the annotations and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system further presents pop-down menus containing auto-completion fields derived from the annotations and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system further presents pop-down menus containing auto-completion fields derived from the annotations and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system further presents pop-down menus containing auto-completion fields derived from the annotations and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system further presents pop-down menus containing auto-completion fields derived from the annotations and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system further presents pop-down menus containing auto-completion fields derived from the annotations and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system further presents pop-down menus containing auto-completion fields derived from the annotations and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system further presents pop-down menus containing auto-completion fields derived from the annotations and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system further presents pop-down menus containing auto-completion fields derived from the annotations and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system further presents pop-down menus containing auto-completion fields derived from the annotations and providing patient matching comprising collecting a set of patient demographic information from a. In embodiments, provided herein is a method where the system further presents pop-down menus containing auto-completion fields derived from the annotations and raising an exception when an unmapped value comes to the PDMP provider. In embodiments, provided herein is a method where the system further presents pop-down menus containing auto-completion fields derived from the annotations and mapping test names to PDMP values on the gateway data provider. In embodiments, provided herein is a method where the system further presents pop-down menus containing auto-completion fields derived from the annotations and mapping test names to PDMP values. In embodiments, provided herein is a method where the system further presents pop-down menus containing auto-completion fields derived from the annotations and dynamically adjusting CRM Layout, Dashboards, or Reports in response to customer activations. In embodiments, provided herein is a method where the system further presents pop-down menus containing auto-completion fields derived from the annotations and technique for detecting doc shopping. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system further presents pop-down menus containing auto-completion fields derived from the annotations and ingesting, by a healthcare insight computing device, patient data of a patient received from one of a plurality of patient data providers; determining, by the and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system further presents pop-down menus containing auto-completion fields derived from the annotations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system further presents pop-down menus containing auto-completion fields derived from the annotations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system further presents pop-down menus containing auto-completion fields derived from the annotations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system further presents pop-down menus containing auto-completion fields derived from the annotations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system further presents pop-down menus containing auto-completion fields derived from the annotations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system further presents pop-down menus containing auto-completion fields derived from the annotations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system presents auto-suggest candidates unique to the laboratory, the doctor, or the type of test. In embodiments, provided herein is a method where the system presents auto-suggest candidates unique to the laboratory, the doctor, or the type of test and for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a method where the system presents auto-suggest candidates unique to the laboratory, the doctor, or the type of test and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method where the system presents auto-suggest candidates unique to the laboratory, the doctor, or the type of test and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the system presents auto-suggest candidates unique to the laboratory, the doctor, or the type of test and where the method further comprises activating a workflow to follow up with hospitals or physicians based on affect analysis. In embodiments, provided herein is a method where the system presents auto-suggest candidates unique to the laboratory, the doctor, or the type of test and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the system presents auto-suggest candidates unique to the laboratory, the doctor, or the type of test and where the system applies rules to recommend a review of test equipment function. In embodiments, provided herein is a method where the system presents auto-suggest candidates unique to the laboratory, the doctor, or the type of test and where the system applies rules to recommend a review of personnel efficiency. In embodiments, provided herein is a method where the system presents auto-suggest candidates unique to the laboratory, the doctor, or the type of test and where the system applies rules to recommend a review of the ordering patterns of a physician, an ER, a location, a patient, a hospital, or a lab. In embodiments, provided herein is a method where the system presents auto-suggest candidates unique to the laboratory, the doctor, or the type of test and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system presents auto-suggest candidates unique to the laboratory, the doctor, or the type of test and where the rules consist of a sequence of testing that should be followed. In embodiments, provided herein is a method where the system presents auto-suggest candidates unique to the laboratory, the doctor, or the type of test and where the system further analyzes the cost impact of the rule violation. In embodiments, provided herein is a method where the system presents auto-suggest candidates unique to the laboratory, the doctor, or the type of test and where the rules relate to efficient blood utilization. In embodiments, provided herein is a method where the system presents auto-suggest candidates unique to the laboratory, the doctor, or the type of test and where the system further analyzes relatively better treatment outcomes for the costs of delivering services comparing utilization of one or more tests relative to under-utilization or omission of the one or more tests. In embodiments, provided herein is a method where the system presents auto-suggest candidates unique to the laboratory, the doctor, or the type of test and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system presents auto-suggest candidates unique to the laboratory, the doctor, or the type of test and where additional information related to the nature of the event is acquired from a news source. In embodiments, provided herein is a method where the system presents auto-suggest candidates unique to the laboratory, the doctor, or the type of test and where a physician or other human agent is prompted to indicate if a large emergency event has occurred and to characterize the type of event. In embodiments, provided herein is a method where the system presents auto-suggest candidates unique to the laboratory, the doctor, or the type of test and where the event is selected from a list comprising fire, earthquake, mass shooting, explosion, automobile accident, building collapse, hurricane. In embodiments, provided herein is a method where the system presents auto-suggest candidates unique to the laboratory, the doctor, or the type of test and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system presents auto-suggest candidates unique to the laboratory, the doctor, or the type of test and where the workflow notifies a physician that a match has been done and provides a countdown for how soon the blood must be utilized. In embodiments, provided herein is a method where the system presents auto-suggest candidates unique to the laboratory, the doctor, or the type of test and where a workflow collects further information to analyze the likelihood that a blood transfusion was correctly ordered. In embodiments, provided herein is a method where the system presents auto-suggest candidates unique to the laboratory, the doctor, or the type of test and where the workflow notifies a physician how much time is left to return a blood bag to a container. In embodiments, provided herein is a method where the system presents auto-suggest candidates unique to the laboratory, the doctor, or the type of test and where the workflow requests additional information from a physician as to why a transfusion was ordered. In embodiments, provided herein is a method where the system presents auto-suggest candidates unique to the laboratory, the doctor, or the type of test and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system presents auto-suggest candidates unique to the laboratory, the doctor, or the type of test and where the system further comprises an education module that informs the prescribing physician which metabolites can be expected based on the patients known prescriptions. In embodiments, provided herein is a method where the system presents auto-suggest candidates unique to the laboratory, the doctor, or the type of test and where the system further comprises a recommendation module that provides the physician with an indicated likelihood that the patient is abusing and a risk report for the physician to work with the patient. In embodiments, provided herein is a method where the system presents auto-suggest candidates unique to the laboratory, the doctor, or the type of test and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system presents auto-suggest candidates unique to the laboratory, the doctor, or the type of test and where the workflow provides intervention or drug protocol instructions to a physician to apply to a patient. In embodiments, provided herein is a method where the system presents auto-suggest candidates unique to the laboratory, the doctor, or the type of test and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system presents auto-suggest candidates unique to the laboratory, the doctor, or the type of test and where the data analyzed is X, Y and the pathology is diabetes. In embodiments, provided herein is a method where the system presents auto-suggest candidates unique to the laboratory, the doctor, or the type of test and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system presents auto-suggest candidates unique to the laboratory, the doctor, or the type of test and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system presents auto-suggest candidates unique to the laboratory, the doctor, or the type of test and where the metadata is a type of physician and the data used to identify the physician is their billing pattern. In embodiments, provided herein is a method where the system presents auto-suggest candidates unique to the laboratory, the doctor, or the type of test and where the metadata is the classification of a test code in a hierarchy. In embodiments, provided herein is a method where the system presents auto-suggest candidates unique to the laboratory, the doctor, or the type of test and where the metadata includes a specific set of fast Healthcare interoperability data formats and elements, other HL7 data format and elements, or the like. In embodiments, provided herein is a method where the system presents auto-suggest candidates unique to the laboratory, the doctor, or the type of test and for generating inferences comprising a parser, a customer identification module, a health inference engine, and a genetic conflict inference engine. In embodiments, provided herein is a method where the system presents auto-suggest candidates unique to the laboratory, the doctor, or the type of test and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system presents auto-suggest candidates unique to the laboratory, the doctor, or the type of test and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system presents auto-suggest candidates unique to the laboratory, the doctor, or the type of test and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system presents auto-suggest candidates unique to the laboratory, the doctor, or the type of test and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system presents auto-suggest candidates unique to the laboratory, the doctor, or the type of test and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system presents auto-suggest candidates unique to the laboratory, the doctor, or the type of test and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system presents auto-suggest candidates unique to the laboratory, the doctor, or the type of test and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system presents auto-suggest candidates unique to the laboratory, the doctor, or the type of test and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system presents auto-suggest candidates unique to the laboratory, the doctor, or the type of test and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system presents auto-suggest candidates unique to the laboratory, the doctor, or the type of test and providing patient matching comprising collecting a set of patient demographic information from a. In embodiments, provided herein is a method where the system presents auto-suggest candidates unique to the laboratory, the doctor, or the type of test and raising an exception when an unmapped value comes to the PDMP provider. In embodiments, provided herein is a method where the system presents auto-suggest candidates unique to the laboratory, the doctor, or the type of test and mapping test names to PDMP values on the gateway data provider. In embodiments, provided herein is a method where the system presents auto-suggest candidates unique to the laboratory, the doctor, or the type of test and mapping test names to PDMP values. In embodiments, provided herein is a method where the system presents auto-suggest candidates unique to the laboratory, the doctor, or the type of test and dynamically adjusting CRM Layout, Dashboards, or Reports in response to customer activations. In embodiments, provided herein is a method where the system presents auto-suggest candidates unique to the laboratory, the doctor, or the type of test and technique for detecting doc shopping. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system presents auto-suggest candidates unique to the laboratory, the doctor, or the type of test and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system presents auto-suggest candidates unique to the laboratory, the doctor, or the type of test and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system presents auto-suggest candidates unique to the laboratory, the doctor, or the type of test and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system presents auto-suggest candidates unique to the laboratory, the doctor, or the type of test and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system presents auto-suggest candidates unique to the laboratory, the doctor, or the type of test and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system presents auto-suggest candidates unique to the laboratory, the doctor, or the type of test and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the method further comprises activating a workflow to follow up with hospitals or physicians based on affect analysis. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system applies rules to recommend a review of test equipment function. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system applies rules to recommend a review of personnel efficiency. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system applies rules to recommend a review of the ordering patterns of a physician, an ER, a location, a patient, a hospital, or a lab. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the rules consist of a sequence of testing that should be followed. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system further analyzes the cost impact of the rule violation. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the rules relate to efficient blood utilization. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system further analyzes relatively better treatment outcomes for the costs of delivering services comparing utilization of one or more tests relative to under-utilization or omission of the one or more tests. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where additional information related to the nature of the event is acquired from a news source. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where a physician or other human agent is prompted to indicate if a large emergency event has occurred and to characterize the type of event. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the event is selected from a list comprising fire, earthquake, mass shooting, explosion, automobile accident, building collapse, hurricane. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the workflow notifies a physician that a match has been done and provides a countdown for how soon the blood must be utilized. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where a workflow collects further information to analyze the likelihood that a blood transfusion was correctly ordered. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the workflow notifies a physician how much time is left to return a blood bag to a container. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the workflow requests additional information from a physician as to why a transfusion was ordered. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system further comprises an education module that informs the prescribing physician which metabolites can be expected based on the patients known prescriptions. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the system further comprises a recommendation module that provides the physician with an indicated likelihood that the patient is abusing and a risk report for the physician to work with the patient. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the workflow provides intervention or drug protocol instructions to a physician to apply to a patient. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the data analyzed is X, Y and the pathology is diabetes. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for normalizing data in a medical database comprising, using machine learning to characterize a set of normalized data, applying an ML classification to a new set of unlabeled data, generating metadata and adding labelling metadata to the uncharacterized data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the metadata is a type of physician and the data used to identify the physician is their billing pattern. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the metadata is the classification of a test code in a hierarchy. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the metadata includes a specific set of fast Healthcare interoperability data formats and elements, other HL7 data format and elements, or the like. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for generating inferences comprising a parser, a customer identification module, a health inference engine, and a genetic conflict inference engine. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and where the data sets are selected from the group comprising health care clinical, claims, genomic, immunochemistry, transactional, ordering data, demographic data, Lifestyle and social health data, patient relationships, and patient behaviors. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and providing patient matching comprising collecting a set of patient demographic information from a. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and raising an exception when an unmapped value comes to the PDMP provider. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and mapping test names to PDMP values on the gateway data provider. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and mapping test names to PDMP values. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and dynamically adjusting CRM Layout, Dashboards, or Reports in response to customer activations. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and technique for detecting doc shopping. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for estimating the potential revenue of a laboratory account comprising a contact module that accumulates information about physicians and accounts, an upsell module that determines additional tests that could be offered based on tests already ordered, a cost module that calculates the effective revenue margin for a set of tests, and an estimator module that extrapolates from existing physician or account data what the total value of an opportunity could be based on past, current, future existing and potential lab tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital management solution.

In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the method further comprises activating a workflow to follow up with hospitals or physicians based on affect analysis. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system applies rules to recommend a review of test equipment function. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system applies rules to recommend a review of personnel efficiency. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system applies rules to recommend a review of the ordering patterns of a physician, an ER, a location, a patient, a hospital, or a lab. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the rules consist of a sequence of testing that should be followed. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system further analyzes the cost impact of the rule violation. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the rules relate to efficient blood utilization. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system further analyzes relatively better treatment outcomes for the costs of delivering services comparing utilization of one or more tests relative to under-utilization or omission of the one or more tests. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where additional information related to the nature of the event is acquired from a news source. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where a physician or other human agent is prompted to indicate if a large emergency event has occurred and to characterize the type of event. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the event is selected from a list comprising fire, earthquake, mass shooting, explosion, automobile accident, building collapse, hurricane. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the workflow notifies a physician that a match has been done and provides a countdown for how soon the blood must be utilized. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where a workflow collects further information to analyze the likelihood that a blood transfusion was correctly ordered. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the workflow notifies a physician how much time is left to return a blood bag to a container. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the workflow requests additional information from a physician as to why a transfusion was ordered. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system further comprises an education module that informs the prescribing physician which metabolites can be expected based on the patients known prescriptions. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the system further comprises a recommendation module that provides the physician with an indicated likelihood that the patient is abusing and a risk report for the physician to work with the patient. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the workflow provides intervention or drug protocol instructions to a physician to apply to a patient. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the data analyzed is X, Y and the pathology is diabetes. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for normalizing data in a medical database comprising, using machine learning to characterize a set of normalized data, applying an ML classification to a new set of unlabeled data, generating metadata and adding labelling metadata to the uncharacterized data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the metadata is a type of physician and the data used to identify the physician is their billing pattern. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the metadata is the classification of a test code in a hierarchy. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the metadata includes a specific set of fast Healthcare interoperability data formats and elements, other HL7 data format and elements, or the like. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for generating inferences comprising a parser, a customer identification module, a health inference engine, and a genetic conflict inference engine. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and where the data sets are selected from the group comprising health care clinical, claims, genomic, immunochemistry, transactional, ordering data, demographic data, Lifestyle and social health data, patient relationships, and patient behaviors. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and providing patient matching comprising collecting a set of patient demographic information from a. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and raising an exception when an unmapped value comes to the PDMP provider. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and mapping test names to PDMP values on the gateway data provider. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and mapping test names to PDMP values. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and dynamically adjusting CRM Layout, Dashboards, or Reports in response to customer activations. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and technique for detecting doc shopping. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by to a raw data cluster, wherein encrypting the data and controlling access to the data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method of integrating laboratory data in a CRM system comprising tracking contacts, physicians, hospitals in a CRM system; tracking lab test data corresponding to hospitals and physicians; providing a direct view all tests integrated with a physician or hospital contact record and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the method further comprises activating a workflow to follow up with hospitals or physicians based on affect analysis. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system applies rules to recommend a review of test equipment function. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system applies rules to recommend a review of personnel efficiency. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system applies rules to recommend a review of the ordering patterns of a physician, an ER, a location, a patient, a hospital, or a lab. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the rules consist of a sequence of testing that should be followed. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system further analyzes the cost impact of the rule violation. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the rules relate to efficient blood utilization. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system further analyzes relatively better treatment outcomes for the costs of delivering services comparing utilization of one or more tests relative to under-utilization or omission of the one or more tests. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where additional information related to the nature of the event is acquired from a news source. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where a physician or other human agent is prompted to indicate if a large emergency event has occurred and to characterize the type of event. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the event is selected from a list comprising fire, earthquake, mass shooting, explosion, automobile accident, building collapse, hurricane. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the workflow notifies a physician that a match has been done and provides a countdown for how soon the blood must be utilized. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where a workflow collects further information to analyze the likelihood that a blood transfusion was correctly ordered. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the workflow notifies a physician how much time is left to return a blood bag to a container. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the workflow requests additional information from a physician as to why a transfusion was ordered. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system further comprises an education module that informs the prescribing physician which metabolites can be expected based on the patients known prescriptions. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the system further comprises a recommendation module that provides the physician with an indicated likelihood that the patient is abusing and a risk report for the physician to work with the patient. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the workflow provides intervention or drug protocol instructions to a physician to apply to a patient. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the data analyzed is X, Y and the pathology is diabetes. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the metadata is a type of physician and the data used to identify the physician is their billing pattern. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the metadata is the classification of a test code in a hierarchy. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the metadata includes a specific set of fast Healthcare interoperability data formats and elements, other HL7 data format and elements, or the like. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for generating inferences comprising a parser, a customer identification module, a health inference engine, and a genetic conflict inference engine. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and where the data sets are selected from the group comprising health care clinical, claims, genomic, immunochemistry, transactional, ordering data, demographic data, Lifestyle and social health data, patient relationships, and patient behaviors. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and providing patient matching comprising collecting a set of patient demographic information from a. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and raising an exception when an unmapped value comes to the PDMP provider. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and mapping test names to PDMP values on the gateway data provider. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and mapping test names to PDMP values. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and dynamically adjusting CRM Layout, Dashboards, or Reports in response to customer activations. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and technique for detecting doc shopping. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the method further comprises a textual analysis module that parses and analyzes the plaintext notes entered by the physician or hospital before and after the test, performs affect analysis on the notes, and categorizes the contact as happy, neutral, or unhappy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the method further comprises activating a workflow to follow up with hospitals or physicians based on affect analysis. In embodiments, provided herein is a method where the method further comprises activating a workflow to follow up with hospitals or physicians based on affect analysis and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method where the method further comprises activating a workflow to follow up with hospitals or physicians based on affect analysis and where the system applies rules to recommend a review of test equipment function. In embodiments, provided herein is a method where the method further comprises activating a workflow to follow up with hospitals or physicians based on affect analysis and where the system applies rules to recommend a review of personnel efficiency. In embodiments, provided herein is a method where the method further comprises activating a workflow to follow up with hospitals or physicians based on affect analysis and where the system applies rules to recommend a review of the ordering patterns of a physician, an ER, a location, a patient, a hospital, or a lab. In embodiments, provided herein is a method where the method further comprises activating a workflow to follow up with hospitals or physicians based on affect analysis and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the method further comprises activating a workflow to follow up with hospitals or physicians based on affect analysis and where the rules consist of a sequence of testing that should be followed. In embodiments, provided herein is a method where the method further comprises activating a workflow to follow up with hospitals or physicians based on affect analysis and where the system further analyzes the cost impact of the rule violation. In embodiments, provided herein is a method where the method further comprises activating a workflow to follow up with hospitals or physicians based on affect analysis and where the rules relate to efficient blood utilization. In embodiments, provided herein is a method where the method further comprises activating a workflow to follow up with hospitals or physicians based on affect analysis and where the system further analyzes relatively better treatment outcomes for the costs of delivering services comparing utilization of one or more tests relative to under-utilization or omission of the one or more tests. In embodiments, provided herein is a method where the method further comprises activating a workflow to follow up with hospitals or physicians based on affect analysis and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the method further comprises activating a workflow to follow up with hospitals or physicians based on affect analysis and where additional information related to the nature of the event is acquired from a news source. In embodiments, provided herein is a method where the method further comprises activating a workflow to follow up with hospitals or physicians based on affect analysis and where a physician or other human agent is prompted to indicate if a large emergency event has occurred and to characterize the type of event. In embodiments, provided herein is a method where the method further comprises activating a workflow to follow up with hospitals or physicians based on affect analysis and where the event is selected from a list comprising fire, earthquake, mass shooting, explosion, automobile accident, building collapse, hurricane. In embodiments, provided herein is a method where the method further comprises activating a workflow to follow up with hospitals or physicians based on affect analysis and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the method further comprises activating a workflow to follow up with hospitals or physicians based on affect analysis and where the workflow notifies a physician that a match has been done and provides a countdown for how soon the blood must be utilized. In embodiments, provided herein is a method where the method further comprises activating a workflow to follow up with hospitals or physicians based on affect analysis and where a workflow collects further information to analyze the likelihood that a blood transfusion was correctly ordered. In embodiments, provided herein is a method where the method further comprises activating a workflow to follow up with hospitals or physicians based on affect analysis and where the workflow notifies a physician how much time is left to return a blood bag to a container. In embodiments, provided herein is a method where the method further comprises activating a workflow to follow up with hospitals or physicians based on affect analysis and where the workflow requests additional information from a physician as to why a transfusion was ordered. In embodiments, provided herein is a method where the method further comprises activating a workflow to follow up with hospitals or physicians based on affect analysis and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the method further comprises activating a workflow to follow up with hospitals or physicians based on affect analysis and where the system further comprises an education module that informs the prescribing physician which metabolites can be expected based on the patients known prescriptions. In embodiments, provided herein is a method where the method further comprises activating a workflow to follow up with hospitals or physicians based on affect analysis and where the system further comprises a recommendation module that provides the physician with an indicated likelihood that the patient is abusing and a risk report for the physician to work with the patient. In embodiments, provided herein is a method where the method further comprises activating a workflow to follow up with hospitals or physicians based on affect analysis and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the method further comprises activating a workflow to follow up with hospitals or physicians based on affect analysis and where the workflow provides intervention or drug protocol instructions to a physician to apply to a patient. In embodiments, provided herein is a method where the method further comprises activating a workflow to follow up with hospitals or physicians based on affect analysis and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the method further comprises activating a workflow to follow up with hospitals or physicians based on affect analysis and where the data analyzed is X, Y and the pathology is diabetes. In embodiments, provided herein is a method where the method further comprises activating a workflow to follow up with hospitals or physicians based on affect analysis and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the method further comprises activating a workflow to follow up with hospitals or physicians based on affect analysis and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the method further comprises activating a workflow to follow up with hospitals or physicians based on affect analysis and where the metadata is a type of physician and the data used to identify the physician is their billing pattern. In embodiments, provided herein is a method where the method further comprises activating a workflow to follow up with hospitals or physicians based on affect analysis and where the metadata is the classification of a test code in a hierarchy. In embodiments, provided herein is a method where the method further comprises activating a workflow to follow up with hospitals or physicians based on affect analysis and where the metadata includes a specific set of fast Healthcare interoperability data formats and elements, other HL7 data format and elements, or the like. In embodiments, provided herein is a method where the method further comprises activating a workflow to follow up with hospitals or physicians based on affect analysis and for generating inferences comprising a parser, a customer identification module, a health inference engine, and a genetic conflict inference engine. In embodiments, provided herein is a method where the method further comprises activating a workflow to follow up with hospitals or physicians based on affect analysis and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the method further comprises activating a workflow to follow up with hospitals or physicians based on affect analysis and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the method further comprises activating a workflow to follow up with hospitals or physicians based on affect analysis and where the data sets are selected from the group comprising health care clinical, claims, genomic, immunochemistry, transactional, ordering data, demographic data, Lifestyle and social health data, patient relationships, and patient behaviors. In embodiments, provided herein is a method where the method further comprises activating a workflow to follow up with hospitals or physicians based on affect analysis and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the method further comprises activating a workflow to follow up with hospitals or physicians based on affect analysis and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the method further comprises activating a workflow to follow up with hospitals or physicians based on affect analysis and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the method further comprises activating a workflow to follow up with hospitals or physicians based on affect analysis and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the method further comprises activating a workflow to follow up with hospitals or physicians based on affect analysis and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the method further comprises activating a workflow to follow up with hospitals or physicians based on affect analysis and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the method further comprises activating a workflow to follow up with hospitals or physicians based on affect analysis and providing patient matching comprising collecting a set of patient demographic information from a. In embodiments, provided herein is a method where the method further comprises activating a workflow to follow up with hospitals or physicians based on affect analysis and raising an exception when an unmapped value comes to the PDMP provider. In embodiments, provided herein is a method where the method further comprises activating a workflow to follow up with hospitals or physicians based on affect analysis and mapping test names to PDMP values on the gateway data provider. In embodiments, provided herein is a method where the method further comprises activating a workflow to follow up with hospitals or physicians based on affect analysis and mapping test names to PDMP values. In embodiments, provided herein is a method where the method further comprises activating a workflow to follow up with hospitals or physicians based on affect analysis and dynamically adjusting CRM Layout, Dashboards, or Reports in response to customer activations. In embodiments, provided herein is a method where the method further comprises activating a workflow to follow up with hospitals or physicians based on affect analysis and technique for detecting doc shopping. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the to a raw data cluster, wherein ingesting the patient data received from the patient data provider comprises staging the patient data in a data staging area. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the method further comprises activating a workflow to follow up with hospitals or physicians based on affect analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the method further comprises activating a workflow to follow up with hospitals or physicians based on affect analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the method further comprises activating a workflow to follow up with hospitals or physicians based on affect analysis and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the method further comprises activating a workflow to follow up with hospitals or physicians based on affect analysis and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the method further comprises activating a workflow to follow up with hospitals or physicians based on affect analysis and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital management solution. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the method further comprises activating a workflow to follow up with hospitals or physicians based on affect analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system applies rules to recommend a review of test equipment function. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system applies rules to recommend a review of personnel efficiency. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system applies rules to recommend a review of the ordering patterns of a physician, an ER, a location, a patient, a hospital, or a lab. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the rules consist of a sequence of testing that should be followed. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system further analyzes the cost impact of the rule violation. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the rules relate to efficient blood utilization. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system further analyzes relatively better treatment outcomes for the costs of delivering services comparing utilization of one or more tests relative to under-utilization or omission of the one or more tests. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where additional information related to the nature of the event is acquired from a news source. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where a physician or other human agent is prompted to indicate if a large emergency event has occurred and to characterize the type of event. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the event is selected from a list comprising fire, earthquake, mass shooting, explosion, automobile accident, building collapse, hurricane. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the workflow notifies a physician that a match has been done and provides a countdown for how soon the blood must be utilized. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where a workflow collects further information to analyze the likelihood that a blood transfusion was correctly ordered. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the workflow notifies a physician how much time is left to return a blood bag to a container. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the workflow requests additional information from a physician as to why a transfusion was ordered. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system further comprises an education module that informs the prescribing physician which metabolites can be expected based on the patients known prescriptions. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the system further comprises a recommendation module that provides the physician with an indicated likelihood that the patient is abusing and a risk report for the physician to work with the patient. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the workflow provides intervention or drug protocol instructions to a physician to apply to a patient. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the data analyzed is X, Y and the pathology is diabetes. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the metadata is a type of physician and the data used to identify the physician is their billing pattern. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the metadata is the classification of a test code in a hierarchy. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the metadata includes a specific set of fast Healthcare interoperability data formats and elements, other HL7 data format and elements, or the like. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for generating inferences comprising a parser, a customer identification module, a health inference engine, and a genetic conflict inference engine. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and providing patient matching comprising collecting a set of patient demographic information from a. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and raising an exception when an unmapped value comes to the PDMP provider. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and mapping test names to PDMP values on the gateway data provider. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and mapping test names to PDMP values. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and dynamically adjusting CRM Layout, Dashboards, or Reports in response to customer activations. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and technique for detecting doc shopping. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital management solution. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, performing a machine learning process on the mapped information, receiving human input indicating an anomalous condition relating to any of the entities, and automatically generating a rule relating to that class of entity and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system applies rules to recommend a review of test equipment function. In embodiments, provided herein is a method where the system applies rules to recommend a review of test equipment function and where the system applies rules to recommend a review of personnel efficiency. In embodiments, provided herein is a method where the system applies rules to recommend a review of test equipment function and where the system applies rules to recommend a review of the ordering patterns of a physician, an ER, a location, a patient, a hospital, or a lab. In embodiments, provided herein is a method where the system applies rules to recommend a review of test equipment function and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system applies rules to recommend a review of test equipment function and where the rules consist of a sequence of testing that should be followed. In embodiments, provided herein is a method where the system applies rules to recommend a review of test equipment function and where the system further analyzes the cost impact of the rule violation. In embodiments, provided herein is a method where the system applies rules to recommend a review of test equipment function and where the rules relate to efficient blood utilization. In embodiments, provided herein is a method where the system applies rules to recommend a review of test equipment function and where the system further analyzes relatively better treatment outcomes for the costs of delivering services comparing utilization of one or more tests relative to under-utilization or omission of the one or more tests. In embodiments, provided herein is a method where the system applies rules to recommend a review of test equipment function and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system applies rules to recommend a review of test equipment function and where additional information related to the nature of the event is acquired from a news source. In embodiments, provided herein is a method where the system applies rules to recommend a review of test equipment function and where a physician or other human agent is prompted to indicate if a large emergency event has occurred and to characterize the type of event. In embodiments, provided herein is a method where the system applies rules to recommend a review of test equipment function and where the event is selected from a list comprising fire, earthquake, mass shooting, explosion, automobile accident, building collapse, hurricane. In embodiments, provided herein is a method where the system applies rules to recommend a review of test equipment function and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system applies rules to recommend a review of test equipment function and where the workflow notifies a physician that a match has been done and provides a countdown for how soon the blood must be utilized. In embodiments, provided herein is a method where the system applies rules to recommend a review of test equipment function and where a workflow collects further information to analyze the likelihood that a blood transfusion was correctly ordered. In embodiments, provided herein is a method where the system applies rules to recommend a review of test equipment function and where the workflow notifies a physician how much time is left to return a blood bag to a container. In embodiments, provided herein is a method where the system applies rules to recommend a review of test equipment function and where the workflow requests additional information from a physician as to why a transfusion was ordered. In embodiments, provided herein is a method where the system applies rules to recommend a review of test equipment function and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system applies rules to recommend a review of test equipment function and where the system further comprises an education module that informs the prescribing physician which metabolites can be expected based on the patients known prescriptions. In embodiments, provided herein is a method where the system applies rules to recommend a review of test equipment function and where the system further comprises a recommendation module that provides the physician with an indicated likelihood that the patient is abusing and a risk report for the physician to work with the patient. In embodiments, provided herein is a method where the system applies rules to recommend a review of test equipment function and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system applies rules to recommend a review of test equipment function and where the workflow provides intervention or drug protocol instructions to a physician to apply to a patient. In embodiments, provided herein is a method where the system applies rules to recommend a review of test equipment function and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system applies rules to recommend a review of test equipment function and where the data analyzed is X, Y and the pathology is diabetes. In embodiments, provided herein is a method where the system applies rules to recommend a review of test equipment function and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system applies rules to recommend a review of test equipment function and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system applies rules to recommend a review of test equipment function and where the metadata is a type of physician and the data used to identify the physician is their billing pattern. In embodiments, provided herein is a method where the system applies rules to recommend a review of test equipment function and where the metadata is the classification of a test code in a hierarchy. In embodiments, provided herein is a method where the system applies rules to recommend a review of test equipment function and where the metadata includes a specific set of fast Healthcare interoperability data formats and elements, other HL7 data format and elements, or the like. In embodiments, provided herein is a method where the system applies rules to recommend a review of test equipment function and for generating inferences comprising a parser, a customer identification module, a health inference engine, and a genetic conflict inference engine. In embodiments, provided herein is a method where the system applies rules to recommend a review of test equipment function and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system applies rules to recommend a review of test equipment function and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system applies rules to recommend a review of test equipment function and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system applies rules to recommend a review of test equipment function and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system applies rules to recommend a review of test equipment function and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system applies rules to recommend a review of test equipment function and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system applies rules to recommend a review of test equipment function and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system applies rules to recommend a review of test equipment function and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system applies rules to recommend a review of test equipment function and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system applies rules to recommend a review of test equipment function and providing patient matching comprising collecting a set of patient demographic information from a. In embodiments, provided herein is a method where the system applies rules to recommend a review of test equipment function and raising an exception when an unmapped value comes to the PDMP provider. In embodiments, provided herein is a method where the system applies rules to recommend a review of test equipment function and mapping test names to PDMP values on the gateway data provider. In embodiments, provided herein is a method where the system applies rules to recommend a review of test equipment function and mapping test names to PDMP values. In embodiments, provided herein is a method where the system applies rules to recommend a review of test equipment function and dynamically adjusting CRM Layout, Dashboards, or Reports in response to customer activations. In embodiments, provided herein is a method where the system applies rules to recommend a review of test equipment function and technique for detecting doc shopping. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system applies rules to recommend a review of test equipment function and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system applies rules to recommend a review of test equipment function and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system applies rules to recommend a review of test equipment function and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system applies rules to recommend a review of test equipment function and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system applies rules to recommend a review of test equipment function and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system applies rules to recommend a review of test equipment function and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system applies rules to recommend a review of personnel efficiency. In embodiments, provided herein is a method where the system applies rules to recommend a review of personnel efficiency and where the system applies rules to recommend a review of the ordering patterns of a physician, an ER, a location, a patient, a hospital, or a lab. In embodiments, provided herein is a method where the system applies rules to recommend a review of personnel efficiency and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system applies rules to recommend a review of personnel efficiency and where the rules consist of a sequence of testing that should be followed. In embodiments, provided herein is a method where the system applies rules to recommend a review of personnel efficiency and where the system further analyzes the cost impact of the rule violation. In embodiments, provided herein is a method where the system applies rules to recommend a review of personnel efficiency and where the rules relate to efficient blood utilization. In embodiments, provided herein is a method where the system applies rules to recommend a review of personnel efficiency and where the system further analyzes relatively better treatment outcomes for the costs of delivering services comparing utilization of one or more tests relative to under-utilization or omission of the one or more tests. In embodiments, provided herein is a method where the system applies rules to recommend a review of personnel efficiency and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system applies rules to recommend a review of personnel efficiency and where additional information related to the nature of the event is acquired from a news source. In embodiments, provided herein is a method where the system applies rules to recommend a review of personnel efficiency and where a physician or other human agent is prompted to indicate if a large emergency event has occurred and to characterize the type of event.

In embodiments, provided herein is a method where the system applies rules to recommend a review of personnel efficiency and where the event is selected from a list comprising fire, earthquake, mass shooting, explosion, automobile accident, building collapse, hurricane. In embodiments, provided herein is a method where the system applies rules to recommend a review of personnel efficiency and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system applies rules to recommend a review of personnel efficiency and where the workflow notifies a physician that a match has been done and provides a countdown for how soon the blood must be utilized. In embodiments, provided herein is a method where the system applies rules to recommend a review of personnel efficiency and where a workflow collects further information to analyze the likelihood that a blood transfusion was correctly ordered. In embodiments, provided herein is a method where the system applies rules to recommend a review of personnel efficiency and where the workflow notifies a physician how much time is left to return a blood bag to a container. In embodiments, provided herein is a method where the system applies rules to recommend a review of personnel efficiency and where the workflow requests additional information from a physician as to why a transfusion was ordered. In embodiments, provided herein is a method where the system applies rules to recommend a review of personnel efficiency and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system applies rules to recommend a review of personnel efficiency and where the system further comprises an education module that informs the prescribing physician which metabolites can be expected based on the patients known prescriptions. In embodiments, provided herein is a method where the system applies rules to recommend a review of personnel efficiency and where the system further comprises a recommendation module that provides the physician with an indicated likelihood that the patient is abusing and a risk report for the physician to work with the patient. In embodiments, provided herein is a method where the system applies rules to recommend a review of personnel efficiency and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system applies rules to recommend a review of personnel efficiency and where the workflow provides intervention or drug protocol instructions to a physician to apply to a patient. In embodiments, provided herein is a method where the system applies rules to recommend a review of personnel efficiency and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system applies rules to recommend a review of personnel efficiency and where the data analyzed is X, Y and the pathology is diabetes. In embodiments, provided herein is a method where the system applies rules to recommend a review of personnel efficiency and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system applies rules to recommend a review of personnel efficiency and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system applies rules to recommend a review of personnel efficiency and where the metadata is a type of physician and the data used to identify the physician is their billing pattern. In embodiments, provided herein is a method where the system applies rules to recommend a review of personnel efficiency and where the metadata is the classification of a test code in a hierarchy.

In embodiments, provided herein is a method where the system applies rules to recommend a review of personnel efficiency and where the metadata includes a specific set of fast Healthcare interoperability data formats and elements, other HL7 data format and elements, or the like. In embodiments, provided herein is a method where the system applies rules to recommend a review of personnel efficiency and for generating inferences comprising a parser, a customer identification module, a health inference engine, and a genetic conflict inference engine. In embodiments, provided herein is a method where the system applies rules to recommend a review of personnel efficiency and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system applies rules to recommend a review of personnel efficiency and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system applies rules to recommend a review of personnel efficiency and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system applies rules to recommend a review of personnel efficiency and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system applies rules to recommend a review of personnel efficiency and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system applies rules to recommend a review of personnel efficiency and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system applies rules to recommend a review of personnel efficiency and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system applies rules to recommend a review of personnel efficiency and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system applies rules to recommend a review of personnel efficiency and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system applies rules to recommend a review of personnel efficiency and providing patient matching comprising collecting a set of patient demographic information from a. In embodiments, provided herein is a method where the system applies rules to recommend a review of personnel efficiency and raising an exception when an unmapped value comes to the PDMP provider. In embodiments, provided herein is a method where the system applies rules to recommend a review of personnel efficiency and mapping test names to PDMP values on the gateway data provider. In embodiments, provided herein is a method where the system applies rules to recommend a review of personnel efficiency and mapping test names to PDMP values. In embodiments, provided herein is a method where the system applies rules to recommend a review of personnel efficiency and dynamically adjusting CRM Layout, Dashboards, or Reports in response to customer activations. In embodiments, provided herein is a method where the system applies rules to recommend a review of personnel efficiency and technique for detecting doc shopping. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system applies rules to recommend a review of personnel efficiency and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system applies rules to recommend a review of personnel efficiency and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system applies rules to recommend a review of personnel efficiency and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system applies rules to recommend a review of personnel efficiency and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system applies rules to recommend a review of personnel efficiency and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system applies rules to recommend a review of personnel efficiency and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system applies rules to recommend a review of the ordering patterns of a physician, an ER, a location, a patient, a hospital, or a lab. In embodiments, provided herein is a method where the system applies rules to recommend a review of the ordering patterns of a physician, an ER, a location, a patient, a hospital, or a lab and for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method where the system applies rules to recommend a review of the ordering patterns of a physician, an ER, a location, a patient, a hospital, or a lab and where the rules consist of a sequence of testing that should be followed. In embodiments, provided herein is a method where the system applies rules to recommend a review of the ordering patterns of a physician, an ER, a location, a patient, a hospital, or a lab and where the system further analyzes the cost impact of the rule violation. In embodiments, provided herein is a method where the system applies rules to recommend a review of the ordering patterns of a physician, an ER, a location, a patient, a hospital, or a lab and where the rules relate to efficient blood utilization. In embodiments, provided herein is a method where the system applies rules to recommend a review of the ordering patterns of a physician, an ER, a location, a patient, a hospital, or a lab and where the system further analyzes relatively better treatment outcomes for the costs of delivering services comparing utilization of one or more tests relative to under-utilization or omission of the one or more tests. In embodiments, provided herein is a method where the system applies rules to recommend a review of the ordering patterns of a physician, an ER, a location, a patient, a hospital, or a lab and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system applies rules to recommend a review of the ordering patterns of a physician, an ER, a location, a patient, a hospital, or a lab and where additional information related to the nature of the event is acquired from a news source. In embodiments, provided herein is a method where the system applies rules to recommend a review of the ordering patterns of a physician, an ER, a location, a patient, a hospital, or a lab and where a physician or other human agent is prompted to indicate if a large emergency event has occurred and to characterize the type of event. In embodiments, provided herein is a method where the system applies rules to recommend a review of the ordering patterns of a physician, an ER, a location, a patient, a hospital, or a lab and where the event is selected from a list comprising fire, earthquake, mass shooting, explosion, automobile accident, building collapse, hurricane. In embodiments, provided herein is a method where the system applies rules to recommend a review of the ordering patterns of a physician, an ER, a location, a patient, a hospital, or a lab and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system applies rules to recommend a review of the ordering patterns of a physician, an ER, a location, a patient, a hospital, or a lab and where the workflow notifies a physician that a match has been done and provides a countdown for how soon the blood must be utilized. In embodiments, provided herein is a method where the system applies rules to recommend a review of the ordering patterns of a physician, an ER, a location, a patient, a hospital, or a lab and where a workflow collects further information to analyze the likelihood that a blood transfusion was correctly ordered. In embodiments, provided herein is a method where the system applies rules to recommend a review of the ordering patterns of a physician, an ER, a location, a patient, a hospital, or a lab and where the workflow notifies a physician how much time is left to return a blood bag to a container. In embodiments, provided herein is a method where the system applies rules to recommend a review of the ordering patterns of a physician, an ER, a location, a patient, a hospital, or a lab and where the workflow requests additional information from a physician as to why a transfusion was ordered. In embodiments, provided herein is a method where the system applies rules to recommend a review of the ordering patterns of a physician, an ER, a location, a patient, a hospital, or a lab and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system applies rules to recommend a review of the ordering patterns of a physician, an ER, a location, a patient, a hospital, or a lab and where the system further comprises an education module that informs the prescribing physician which metabolites can be expected based on the patients known prescriptions. In embodiments, provided herein is a method where the system applies rules to recommend a review of the ordering patterns of a physician, an ER, a location, a patient, a hospital, or a lab and where the system further comprises a recommendation module that provides the physician with an indicated likelihood that the patient is abusing and a risk report for the physician to work with the patient. In embodiments, provided herein is a method where the system applies rules to recommend a review of the ordering patterns of a physician, an ER, a location, a patient, a hospital, or a lab and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system applies rules to recommend a review of the ordering patterns of a physician, an ER, a location, a patient, a hospital, or a lab and where the workflow provides intervention or drug protocol instructions to a physician to apply to a patient. In embodiments, provided herein is a method where the system applies rules to recommend a review of the ordering patterns of a physician, an ER, a location, a patient, a hospital, or a lab and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system applies rules to recommend a review of the ordering patterns of a physician, an ER, a location, a patient, a hospital, or a lab and where the data analyzed is X, Y and the pathology is diabetes. In embodiments, provided herein is a method where the system applies rules to recommend a review of the ordering patterns of a physician, an ER, a location, a patient, a hospital, or a lab and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system applies rules to recommend a review of the ordering patterns of a physician, an ER, a location, a patient, a hospital, or a lab and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system applies rules to recommend a review of the ordering patterns of a physician, an ER, a location, a patient, a hospital, or a lab and where the metadata is a type of physician and the data used to identify the physician is their billing pattern. In embodiments, provided herein is a method where the system applies rules to recommend a review of the ordering patterns of a physician, an ER, a location, a patient, a hospital, or a lab and where the metadata is the classification of a test code in a hierarchy. In embodiments, provided herein is a method where the system applies rules to recommend a review of the ordering patterns of a physician, an ER, a location, a patient, a hospital, or a lab and where the metadata includes a specific set of fast Healthcare interoperability data formats and elements, other HL7 data format and elements, or the like. In embodiments, provided herein is a method where the system applies rules to recommend a review of the ordering patterns of a physician, an ER, a location, a patient, a hospital, or a lab and for generating inferences comprising a parser, a customer identification module, a health inference engine, and a genetic conflict inference engine. In embodiments, provided herein is a method where the system applies rules to recommend a review of the ordering patterns of a physician, an ER, a location, a patient, a hospital, or a lab and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system applies rules to recommend a review of the ordering patterns of a physician, an ER, a location, a patient, a hospital, or a lab and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system applies rules to recommend a review of the ordering patterns of a physician, an ER, a location, a patient, a hospital, or a lab and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system applies rules to recommend a review of the ordering patterns of a physician, an ER, a location, a patient, a hospital, or a lab and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system applies rules to recommend a review of the ordering patterns of a physician, an ER, a location, a patient, a hospital, or a lab and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system applies rules to recommend a review of the ordering patterns of a physician, an ER, a location, a patient, a hospital, or a lab and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system applies rules to recommend a review of the ordering patterns of a physician, an ER, a location, a patient, a hospital, or a lab and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system applies rules to recommend a review of the ordering patterns of a physician, an ER, a location, a patient, a hospital, or a lab and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system applies rules to recommend a review of the ordering patterns of a physician, an ER, a location, a patient, a hospital, or a lab and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system applies rules to recommend a review of the ordering patterns of a physician, an ER, a location, a patient, a hospital, or a lab and providing patient matching comprising collecting a set of patient demographic information from a. In embodiments, provided herein is a method where the system applies rules to recommend a review of the ordering patterns of a physician, an ER, a location, a patient, a hospital, or a lab and raising an exception when an unmapped value comes to the PDMP provider. In embodiments, provided herein is a method where the system applies rules to recommend a review of the ordering patterns of a physician, an ER, a location, a patient, a hospital, or a lab and mapping test names to PDMP values on the gateway data provider. In embodiments, provided herein is a method where the system applies rules to recommend a review of the ordering patterns of a physician, an ER, a location, a patient, a hospital, or a lab and mapping test names to PDMP values. In embodiments, provided herein is a method where the system applies rules to recommend a review of the ordering patterns of a physician, an ER, a location, a patient, a hospital, or a lab and dynamically adjusting CRM Layout, Dashboards, or Reports in response to customer activations. In embodiments, provided herein is a method where the system applies rules to recommend a review of the ordering patterns of a physician, an ER, a location, a patient, a hospital, or a lab and technique for detecting doc shopping. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system applies rules to recommend a review of the ordering patterns of a physician, an ER, a location, a patient, a hospital, or a lab and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system applies rules to recommend a review of the ordering patterns of a physician, an ER, a location, a patient, a hospital, or a lab and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system applies rules to recommend a review of the ordering patterns of a physician, an ER, a location, a patient, a hospital, or a lab and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system applies rules to recommend a review of the ordering patterns of a physician, an ER, a location, a patient, a hospital, or a lab and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system applies rules to recommend a review of the ordering patterns of a physician, an ER, a location, a patient, a hospital, or a lab and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system applies rules to recommend a review of the ordering patterns of a physician, an ER, a location, a patient, a hospital, or a lab and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the rules consist of a sequence of testing that should be followed. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the system further analyzes the cost impact of the rule violation. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the rules relate to efficient blood utilization. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the system further analyzes relatively better treatment outcomes for the costs of delivering services comparing utilization of one or more tests relative to under-utilization or omission of the one or more tests. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where additional information related to the nature of the event is acquired from a news source. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where a physician or other human agent is prompted to indicate if a large emergency event has occurred and to characterize the type of event. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the event is selected from a list comprising fire, earthquake, mass shooting, explosion, automobile accident, building collapse, hurricane. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the workflow notifies a physician that a match has been done and provides a countdown for how soon the blood must be utilized. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where a workflow collects further information to analyze the likelihood that a blood transfusion was correctly ordered. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the workflow notifies a physician how much time is left to return a blood bag to a container. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the workflow requests additional information from a physician as to why a transfusion was ordered. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the system further comprises an education module that informs the prescribing physician which metabolites can be expected based on the patients known prescriptions. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the system further comprises a recommendation module that provides the physician with an indicated likelihood that the patient is abusing and a risk report for the physician to work with the patient. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the workflow provides intervention or drug protocol instructions to a physician to apply to a patient. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the data analyzed is X, Y and the pathology is diabetes. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the metadata is a type of physician and the data used to identify the physician is their billing pattern. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the metadata is the classification of a test code in a hierarchy. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the metadata includes a specific set of fast Healthcare interoperability data formats and elements, other HL7 data format and elements, or the like. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for generating inferences comprising a parser, a customer identification module, a health inference engine, and a genetic conflict inference engine. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and where the data sets are selected from the group comprising health care clinical, claims, genomic, immunochemistry, transactional, ordering data, demographic data, Lifestyle and social health data, patient relationships, and patient behaviors. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and providing patient matching comprising collecting a set of patient demographic information from a. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and raising an exception when an unmapped value comes to the PDMP provider. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and mapping test names to PDMP values on the gateway data provider. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and mapping test names to PDMP values. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and dynamically adjusting CRM Layout, Dashboards, or Reports in response to customer activations. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and technique for detecting doc shopping. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for analyzing the efficiency of a test laboratory comprising aggregating transaction information about a plurality of laboratories over time, analyzing volume and type of test, compiling a set of signals relating to test issues, speed, turnaround time, performance, or personnel, mapping the data to an ontology/entity module containing descriptions of the actors (labs, doctors, ERs, hospitals) in the system, applying a set of human-generated rules to the data, and using the entity model, automatically determining the source of a violation of one of the rules and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the rules consist of a sequence of testing that should be followed. In embodiments, provided herein is a method where the rules consist of a sequence of testing that should be followed and where the system further analyzes the cost impact of the rule violation. In embodiments, provided herein is a method where the rules consist of a sequence of testing that should be followed and where the rules relate to efficient blood utilization. In embodiments, provided herein is a method where the rules consist of a sequence of testing that should be followed and where the system further analyzes relatively better treatment outcomes for the costs of delivering services comparing utilization of one or more tests relative to under-utilization or omission of the one or more tests. In embodiments, provided herein is a method where the rules consist of a sequence of testing that should be followed and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the rules consist of a sequence of testing that should be followed and where additional information related to the nature of the event is acquired from a news source. In embodiments, provided herein is a method where the rules consist of a sequence of testing that should be followed and where a physician or other human agent is prompted to indicate if a large emergency event has occurred and to characterize the type of event. In embodiments, provided herein is a method where the rules consist of a sequence of testing that should be followed and where the event is selected from a list comprising fire, earthquake, mass shooting, explosion, automobile accident, building collapse, hurricane. In embodiments, provided herein is a method where the rules consist of a sequence of testing that should be followed and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the rules consist of a sequence of testing that should be followed and where the workflow notifies a physician that a match has been done and provides a countdown for how soon the blood must be utilized. In embodiments, provided herein is a method where the rules consist of a sequence of testing that should be followed and where a workflow collects further information to analyze the likelihood that a blood transfusion was correctly ordered. In embodiments, provided herein is a method where the rules consist of a sequence of testing that should be followed and where the workflow notifies a physician how much time is left to return a blood bag to a container. In embodiments, provided herein is a method where the rules consist of a sequence of testing that should be followed and where the workflow requests additional information from a physician as to why a transfusion was ordered. In embodiments, provided herein is a method where the rules consist of a sequence of testing that should be followed and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the rules consist of a sequence of testing that should be followed and where the system further comprises an education module that informs the prescribing physician which metabolites can be expected based on the patients known prescriptions. In embodiments, provided herein is a method where the rules consist of a sequence of testing that should be followed and where the system further comprises a recommendation module that provides the physician with an indicated likelihood that the patient is abusing and a risk report for the physician to work with the patient. In embodiments, provided herein is a method where the rules consist of a sequence of testing that should be followed and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the rules consist of a sequence of testing that should be followed and where the workflow provides intervention or drug protocol instructions to a physician to apply to a patient. In embodiments, provided herein is a method where the rules consist of a sequence of testing that should be followed and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the rules consist of a sequence of testing that should be followed and where the data analyzed is X, Y and the pathology is diabetes. In embodiments, provided herein is a method where the rules consist of a sequence of testing that should be followed and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the rules consist of a sequence of testing that should be followed and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the rules consist of a sequence of testing that should be followed and where the metadata is a type of physician and the data used to identify the physician is their billing pattern. In embodiments, provided herein is a method where the rules consist of a sequence of testing that should be followed and where the metadata is the classification of a test code in a hierarchy. In embodiments, provided herein is a method where the rules consist of a sequence of testing that should be followed and where the metadata includes a specific set of fast Healthcare interoperability data formats and elements, other HL7 data format and elements, or the like. In embodiments, provided herein is a method where the rules consist of a sequence of testing that should be followed and for generating inferences comprising a parser, a customer identification module, a health inference engine, and a genetic conflict inference engine. In embodiments, provided herein is a method where the rules consist of a sequence of testing that should be followed and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the rules consist of a sequence of testing that should be followed and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the rules consist of a sequence of testing that should be followed and where the data sets are selected from the group comprising health care clinical, claims, genomic, immunochemistry, transactional, ordering data, demographic data, Lifestyle and social health data, patient relationships, and patient behaviors. In embodiments, provided herein is a method where the rules consist of a sequence of testing that should be followed and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the rules consist of a sequence of testing that should be followed and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the rules consist of a sequence of testing that should be followed and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the rules consist of a sequence of testing that should be followed and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the rules consist of a sequence of testing that should be followed and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the rules consist of a sequence of testing that should be followed and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the rules consist of a sequence of testing that should be followed and providing patient matching comprising collecting a set of patient demographic information from a. In embodiments, provided herein is a method where the rules consist of a sequence of testing that should be followed and raising an exception when an unmapped value comes to the PDMP provider. In embodiments, provided herein is a method where the rules consist of a sequence of testing that should be followed and mapping test names to PDMP values on the gateway data provider. In embodiments, provided herein is a method where the rules consist of a sequence of testing that should be followed and mapping test names to PDMP values. In embodiments, provided herein is a method where the rules consist of a sequence of testing that should be followed and dynamically adjusting CRM Layout, Dashboards, or Reports in response to customer activations. In embodiments, provided herein is a method where the rules consist of a sequence of testing that should be followed and technique for detecting doc shopping. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the rules consist of a sequence of testing that should be followed and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the rules consist of a sequence of testing that should be followed and receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the rules consist of a sequence of testing that should be followed and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the rules consist of a sequence of testing that should be followed and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the rules consist of a sequence of testing that should be followed and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the rules consist of a sequence of testing that should be followed and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the rules consist of a sequence of testing that should be followed and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system further analyzes the cost impact of the rule violation. In embodiments, provided herein is a method where the system further analyzes the cost impact of the rule violation and where the rules relate to efficient blood utilization. In embodiments, provided herein is a method where the system further analyzes the cost impact of the rule violation and where the system further analyzes relatively better treatment outcomes for the costs of delivering services comparing utilization of one or more tests relative to under-utilization or omission of the one or more tests. In embodiments, provided herein is a method where the system further analyzes the cost impact of the rule violation and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system further analyzes the cost impact of the rule violation and where additional information related to the nature of the event is acquired from a news source. In embodiments, provided herein is a method where the system further analyzes the cost impact of the rule violation and where a physician or other human agent is prompted to indicate if a large emergency event has occurred and to characterize the type of event. In embodiments, provided herein is a method where the system further analyzes the cost impact of the rule violation and where the event is selected from a list comprising fire, earthquake, mass shooting, explosion, automobile accident, building collapse, hurricane. In embodiments, provided herein is a method where the system further analyzes the cost impact of the rule violation and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system further analyzes the cost impact of the rule violation and where the workflow notifies a physician that a match has been done and provides a countdown for how soon the blood must be utilized. In embodiments, provided herein is a method where the system further analyzes the cost impact of the rule violation and where a workflow collects further information to analyze the likelihood that a blood transfusion was correctly ordered. In embodiments, provided herein is a method where the system further analyzes the cost impact of the rule violation and where the workflow notifies a physician how much time is left to return a blood bag to a container. In embodiments, provided herein is a method where the system further analyzes the cost impact of the rule violation and where the workflow requests additional information from a physician as to why a transfusion was ordered. In embodiments, provided herein is a method where the system further analyzes the cost impact of the rule violation and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system further analyzes the cost impact of the rule violation and where the system further comprises an education module that informs the prescribing physician which metabolites can be expected based on the patients known prescriptions. In embodiments, provided herein is a method where the system further analyzes the cost impact of the rule violation and where the system further comprises a recommendation module that provides the physician with an indicated likelihood that the patient is abusing and a risk report for the physician to work with the patient. In embodiments, provided herein is a method where the system further analyzes the cost impact of the rule violation and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system further analyzes the cost impact of the rule violation and where the workflow provides intervention or drug protocol instructions to a physician to apply to a patient. In embodiments, provided herein is a method where the system further analyzes the cost impact of the rule violation and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system further analyzes the cost impact of the rule violation and where the data analyzed is X, Y and the pathology is diabetes. In embodiments, provided herein is a method where the system further analyzes the cost impact of the rule violation and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system further analyzes the cost impact of the rule violation and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system further analyzes the cost impact of the rule violation and where the metadata is a type of physician and the data used to identify the physician is their billing pattern. In embodiments, provided herein is a method where the system further analyzes the cost impact of the rule violation and where the metadata is the classification of a test code in a hierarchy. In embodiments, provided herein is a method where the system further analyzes the cost impact of the rule violation and where the metadata includes a specific set of fast Healthcare interoperability data formats and elements, other HL7 data format and elements, or the like. In embodiments, provided herein is a method where the system further analyzes the cost impact of the rule violation and for generating inferences comprising a parser, a customer identification module, a health inference engine, and a genetic conflict inference engine. In embodiments, provided herein is a method where the system further analyzes the cost impact of the rule violation and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system further analyzes the cost impact of the rule violation and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system further analyzes the cost impact of the rule violation and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system further analyzes the cost impact of the rule violation and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system further analyzes the cost impact of the rule violation and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system further analyzes the cost impact of the rule violation and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system further analyzes the cost impact of the rule violation and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system further analyzes the cost impact of the rule violation and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system further analyzes the cost impact of the rule violation and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system further analyzes the cost impact of the rule violation and providing patient matching comprising collecting a set of patient demographic information from a. In embodiments, provided herein is a method where the system further analyzes the cost impact of the rule violation and raising an exception when an unmapped value comes to the PDMP provider. In embodiments, provided herein is a method where the system further analyzes the cost impact of the rule violation and mapping test names to PDMP values on the gateway data provider. In embodiments, provided herein is a method where the system further analyzes the cost impact of the rule violation and mapping test names to PDMP values. In embodiments, provided herein is a method where the system further analyzes the cost impact of the rule violation and dynamically adjusting CRM Layout, Dashboards, or Reports in response to customer activations. In embodiments, provided herein is a method where the system further analyzes the cost impact of the rule violation and technique for detecting doc shopping. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system further analyzes the cost impact of the rule violation and ingesting, by a healthcare insight computing device, patient data of a patient received from one of a plurality of patient data providers; determining, by to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system further analyzes the cost impact of the rule violation and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system further analyzes the cost impact of the rule violation and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system further analyzes the cost impact of the rule violation and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system further analyzes the cost impact of the rule violation and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system further analyzes the cost impact of the rule violation and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system further analyzes the cost impact of the rule violation and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the rules relate to efficient blood utilization. In embodiments, provided herein is a method where the rules relate to efficient blood utilization and where the system further analyzes relatively better treatment outcomes for the costs of delivering services comparing utilization of one or more tests relative to under-utilization or omission of the one or more tests. In embodiments, provided herein is a method where the rules relate to efficient blood utilization and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the rules relate to efficient blood utilization and where additional information related to the nature of the event is acquired from a news source. In embodiments, provided herein is a method where the rules relate to efficient blood utilization and where a physician or other human agent is prompted to indicate if a large emergency event has occurred and to characterize the type of event. In embodiments, provided herein is a method where the rules relate to efficient blood utilization and where the event is selected from a list comprising fire, earthquake, mass shooting, explosion, automobile accident, building collapse, hurricane. In embodiments, provided herein is a method where the rules relate to efficient blood utilization and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the rules relate to efficient blood utilization and where the workflow notifies a physician that a match has been done and provides a countdown for how soon the blood must be utilized. In embodiments, provided herein is a method where the rules relate to efficient blood utilization and where a workflow collects further information to analyze the likelihood that a blood transfusion was correctly ordered. In embodiments, provided herein is a method where the rules relate to efficient blood utilization and where the workflow notifies a physician how much time is left to return a blood bag to a container. In embodiments, provided herein is a method where the rules relate to efficient blood utilization and where the workflow requests additional information from a physician as to why a transfusion was ordered. In embodiments, provided herein is a method where the rules relate to efficient blood utilization and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the rules relate to efficient blood utilization and where the system further comprises an education module that informs the prescribing physician which metabolites can be expected based on the patients known prescriptions. In embodiments, provided herein is a method where the rules relate to efficient blood utilization and where the system further comprises a recommendation module that provides the physician with an indicated likelihood that the patient is abusing and a risk report for the physician to work with the patient. In embodiments, provided herein is a method where the rules relate to efficient blood utilization and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the rules relate to efficient blood utilization and where the workflow provides intervention or drug protocol instructions to a physician to apply to a patient. In embodiments, provided herein is a method where the rules relate to efficient blood utilization and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the rules relate to efficient blood utilization and where the data analyzed is X, Y and the pathology is diabetes. In embodiments, provided herein is a method where the rules relate to efficient blood utilization and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the rules relate to efficient blood utilization and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the rules relate to efficient blood utilization and where the metadata is a type of physician and the data used to identify the physician is their billing pattern. In embodiments, provided herein is a method where the rules relate to efficient blood utilization and where the metadata is the classification of a test code in a hierarchy. In embodiments, provided herein is a method where the rules relate to efficient blood utilization and where the metadata includes a specific set of fast Healthcare interoperability data formats and elements, other HL7 data format and elements, or the like. In embodiments, provided herein is a method where the rules relate to efficient blood utilization and for generating inferences comprising a parser, a customer identification module, a health inference engine, and a genetic conflict inference engine. In embodiments, provided herein is a method where the rules relate to efficient blood utilization and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the rules relate to efficient blood utilization and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the rules relate to efficient blood utilization and where the data sets are selected from the group comprising health care clinical, claims, genomic, immunochemistry, transactional, ordering data, demographic data, Lifestyle and social health data, patient relationships, and patient behaviors. In embodiments, provided herein is a method where the rules relate to efficient blood utilization and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the rules relate to efficient blood utilization and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the rules relate to efficient blood utilization and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the rules relate to efficient blood utilization and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the rules relate to efficient blood utilization and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the rules relate to efficient blood utilization and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the rules relate to efficient blood utilization and providing patient matching comprising collecting a set of patient demographic information from a. In embodiments, provided herein is a method where the rules relate to efficient blood utilization and raising an exception when an unmapped value comes to the PDMP provider. In embodiments, provided herein is a method where the rules relate to efficient blood utilization and mapping test names to PDMP values on the gateway data provider. In embodiments, provided herein is a method where the rules relate to efficient blood utilization and mapping test names to PDMP values. In embodiments, provided herein is a method where the rules relate to efficient blood utilization and dynamically adjusting CRM Layout, Dashboards, or Reports in response to customer activations. In embodiments, provided herein is a method where the rules relate to efficient blood utilization and technique for detecting doc shopping. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the rules relate to efficient blood utilization and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the rules relate to efficient blood utilization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the rules relate to efficient blood utilization and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the rules relate to efficient blood utilization and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the rules relate to efficient blood utilization and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the rules relate to efficient blood utilization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the system further analyzes relatively better treatment outcomes for the costs of delivering services comparing utilization of one or more tests relative to under-utilization or omission of the one or more tests. In embodiments, provided herein is a method where the system further analyzes relatively better treatment outcomes for the costs of delivering services comparing utilization of one or more tests relative to under-utilization or omission of the one or more tests and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method where the system further analyzes relatively better treatment outcomes for the costs of delivering services comparing utilization of one or more tests relative to under-utilization or omission of the one or more tests and where additional information related to the nature of the event is acquired from a news source. In embodiments, provided herein is a method where the system further analyzes relatively better treatment outcomes for the costs of delivering services comparing utilization of one or more tests relative to under-utilization or omission of the one or more tests and where a physician or other human agent is prompted to indicate if a large emergency event has occurred and to characterize the type of event. In embodiments, provided herein is a method where the system further analyzes relatively better treatment outcomes for the costs of delivering services comparing utilization of one or more tests relative to under-utilization or omission of the one or more tests and where the event is selected from a list comprising fire, earthquake, mass shooting, explosion, automobile accident, building collapse, hurricane. In embodiments, provided herein is a method where the system further analyzes relatively better treatment outcomes for the costs of delivering services comparing utilization of one or more tests relative to under-utilization or omission of the one or more tests and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the system further analyzes relatively better treatment outcomes for the costs of delivering services comparing utilization of one or more tests relative to under-utilization or omission of the one or more tests and where the workflow notifies a physician that a match has been done and provides a countdown for how soon the blood must be utilized. In embodiments, provided herein is a method where the system further analyzes relatively better treatment outcomes for the costs of delivering services comparing utilization of one or more tests relative to under-utilization or omission of the one or more tests and where a workflow collects further information to analyze the likelihood that a blood transfusion was correctly ordered. In embodiments, provided herein is a method where the system further analyzes relatively better treatment outcomes for the costs of delivering services comparing utilization of one or more tests relative to under-utilization or omission of the one or more tests and where the workflow notifies a physician how much time is left to return a blood bag to a container. In embodiments, provided herein is a method where the system further analyzes relatively better treatment outcomes for the costs of delivering services comparing utilization of one or more tests relative to under-utilization or omission of the one or more tests and where the workflow requests additional information from a physician as to why a transfusion was ordered. In embodiments, provided herein is a method where the system further analyzes relatively better treatment outcomes for the costs of delivering services comparing utilization of one or more tests relative to under-utilization or omission of the one or more tests and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the system further analyzes relatively better treatment outcomes for the costs of delivering services comparing utilization of one or more tests relative to under-utilization or omission of the one or more tests and where the system further comprises an education module that informs the prescribing physician which metabolites can be expected based on the patients known prescriptions. In embodiments, provided herein is a method where the system further analyzes relatively better treatment outcomes for the costs of delivering services comparing utilization of one or more tests relative to under-utilization or omission of the one or more tests and where the system further comprises a recommendation module that provides the physician with an indicated likelihood that the patient is abusing and a risk report for the physician to work with the patient. In embodiments, provided herein is a method where the system further analyzes relatively better treatment outcomes for the costs of delivering services comparing utilization of one or more tests relative to under-utilization or omission of the one or more tests and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the system further analyzes relatively better treatment outcomes for the costs of delivering services comparing utilization of one or more tests relative to under-utilization or omission of the one or more tests and where the workflow provides intervention or drug protocol instructions to a physician to apply to a patient. In embodiments, provided herein is a method where the system further analyzes relatively better treatment outcomes for the costs of delivering services comparing utilization of one or more tests relative to under-utilization or omission of the one or more tests and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the system further analyzes relatively better treatment outcomes for the costs of delivering services comparing utilization of one or more tests relative to under-utilization or omission of the one or more tests and where the data analyzed is X, Y and the pathology is diabetes. In embodiments, provided herein is a method where the system further analyzes relatively better treatment outcomes for the costs of delivering services comparing utilization of one or more tests relative to under-utilization or omission of the one or more tests and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the system further analyzes relatively better treatment outcomes for the costs of delivering services comparing utilization of one or more tests relative to under-utilization or omission of the one or more tests and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the system further analyzes relatively better treatment outcomes for the costs of delivering services comparing utilization of one or more tests relative to under-utilization or omission of the one or more tests and where the metadata is a type of physician and the data used to identify the physician is their billing pattern. In embodiments, provided herein is a method where the system further analyzes relatively better treatment outcomes for the costs of delivering services comparing utilization of one or more tests relative to under-utilization or omission of the one or more tests and where the metadata is the classification of a test code in a hierarchy. In embodiments, provided herein is a method where the system further analyzes relatively better treatment outcomes for the costs of delivering services comparing utilization of one or more tests relative to under-utilization or omission of the one or more tests and where the metadata includes a specific set of fast Healthcare interoperability data formats and elements, other HL7 data format and elements, or the like. In embodiments, provided herein is a method where the system further analyzes relatively better treatment outcomes for the costs of delivering services comparing utilization of one or more tests relative to under-utilization or omission of the one or more tests and for generating inferences comprising a parser, a customer identification module, a health inference engine, and a genetic conflict inference engine. In embodiments, provided herein is a method where the system further analyzes relatively better treatment outcomes for the costs of delivering services comparing utilization of one or more tests relative to under-utilization or omission of the one or more tests and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the system further analyzes relatively better treatment outcomes for the costs of delivering services comparing utilization of one or more tests relative to under-utilization or omission of the one or more tests and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the system further analyzes relatively better treatment outcomes for the costs of delivering services comparing utilization of one or more tests relative to under-utilization or omission of the one or more tests and where the data sets are selected from the group comprising health care clinical, claims, genomic, immunochemistry, transactional, ordering data, demographic data, Lifestyle and social health data, patient relationships, and patient behaviors. In embodiments, provided herein is a method where the system further analyzes relatively better treatment outcomes for the costs of delivering services comparing utilization of one or more tests relative to under-utilization or omission of the one or more tests and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the system further analyzes relatively better treatment outcomes for the costs of delivering services comparing utilization of one or more tests relative to under-utilization or omission of the one or more tests and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the system further analyzes relatively better treatment outcomes for the costs of delivering services comparing utilization of one or more tests relative to under-utilization or omission of the one or more tests and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the system further analyzes relatively better treatment outcomes for the costs of delivering services comparing utilization of one or more tests relative to under-utilization or omission of the one or more tests and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the system further analyzes relatively better treatment outcomes for the costs of delivering services comparing utilization of one or more tests relative to under-utilization or omission of the one or more tests and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the system further analyzes relatively better treatment outcomes for the costs of delivering services comparing utilization of one or more tests relative to under-utilization or omission of the one or more tests and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the system further analyzes relatively better treatment outcomes for the costs of delivering services comparing utilization of one or more tests relative to under-utilization or omission of the one or more tests and providing patient matching comprising collecting a set of patient demographic information from a. In embodiments, provided herein is a method where the system further analyzes relatively better treatment outcomes for the costs of delivering services comparing utilization of one or more tests relative to under-utilization or omission of the one or more tests and raising an exception when an unmapped value comes to the PDMP provider. In embodiments, provided herein is a method where the system further analyzes relatively better treatment outcomes for the costs of delivering services comparing utilization of one or more tests relative to under-utilization or omission of the one or more tests and mapping test names to PDMP values on the gateway data provider. In embodiments, provided herein is a method where the system further analyzes relatively better treatment outcomes for the costs of delivering services comparing utilization of one or more tests relative to under-utilization or omission of the one or more tests and mapping test names to PDMP values. In embodiments, provided herein is a method where the system further analyzes relatively better treatment outcomes for the costs of delivering services comparing utilization of one or more tests relative to under-utilization or omission of the one or more tests and dynamically adjusting CRM Layout, Dashboards, or Reports in response to customer activations. In embodiments, provided herein is a method where the system further analyzes relatively better treatment outcomes for the costs of delivering services comparing utilization of one or more tests relative to under-utilization or omission of the one or more tests and technique for detecting doc shopping. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the system further analyzes relatively better treatment outcomes for the costs of delivering services comparing utilization of one or more tests relative to under-utilization or omission of the one or more tests and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the system further analyzes relatively better treatment outcomes for the costs of delivering services comparing utilization of one or more tests relative to under-utilization or omission of the one or more tests and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the system further analyzes relatively better treatment outcomes for the costs of delivering services comparing utilization of one or more tests relative to under-utilization or omission of the one or more tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the system further analyzes relatively better treatment outcomes for the costs of delivering services comparing utilization of one or more tests relative to under-utilization or omission of the one or more tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the system further analyzes relatively better treatment outcomes for the costs of delivering services comparing utilization of one or more tests relative to under-utilization or omission of the one or more tests and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the system further analyzes relatively better treatment outcomes for the costs of delivering services comparing utilization of one or more tests relative to under-utilization or omission of the one or more tests and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where additional information related to the nature of the event is acquired from a news source. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where a physician or other human agent is prompted to indicate if a large emergency event has occurred and to characterize the type of event. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the event is selected from a list comprising fire, earthquake, mass shooting, explosion, automobile accident, building collapse, hurricane. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the workflow notifies a physician that a match has been done and provides a countdown for how soon the blood must be utilized. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where a workflow collects further information to analyze the likelihood that a blood transfusion was correctly ordered. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the workflow notifies a physician how much time is left to return a blood bag to a container. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the workflow requests additional information from a physician as to why a transfusion was ordered. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the system further comprises an education module that informs the prescribing physician which metabolites can be expected based on the patients known prescriptions. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the system further comprises a recommendation module that provides the physician with an indicated likelihood that the patient is abusing and a risk report for the physician to work with the patient. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the workflow provides intervention or drug protocol instructions to a physician to apply to a patient. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the data analyzed is X, Y and the pathology is diabetes. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the metadata is a type of physician and the data used to identify the physician is their billing pattern. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the metadata is the classification of a test code in a hierarchy. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the metadata includes a specific set of fast Healthcare interoperability data formats and elements, other HL7 data format and elements, or the like. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for generating inferences comprising a parser, a customer identification module, a health inference engine, and a genetic conflict inference engine. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and where the data sets are selected from the group comprising health care clinical, claims, genomic, immunochemistry, transactional, ordering data, demographic data, Lifestyle and social health data, patient relationships, and patient behaviors. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and providing patient matching comprising collecting a set of patient demographic information from a. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and raising an exception when an unmapped value comes to the PDMP provider. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and mapping test names to PDMP values on the gateway data provider. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and mapping test names to PDMP values. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and dynamically adjusting CRM Layout, Dashboards, or Reports in response to customer activations. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and technique for detecting doc shopping. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, matching an increasing order curve to a category of geographically-based incident using on a statistical model, acquiring external information to supplement the match, and running a scarce resource allocation distribution model based on the modelled incident and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where additional information related to the nature of the event is acquired from a news source. In embodiments, provided herein is a method where additional information related to the nature of the event is acquired from a news source and where a physician or other human agent is prompted to indicate if a large emergency event has occurred and to characterize the type of event. In embodiments, provided herein is a method where additional information related to the nature of the event is acquired from a news source and where the event is selected from a list comprising fire, earthquake, mass shooting, explosion, automobile accident, building collapse, hurricane. In embodiments, provided herein is a method where additional information related to the nature of the event is acquired from a news source and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where additional information related to the nature of the event is acquired from a news source and where the workflow notifies a physician that a match has been done and provides a countdown for how soon the blood must be utilized. In embodiments, provided herein is a method where additional information related to the nature of the event is acquired from a news source and where a workflow collects further information to analyze the likelihood that a blood transfusion was correctly ordered. In embodiments, provided herein is a method where additional information related to the nature of the event is acquired from a news source and where the workflow notifies a physician how much time is left to return a blood bag to a container. In embodiments, provided herein is a method where additional information related to the nature of the event is acquired from a news source and where the workflow requests additional information from a physician as to why a transfusion was ordered. In embodiments, provided herein is a method where additional information related to the nature of the event is acquired from a news source and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where additional information related to the nature of the event is acquired from a news source and where the system further comprises an education module that informs the prescribing physician which metabolites can be expected based on the patients known prescriptions. In embodiments, provided herein is a method where additional information related to the nature of the event is acquired from a news source and where the system further comprises a recommendation module that provides the physician with an indicated likelihood that the patient is abusing and a risk report for the physician to work with the patient. In embodiments, provided herein is a method where additional information related to the nature of the event is acquired from a news source and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where additional information related to the nature of the event is acquired from a news source and where the workflow provides intervention or drug protocol instructions to a physician to apply to a patient. In embodiments, provided herein is a method where additional information related to the nature of the event is acquired from a news source and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where additional information related to the nature of the event is acquired from a news source and where the data analyzed is X, Y and the pathology is diabetes. In embodiments, provided herein is a method where additional information related to the nature of the event is acquired from a news source and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where additional information related to the nature of the event is acquired from a news source and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where additional information related to the nature of the event is acquired from a news source and where the metadata is a type of physician and the data used to identify the physician is their billing pattern. In embodiments, provided herein is a method where additional information related to the nature of the event is acquired from a news source and where the metadata is the classification of a test code in a hierarchy. In embodiments, provided herein is a method where additional information related to the nature of the event is acquired from a news source and where the metadata includes a specific set of fast Healthcare interoperability data formats and elements, other HL7 data format and elements, or the like. In embodiments, provided herein is a method where additional information related to the nature of the event is acquired from a news source and for generating inferences comprising a parser, a customer identification module, a health inference engine, and a genetic conflict inference engine. In embodiments, provided herein is a method where additional information related to the nature of the event is acquired from a news source and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where additional information related to the nature of the event is acquired from a news source and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where additional information related to the nature of the event is acquired from a news source and where the data sets are selected from the group comprising health care clinical, claims, genomic, immunochemistry, transactional, ordering data, demographic data, Lifestyle and social health data, patient relationships, and patient behaviors. In embodiments, provided herein is a method where additional information related to the nature of the event is acquired from a news source and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where additional information related to the nature of the event is acquired from a news source and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where additional information related to the nature of the event is acquired from a news source and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where additional information related to the nature of the event is acquired from a news source and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where additional information related to the nature of the event is acquired from a news source and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where additional information related to the nature of the event is acquired from a news source and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where additional information related to the nature of the event is acquired from a news source and providing patient matching comprising collecting a set of patient demographic information from a. In embodiments, provided herein is a method where additional information related to the nature of the event is acquired from a news source and raising an exception when an unmapped value comes to the PDMP provider. In embodiments, provided herein is a method where additional information related to the nature of the event is acquired from a news source and mapping test names to PDMP values on the gateway data provider. In embodiments, provided herein is a method where additional information related to the nature of the event is acquired from a news source and mapping test names to PDMP values. In embodiments, provided herein is a method where additional information related to the nature of the event is acquired from a news source and dynamically adjusting CRM Layout, Dashboards, or Reports in response to customer activations. In embodiments, provided herein is a method where additional information related to the nature of the event is acquired from a news source and technique for detecting doc shopping. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where additional information related to the nature of the event is acquired from a news source and ingesting, by a healthcare insight computing device, patient data of a patient received from one of a plurality of patient data providers; determining, by to a raw data cluster, wherein enriching the correlated patient data comprises supplementing the ingested patient data with referential data. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where additional information related to the nature of the event is acquired from a news source and ingesting, by a healthcare insight computing device, patient data of a patient received from one of a plurality of patient data providers; determining, by the and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where additional information related to the nature of the event is acquired from a news source and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where additional information related to the nature of the event is acquired from a news source and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where additional information related to the nature of the event is acquired from a news source and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where additional information related to the nature of the event is acquired from a news source and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where additional information related to the nature of the event is acquired from a news source and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where additional information related to the nature of the event is acquired from a news source and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where a physician or other human agent is prompted to indicate if a large emergency event has occurred and to characterize the type of event. In embodiments, provided herein is a method where a physician or other human agent is prompted to indicate if a large emergency event has occurred and to characterize the type of event and where the event is selected from a list comprising fire, earthquake, mass shooting, explosion, automobile accident, building collapse, hurricane. In embodiments, provided herein is a method where a physician or other human agent is prompted to indicate if a large emergency event has occurred and to characterize the type of event and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where a physician or other human agent is prompted to indicate if a large emergency event has occurred and to characterize the type of event and where the workflow notifies a physician that a match has been done and provides a countdown for how soon the blood must be utilized. In embodiments, provided herein is a method where a physician or other human agent is prompted to indicate if a large emergency event has occurred and to characterize the type of event and where a workflow collects further information to analyze the likelihood that a blood transfusion was correctly ordered. In embodiments, provided herein is a method where a physician or other human agent is prompted to indicate if a large emergency event has occurred and to characterize the type of event and where the workflow notifies a physician how much time is left to return a blood bag to a container. In embodiments, provided herein is a method where a physician or other human agent is prompted to indicate if a large emergency event has occurred and to characterize the type of event and where the workflow requests additional information from a physician as to why a transfusion was ordered. In embodiments, provided herein is a method where a physician or other human agent is prompted to indicate if a large emergency event has occurred and to characterize the type of event and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where a physician or other human agent is prompted to indicate if a large emergency event has occurred and to characterize the type of event and where the system further comprises an education module that informs the prescribing physician which metabolites can be expected based on the patients known prescriptions. In embodiments, provided herein is a method where a physician or other human agent is prompted to indicate if a large emergency event has occurred and to characterize the type of event and where the system further comprises a recommendation module that provides the physician with an indicated likelihood that the patient is abusing and a risk report for the physician to work with the patient. In embodiments, provided herein is a method where a physician or other human agent is prompted to indicate if a large emergency event has occurred and to characterize the type of event and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where a physician or other human agent is prompted to indicate if a large emergency event has occurred and to characterize the type of event and where the workflow provides intervention or drug protocol instructions to a physician to apply to a patient. In embodiments, provided herein is a method where a physician or other human agent is prompted to indicate if a large emergency event has occurred and to characterize the type of event and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where a physician or other human agent is prompted to indicate if a large emergency event has occurred and to characterize the type of event and where the data analyzed is X, Y and the pathology is diabetes. In embodiments, provided herein is a method where a physician or other human agent is prompted to indicate if a large emergency event has occurred and to characterize the type of event and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where a physician or other human agent is prompted to indicate if a large emergency event has occurred and to characterize the type of event and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where a physician or other human agent is prompted to indicate if a large emergency event has occurred and to characterize the type of event and where the metadata is a type of physician and the data used to identify the physician is their billing pattern. In embodiments, provided herein is a method where a physician or other human agent is prompted to indicate if a large emergency event has occurred and to characterize the type of event and where the metadata is the classification of a test code in a hierarchy. In embodiments, provided herein is a method where a physician or other human agent is prompted to indicate if a large emergency event has occurred and to characterize the type of event and where the metadata includes a specific set of fast Healthcare interoperability data formats and elements, other HL7 data format and elements, or the like. In embodiments, provided herein is a method where a physician or other human agent is prompted to indicate if a large emergency event has occurred and to characterize the type of event and for generating inferences comprising a parser, a customer identification module, a health inference engine, and a genetic conflict inference engine. In embodiments, provided herein is a method where a physician or other human agent is prompted to indicate if a large emergency event has occurred and to characterize the type of event and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where a physician or other human agent is prompted to indicate if a large emergency event has occurred and to characterize the type of event and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where a physician or other human agent is prompted to indicate if a large emergency event has occurred and to characterize the type of event and where the data sets are selected from the group comprising health care clinical, claims, genomic, immunochemistry, transactional, ordering data, demographic data, Lifestyle and social health data, patient relationships, and patient behaviors. In embodiments, provided herein is a method where a physician or other human agent is prompted to indicate if a large emergency event has occurred and to characterize the type of event and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where a physician or other human agent is prompted to indicate if a large emergency event has occurred and to characterize the type of event and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where a physician or other human agent is prompted to indicate if a large emergency event has occurred and to characterize the type of event and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where a physician or other human agent is prompted to indicate if a large emergency event has occurred and to characterize the type of event and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where a physician or other human agent is prompted to indicate if a large emergency event has occurred and to characterize the type of event and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where a physician or other human agent is prompted to indicate if a large emergency event has occurred and to characterize the type of event and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where a physician or other human agent is prompted to indicate if a large emergency event has occurred and to characterize the type of event and providing patient matching comprising collecting a set of patient demographic information from a. In embodiments, provided herein is a method where a physician or other human agent is prompted to indicate if a large emergency event has occurred and to characterize the type of event and raising an exception when an unmapped value comes to the PDMP provider. In embodiments, provided herein is a method where a physician or other human agent is prompted to indicate if a large emergency event has occurred and to characterize the type of event and mapping test names to PDMP values on the gateway data provider. In embodiments, provided herein is a method where a physician or other human agent is prompted to indicate if a large emergency event has occurred and to characterize the type of event and mapping test names to PDMP values. In embodiments, provided herein is a method where a physician or other human agent is prompted to indicate if a large emergency event has occurred and to characterize the type of event and dynamically adjusting CRM Layout, Dashboards, or Reports in response to customer activations. In embodiments, provided herein is a method where a physician or other human agent is prompted to indicate if a large emergency event has occurred and to characterize the type of event and technique for detecting doc shopping. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where a physician or other human agent is prompted to indicate if a large emergency event has occurred and to characterize the type of event and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where a physician or other human agent is prompted to indicate if a large emergency event has occurred and to characterize the type of event and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where a physician or other human agent is prompted to indicate if a large emergency event has occurred and to characterize the type of event and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where a physician or other human agent is prompted to indicate if a large emergency event has occurred and to characterize the type of event and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where a physician or other human agent is prompted to indicate if a large emergency event has occurred and to characterize the type of event and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where a physician or other human agent is prompted to indicate if a large emergency event has occurred and to characterize the type of event and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the event is selected from a list comprising fire, earthquake, mass shooting, explosion, automobile accident, building collapse, hurricane. In embodiments, provided herein is a method where the event is selected from a list comprising fire, earthquake, mass shooting, explosion, automobile accident, building collapse, hurricane and for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method where the event is selected from a list comprising fire, earthquake, mass shooting, explosion, automobile accident, building collapse, hurricane and where the workflow notifies a physician that a match has been done and provides a countdown for how soon the blood must be utilized. In embodiments, provided herein is a method where the event is selected from a list comprising fire, earthquake, mass shooting, explosion, automobile accident, building collapse, hurricane and where a workflow collects further information to analyze the likelihood that a blood transfusion was correctly ordered. In embodiments, provided herein is a method where the event is selected from a list comprising fire, earthquake, mass shooting, explosion, automobile accident, building collapse, hurricane and where the workflow notifies a physician how much time is left to return a blood bag to a container. In embodiments, provided herein is a method where the event is selected from a list comprising fire, earthquake, mass shooting, explosion, automobile accident, building collapse, hurricane and where the workflow requests additional information from a physician as to why a transfusion was ordered. In embodiments, provided herein is a method where the event is selected from a list comprising fire, earthquake, mass shooting, explosion, automobile accident, building collapse, hurricane and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the event is selected from a list comprising fire, earthquake, mass shooting, explosion, automobile accident, building collapse, hurricane and where the system further comprises an education module that informs the prescribing physician which metabolites can be expected based on the patients known prescriptions. In embodiments, provided herein is a method where the event is selected from a list comprising fire, earthquake, mass shooting, explosion, automobile accident, building collapse, hurricane and where the system further comprises a recommendation module that provides the physician with an indicated likelihood that the patient is abusing and a risk report for the physician to work with the patient. In embodiments, provided herein is a method where the event is selected from a list comprising fire, earthquake, mass shooting, explosion, automobile accident, building collapse, hurricane and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the event is selected from a list comprising fire, earthquake, mass shooting, explosion, automobile accident, building collapse, hurricane and where the workflow provides intervention or drug protocol instructions to a physician to apply to a patient. In embodiments, provided herein is a method where the event is selected from a list comprising fire, earthquake, mass shooting, explosion, automobile accident, building collapse, hurricane and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the event is selected from a list comprising fire, earthquake, mass shooting, explosion, automobile accident, building collapse, hurricane and where the data analyzed is X, Y and the pathology is diabetes. In embodiments, provided herein is a method where the event is selected from a list comprising fire, earthquake, mass shooting, explosion, automobile accident, building collapse, hurricane and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the event is selected from a list comprising fire, earthquake, mass shooting, explosion, automobile accident, building collapse, hurricane and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the event is selected from a list comprising fire, earthquake, mass shooting, explosion, automobile accident, building collapse, hurricane and where the metadata is a type of physician and the data used to identify the physician is their billing pattern. In embodiments, provided herein is a method where the event is selected from a list comprising fire, earthquake, mass shooting, explosion, automobile accident, building collapse, hurricane and where the metadata is the classification of a test code in a hierarchy. In embodiments, provided herein is a method where the event is selected from a list comprising fire, earthquake, mass shooting, explosion, automobile accident, building collapse, hurricane and where the metadata includes a specific set of fast Healthcare interoperability data formats and elements, other HL7 data format and elements, or the like. In embodiments, provided herein is a method where the event is selected from a list comprising fire, earthquake, mass shooting, explosion, automobile accident, building collapse, hurricane and for generating inferences comprising a parser, a customer identification module, a health inference engine, and a genetic conflict inference engine. In embodiments, provided herein is a method where the event is selected from a list comprising fire, earthquake, mass shooting, explosion, automobile accident, building collapse, hurricane and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the event is selected from a list comprising fire, earthquake, mass shooting, explosion, automobile accident, building collapse, hurricane and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the event is selected from a list comprising fire, earthquake, mass shooting, explosion, automobile accident, building collapse, hurricane and where the data sets are selected from the group comprising health care clinical, claims, genomic, immunochemistry, transactional, ordering data, demographic data, Lifestyle and social health data, patient relationships, and patient behaviors. In embodiments, provided herein is a method where the event is selected from a list comprising fire, earthquake, mass shooting, explosion, automobile accident, building collapse, hurricane and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the event is selected from a list comprising fire, earthquake, mass shooting, explosion, automobile accident, building collapse, hurricane and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the event is selected from a list comprising fire, earthquake, mass shooting, explosion, automobile accident, building collapse, hurricane and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the event is selected from a list comprising fire, earthquake, mass shooting, explosion, automobile accident, building collapse, hurricane and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the event is selected from a list comprising fire, earthquake, mass shooting, explosion, automobile accident, building collapse, hurricane and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the event is selected from a list comprising fire, earthquake, mass shooting, explosion, automobile accident, building collapse, hurricane and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the event is selected from a list comprising fire, earthquake, mass shooting, explosion, automobile accident, building collapse, hurricane and providing patient matching comprising collecting a set of patient demographic information from a. In embodiments, provided herein is a method where the event is selected from a list comprising fire, earthquake, mass shooting, explosion, automobile accident, building collapse, hurricane and raising an exception when an unmapped value comes to the PDMP provider. In embodiments, provided herein is a method where the event is selected from a list comprising fire, earthquake, mass shooting, explosion, automobile accident, building collapse, hurricane and mapping test names to PDMP values on the gateway data provider. In embodiments, provided herein is a method where the event is selected from a list comprising fire, earthquake, mass shooting, explosion, automobile accident, building collapse, hurricane and mapping test names to PDMP values. In embodiments, provided herein is a method where the event is selected from a list comprising fire, earthquake, mass shooting, explosion, automobile accident, building collapse, hurricane and dynamically adjusting CRM Layout, Dashboards, or Reports in response to customer activations. In embodiments, provided herein is a method where the event is selected from a list comprising fire, earthquake, mass shooting, explosion, automobile accident, building collapse, hurricane and technique for detecting doc shopping. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the event is selected from a list comprising fire, earthquake, mass shooting, explosion, automobile accident, building collapse, hurricane and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the event is selected from a list comprising fire, earthquake, mass shooting, explosion, automobile accident, building collapse, hurricane and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the event is selected from a list comprising fire, earthquake, mass shooting, explosion, automobile accident, building collapse, hurricane and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the event is selected from a list comprising fire, earthquake, mass shooting, explosion, automobile accident, building collapse, hurricane and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the event is selected from a list comprising fire, earthquake, mass shooting, explosion, automobile accident, building collapse, hurricane and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the event is selected from a list comprising fire, earthquake, mass shooting, explosion, automobile accident, building collapse, hurricane and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the workflow notifies a physician that a match has been done and provides a countdown for how soon the blood must be utilized. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where a workflow collects further information to analyze the likelihood that a blood transfusion was correctly ordered. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the workflow notifies a physician how much time is left to return a blood bag to a container. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the workflow requests additional information from a physician as to why a transfusion was ordered. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the system further comprises an education module that informs the prescribing physician which metabolites can be expected based on the patients known prescriptions. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the system further comprises a recommendation module that provides the physician with an indicated likelihood that the patient is abusing and a risk report for the physician to work with the patient. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the workflow provides intervention or drug protocol instructions to a physician to apply to a patient. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the data analyzed is X, Y and the pathology is diabetes. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the metadata is a type of physician and the data used to identify the physician is their billing pattern. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the metadata is the classification of a test code in a hierarchy. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the metadata includes a specific set of fast Healthcare interoperability data formats and elements, other HL7 data format and elements, or the like. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for generating inferences comprising a parser, a customer identification module, a health inference engine, and a genetic conflict inference engine. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and where the data sets are selected from the group comprising health care clinical, claims, genomic, immunochemistry, transactional, ordering data, demographic data, Lifestyle and social health data, patient relationships, and patient behaviors. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and providing patient matching comprising collecting a set of patient demographic information from a. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and raising an exception when an unmapped value comes to the PDMP provider. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and mapping test names to PDMP values on the gateway data provider. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and mapping test names to PDMP values. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and dynamically adjusting CRM Layout, Dashboards, or Reports in response to customer activations. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and technique for detecting doc shopping. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for increasing the efficiency of blood use comprising instrumenting a plurality of hospitals, ERs or other facilities for a related geographic area, receiving continual data relating to the ongoing ordering of blood or other supplies, monitoring a rate of ordering of blood or other supplies, calculating a CT ratio based on blood match testing and subsequent use of the blood, and triggering a workflow when a rule is triggered by the value data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the workflow notifies a physician that a match has been done and provides a countdown for how soon the blood must be utilized. In embodiments, provided herein is a method where the workflow notifies a physician that a match has been done and provides a countdown for how soon the blood must be utilized and where a workflow collects further information to analyze the likelihood that a blood transfusion was correctly ordered. In embodiments, provided herein is a method where the workflow notifies a physician that a match has been done and provides a countdown for how soon the blood must be utilized and where the workflow notifies a physician how much time is left to return a blood bag to a container. In embodiments, provided herein is a method where the workflow notifies a physician that a match has been done and provides a countdown for how soon the blood must be utilized and where the workflow requests additional information from a physician as to why a transfusion was ordered. In embodiments, provided herein is a method where the workflow notifies a physician that a match has been done and provides a countdown for how soon the blood must be utilized and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the workflow notifies a physician that a match has been done and provides a countdown for how soon the blood must be utilized and where the system further comprises an education module that informs the prescribing physician which metabolites can be expected based on the patients known prescriptions. In embodiments, provided herein is a method where the workflow notifies a physician that a match has been done and provides a countdown for how soon the blood must be utilized and where the system further comprises a recommendation module that provides the physician with an indicated likelihood that the patient is abusing and a risk report for the physician to work with the patient. In embodiments, provided herein is a method where the workflow notifies a physician that a match has been done and provides a countdown for how soon the blood must be utilized and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the workflow notifies a physician that a match has been done and provides a countdown for how soon the blood must be utilized and where the workflow provides intervention or drug protocol instructions to a physician to apply to a patient. In embodiments, provided herein is a method where the workflow notifies a physician that a match has been done and provides a countdown for how soon the blood must be utilized and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the workflow notifies a physician that a match has been done and provides a countdown for how soon the blood must be utilized and where the data analyzed is X, Y and the pathology is diabetes. In embodiments, provided herein is a method where the workflow notifies a physician that a match has been done and provides a countdown for how soon the blood must be utilized and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the workflow notifies a physician that a match has been done and provides a countdown for how soon the blood must be utilized and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the workflow notifies a physician that a match has been done and provides a countdown for how soon the blood must be utilized and where the metadata is a type of physician and the data used to identify the physician is their billing pattern. In embodiments, provided herein is a method where the workflow notifies a physician that a match has been done and provides a countdown for how soon the blood must be utilized and where the metadata is the classification of a test code in a hierarchy. In embodiments, provided herein is a method where the workflow notifies a physician that a match has been done and provides a countdown for how soon the blood must be utilized and where the metadata includes a specific set of fast Healthcare interoperability data formats and elements, other HL7 data format and elements, or the like. In embodiments, provided herein is a method where the workflow notifies a physician that a match has been done and provides a countdown for how soon the blood must be utilized and for generating inferences comprising a parser, a customer identification module, a health inference engine, and a genetic conflict inference engine. In embodiments, provided herein is a method where the workflow notifies a physician that a match has been done and provides a countdown for how soon the blood must be utilized and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the workflow notifies a physician that a match has been done and provides a countdown for how soon the blood must be utilized and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the workflow notifies a physician that a match has been done and provides a countdown for how soon the blood must be utilized and where the data sets are selected from the group comprising health care clinical, claims, genomic, immunochemistry, transactional, ordering data, demographic data, Lifestyle and social health data, patient relationships, and patient behaviors. In embodiments, provided herein is a method where the workflow notifies a physician that a match has been done and provides a countdown for how soon the blood must be utilized and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the workflow notifies a physician that a match has been done and provides a countdown for how soon the blood must be utilized and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the workflow notifies a physician that a match has been done and provides a countdown for how soon the blood must be utilized and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the workflow notifies a physician that a match has been done and provides a countdown for how soon the blood must be utilized and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the workflow notifies a physician that a match has been done and provides a countdown for how soon the blood must be utilized and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the workflow notifies a physician that a match has been done and provides a countdown for how soon the blood must be utilized and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the workflow notifies a physician that a match has been done and provides a countdown for how soon the blood must be utilized and providing patient matching comprising collecting a set of patient demographic information from a. In embodiments, provided herein is a method where the workflow notifies a physician that a match has been done and provides a countdown for how soon the blood must be utilized and raising an exception when an unmapped value comes to the PDMP provider. In embodiments, provided herein is a method where the workflow notifies a physician that a match has been done and provides a countdown for how soon the blood must be utilized and mapping test names to PDMP values on the gateway data provider. In embodiments, provided herein is a method where the workflow notifies a physician that a match has been done and provides a countdown for how soon the blood must be utilized and mapping test names to PDMP values. In embodiments, provided herein is a method where the workflow notifies a physician that a match has been done and provides a countdown for how soon the blood must be utilized and dynamically adjusting CRM Layout, Dashboards, or Reports in response to customer activations. In embodiments, provided herein is a method where the workflow notifies a physician that a match has been done and provides a countdown for how soon the blood must be utilized and technique for detecting doc shopping. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the workflow notifies a physician that a match has been done and provides a countdown for how soon the blood must be utilized and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the workflow notifies a physician that a match has been done and provides a countdown for how soon the blood must be utilized and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the workflow notifies a physician that a match has been done and provides a countdown for how soon the blood must be utilized and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the workflow notifies a physician that a match has been done and provides a countdown for how soon the blood must be utilized and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the workflow notifies a physician that a match has been done and provides a countdown for how soon the blood must be utilized and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the workflow notifies a physician that a match has been done and provides a countdown for how soon the blood must be utilized and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where a workflow collects further information to analyze the likelihood that a blood transfusion was correctly ordered. In embodiments, provided herein is a method where a workflow collects further information to analyze the likelihood that a blood transfusion was correctly ordered and where the workflow notifies a physician how much time is left to return a blood bag to a container. In embodiments, provided herein is a method where a workflow collects further information to analyze the likelihood that a blood transfusion was correctly ordered and where the workflow requests additional information from a physician as to why a transfusion was ordered. In embodiments, provided herein is a method where a workflow collects further information to analyze the likelihood that a blood transfusion was correctly ordered and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where a workflow collects further information to analyze the likelihood that a blood transfusion was correctly ordered and where the system further comprises an education module that informs the prescribing physician which metabolites can be expected based on the patients known prescriptions. In embodiments, provided herein is a method where a workflow collects further information to analyze the likelihood that a blood transfusion was correctly ordered and where the system further comprises a recommendation module that provides the physician with an indicated likelihood that the patient is abusing and a risk report for the physician to work with the patient. In embodiments, provided herein is a method where a workflow collects further information to analyze the likelihood that a blood transfusion was correctly ordered and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where a workflow collects further information to analyze the likelihood that a blood transfusion was correctly ordered and where the workflow provides intervention or drug protocol instructions to a physician to apply to a patient. In embodiments, provided herein is a method where a workflow collects further information to analyze the likelihood that a blood transfusion was correctly ordered and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where a workflow collects further information to analyze the likelihood that a blood transfusion was correctly ordered and where the data analyzed is X, Y and the pathology is diabetes. In embodiments, provided herein is a method where a workflow collects further information to analyze the likelihood that a blood transfusion was correctly ordered and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where a workflow collects further information to analyze the likelihood that a blood transfusion was correctly ordered and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where a workflow collects further information to analyze the likelihood that a blood transfusion was correctly ordered and where the metadata is a type of physician and the data used to identify the physician is their billing pattern. In embodiments, provided herein is a method where a workflow collects further information to analyze the likelihood that a blood transfusion was correctly ordered and where the metadata is the classification of a test code in a hierarchy. In embodiments, provided herein is a method where a workflow collects further information to analyze the likelihood that a blood transfusion was correctly ordered and where the metadata includes a specific set of fast Healthcare interoperability data formats and elements, other HL7 data format and elements, or the like. In embodiments, provided herein is a method where a workflow collects further information to analyze the likelihood that a blood transfusion was correctly ordered and for generating inferences comprising a parser, a customer identification module, a health inference engine, and a genetic conflict inference engine. In embodiments, provided herein is a method where a workflow collects further information to analyze the likelihood that a blood transfusion was correctly ordered and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where a workflow collects further information to analyze the likelihood that a blood transfusion was correctly ordered and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where a workflow collects further information to analyze the likelihood that a blood transfusion was correctly ordered and where the data sets are selected from the group comprising health care clinical, claims, genomic, immunochemistry, transactional, ordering data, demographic data, Lifestyle and social health data, patient relationships, and patient behaviors. In embodiments, provided herein is a method where a workflow collects further information to analyze the likelihood that a blood transfusion was correctly ordered and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where a workflow collects further information to analyze the likelihood that a blood transfusion was correctly ordered and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where a workflow collects further information to analyze the likelihood that a blood transfusion was correctly ordered and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where a workflow collects further information to analyze the likelihood that a blood transfusion was correctly ordered and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where a workflow collects further information to analyze the likelihood that a blood transfusion was correctly ordered and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where a workflow collects further information to analyze the likelihood that a blood transfusion was correctly ordered and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where a workflow collects further information to analyze the likelihood that a blood transfusion was correctly ordered and providing patient matching comprising collecting a set of patient demographic information from a. In embodiments, provided herein is a method where a workflow collects further information to analyze the likelihood that a blood transfusion was correctly ordered and raising an exception when an unmapped value comes to the PDMP provider. In embodiments, provided herein is a method where a workflow collects further information to analyze the likelihood that a blood transfusion was correctly ordered and mapping test names to PDMP values on the gateway data provider. In embodiments, provided herein is a method where a workflow collects further information to analyze the likelihood that a blood transfusion was correctly ordered and mapping test names to PDMP values. In embodiments, provided herein is a method where a workflow collects further information to analyze the likelihood that a blood transfusion was correctly ordered and dynamically adjusting CRM Layout, Dashboards, or Reports in response to customer activations. In embodiments, provided herein is a method where a workflow collects further information to analyze the likelihood that a blood transfusion was correctly ordered and technique for detecting doc shopping. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where a workflow collects further information to analyze the likelihood that a blood transfusion was correctly ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where a workflow collects further information to analyze the likelihood that a blood transfusion was correctly ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where a workflow collects further information to analyze the likelihood that a blood transfusion was correctly ordered and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where a workflow collects further information to analyze the likelihood that a blood transfusion was correctly ordered and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where a workflow collects further information to analyze the likelihood that a blood transfusion was correctly ordered and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where a workflow collects further information to analyze the likelihood that a blood transfusion was correctly ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the workflow notifies a physician how much time is left to return a blood bag to a container. In embodiments, provided herein is a method where the workflow notifies a physician how much time is left to return a blood bag to a container and where the workflow requests additional information from a physician as to why a transfusion was ordered. In embodiments, provided herein is a method where the workflow notifies a physician how much time is left to return a blood bag to a container and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the workflow notifies a physician how much time is left to return a blood bag to a container and where the system further comprises an education module that informs the prescribing physician which metabolites can be expected based on the patients known prescriptions. In embodiments, provided herein is a method where the workflow notifies a physician how much time is left to return a blood bag to a container and where the system further comprises a recommendation module that provides the physician with an indicated likelihood that the patient is abusing and a risk report for the physician to work with the patient. In embodiments, provided herein is a method where the workflow notifies a physician how much time is left to return a blood bag to a container and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the workflow notifies a physician how much time is left to return a blood bag to a container and where the workflow provides intervention or drug protocol instructions to a physician to apply to a patient. In embodiments, provided herein is a method where the workflow notifies a physician how much time is left to return a blood bag to a container and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the workflow notifies a physician how much time is left to return a blood bag to a container and where the data analyzed is X, Y and the pathology is diabetes. In embodiments, provided herein is a method where the workflow notifies a physician how much time is left to return a blood bag to a container and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the workflow notifies a physician how much time is left to return a blood bag to a container and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the workflow notifies a physician how much time is left to return a blood bag to a container and where the metadata is a type of physician and the data used to identify the physician is their billing pattern. In embodiments, provided herein is a method where the workflow notifies a physician how much time is left to return a blood bag to a container and where the metadata is the classification of a test code in a hierarchy. In embodiments, provided herein is a method where the workflow notifies a physician how much time is left to return a blood bag to a container and where the metadata includes a specific set of fast Healthcare interoperability data formats and elements, other HL7 data format and elements, or the like. In embodiments, provided herein is a method where the workflow notifies a physician how much time is left to return a blood bag to a container and for generating inferences comprising a parser, a customer identification module, a health inference engine, and a genetic conflict inference engine. In embodiments, provided herein is a method where the workflow notifies a physician how much time is left to return a blood bag to a container and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the workflow notifies a physician how much time is left to return a blood bag to a container and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the workflow notifies a physician how much time is left to return a blood bag to a container and where the data sets are selected from the group comprising health care clinical, claims, genomic, immunochemistry, transactional, ordering data, demographic data, Lifestyle and social health data, patient relationships, and patient behaviors. In embodiments, provided herein is a method where the workflow notifies a physician how much time is left to return a blood bag to a container and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the workflow notifies a physician how much time is left to return a blood bag to a container and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the workflow notifies a physician how much time is left to return a blood bag to a container and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the workflow notifies a physician how much time is left to return a blood bag to a container and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the workflow notifies a physician how much time is left to return a blood bag to a container and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the workflow notifies a physician how much time is left to return a blood bag to a container and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the workflow notifies a physician how much time is left to return a blood bag to a container and providing patient matching comprising collecting a set of patient demographic information from a. In embodiments, provided herein is a method where the workflow notifies a physician how much time is left to return a blood bag to a container and raising an exception when an unmapped value comes to the PDMP provider. In embodiments, provided herein is a method where the workflow notifies a physician how much time is left to return a blood bag to a container and mapping test names to PDMP values on the gateway data provider. In embodiments, provided herein is a method where the workflow notifies a physician how much time is left to return a blood bag to a container and mapping test names to PDMP values. In embodiments, provided herein is a method where the workflow notifies a physician how much time is left to return a blood bag to a container and dynamically adjusting CRM Layout, Dashboards, or Reports in response to customer activations. In embodiments, provided herein is a method where the workflow notifies a physician how much time is left to return a blood bag to a container and technique for detecting doc shopping. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the workflow notifies a physician how much time is left to return a blood bag to a container and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the workflow notifies a physician how much time is left to return a blood bag to a container and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the workflow notifies a physician how much time is left to return a blood bag to a container and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the workflow notifies a physician how much time is left to return a blood bag to a container and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the workflow notifies a physician how much time is left to return a blood bag to a container and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the workflow notifies a physician how much time is left to return a blood bag to a container and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the workflow requests additional information from a physician as to why a transfusion was ordered. In embodiments, provided herein is a method where the workflow requests additional information from a physician as to why a transfusion was ordered and for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a method where the workflow requests additional information from a physician as to why a transfusion was ordered and where the system further comprises an education module that informs the prescribing physician which metabolites can be expected based on the patients known prescriptions. In embodiments, provided herein is a method where the workflow requests additional information from a physician as to why a transfusion was ordered and where the system further comprises a recommendation module that provides the physician with an indicated likelihood that the patient is abusing and a risk report for the physician to work with the patient. In embodiments, provided herein is a method where the workflow requests additional information from a physician as to why a transfusion was ordered and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method where the workflow requests additional information from a physician as to why a transfusion was ordered and where the workflow provides intervention or drug protocol instructions to a physician to apply to a patient. In embodiments, provided herein is a method where the workflow requests additional information from a physician as to why a transfusion was ordered and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the workflow requests additional information from a physician as to why a transfusion was ordered and where the data analyzed is X, Y and the pathology is diabetes. In embodiments, provided herein is a method where the workflow requests additional information from a physician as to why a transfusion was ordered and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the workflow requests additional information from a physician as to why a transfusion was ordered and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the workflow requests additional information from a physician as to why a transfusion was ordered and where the metadata is a type of physician and the data used to identify the physician is their billing pattern. In embodiments, provided herein is a method where the workflow requests additional information from a physician as to why a transfusion was ordered and where the metadata is the classification of a test code in a hierarchy. In embodiments, provided herein is a method where the workflow requests additional information from a physician as to why a transfusion was ordered and where the metadata includes a specific set of fast Healthcare interoperability data formats and elements, other HL7 data format and elements, or the like. In embodiments, provided herein is a method where the workflow requests additional information from a physician as to why a transfusion was ordered and for generating inferences comprising a parser, a customer identification module, a health inference engine, and a genetic conflict inference engine. In embodiments, provided herein is a method where the workflow requests additional information from a physician as to why a transfusion was ordered and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the workflow requests additional information from a physician as to why a transfusion was ordered and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the workflow requests additional information from a physician as to why a transfusion was ordered and where the data sets are selected from the group comprising health care clinical, claims, genomic, immunochemistry, transactional, ordering data, demographic data, Lifestyle and social health data, patient relationships, and patient behaviors. In embodiments, provided herein is a method where the workflow requests additional information from a physician as to why a transfusion was ordered and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the workflow requests additional information from a physician as to why a transfusion was ordered and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the workflow requests additional information from a physician as to why a transfusion was ordered and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the workflow requests additional information from a physician as to why a transfusion was ordered and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the workflow requests additional information from a physician as to why a transfusion was ordered and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the workflow requests additional information from a physician as to why a transfusion was ordered and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the workflow requests additional information from a physician as to why a transfusion was ordered and providing patient matching comprising collecting a set of patient demographic information from a. In embodiments, provided herein is a method where the workflow requests additional information from a physician as to why a transfusion was ordered and raising an exception when an unmapped value comes to the PDMP provider. In embodiments, provided herein is a method where the workflow requests additional information from a physician as to why a transfusion was ordered and mapping test names to PDMP values on the gateway data provider. In embodiments, provided herein is a method where the workflow requests additional information from a physician as to why a transfusion was ordered and mapping test names to PDMP values. In embodiments, provided herein is a method where the workflow requests additional information from a physician as to why a transfusion was ordered and dynamically adjusting CRM Layout, Dashboards, or Reports in response to customer activations. In embodiments, provided herein is a method where the workflow requests additional information from a physician as to why a transfusion was ordered and technique for detecting doc shopping. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the workflow requests additional information from a physician as to why a transfusion was ordered and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the workflow requests additional information from a physician as to why a transfusion was ordered and ingesting, by a healthcare insight computing device, patient data of a patient received from one of a plurality of patient data providers; determining, by insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the workflow requests additional information from a physician as to why a transfusion was ordered and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the workflow requests additional information from a physician as to why a transfusion was ordered and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the workflow requests additional information from a physician as to why a transfusion was ordered and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the workflow requests additional information from a physician as to why a transfusion was ordered and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the workflow requests additional information from a physician as to why a transfusion was ordered and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the workflow requests additional information from a physician as to why a transfusion was ordered and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital management solution.

In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the system further comprises an education module that informs the prescribing physician which metabolites can be expected based on the patients known prescriptions. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the system further comprises a recommendation module that provides the physician with an indicated likelihood that the patient is abusing and a risk report for the physician to work with the patient. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the workflow provides intervention or drug protocol instructions to a physician to apply to a patient. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the data analyzed is X, Y and the pathology is diabetes. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and for normalizing data in a medical database comprising, using machine learning to characterize a set of normalized data, applying an ML classification to a new set of unlabeled data, generating metadata and adding labelling metadata to the uncharacterized data. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the metadata is a type of physician and the data used to identify the physician is their billing pattern. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the metadata is the classification of a test code in a hierarchy. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the metadata includes a specific set of fast Healthcare interoperability data formats and elements, other HL7 data format and elements, or the like. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and for generating inferences comprising a parser, a customer identification module, a health inference engine, and a genetic conflict inference engine. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and where the data sets are selected from the group comprising health care clinical, claims, genomic, immunochemistry, transactional, ordering data, demographic data, Lifestyle and social health data, patient relationships, and patient behaviors. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and providing patient matching comprising collecting a set of patient demographic information from a. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and raising an exception when an unmapped value comes to the PDMP provider. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and mapping test names to PDMP values on the gateway data provider. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and mapping test names to PDMP values. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and dynamically adjusting CRM Layout, Dashboards, or Reports in response to customer activations. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and technique for detecting doc shopping. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for automated, intelligent drug consistency reporting comprising a lab data collection module that integrates patient drug toxicology data, user reported symptoms, and patient prescriptions; a consistency module that applies a set of rules and algorithms to determine the if the metabolites of the toxicology test are consistent with the known patient prescription; and an interaction module that analyzes the detected metabolites to see if they indicate a potential adverse reaction and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises an education module that informs the prescribing physician which metabolites can be expected based on the patients known prescriptions. In embodiments, provided herein is a system where the system further comprises an education module that informs the prescribing physician which metabolites can be expected based on the patients known prescriptions and where the system further comprises a recommendation module that provides the physician with an indicated likelihood that the patient is abusing and a risk report for the physician to work with the patient. In embodiments, provided herein is a system where the system further comprises an education module that informs the prescribing physician which metabolites can be expected based on the patients known prescriptions and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises an education module that informs the prescribing physician which metabolites can be expected based on the patients known prescriptions and where the workflow provides intervention or drug protocol instructions to a physician to apply to a patient. In embodiments, provided herein is a system where the system further comprises an education module that informs the prescribing physician which metabolites can be expected based on the patients known prescriptions and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises an education module that informs the prescribing physician which metabolites can be expected based on the patients known prescriptions and where the data analyzed is X, Y and the pathology is diabetes. In embodiments, provided herein is a system where the system further comprises an education module that informs the prescribing physician which metabolites can be expected based on the patients known prescriptions and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises an education module that informs the prescribing physician which metabolites can be expected based on the patients known prescriptions and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises an education module that informs the prescribing physician which metabolites can be expected based on the patients known prescriptions and where the metadata is a type of physician and the data used to identify the physician is their billing pattern. In embodiments, provided herein is a system where the system further comprises an education module that informs the prescribing physician which metabolites can be expected based on the patients known prescriptions and where the metadata is the classification of a test code in a hierarchy. In embodiments, provided herein is a system where the system further comprises an education module that informs the prescribing physician which metabolites can be expected based on the patients known prescriptions and where the metadata includes a specific set of fast Healthcare interoperability data formats and elements, other HL7 data format and elements, or the like. In embodiments, provided herein is a system where the system further comprises an education module that informs the prescribing physician which metabolites can be expected based on the patients known prescriptions and for generating inferences comprising a parser, a customer identification module, a health inference engine, and a genetic conflict inference engine. In embodiments, provided herein is a system where the system further comprises an education module that informs the prescribing physician which metabolites can be expected based on the patients known prescriptions and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises an education module that informs the prescribing physician which metabolites can be expected based on the patients known prescriptions and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises an education module that informs the prescribing physician which metabolites can be expected based on the patients known prescriptions and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises an education module that informs the prescribing physician which metabolites can be expected based on the patients known prescriptions and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises an education module that informs the prescribing physician which metabolites can be expected based on the patients known prescriptions and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises an education module that informs the prescribing physician which metabolites can be expected based on the patients known prescriptions and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises an education module that informs the prescribing physician which metabolites can be expected based on the patients known prescriptions and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises an education module that informs the prescribing physician which metabolites can be expected based on the patients known prescriptions and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises an education module that informs the prescribing physician which metabolites can be expected based on the patients known prescriptions and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises an education module that informs the prescribing physician which metabolites can be expected based on the patients known prescriptions and providing patient matching comprising collecting a set of patient demographic information from a. In embodiments, provided herein is a system where the system further comprises an education module that informs the prescribing physician which metabolites can be expected based on the patients known prescriptions and raising an exception when an unmapped value comes to the PDMP provider. In embodiments, provided herein is a system where the system further comprises an education module that informs the prescribing physician which metabolites can be expected based on the patients known prescriptions and mapping test names to PDMP values on the gateway data provider. In embodiments, provided herein is a system where the system further comprises an education module that informs the prescribing physician which metabolites can be expected based on the patients known prescriptions and mapping test names to PDMP values. In embodiments, provided herein is a system where the system further comprises an education module that informs the prescribing physician which metabolites can be expected based on the patients known prescriptions and dynamically adjusting CRM Layout, Dashboards, or Reports in response to customer activations. In embodiments, provided herein is a system where the system further comprises an education module that informs the prescribing physician which metabolites can be expected based on the patients known prescriptions and technique for detecting doc shopping. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises an education module that informs the prescribing physician which metabolites can be expected based on the patients known prescriptions and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises an education module that informs the prescribing physician which metabolites can be expected based on the patients known prescriptions and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises an education module that informs the prescribing physician which metabolites can be expected based on the patients known prescriptions and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises an education module that informs the prescribing physician which metabolites can be expected based on the patients known prescriptions and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises an education module that informs the prescribing physician which metabolites can be expected based on the patients known prescriptions and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises an education module that informs the prescribing physician which metabolites can be expected based on the patients known prescriptions and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system where the system further comprises a recommendation module that provides the physician with an indicated likelihood that the patient is abusing and a risk report for the physician to work with the patient. In embodiments, provided herein is a system where the system further comprises a recommendation module that provides the physician with an indicated likelihood that the patient is abusing and a risk report for the physician to work with the patient and of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a system where the system further comprises a recommendation module that provides the physician with an indicated likelihood that the patient is abusing and a risk report for the physician to work with the patient and where the workflow provides intervention or drug protocol instructions to a physician to apply to a patient. In embodiments, provided herein is a system where the system further comprises a recommendation module that provides the physician with an indicated likelihood that the patient is abusing and a risk report for the physician to work with the patient and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a system where the system further comprises a recommendation module that provides the physician with an indicated likelihood that the patient is abusing and a risk report for the physician to work with the patient and where the data analyzed is X, Y and the pathology is diabetes. In embodiments, provided herein is a system where the system further comprises a recommendation module that provides the physician with an indicated likelihood that the patient is abusing and a risk report for the physician to work with the patient and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system where the system further comprises a recommendation module that provides the physician with an indicated likelihood that the patient is abusing and a risk report for the physician to work with the patient and for normalizing data in a medical database comprising, using machine learning to characterize a set of normalized data, applying an ML classification to a new set of unlabeled data, generating metadata and adding labelling metadata to the uncharacterized data. In embodiments, provided herein is a system where the system further comprises a recommendation module that provides the physician with an indicated likelihood that the patient is abusing and a risk report for the physician to work with the patient and where the metadata is a type of physician and the data used to identify the physician is their billing pattern. In embodiments, provided herein is a system where the system further comprises a recommendation module that provides the physician with an indicated likelihood that the patient is abusing and a risk report for the physician to work with the patient and where the metadata is the classification of a test code in a hierarchy. In embodiments, provided herein is a system where the system further comprises a recommendation module that provides the physician with an indicated likelihood that the patient is abusing and a risk report for the physician to work with the patient and where the metadata includes a specific set of fast Healthcare interoperability data formats and elements, other HL7 data format and elements, or the like. In embodiments, provided herein is a system where the system further comprises a recommendation module that provides the physician with an indicated likelihood that the patient is abusing and a risk report for the physician to work with the patient and for generating inferences comprising a parser, a customer identification module, a health inference engine, and a genetic conflict inference engine. In embodiments, provided herein is a system where the system further comprises a recommendation module that provides the physician with an indicated likelihood that the patient is abusing and a risk report for the physician to work with the patient and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system where the system further comprises a recommendation module that provides the physician with an indicated likelihood that the patient is abusing and a risk report for the physician to work with the patient and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system where the system further comprises a recommendation module that provides the physician with an indicated likelihood that the patient is abusing and a risk report for the physician to work with the patient and where the data sets are selected from the group comprising health care clinical, claims, genomic, immunochemistry, transactional, ordering data, demographic data, Lifestyle and social health data, patient relationships, and patient behaviors. In embodiments, provided herein is a system where the system further comprises a recommendation module that provides the physician with an indicated likelihood that the patient is abusing and a risk report for the physician to work with the patient and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system where the system further comprises a recommendation module that provides the physician with an indicated likelihood that the patient is abusing and a risk report for the physician to work with the patient and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system where the system further comprises a recommendation module that provides the physician with an indicated likelihood that the patient is abusing and a risk report for the physician to work with the patient and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system where the system further comprises a recommendation module that provides the physician with an indicated likelihood that the patient is abusing and a risk report for the physician to work with the patient and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system where the system further comprises a recommendation module that provides the physician with an indicated likelihood that the patient is abusing and a risk report for the physician to work with the patient and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system where the system further comprises a recommendation module that provides the physician with an indicated likelihood that the patient is abusing and a risk report for the physician to work with the patient and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system where the system further comprises a recommendation module that provides the physician with an indicated likelihood that the patient is abusing and a risk report for the physician to work with the patient and providing patient matching comprising collecting a set of patient demographic information from a. In embodiments, provided herein is a system where the system further comprises a recommendation module that provides the physician with an indicated likelihood that the patient is abusing and a risk report for the physician to work with the patient and raising an exception when an unmapped value comes to the PDMP provider. In embodiments, provided herein is a system where the system further comprises a recommendation module that provides the physician with an indicated likelihood that the patient is abusing and a risk report for the physician to work with the patient and mapping test names to PDMP values on the gateway data provider. In embodiments, provided herein is a system where the system further comprises a recommendation module that provides the physician with an indicated likelihood that the patient is abusing and a risk report for the physician to work with the patient and mapping test names to PDMP values. In embodiments, provided herein is a system where the system further comprises a recommendation module that provides the physician with an indicated likelihood that the patient is abusing and a risk report for the physician to work with the patient and dynamically adjusting CRM Layout, Dashboards, or Reports in response to customer activations. In embodiments, provided herein is a system where the system further comprises a recommendation module that provides the physician with an indicated likelihood that the patient is abusing and a risk report for the physician to work with the patient and technique for detecting doc shopping. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system where the system further comprises a recommendation module that provides the physician with an indicated likelihood that the patient is abusing and a risk report for the physician to work with the patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system where the system further comprises a recommendation module that provides the physician with an indicated likelihood that the patient is abusing and a risk report for the physician to work with the patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system where the system further comprises a recommendation module that provides the physician with an indicated likelihood that the patient is abusing and a risk report for the physician to work with the patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system where the system further comprises a recommendation module that provides the physician with an indicated likelihood that the patient is abusing and a risk report for the physician to work with the patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system where the system further comprises a recommendation module that provides the physician with an indicated likelihood that the patient is abusing and a risk report for the physician to work with the patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system where the system further comprises a recommendation module that provides the physician with an indicated likelihood that the patient is abusing and a risk report for the physician to work with the patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital management solution.

In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and where the workflow provides intervention or drug protocol instructions to a physician to apply to a patient. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and where the data analyzed is X, Y and the pathology is diabetes. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and for normalizing data in a medical database comprising, using machine learning to characterize a set of normalized data, applying an ML classification to a new set of unlabeled data, generating metadata and adding labelling metadata to the uncharacterized data. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and where the metadata is a type of physician and the data used to identify the physician is their billing pattern. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and where the metadata is the classification of a test code in a hierarchy. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and where the metadata includes a specific set of fast Healthcare interoperability data formats and elements, other HL7 data format and elements, or the like. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and for generating inferences comprising a parser, a customer identification module, a health inference engine, and a genetic conflict inference engine. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and where the data sets are selected from the group comprising health care clinical, claims, genomic, immunochemistry, transactional, ordering data, demographic data, Lifestyle and social health data, patient relationships, and patient behaviors. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and providing patient matching comprising collecting a set of patient demographic information from a. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and raising an exception when an unmapped value comes to the PDMP provider. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and mapping test names to PDMP values on the gateway data provider. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and mapping test names to PDMP values. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and dynamically adjusting CRM Layout, Dashboards, or Reports in response to customer activations. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and technique for detecting doc shopping. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method of reducing hospital risk comprising monitoring the aggregate PDMP data for all patients of that hospital and each of the prescribing physicians in that hospital, applying a set of periodically updated heuristics that detect anomalous patterns of prescription, mapping the anomalous prescription activity to patients and to physicians, identifying an inconsistency based on the anomalous prescription activity, and activating a workflow to notify a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the workflow provides intervention or drug protocol instructions to a physician to apply to a patient. In embodiments, provided herein is a method where the workflow provides intervention or drug protocol instructions to a physician to apply to a patient and for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method where the workflow provides intervention or drug protocol instructions to a physician to apply to a patient and where the data analyzed is X, Y and the pathology is diabetes. In embodiments, provided herein is a method where the workflow provides intervention or drug protocol instructions to a physician to apply to a patient and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the workflow provides intervention or drug protocol instructions to a physician to apply to a patient and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the workflow provides intervention or drug protocol instructions to a physician to apply to a patient and where the metadata is a type of physician and the data used to identify the physician is their billing pattern. In embodiments, provided herein is a method where the workflow provides intervention or drug protocol instructions to a physician to apply to a patient and where the metadata is the classification of a test code in a hierarchy. In embodiments, provided herein is a method where the workflow provides intervention or drug protocol instructions to a physician to apply to a patient and where the metadata includes a specific set of fast Healthcare interoperability data formats and elements, other HL7 data format and elements, or the like. In embodiments, provided herein is a method where the workflow provides intervention or drug protocol instructions to a physician to apply to a patient and for generating inferences comprising a parser, a customer identification module, a health inference engine, and a genetic conflict inference engine. In embodiments, provided herein is a method where the workflow provides intervention or drug protocol instructions to a physician to apply to a patient and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the workflow provides intervention or drug protocol instructions to a physician to apply to a patient and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the workflow provides intervention or drug protocol instructions to a physician to apply to a patient and where the data sets are selected from the group comprising health care clinical, claims, genomic, immunochemistry, transactional, ordering data, demographic data, Lifestyle and social health data, patient relationships, and patient behaviors. In embodiments, provided herein is a method where the workflow provides intervention or drug protocol instructions to a physician to apply to a patient and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the workflow provides intervention or drug protocol instructions to a physician to apply to a patient and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the workflow provides intervention or drug protocol instructions to a physician to apply to a patient and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the workflow provides intervention or drug protocol instructions to a physician to apply to a patient and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the workflow provides intervention or drug protocol instructions to a physician to apply to a patient and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the workflow provides intervention or drug protocol instructions to a physician to apply to a patient and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the workflow provides intervention or drug protocol instructions to a physician to apply to a patient and providing patient matching comprising collecting a set of patient demographic information from a. In embodiments, provided herein is a method where the workflow provides intervention or drug protocol instructions to a physician to apply to a patient and raising an exception when an unmapped value comes to the PDMP provider. In embodiments, provided herein is a method where the workflow provides intervention or drug protocol instructions to a physician to apply to a patient and mapping test names to PDMP values on the gateway data provider. In embodiments, provided herein is a method where the workflow provides intervention or drug protocol instructions to a physician to apply to a patient and mapping test names to PDMP values. In embodiments, provided herein is a method where the workflow provides intervention or drug protocol instructions to a physician to apply to a patient and dynamically adjusting CRM Layout, Dashboards, or Reports in response to customer activations. In embodiments, provided herein is a method where the workflow provides intervention or drug protocol instructions to a physician to apply to a patient and technique for detecting doc shopping. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the workflow provides intervention or drug protocol instructions to a physician to apply to a patient and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the workflow provides intervention or drug protocol instructions to a physician to apply to a patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the workflow provides intervention or drug protocol instructions to a physician to apply to a patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the workflow provides intervention or drug protocol instructions to a physician to apply to a patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the workflow provides intervention or drug protocol instructions to a physician to apply to a patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the workflow provides intervention or drug protocol instructions to a physician to apply to a patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the workflow provides intervention or drug protocol instructions to a physician to apply to a patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and where the data analyzed is X, Y and the pathology is diabetes. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and for normalizing data in a medical database comprising, using machine learning to characterize a set of normalized data, applying an ML classification to a new set of unlabeled data, generating metadata and adding labelling metadata to the uncharacterized data. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and where the metadata is a type of physician and the data used to identify the physician is their billing pattern. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and where the metadata is the classification of a test code in a hierarchy. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and where the metadata includes a specific set of fast Healthcare interoperability data formats and elements, other HL7 data format and elements, or the like. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and for generating inferences comprising a parser, a customer identification module, a health inference engine, and a genetic conflict inference engine. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and where the data sets are selected from the group comprising health care clinical, claims, genomic, immunochemistry, transactional, ordering data, demographic data, Lifestyle and social health data, patient relationships, and patient behaviors. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and providing patient matching comprising collecting a set of patient demographic information from a. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and raising an exception when an unmapped value comes to the PDMP provider. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and mapping test names to PDMP values on the gateway data provider. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and mapping test names to PDMP values. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and dynamically adjusting CRM Layout, Dashboards, or Reports in response to customer activations. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and technique for detecting doc shopping. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for making inferences from a set of patient test data comprising, running a machine learning algorithm on a set of data containing millions of records of patient test data and corresponding outcomes, correlating the patient outcomes with signals from the patient test data, and determining the likelihood of a particular pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the data analyzed is X, Y and the pathology is diabetes. In embodiments, provided herein is a method where the data analyzed is X, Y and the pathology is diabetes and for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a method where the data analyzed is X, Y and the pathology is diabetes and for normalizing data in a medical database comprising, using machine learning to characterize a set of normalized data, applying an ML classification to a new set of unlabeled data, generating metadata and adding labelling metadata to the uncharacterized data. In embodiments, provided herein is a method where the data analyzed is X, Y and the pathology is diabetes and where the metadata is a type of physician and the data used to identify the physician is their billing pattern. In embodiments, provided herein is a method where the data analyzed is X, Y and the pathology is diabetes and where the metadata is the classification of a test code in a hierarchy. In embodiments, provided herein is a method where the data analyzed is X, Y and the pathology is diabetes and where the metadata includes a specific set of fast Healthcare interoperability data formats and elements, other HL7 data format and elements, or the like. In embodiments, provided herein is a method where the data analyzed is X, Y and the pathology is diabetes and for generating inferences comprising a parser, a customer identification module, a health inference engine, and a genetic conflict inference engine. In embodiments, provided herein is a method where the data analyzed is X, Y and the pathology is diabetes and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the data analyzed is X, Y and the pathology is diabetes and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the data analyzed is X, Y and the pathology is diabetes and where the data sets are selected from the group comprising health care clinical, claims, genomic, immunochemistry, transactional, ordering data, demographic data, Lifestyle and social health data, patient relationships, and patient behaviors. In embodiments, provided herein is a method where the data analyzed is X, Y and the pathology is diabetes and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the data analyzed is X, Y and the pathology is diabetes and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the data analyzed is X, Y and the pathology is diabetes and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the data analyzed is X, Y and the pathology is diabetes and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the data analyzed is X, Y and the pathology is diabetes and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the data analyzed is X, Y and the pathology is diabetes and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the data analyzed is X, Y and the pathology is diabetes and providing patient matching comprising collecting a set of patient demographic information from a. In embodiments, provided herein is a method where the data analyzed is X, Y and the pathology is diabetes and raising an exception when an unmapped value comes to the PDMP provider. In embodiments, provided herein is a method where the data analyzed is X, Y and the pathology is diabetes and mapping test names to PDMP values on the gateway data provider. In embodiments, provided herein is a method where the data analyzed is X, Y and the pathology is diabetes and mapping test names to PDMP values. In embodiments, provided herein is a method where the data analyzed is X, Y and the pathology is diabetes and dynamically adjusting CRM Layout, Dashboards, or Reports in response to customer activations. In embodiments, provided herein is a method where the data analyzed is X, Y and the pathology is diabetes and technique for detecting doc shopping. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the data analyzed is X, Y and the pathology is diabetes and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the data analyzed is X, Y and the pathology is diabetes and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the data analyzed is X, Y and the pathology is diabetes and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the data analyzed is X, Y and the pathology is diabetes and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the data analyzed is X, Y and the pathology is diabetes and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the data analyzed is X, Y and the pathology is diabetes and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and for normalizing data in a medical database comprising, using machine learning to characterize a set of normalized data, applying an ML classification to a new set of unlabeled data, generating metadata and adding labelling metadata to the uncharacterized data. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and where the metadata is a type of physician and the data used to identify the physician is their billing pattern. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and where the metadata is the classification of a test code in a hierarchy. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and where the metadata includes a specific set of fast Healthcare interoperability data formats and elements, other HL7 data format and elements, or the like. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and for generating inferences comprising a parser, a customer identification module, a health inference engine, and a genetic conflict inference engine. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and where the data sets are selected from the group comprising health care clinical, claims, genomic, immunochemistry, transactional, ordering data, demographic data, Lifestyle and social health data, patient relationships, and patient behaviors. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and providing patient matching comprising collecting a set of patient demographic information from a. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and raising an exception when an unmapped value comes to the PDMP provider. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and mapping test names to PDMP values on the gateway data provider. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and mapping test names to PDMP values. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and dynamically adjusting CRM Layout, Dashboards, or Reports in response to customer activations. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and technique for detecting doc shopping. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by to a raw data cluster, wherein encrypting the data and controlling access to the data. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for making inferences from a set of patient test data comprising a machine learning module for analyzing a set of data containing millions of records of laboratory testing data including corresponding outcomes, a correlation module for correlating the patient outcomes with signals from the patient test data, analyzing the testing data corresponding to a set of patients, and providing a listing of the set of patients most likely to have that pathology and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for normalizing data in a medical database comprising, using machine learning to characterize a set of normalized data, applying an ML classification to a new set of unlabeled data, generating metadata and adding labelling metadata to the uncharacterized data. In embodiments, provided herein is a method for normalizing data in a medical database comprising, using machine learning to characterize a set of normalized data, applying an ML classification to a new set of unlabeled data, generating metadata and adding labelling metadata to the uncharacterized data and where the metadata is a type of physician and the data used to identify the physician is their billing pattern. In embodiments, provided herein is a method for normalizing data in a medical database comprising, using machine learning to characterize a set of normalized data, applying an ML classification to a new set of unlabeled data, generating metadata and adding labelling metadata to the uncharacterized data and where the metadata is the classification of a test code in a hierarchy. In embodiments, provided herein is a method for normalizing data in a medical database comprising, using machine learning to characterize a set of normalized data, applying an ML classification to a new set of unlabeled data, generating metadata and adding labelling metadata to the uncharacterized data and where the metadata includes a specific set of fast Healthcare interoperability data formats and elements, other HL7 data format and elements, or the like. In embodiments, provided herein is a method for normalizing data in a medical database comprising, using machine learning to characterize a set of normalized data, applying an ML classification to a new set of unlabeled data, generating metadata and adding labelling metadata to the uncharacterized data and for generating inferences comprising a parser, a customer identification module, a health inference engine, and a genetic conflict inference engine. In embodiments, provided herein is a method for normalizing data in a medical database comprising, using machine learning to characterize a set of normalized data, applying an ML classification to a new set of unlabeled data, generating metadata and adding labelling metadata to the uncharacterized data and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method for normalizing data in a medical database comprising, using machine learning to characterize a set of normalized data, applying an ML classification to a new set of unlabeled data, generating metadata and adding labelling metadata to the uncharacterized data and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method for normalizing data in a medical database comprising, using machine learning to characterize a set of normalized data, applying an ML classification to a new set of unlabeled data, generating metadata and adding labelling metadata to the uncharacterized data and where the data sets are selected from the group comprising health care clinical, claims, genomic, immunochemistry, transactional, ordering data, demographic data, Lifestyle and social health data, patient relationships, and patient behaviors. In embodiments, provided herein is a method for normalizing data in a medical database comprising, using machine learning to characterize a set of normalized data, applying an ML classification to a new set of unlabeled data, generating metadata and adding labelling metadata to the uncharacterized data and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method for normalizing data in a medical database comprising, using machine learning to characterize a set of normalized data, applying an ML classification to a new set of unlabeled data, generating metadata and adding labelling metadata to the uncharacterized data and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method for normalizing data in a medical database comprising, using machine learning to characterize a set of normalized data, applying an ML classification to a new set of unlabeled data, generating metadata and adding labelling metadata to the uncharacterized data and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method for normalizing data in a medical database comprising, using machine learning to characterize a set of normalized data, applying an ML classification to a new set of unlabeled data, generating metadata and adding labelling metadata to the uncharacterized data and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for normalizing data in a medical database comprising, using machine learning to characterize a set of normalized data, applying an ML classification to a new set of unlabeled data, generating metadata and adding labelling metadata to the uncharacterized data and providing patient matching comprising collecting a set of patient demographic information from a. In embodiments, provided herein is a method for normalizing data in a medical database comprising, using machine learning to characterize a set of normalized data, applying an ML classification to a new set of unlabeled data, generating metadata and adding labelling metadata to the uncharacterized data and raising an exception when an unmapped value comes to the PDMP provider. In embodiments, provided herein is a method for normalizing data in a medical database comprising, using machine learning to characterize a set of normalized data, applying an ML classification to a new set of unlabeled data, generating metadata and adding labelling metadata to the uncharacterized data and mapping test names to PDMP values on the gateway data provider. In embodiments, provided herein is a method for normalizing data in a medical database comprising, using machine learning to characterize a set of normalized data, applying an ML classification to a new set of unlabeled data, generating metadata and adding labelling metadata to the uncharacterized data and mapping test names to PDMP values. In embodiments, provided herein is a method for normalizing data in a medical database comprising, using machine learning to characterize a set of normalized data, applying an ML classification to a new set of unlabeled data, generating metadata and adding labelling metadata to the uncharacterized data and dynamically adjusting CRM Layout, Dashboards, or Reports in response to customer activations. In embodiments, provided herein is a method for normalizing data in a medical database comprising, using machine learning to characterize a set of normalized data, applying an ML classification to a new set of unlabeled data, generating metadata and adding labelling metadata to the uncharacterized data and technique for detecting doc shopping. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for normalizing data in a medical database comprising, using machine learning to characterize a set of normalized data, applying an ML classification to a new set of unlabeled data, generating metadata and adding labelling metadata to the uncharacterized data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for normalizing data in a medical database comprising, using machine learning to characterize a set of normalized data, applying an ML classification to a new set of unlabeled data, generating metadata and adding labelling metadata to the uncharacterized data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for normalizing data in a medical database comprising, using machine learning to characterize a set of normalized data, applying an ML classification to a new set of unlabeled data, generating metadata and adding labelling metadata to the uncharacterized data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the metadata is a type of physician and the data used to identify the physician is their billing pattern. In embodiments, provided herein is a method where the metadata is a type of physician and the data used to identify the physician is their billing pattern and where the metadata is the classification of a test code in a hierarchy. In embodiments, provided herein is a method where the metadata is a type of physician and the data used to identify the physician is their billing pattern and where the metadata includes a specific set of fast Healthcare interoperability data formats and elements, other HL7 data format and elements, or the like. In embodiments, provided herein is a method where the metadata is a type of physician and the data used to identify the physician is their billing pattern and for generating inferences comprising a parser, a customer identification module, a health inference engine, and a genetic conflict inference engine. In embodiments, provided herein is a method where the metadata is a type of physician and the data used to identify the physician is their billing pattern and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the metadata is a type of physician and the data used to identify the physician is their billing pattern and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the metadata is a type of physician and the data used to identify the physician is their billing pattern and where the data sets are selected from the group comprising health care clinical, claims, genomic, immunochemistry, transactional, ordering data, demographic data, Lifestyle and social health data, patient relationships, and patient behaviors. In embodiments, provided herein is a method where the metadata is a type of physician and the data used to identify the physician is their billing pattern and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the metadata is a type of physician and the data used to identify the physician is their billing pattern and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the metadata is a type of physician and the data used to identify the physician is their billing pattern and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the metadata is a type of physician and the data used to identify the physician is their billing pattern and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the metadata is a type of physician and the data used to identify the physician is their billing pattern and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the metadata is a type of physician and the data used to identify the physician is their billing pattern and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the metadata is a type of physician and the data used to identify the physician is their billing pattern and providing patient matching comprising collecting a set of patient demographic information from a. In embodiments, provided herein is a method where the metadata is a type of physician and the data used to identify the physician is their billing pattern and raising an exception when an unmapped value comes to the PDMP provider. In embodiments, provided herein is a method where the metadata is a type of physician and the data used to identify the physician is their billing pattern and mapping test names to PDMP values on the gateway data provider. In embodiments, provided herein is a method where the metadata is a type of physician and the data used to identify the physician is their billing pattern and mapping test names to PDMP values. In embodiments, provided herein is a method where the metadata is a type of physician and the data used to identify the physician is their billing pattern and dynamically adjusting CRM Layout, Dashboards, or Reports in response to customer activations. In embodiments, provided herein is a method where the metadata is a type of physician and the data used to identify the physician is their billing pattern and technique for detecting doc shopping. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the metadata is a type of physician and the data used to identify the physician is their billing pattern and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the metadata is a type of physician and the data used to identify the physician is their billing pattern and receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the metadata is a type of physician and the data used to identify the physician is their billing pattern and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the metadata is a type of physician and the data used to identify the physician is their billing pattern and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the metadata is a type of physician and the data used to identify the physician is their billing pattern and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the metadata is a type of physician and the data used to identify the physician is their billing pattern and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the metadata is a type of physician and the data used to identify the physician is their billing pattern and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the metadata is the classification of a test code in a hierarchy. In embodiments, provided herein is a method where the metadata is the classification of a test code in a hierarchy and where the metadata includes a specific set of fast Healthcare interoperability data formats and elements, other HL7 data format and elements, or the like. In embodiments, provided herein is a method where the metadata is the classification of a test code in a hierarchy and for generating inferences comprising a parser, a customer identification module, a health inference engine, and a genetic conflict inference engine. In embodiments, provided herein is a method where the metadata is the classification of a test code in a hierarchy and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the metadata is the classification of a test code in a hierarchy and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the metadata is the classification of a test code in a hierarchy and where the data sets are selected from the group comprising health care clinical, claims, genomic, immunochemistry, transactional, ordering data, demographic data, Lifestyle and social health data, patient relationships, and patient behaviors. In embodiments, provided herein is a method where the metadata is the classification of a test code in a hierarchy and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the metadata is the classification of a test code in a hierarchy and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the metadata is the classification of a test code in a hierarchy and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the metadata is the classification of a test code in a hierarchy and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the metadata is the classification of a test code in a hierarchy and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the metadata is the classification of a test code in a hierarchy and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the metadata is the classification of a test code in a hierarchy and providing patient matching comprising collecting a set of patient demographic information from a. In embodiments, provided herein is a method where the metadata is the classification of a test code in a hierarchy and raising an exception when an unmapped value comes to the PDMP provider. In embodiments, provided herein is a method where the metadata is the classification of a test code in a hierarchy and mapping test names to PDMP values on the gateway data provider. In embodiments, provided herein is a method where the metadata is the classification of a test code in a hierarchy and mapping test names to PDMP values. In embodiments, provided herein is a method where the metadata is the classification of a test code in a hierarchy and dynamically adjusting CRM Layout, Dashboards, or Reports in response to customer activations. In embodiments, provided herein is a method where the metadata is the classification of a test code in a hierarchy and technique for detecting doc shopping. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the metadata is the classification of a test code in a hierarchy and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata is the classification of a test code in a hierarchy and receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the metadata is the classification of a test code in a hierarchy and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the metadata is the classification of a test code in a hierarchy and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the metadata is the classification of a test code in a hierarchy and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the metadata is the classification of a test code in a hierarchy and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the metadata is the classification of a test code in a hierarchy and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the metadata includes a specific set of fast Healthcare interoperability data formats and elements, other HL7 data format and elements, or the like. In embodiments, provided herein is a method where the metadata includes a specific set of fast Healthcare interoperability data formats and elements, other HL7 data format and elements, or the like and for generating inferences comprising a parser, a customer identification module, a health inference engine, and a genetic conflict inference engine. In embodiments, provided herein is a method where the metadata includes a specific set of fast Healthcare interoperability data formats and elements, other HL7 data format and elements, or the like and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method where the metadata includes a specific set of fast Healthcare interoperability data formats and elements, other HL7 data format and elements, or the like and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method where the metadata includes a specific set of fast Healthcare interoperability data formats and elements, other HL7 data format and elements, or the like and where the data sets are selected from the group comprising health care clinical, claims, genomic, immunochemistry, transactional, ordering data, demographic data, Lifestyle and social health data, patient relationships, and patient behaviors. In embodiments, provided herein is a method where the metadata includes a specific set of fast Healthcare interoperability data formats and elements, other HL7 data format and elements, or the like and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the metadata includes a specific set of fast Healthcare interoperability data formats and elements, other HL7 data format and elements, or the like and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the metadata includes a specific set of fast Healthcare interoperability data formats and elements, other HL7 data format and elements, or the like and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the metadata includes a specific set of fast Healthcare interoperability data formats and elements, other HL7 data format and elements, or the like and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the metadata includes a specific set of fast Healthcare interoperability data formats and elements, other HL7 data format and elements, or the like and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the metadata includes a specific set of fast Healthcare interoperability data formats and elements, other HL7 data format and elements, or the like and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the metadata includes a specific set of fast Healthcare interoperability data formats and elements, other HL7 data format and elements, or the like and providing patient matching comprising collecting a set of patient demographic information from a. In embodiments, provided herein is a method where the metadata includes a specific set of fast Healthcare interoperability data formats and elements, other HL7 data format and elements, or the like and raising an exception when an unmapped value comes to the PDMP provider. In embodiments, provided herein is a method where the metadata includes a specific set of fast Healthcare interoperability data formats and elements, other HL7 data format and elements, or the like and mapping test names to PDMP values on the gateway data provider. In embodiments, provided herein is a method where the metadata includes a specific set of fast Healthcare interoperability data formats and elements, other HL7 data format and elements, or the like and mapping test names to PDMP values. In embodiments, provided herein is a method where the metadata includes a specific set of fast Healthcare interoperability data formats and elements, other HL7 data format and elements, or the like and dynamically adjusting CRM Layout, Dashboards, or Reports in response to customer activations. In embodiments, provided herein is a method where the metadata includes a specific set of fast Healthcare interoperability data formats and elements, other HL7 data format and elements, or the like and technique for detecting doc shopping. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the metadata includes a specific set of fast Healthcare interoperability data formats and elements, other HL7 data format and elements, or the like and ingesting, by a healthcare insight computing device, patient data of a patient received from one of a plurality of patient data providers; determining, by data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the metadata includes a specific set of fast Healthcare interoperability data formats and elements, other HL7 data format and elements, or the like and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the metadata includes a specific set of fast Healthcare interoperability data formats and elements, other HL7 data format and elements, or the like and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the metadata includes a specific set of fast Healthcare interoperability data formats and elements, other HL7 data format and elements, or the like and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the metadata includes a specific set of fast Healthcare interoperability data formats and elements, other HL7 data format and elements, or the like and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the metadata includes a specific set of fast Healthcare interoperability data formats and elements, other HL7 data format and elements, or the like and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for generating inferences comprising a parser, a customer identification module, a health inference engine, and a genetic conflict inference engine. In embodiments, provided herein is a system for generating inferences comprising a parser, a customer identification module, a health inference engine, and a genetic conflict inference engine and improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a system for generating inferences comprising a parser, a customer identification module, a health inference engine, and a genetic conflict inference engine and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a system for generating inferences comprising a parser, a customer identification module, a health inference engine, and a genetic conflict inference engine and where the data sets are selected from the group comprising health care clinical, claims, genomic, immunochemistry, transactional, ordering data, demographic data, Lifestyle and social health data, patient relationships, and patient behaviors. In embodiments, provided herein is a system for generating inferences comprising a parser, a customer identification module, a health inference engine, and a genetic conflict inference engine and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for generating inferences comprising a parser, a customer identification module, a health inference engine, and a genetic conflict inference engine and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for generating inferences comprising a parser, a customer identification module, a health inference engine, and a genetic conflict inference engine and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for generating inferences comprising a parser, a customer identification module, a health inference engine, and a genetic conflict inference engine and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for generating inferences comprising a parser, a customer identification module, a health inference engine, and a genetic conflict inference engine and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for generating inferences comprising a parser, a customer identification module, a health inference engine, and a genetic conflict inference engine and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for generating inferences comprising a parser, a customer identification module, a health inference engine, and a genetic conflict inference engine and providing patient matching comprising collecting a set of patient demographic information from a. In embodiments, provided herein is a system for generating inferences comprising a parser, a customer identification module, a health inference engine, and a genetic conflict inference engine and raising an exception when an unmapped value comes to the PDMP provider. In embodiments, provided herein is a system for generating inferences comprising a parser, a customer identification module, a health inference engine, and a genetic conflict inference engine and mapping test names to PDMP values on the gateway data provider. In embodiments, provided herein is a system for generating inferences comprising a parser, a customer identification module, a health inference engine, and a genetic conflict inference engine and mapping test names to PDMP values. In embodiments, provided herein is a system for generating inferences comprising a parser, a customer identification module, a health inference engine, and a genetic conflict inference engine and dynamically adjusting CRM Layout, Dashboards, or Reports in response to customer activations. In embodiments, provided herein is a system for generating inferences comprising a parser, a customer identification module, a health inference engine, and a genetic conflict inference engine and technique for detecting doc shopping. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the to a raw data cluster, wherein applying an algorithm selected from the set comprising transformation algorithms, normalization operations, refinement operations. In embodiments, provided herein is a system for generating inferences comprising a parser, a customer identification module, a health inference engine, and a genetic conflict inference engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for generating inferences comprising a parser, a customer identification module, a health inference engine, and a genetic conflict inference engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for generating inferences comprising a parser, a customer identification module, a health inference engine, and a genetic conflict inference engine and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for generating inferences comprising a parser, a customer identification module, a health inference engine, and a genetic conflict inference engine and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for generating inferences comprising a parser, a customer identification module, a health inference engine, and a genetic conflict inference engine and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for generating inferences comprising a parser, a customer identification module, a health inference engine, and a genetic conflict inference engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and where the data sets are selected from the group comprising health care clinical, claims, genomic, immunochemistry, transactional, ordering data, demographic data, Lifestyle and social health data, patient relationships, and patient behaviors. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and providing patient matching comprising collecting a set of patient demographic information from a. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and raising an exception when an unmapped value comes to the PDMP provider. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and mapping test names to PDMP values on the gateway data provider. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and mapping test names to PDMP values. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and dynamically adjusting CRM Layout, Dashboards, or Reports in response to customer activations. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and technique for detecting doc shopping. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a health information from a healthcare communication source at a healthcare relationship system; categorizing the health information into at least one security information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method improving health care outcomes for patients comprising, importing a plurality of high fidelity clinical lab data with corresponding patient claims data, parsing and normalizing analyzing the information using a machine learning system, developing correlations between patient data and lab results, and using the resulting data to inform a physician and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and where the data sets are selected from the group comprising health care clinical, claims, genomic, immunochemistry, transactional, ordering data, demographic data, Lifestyle and social health data, patient relationships, and patient behaviors. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and providing patient matching comprising collecting a set of patient demographic information from a. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and raising an exception when an unmapped value comes to the PDMP provider. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and mapping test names to PDMP values on the gateway data provider. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and mapping test names to PDMP values. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and dynamically adjusting CRM Layout, Dashboards, or Reports in response to customer activations. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and technique for detecting doc shopping. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers.

In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method of performing risk stratification of patients comprising, assembling data sets relating to a plurality of patients, ingesting the data into a matrix, making the data accessible to a high-speed search system, importing the data into a machine learning system, and performing data mining on the data to determine if a patient has an increased risk of developing a pathology of a particular kind based on the analysis and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method where the data sets are selected from the group comprising health care clinical, claims, genomic, immunochemistry, transactional, ordering data, demographic data, Lifestyle and social health data, patient relationships, and patient behaviors. In embodiments, provided herein is a method where the data sets are selected from the group comprising health care clinical, claims, genomic, immunochemistry, transactional, ordering data, demographic data, Lifestyle and social health data, patient relationships, and patient behaviors and for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a method where the data sets are selected from the group comprising health care clinical, claims, genomic, immunochemistry, transactional, ordering data, demographic data, Lifestyle and social health data, patient relationships, and patient behaviors and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a method where the data sets are selected from the group comprising health care clinical, claims, genomic, immunochemistry, transactional, ordering data, demographic data, Lifestyle and social health data, patient relationships, and patient behaviors and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method where the data sets are selected from the group comprising health care clinical, claims, genomic, immunochemistry, transactional, ordering data, demographic data, Lifestyle and social health data, patient relationships, and patient behaviors and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method where the data sets are selected from the group comprising health care clinical, claims, genomic, immunochemistry, transactional, ordering data, demographic data, Lifestyle and social health data, patient relationships, and patient behaviors and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method where the data sets are selected from the group comprising health care clinical, claims, genomic, immunochemistry, transactional, ordering data, demographic data, Lifestyle and social health data, patient relationships, and patient behaviors and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method where the data sets are selected from the group comprising health care clinical, claims, genomic, immunochemistry, transactional, ordering data, demographic data, Lifestyle and social health data, patient relationships, and patient behaviors and providing patient matching comprising collecting a set of patient demographic information from a. In embodiments, provided herein is a method where the data sets are selected from the group comprising health care clinical, claims, genomic, immunochemistry, transactional, ordering data, demographic data, Lifestyle and social health data, patient relationships, and patient behaviors and raising an exception when an unmapped value comes to the PDMP provider. In embodiments, provided herein is a method where the data sets are selected from the group comprising health care clinical, claims, genomic, immunochemistry, transactional, ordering data, demographic data, Lifestyle and social health data, patient relationships, and patient behaviors and mapping test names to PDMP values on the gateway data provider. In embodiments, provided herein is a method where the data sets are selected from the group comprising health care clinical, claims, genomic, immunochemistry, transactional, ordering data, demographic data, Lifestyle and social health data, patient relationships, and patient behaviors and mapping test names to PDMP values. In embodiments, provided herein is a method where the data sets are selected from the group comprising health care clinical, claims, genomic, immunochemistry, transactional, ordering data, demographic data, Lifestyle and social health data, patient relationships, and patient behaviors and dynamically adjusting CRM Layout, Dashboards, or Reports in response to customer activations. In embodiments, provided herein is a method where the data sets are selected from the group comprising health care clinical, claims, genomic, immunochemistry, transactional, ordering data, demographic data, Lifestyle and social health data, patient relationships, and patient behaviors and technique for detecting doc shopping. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method where the data sets are selected from the group comprising health care clinical, claims, genomic, immunochemistry, transactional, ordering data, demographic data, Lifestyle and social health data, patient relationships, and patient behaviors and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method where the data sets are selected from the group comprising health care clinical, claims, genomic, immunochemistry, transactional, ordering data, demographic data, Lifestyle and social health data, patient relationships, and patient behaviors and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method where the data sets are selected from the group comprising health care clinical, claims, genomic, immunochemistry, transactional, ordering data, demographic data, Lifestyle and social health data, patient relationships, and patient behaviors and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and providing patient matching comprising collecting a set of patient demographic information from a. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and raising an exception when an unmapped value comes to the PDMP provider. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and mapping test names to PDMP values on the gateway data provider. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and mapping test names to PDMP values. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and dynamically adjusting CRM Layout, Dashboards, or Reports in response to customer activations. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and technique for detecting doc shopping. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by to a raw data cluster, wherein applying an algorithm selected from the set comprising transformation algorithms, normalization operations, refinement operations. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for implementing sub-second response to elastic search queries comprising a transactional store (mySQL) for serving ETL data, a fast data warehouse (Snowflake) for data modeling, a plurality of Application load balancers, a plurality of cloud-hosted Docker containers, a content delivery network, a geospatial content server, and a session caching engine and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and providing patient matching comprising collecting a set of patient demographic information from a. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and raising an exception when an unmapped value comes to the PDMP provider. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and mapping test names to PDMP values on the gateway data provider. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and mapping test names to PDMP values. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and dynamically adjusting CRM Layout, Dashboards, or Reports in response to customer activations. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and technique for detecting doc shopping. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a system for data ingestion via an enterprise service bus comprising, a set of native cloud services in an enterprise service but workflow, and a notification/event module that takes in Health-level 7 data, converts it to a set of S3 objects, and sends out notification and events and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and providing patient matching comprising collecting a set of patient demographic information from a. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and raising an exception when an unmapped value comes to the PDMP provider. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and mapping test names to PDMP values on the gateway data provider. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and mapping test names to PDMP values. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and dynamically adjusting CRM Layout, Dashboards, or Reports in response to customer activations. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and technique for detecting doc shopping. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and ingesting, by a healthcare insight computing device, patient data of a patient received from one of a plurality of patient data providers; determining, by to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab payable provider, a billing provider, and a customer relationship management solution. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method measuring health at interface connections comprising using a machine learning technique to determine a set of expected communication frequencies for a plurality of interface connections, assigning an expected interval to each connection, and creating an alert when the expected communication frequency is not maintained on a particular interface connection and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and providing patient matching comprising collecting a set of patient demographic information from a. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and raising an exception when an unmapped value comes to the PDMP provider. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and mapping test names to PDMP values on the gateway data provider. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and mapping test names to PDMP values. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and dynamically adjusting CRM Layout, Dashboards, or Reports in response to customer activations. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and technique for detecting doc shopping. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and providing patient matching comprising collecting a set of patient demographic information from a. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and raising an exception when an unmapped value comes to the PDMP provider. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and mapping test names to PDMP values on the gateway data provider. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and mapping test names to PDMP values. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and dynamically adjusting CRM Layout, Dashboards, or Reports in response to customer activations. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and technique for detecting doc shopping. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a device that provides toxicology information corresponding to a laboratory report associated with a PDMP, the device comprising a memory; and a processor, the processor configured at least to: identify a requested laboratory report; associate the laboratory report with a PDMP; identify one or more laboratory result data; trigger a DCA service corresponding to the PDMP; send the one or more laboratory result data to a destination corresponding to the laboratory report; and send one or more parameters associated with the DCA to the destination corresponding to the laboratory report, wherein the processor is further configured such that the one or more parameters associated with the DCA include at least one of: No PDMP Match, Direct PDMP Match, Indirect PDMP Match, Indeterminant, and/or non-applicable and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and providing patient matching comprising collecting a set of patient demographic information from a. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and raising an exception when an unmapped value comes to the PDMP provider. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and mapping test names to PDMP values on the gateway data provider. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and mapping test names to PDMP values. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and dynamically adjusting CRM Layout, Dashboards, or Reports in response to customer activations. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and technique for detecting doc shopping. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for providing drug consistency awareness, comprising retrieving a patient lab result, retrieving a PDMP patient record, applying a consistency rule engine to compare the patient lab results to expected values, and providing a result selected from a list comprising no match, direct match, indirect match, indeterminant, N/A and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for providing patient matching comprising collecting a set of patient demographic information from a. In embodiments, provided herein is a method for providing patient matching comprising collecting a set of patient demographic information from a and raising an exception when an unmapped value comes to the PDMP provider. In embodiments, provided herein is a method for providing patient matching comprising collecting a set of patient demographic information from a and mapping test names to PDMP values on the gateway data provider. In embodiments, provided herein is a method for providing patient matching comprising collecting a set of patient demographic information from a and mapping test names to PDMP values. In embodiments, provided herein is a method for providing patient matching comprising collecting a set of patient demographic information from a and dynamically adjusting CRM Layout, Dashboards, or Reports in response to customer activations. In embodiments, provided herein is a method for providing patient matching comprising collecting a set of patient demographic information from a and technique for detecting doc shopping. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for providing patient matching comprising collecting a set of patient demographic information from a and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for providing patient matching comprising collecting a set of patient demographic information from a and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for providing patient matching comprising collecting a set of patient demographic information from a and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for providing patient matching comprising collecting a set of patient demographic information from a and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for providing patient matching comprising collecting a set of patient demographic information from a and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for providing patient matching comprising collecting a set of patient demographic information from a and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for raising an exception when an unmapped value comes to the PDMP provider. In embodiments, provided herein is a method for raising an exception when an unmapped value comes to the PDMP provider and mapping test names to PDMP values on the gateway data provider. In embodiments, provided herein is a method for raising an exception when an unmapped value comes to the PDMP provider and mapping test names to PDMP values. In embodiments, provided herein is a method for raising an exception when an unmapped value comes to the PDMP provider and dynamically adjusting CRM Layout, Dashboards, or Reports in response to customer activations. In embodiments, provided herein is a method for raising an exception when an unmapped value comes to the PDMP provider and technique for detecting doc shopping. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for raising an exception when an unmapped value comes to the PDMP provider and ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for raising an exception when an unmapped value comes to the PDMP provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for raising an exception when an unmapped value comes to the PDMP provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for raising an exception when an unmapped value comes to the PDMP provider and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for raising an exception when an unmapped value comes to the PDMP provider and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for raising an exception when an unmapped value comes to the PDMP provider and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for raising an exception when an unmapped value comes to the PDMP provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for mapping test names to PDMP values on the gateway data provider. In embodiments, provided herein is a method for mapping test names to PDMP values on the gateway data provider and mapping test names to PDMP values. In embodiments, provided herein is a method for mapping test names to PDMP values on the gateway data provider and dynamically adjusting CRM Layout, Dashboards, or Reports in response to customer activations. In embodiments, provided herein is a method for mapping test names to PDMP values on the gateway data provider and technique for detecting doc shopping. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for mapping test names to PDMP values on the gateway data provider and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for mapping test names to PDMP values on the gateway data provider and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for mapping test names to PDMP values on the gateway data provider and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for mapping test names to PDMP values on the gateway data provider and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for mapping test names to PDMP values on the gateway data provider and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for mapping test names to PDMP values on the gateway data provider and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for mapping test names to PDMP values. In embodiments, provided herein is a method for mapping test names to PDMP values and dynamically adjusting CRM Layout, Dashboards, or Reports in response to customer activations. In embodiments, provided herein is a method for mapping test names to PDMP values and technique for detecting doc shopping. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for mapping test names to PDMP values and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab payable provider, a billing provider, and a customer relationship management solution. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for mapping test names to PDMP values and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for mapping test names to PDMP values and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for mapping test names to PDMP values and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for mapping test names to PDMP values and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for mapping test names to PDMP values and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for dynamically adjusting CRM Layout, Dashboards, or Reports in response to customer activations. In embodiments, provided herein is a method for dynamically adjusting CRM Layout, Dashboards, or Reports in response to customer activations and technique for detecting doc shopping. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for dynamically adjusting CRM Layout, Dashboards, or Reports in response to customer activations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for dynamically adjusting CRM Layout, Dashboards, or Reports in response to customer activations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for dynamically adjusting CRM Layout, Dashboards, or Reports in response to customer activations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for dynamically adjusting CRM Layout, Dashboards, or Reports in response to customer activations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for dynamically adjusting CRM Layout, Dashboards, or Reports in response to customer activations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for dynamically adjusting CRM Layout, Dashboards, or Reports in response to customer activations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for technique for detecting doc shopping. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for technique for detecting doc shopping and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for technique for detecting doc shopping and ingesting, by a healthcare insight computing device, patient data of a patient received from one of a plurality of patient data providers; determining, by to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for technique for detecting doc shopping and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for technique for detecting doc shopping and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for technique for detecting doc shopping and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for technique for detecting doc shopping and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for technique for detecting doc shopping and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for determining healthcare relationships, the method comprising ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein ingesting the patient data received from the patient data provider comprises staging the patient data in a data staging area and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the to a raw data cluster, wherein extracting, by the healthcare insight computing device, at least a portion of the patient data from the raw data cluster in response to having received a request to generate a report associated with the patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein determining the one or more relationships between the ingested patient data and the previously ingested patient data comprises determining the one or more relationships between the ingested patient data and the previously ingested patient data that has been received from another of the plurality of patient data providers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by to a raw data cluster, wherein extracting, by the healthcare insight computing device, at least a portion of the patient data from the raw data cluster in response to having received a request to generate a report associated with the patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for determining healthcare relationships, the method comprising ingesting, by a healthcare insight computing device, patient data of a patient received from one of a plurality of patient data providers; determining, by the to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the to a raw data cluster, wherein extracting, by the healthcare insight computing device, at least a portion of the patient data from the raw data cluster in response to having received a request to generate a report associated with the patient data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein extracting, by the healthcare insight computing device, at least a portion of the patient data from the raw data cluster in response to having received a request to generate a report associated with the patient data, wherein de-identifying, by the healthcare insight computing device, the extracted portion of the patient data; aggregating, by the healthcare insight computing device, the de-identified patient data as a function of the requested report; and presenting, by the healthcare insight computing device, a visual representation of the de-identified patient data as a function of the requested report and aggregated, de-identified patient data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, further comprising detecting, by the healthcare insight computing device, a previously identified relationship has been broken; and identifying, by the healthcare insight computing device, an alternative data path to form a new relationship with the patient data that was associated with the previously identified relationship and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein the data closer engine finding an optimal query and rerouting the query to find an alternative connection path and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein finding the shortest path in a weighted graph applied to database referential integrity (e.g., using the uniform-cost search (UCS) algorithm), and calculating the “distance” cost between two tables of patient data based on the count of the rows in the union of two tables, a count of industry identifiers used, and a granularity match score and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein assigning a confidence level of the detected relationship based on as additional patient data being correlated across the different sources of patient data sets associated with that patient, wherein a report generation engine performs aggregation, rollups, metric calculations, algorithms, confidence scoring, and data manipulation of the patient data for reporting, dashboards, and data mining and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein encrypting the data and controlling access to the data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein applying an algorithm selected from the set comprising transformation algorithms, normalization operations, refinement operations and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a method for determining healthcare relationships, the method comprising ingesting, by a healthcare insight computing device, patient data of a patient received from one of a plurality of patient data providers; determining, by the healthcare insight computing device, one or more relationships between the ingested patient data and previously ingested patient data associated with the patient; correlating, by the healthcare insight computing device, the ingested data as a function of the determined relationships; enriching, by the healthcare insight computing device, the correlated patient data to form a new data element; and transmitting, by the healthcare insight computing device, the new data element to a raw data cluster, wherein enriching the correlated patient data comprises supplementing the ingested patient data with referential data, wherein establishing relationships between data sets selected from the list comprising financial data, drug schedules, industry reference data, geographic data and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: assigning the at least one security role to the user in a database based on a job performed by the user at a healthcare organization; and assigning at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, wherein the granting or denying step is further based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security information at a healthcare relationship management system, the user being associated with at least one security role; and granting or denying access to the health information for the user at the healthcare relationship management system based on an evaluation of the at least one category against the at least one security role, further comprising: generating a communication within the healthcare relationship management system, the communication being directed to a second user and comprising the health information; and generating a rendering of the communication to the second user, wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page is a business intelligence report and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a health information from a healthcare communication source at a healthcare relationship system; categorizing the health information into at least one security category based on one or more privacy rules; receiving a request from a user for the health information at a healthcare relationship management system, the user being associated with at least one security role and at least one organization node; and transmitting a web page based on the request, wherein the web page comprises the health information only when the at least one security role and the at least one organization node authorize the user to view the health information based on the at least one security category, wherein the web page comprises a communication within the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the external communication is an email and the one or more identifiers are email headers and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising receiving a communication request at a healthcare relationship management system, the communication request being associated with an external communication including one or more identifiers; creating a communication within the healthcare relationship management system based on the communication request; and associating the communication with one or more health information records in a database based on the one or more identifiers, wherein the one or more identifiers are reserved characters or keywords identified in the healthcare relationship management system and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to grant or deny access based on the at least one node and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role, retrieve the health information from the database, and grant or deny access to the health information for the user device at the healthcare relationship management system based on an evaluation of the at least one category against the at least one security role, wherein the healthcare communication source is selected from one of a lab information system, radiology information system, hospital information system, electronic medical record, data warehouse, revenue cycle management system, general ledger, accounts payable provider, a billing provider, and a customer relationship management solution, wherein the server is further configured to assign the at least one security role to the user in the database based on a job performed by the user at a healthcare organization, and assign at least one node to the user in the database, the node corresponding to a hierarchical view of the healthcare organization, wherein the server is further configured to generate a communication, the communication being directed to a second user and comprising the health information and transmit the communication to the user device, wherein the user device is configured to render the wherein the rendering includes a redaction over the health information in the event that the second user is unauthorized to view the health information and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category. In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category and a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

In embodiments, provided herein is a computerized method for healthcare relationship management, the method comprising a user device operated by a user; a database, the database configured to receive a health information from a healthcare communication source and categorize the health information into at least one security category based on one or more privacy rules; a server, the server configured to receive a request from a user device for the health information at a healthcare relationship management system, the user being associated with at least one security role and at least one organization node, retrieve the health information from the database, and transmit a web page to the user device, wherein the web page comprises the health information only when the at least one security role and the at least one organization node authorize the user to view the health information based on the at least one security category, wherein the healthcare communication source is selected from one of a lab information system, radiology information system, hospital information system, electronic medical record, data warehouse, revenue cycle management system, general ledger, accounts payable provider, a billing provider, and a customer relationship management solution.

Detailed embodiments of the present disclosure are disclosed herein; however, it is to be understood that the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The terms “a” or “an,” as used herein, are defined as one or more than one. The term “another,” as used herein, is defined as at least a second or more. The terms “including” and/or “having,” as used herein, are defined as comprising (i.e., open transition).

While only a few embodiments of the present disclosure have been shown and described, it will be obvious to those skilled in the art that many changes and modifications may be made thereunto without departing from the spirit and scope of the present disclosure as described in the following claims. All patent applications and patents, both foreign and domestic, and all other publications referenced herein are incorporated herein in their entireties to the full extent permitted by law.

The methods and systems described herein may be deployed in part or in whole through a machine that executes computer software, program codes, and/or instructions on a processor. The present disclosure may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platforms. A processor may be any kind of computational or processing device capable of executing program instructions, codes, binary instructions and the like, including a central processing unit (CPU), a general processing unit (GPU), a logic board, a chip (e.g., a graphics chip, a video processing chip, a data compression chip, or the like), a chipset, a controller, a system-on-chip (e.g., an RF system on chip, an AI system on chip, a video processing system on chip, or others), an integrated circuit, an application specific integrated circuit (ASIC), a field programmable gate array (FPGA), an approximate computing processor, a quantum computing processor, a parallel computing processor, a neural network processor, or other type of processor. The processor may be or may include a signal processor, digital processor, data processor, embedded processor, microprocessor or any variant such as a co-processor (math co-processor, graphic co-processor, communication co-processor, video co-processor, AI co-processor, and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor, or any machine utilizing one, may include non-transitory memory that stores methods, codes, instructions and programs as described herein and elsewhere. The processor may access a non-transitory storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache, network-attached storage, server-based storage, and the like.

A processor may include one or more cores that may enhance speed and performance of a multiprocessor. In embodiments, the process may be a dual core processor, quad core processors, other chip-level multiprocessor and the like that combine two or more independent cores (sometimes called a die).

The methods and systems described herein may be deployed in part or in whole through a machine that executes computer software on a server, client, firewall, gateway, hub, router, switch, infrastructure-as-a-service, platform-as-a-service, or other such computer and/or networking hardware or system. The software may be associated with a server that may include a file server, print server, domain server, internet server, intranet server, cloud server, infrastructure-as-a-service server, platform-as-a-service server, web server, and other variants such as secondary server, host server, distributed server, failover server, backup server, server farm, and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, social networks, and the like. Additionally, this coupling and/or connection may facilitate remote execution of programs across the network. The networking of some or all of these devices may facilitate parallel processing of a program or method at one or more locations without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for the execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of programs across the network. The networking of some or all of these devices may facilitate parallel processing of a program or method at one or more locations without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements. The methods and systems described herein may be adapted for use with any kind of private, community, or hybrid cloud computing network or cloud computing environment, including those which involve features of software as a service (SaaS), platform as a service (PaaS), and/or infrastructure as a service (IaaS).

The methods, program codes, and instructions described herein and elsewhere may be implemented on a cellular network with multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, 4G, 5G, LTE, EVDO, mesh, or other network types.

The methods, program codes, and instructions described herein and elsewhere may be implemented on or through mobile devices. The mobile devices may include navigation devices, cell phones, mobile phones, mobile personal digital assistants, laptops, palmtops, netbooks, pagers, electronic book readers, music players and the like. These devices may include, apart from other components, a storage medium such as flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, network-attached storage, network storage, NVME-accessible storage, PCIE connected storage, distributed storage, and the like.

The methods and systems described herein may transform physical and/or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable code using a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices, artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable devices, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions. Computer software may employ virtualization, virtual machines, containers, and other capabilities.

Thus, in one aspect, methods described above and combinations thereof may be embodied in computer executable code that, when executing on one or more computing devices, performs the steps thereof. In another aspect, the methods may be embodied in systems that perform the steps thereof and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “with,” “including,” and “containing” are to be construed as open-ended terms (i.e., meaning “including, but not limited to,”) unless otherwise noted. Recitations of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. The term “set” may include a set with a single member. No language in the specification should be construed as indicating any non-claimed element as essential to the practice of the disclosure.

While the foregoing written description enables one skilled to make and use what is considered presently to be the best mode thereof, those skilled in the art will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

All documents referenced herein are hereby incorporated by reference as if fully set forth herein.

What is claimed is:

1. A computer implemented method for detecting misuse of a controlled medication of a patient, the method comprising:
obtaining, by a processing system, lab test results of the patient from a lab testing system;
obtaining, by the processing system, patient attributes of the patient from one or more patient data sources;
generating, by the processing system, a usage profile corresponding to the patient based on the lab test results of the patient and the patient attributes;
determining, by the processing system, whether the usage profile is indicative of potential misuse of the controlled medication based on one or more features of the usage profile; and
in response to determining potential misuse of the controlled medication, transmitting a notification that indicates the potential misuse by the patient.

2. The computer implemented method of claim 1, wherein the potential misuse of the controlled medication is overuse of the controlled medication.

3. The computer implemented method of claim 1, wherein the potential misuse of the controlled medication is underuse of the controlled medication.

4. The computer implemented method of claim 1, wherein generating the usage profile includes combining multiple test results of the patient to obtain a history of lab results of the patient.

5. The computer implemented method of claim 1, wherein the patient attributes include two or more of: an age of the patient, a weight of the patient, a body type of the patient, and an activity level of a patient.

6. The computer implemented method of claim 5, wherein the patient attributes are obtained from an electronic medical record database of a healthcare system associated with a clinic of the patient.

7. The computer implemented method of claim 5, wherein the patient attributes are obtained from an insurer database of an insurance system associated with an insurance provider of the patient.

8. The computer implemented method of claim 1, wherein determining whether the usage profile is indicative of potential misuse includes:
identifying a set of features based on the usage profile;
inputting the set of features into a machine learned classification model that is trained to classify instances of potential misuse of the classified medication;
obtaining a classification from the machine learned classification model and a confidence score indicating a degree of confidence in the classification determined by the machine learned classification model; and
determining whether the usage profile is indicative of the potential misuse based on the classification and the confidence score.

9. The computer implemented method of claim 1, wherein determining whether the usage profile is indicative of potential misuse includes:
identifying a set of features based on the usage profile;
clustering the usage profile with a plurality of other usage profiles using a clustering algorithm, each other usage profile respectively corresponding to a respective previous patient that was prescribed the controlled medication and deemed either to be indicative of potential misuse of the controlled medication or of proper use of the controlled medication;
determining a cluster of the usage profile of the patient to which the usage profile was clustered, wherein the cluster includes a subset of the plurality of other usage profiles;
determining whether the usage profile is indicative of potential misuse of the controlled medication based on the other usage profiles in the subset of the plurality of other usage profiles.

10. The computer implemented method of claim 1, wherein determining whether the usage profile is indicative of potential misuse includes:
identifying a set of features based on the usage profile; and
applying a set of rules to the features to determine whether the usage profile is indicative of potential misuse.

11. The computer implemented method of claim 1, wherein the lab test results include results from a urine analysis test.

12. The computer implemented method of claim 1, wherein the lab test results include results from a blood test.

13. A computer implemented method for recommending a lab test for a patient, the method comprising:
obtaining, by a processing system, a proposed prescription for the patient from an external data source, the proposed prescription indicating a medication;
obtaining, by the processing system, patient attributes for the patient, including a diagnosis of the patient;
determining, by the processing system, whether to recommend one or more different lab tests for the patient prior to the patient beginning the proposed prescription based on the proposed prescription and the patient attributes;
in response to determining to recommend one or more different lab tests for the patient, providing, by the processing system, a testing recommendation to a customer relationship management system, wherein the testing recommendation indicates the one or more different tests that are recommended for the patient, wherein the customer relationship management system transmits the testing recommendation to a healthcare system of a clinic of the patient.

14. The computer implemented method of claim 13, wherein determining whether to recommend the one or more different lab tests includes:
identifying a set of features based on the proposed prescription and the patient attributes;
inputting the set of features into one or more machine learned models that are respectively trained to determine whether to recommend a respective lab test;
obtaining one or more respective recommendations from the one or more respective machine learned models based on the set of features, wherein each respective recommendation indicates whether the respective lab test should be performed for the patient given the patient attributes and has a confidence score that indicates a degree of confidence in the recommendation; and
for each recommendation, determining whether to recommend the respective lab test indicated therein based on the confidence score of the recommendation.

15. The computer implemented method of claim 14, wherein each of the one or more machine learned models corresponds to the medication.

16. The computer implemented method of claim 14, wherein each of the one or more machine learned models is trained on a plurality of training data samples that respectively correspond to a plurality of previous patients that were prescribed the medication, wherein each training data sample includes respective patient attributes of the respective previous patient and an outcome related to the medication for the previous patient.

17. The computer implemented method of claim 16, wherein each training data sample further includes one or more lab test results of the respective previous patient.

18. The computer implemented method of claim 13, wherein determining whether to recommend the one or more different lab tests includes:
identifying a set of features based on the proposed prescription and the patient attributes;
inputting the set of features into a machine learned model that is trained to determine whether to recommend one or more of a plurality of different lab tests given a set of patient attributes;
obtaining a recommendation and a confidence score corresponding to the recommendation from the machine learned model based on the set of features, wherein the recommendation indicates any of the plurality of different lab tests that should be performed for the patient given the patient attributes, and the confidence score indicates a degree of confidence in the recommendation; and
determining whether to accept the recommendation based on the confidence score of the recommendation.

19. The computer implemented method of claim 18, wherein the machine learned model corresponds to the medication.

20. The computer implemented method of claim 18, wherein the machine learned model is trained on a plurality of training data samples that respectively correspond to a plurality of previous patients that were prescribed the medication, wherein each training data sample includes respective patient attributes of the respective previous patient and an outcome related to the medication for the previous patient.

21. The computer implemented method of claim 20, wherein each training data sample further includes one or more lab test results of the respective previous patient.

22. The computer implemented method of claim 13, wherein determining whether to recommend the one or more different lab tests includes:
identifying a set of features based on the usage profile; and
applying a set of rules to the features to determine whether to recommend the one or more different lab tests based on the set of features and one or more conditions defined in the set of rules.

23. The computer implemented method of claim 13, wherein the different lab tests include at least one of a genetic test, a blood test, and a urine analysis test.
